(19) United States
(12) Patent Application Publication
(10) Pub. No.: US 2026/0354380 A1
(43) Pub. Date: Nov. 12, 2026

(54) PYRAZOLO PESTICIDAL COMPOUNDS

(71) Applicant: BASF SE, Ludwigshafen (DE)

(72) Inventors: Rupsha Chaudhuri, Navi Mumbai (IN); Pulakesh Maity, Navi Mumbai (IN); Christian Defieber, Ludwigshafen (DE); Ashokkumar Adisechan, Navi Mumbai (IN); Christian Harald Winter, Ludwigshafen (DE); Kishor Handore, Navi Mumbai (IN); Karsten Koerber, Ludwigshafen (DE); Matthew Charles Linford Wakeham, Ludwigshafen (DE); Devendra Vyas, Durham, NC (US)

(21) Appl. No.: 19/099,562

(22) PCT Filed: Jul. 31, 2023
(86) PCT No.: PCT/EP2023/071106

§ 371 Date: Jan. 29, 2025

(30) Foreign Application Priority Data
Aug. 2, 2022 (EP) 22188207.9

(51) Int. Cl. A01N 43/78; A01P 7/04; C07D 417/12

(52) U.S. Cl. CPC A01N 43/78; A01P 7/04; C07D 417/12

(57) ABSTRACT

The present invention relates to the compounds of formula (I), and the N-oxides, stereoisomers, tautomers and agriculturally or veterinarily acceptable salts thereof wherein the variables are defined according to the description, Formula (I), (I). The compounds of formula (I), as well as the N-oxides, stereoisomers, tautomers and agriculturally or veterinarily acceptable salts thereof, are useful for combating or controlling invertebrate pests, in particular arthropod pests and nematodes. The invention also relates to a method for controlling invertebrate pests by using these compounds and to plant propagation material and to an agricultural and a veterinary composition comprising said compounds.

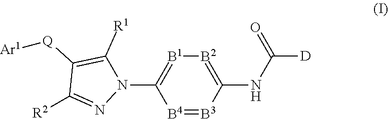

Invertebrate pests and in particular insects, arachnids and nematodes destroy growing and harvested crops and attack wooden dwelling and commercial structures, thereby causing large economic loss to the food supply and to property. Accordingly, there is an ongoing need for new agents for combating invertebrate pests.

Carbamoylated and thiocarbamoylated oxime derivatives are known for pesticidal use, for example, in patent publications WO 2016/156076, semi-carbazones and thiosemicarbazones derivatives are known for pesticidal use in patent publication WO 2016/116445 and pyrazolo pesticidal compounds are known for pesticidal use in patent publication WO2021/013561.

Due to the ability of target pests to develop resistance to pesticidally-active agents, there is an ongoing need to identify further compounds, which are suitable for combating invertebrate pests such as insects, arachnids and nematodes. Furthermore, there is a need for new compounds having a high pesticidal activity and showing a broad activity spectrum against a large number of different invertebrate pests, especially against difficult to control insects, arachnids and nematodes.

It is therefore an object of the present invention to identify and provide compounds, which exhibit a high pesticidal activity and have a broad activity spectrum against invertebrate pests.

It has been found that these objects can be achieved by substituted bicyclic compounds of formula I, as depicted and defined below, including their stereoisomers, their salts, in particular their agriculturally or veterinarily acceptable salts, their tautomers and their N-oxides.

In a first aspect, the present invention relates to the Compounds of the formula I

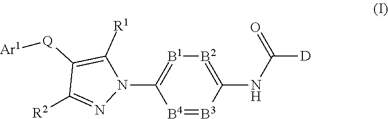

Wherein
Q is —C(═O)—N(R⁵)—, or —N(R⁵)—C(═O)—;
R⁵ is H, C₁-C₆-alkyl, C₃-C₆-cycloalkyl, C₁-C₆-alkyl-C₁-C₆-alkoxy or C₁-C₆-alkyl-C₃-C₆-cycloalkyl, phenyl, 5- or 6-membered heteroaryl, —CH₂-phenyl, —CH₂-5- or 6-membered heteroaryl, 1,3-dioxolan-2-ylmethyl, or halogen, wherein the alkyl, cycloalkyl, phenyl and heteroaryl moieties are unsubstituted or substituted with halogen or CN;
R¹ is H, C₁-C₆-alkyl, C₃-C₆-cycloalkyl, halogen, or NR⁶R⁷, wherein the alkyl and cycloalkyl moieties are unsubstituted or substituted with halogen or CN;
R² is H, C₁-C₆-alkyl, C₃-C₆-cycloalkyl, or halogen, wherein the alkyl and cycloalkyl moieties are unsubstituted or substituted with halogen or CN;
B¹ is N or CR^{B1};
B² is N or CR^{B2};
B³ is N or CR^{B3};
B⁴ is CR^{B4};
R^{B1}, R^{B2}, R^{B3}, and R^{B4} independently of each other are H, halogen, CN, C₁-C₆-alkyl, C₃-C₆-cycloalkyl, or C₁-C₆-alkoxy, wherein the alkyl, alkoxy, and cycloalkyl moieties are unsubstituted or substituted with halogen;
D is the moiety DA or DB,

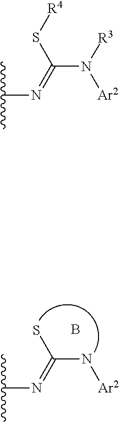

R³ is H, C₁-C₆-alkyl, or C₃-C₆-cycloalkyl, wherein the alkyl and cycloalkyl moieties are unsubstituted or substituted with halogen or CN;
R⁴ is H, C₁-C₆-alkyl, or C₃-C₆-cycloalkyl, wherein the alkyl and cycloalkyl moieties are unsubstituted or substituted with halogen, —O—(C═O)—C₁-C₆-alkyl, —O—(C═O)—C₁-C₆-alkoxy, or CN;
B is a 5- or 6-membered carbocyclic group, wherein 1 or 2 CH₂ moieties of the carbocyclic group may be replaced by a carbonyl group, O, or S, wherein the carbocyclic group is unsubstituted or substituted with Rh;
Ar¹ is phenyl or 5- or 6-membered heteroaryl, which are unsubstituted or substituted with R^{Ar1}, wherein
R^{Ar1} is halogen, SF₅, NO₂, OH, CN, C₁-C₆-alkyl, C₁-C₆-alkoxy, C₃-C₆-cycloalkyl, C₃-C₆-heterocyclyl, C₃-C₆-cycloalkoxy, C₂-C₆-alkenyl, C₂-C₆-alkynyl wherein the alkyl, alkoxy, alkenyl, alkynyl, cycloalkyl, C₃-C₆-heterocyclyl, and cycloalkoxy moieties are unsubstituted or substituted with R^{f};
C(═O)—OR^{a}, NR^{b}R^{c}, C₁-C₆-alkylene-CN, C(═O)—NR^{b}R^{c}, C(═O)—R^{d}, NHS(═O)ₘR^{e}, S(═O)ₘR^{e}, N═S(═O)—(C₁-C₆-alkyl)₂, SO₂NR^{b}R^{c}, or S(═O)ₘR^{e};
R⁶ and R⁷ are, identical or different, H, C₁-C₆-alkyl, C₃-C₆-cycloalkyl, phenyl, —CH₂-phenyl, 5- or 6-membered heteroaryl, —CH₂-5- or 6-membered heteroaryl, 1,3-dioxolan-2-ylmethyl, or 2-(methylamino)-2-oxo-ethyl, wherein the alkyl, cycloalkyl, phenyl and heteroaryl moieties are unsubstituted or substituted with halogen, CN, C₁-C₆-alkyl or C₁-C₆-alkoxy;
Ar² is phenyl or 5- or 6-membered heteroaryl, which are unsubstituted or substituted with R^{Ar2}, wherein
R^{Ar2} is halogen, CN, —SCN, —SF₅, C₁-C₆-alkyl, C₁-C₆-alkoxy, C₂-C₆-alkenyl, C₂-C₆-alkynyl, C₁-C₆-alkoxy-C₁-C₄-alkyl, C₁-C₆-alkoxy-C₁-C₄-alkoxy, C₃-C₆-cycloalkyl, C₃-C₆-cycloalkoxy, C₃-C₆-cycloalkyl-C₁-C₄-alkyl, C₃-C₆-cycloalkoxy-C₁-C₄-alkyl, wherein the alkyl, alkoxy, alkenyl, alkynyl, cycloalkyl and cycloalkoxy moieties are unsubstituted or substituted with halogen; C(═O)—OR^{a}, NR^{b}R^{c}, C₁-C₆-alkylene-CN, C(═O)—NR^{b}R^{c};
R^{a}, R^{b} and R^{c} are, identical or different, H, C₁-C₆-alkyl, C₂-C₆-alkenyl, C₃-C₆-cycloalkyl, C₃-C₆-cycloalkyl-C₁-C₄-alkyl, —C(═O)—C₁-C₆-alkyl wherein the alkyl, alkenyl, and cycloalkyl moieties are unsubstituted or substituted with halogen;
R^{d} is H, C₁-C₆-alkyl;
R^{e} is C₁-C₆-alkyl, C₃-C₆-cycloalkyl, C₃-C₆-cycloalkyl-C₁-C₄-alkyl, wherein the alkyl, cycloalkyl moieties are unsubstituted or substituted with halogen or CN;
R^{f} is halogen, OH, CN, SCN, —SF₅, C₁-C₆-alkyl, C₁-C₆-alkoxy, C₂-C₆-alkenyl, C₂-C₆-alkynyl, C₁-C₆-alkoxy-C₁-C₄-alkyl, C₁-C₆-alkoxy-C₁-C₄-alkoxy, C₃-C₆-cycloalkyl, C₃-C₆-cycloalkoxy, C₃-C₆-cycloalkyl-C₁-C₄-alkyl, C₃-C₆-cycloalkoxy-C₁-C₄-alkyl, wherein the alkyl, alkoxy, alkenyl, alkynyl, cycloalkyl and cycloalkoxy moieties are unsubstituted or substituted with halogen;
R^{h} is halogen, C₁-C₆-alkyl, or C₁-C₆-alkoxy;
m is 0, 1, or 2.
   and the N-oxides, stereoisomers, tautomers and agriculturally or veterinarily acceptable salts thereof.

Moreover, the present invention also relates to processes and intermediates for preparing compounds of formula I and to active compound combinations comprising them. Moreover, the present invention relates to agricultural or veterinary compositions comprising the compounds of formula I, and to the use of the compounds of formula I or compositions comprising them for combating or controlling invertebrate pests and/or for protecting crops, plants, plant propagation material and/or growing plants from attack and/or infestation by invertebrate pests. The present invention also relates to methods of applying the compounds of formula I. The present invention also relates to method for protecting crops, plants, plant propagation material and/or growing plants from attack or infestation by invertebrate pests comprising contacting or treating the crops, plants, plant propagation material and growing plants, or soil, material, surface, space, area or water in which the crops, plants, plant propagation material is stored or the plant is growing, with a pesticidally effective amount of at least one compound of formula (I) as defined above or a composition comprising at least one compound of formula (I);
Furthermore, the present invention relates to seed comprising compounds of formula I. Wherein the compounds of formula I includes N-oxides, stereoisomers, tautomers and agriculturally or veterinarily acceptable salts thereof.

With due modification of the starting compounds, the compounds of formula I can be prepared by procedures as given in below schemes.

### General Procedure

The compounds of the formula (I) can be prepared by methods of organic chemistry, e.g, by the methods described herein after in schemes 1 to 19 in the synthesis description of the examples. In the schemes 1 to 19 the radicals Ar¹, B¹, B², B³, B⁴, Q, R¹, R², R³, R⁴, R⁵, R⁶, R⁷, R^{h} and Ar² are as defined above for formula (I), unless otherwise specified.

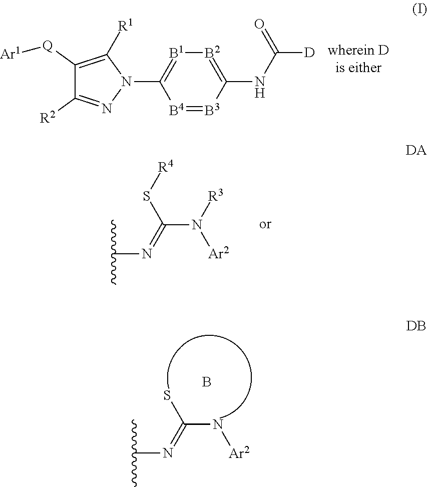

Compounds of formula (I), wherein R⁴ is not H are the compounds of formula (Ia) can be prepared by analogy to the methods described in WO 2021/011722 or methods described in Scheme 1.

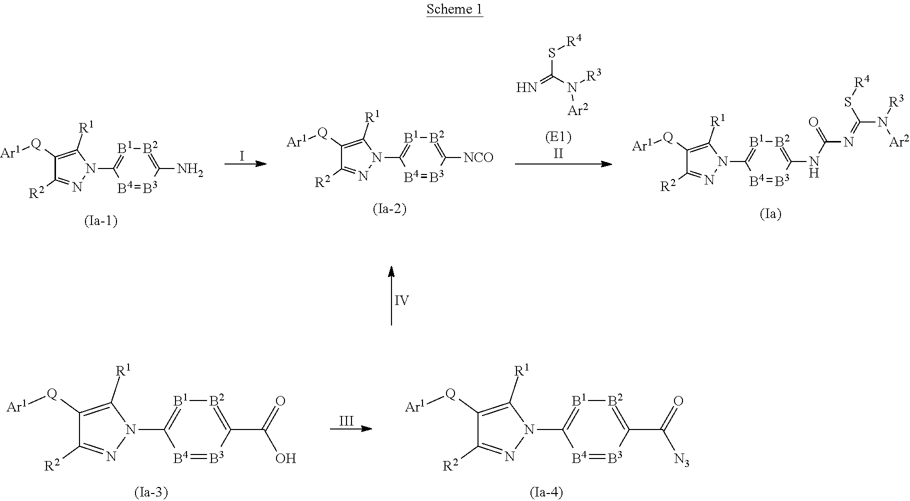

In one embodiment of Scheme 1, compounds of formula (Ia-2) are reacted directly with a compound of formula (E1) in the presence of an inorganic base to form a compound of formula (Ia). An isocyanate compound of formula (Ia-2) can be generated in situ from either an amine of the formula (Ia-1) by using one of the common reagents such as phosgene, diphosgene, triphosgene or carbonyldiimidazole (Step I) in a mixed solvent system and in the presence of a base as described in March's Advanced Organic Chemistry 6^{th} edition, Michael B. Smith and Jerry March.

According to another embodiment of Scheme 1, an isocyanate compound of formula (Ia-2) is generated via a Curtius rearrangement of an acyl azide (Ia-4), e.g. by analogy to the method described in WO 2014/204622. The acyl azide of the formula (Ia-4) can be prepared from the corresponding carboxylic acid precursor of formula (Ia-3) by treatment with ethyl chloroformate and sodium azide in the presence of an amine base such as triethylamine, or with diphenylphosphoryl azide in the presence of an amine base such as triethylamine.

Compounds of formula (I), wherein the compounds of formula (Ia) can also be prepared by analogy to the methods described in WO 2021/011722 or methods described in Scheme 2.

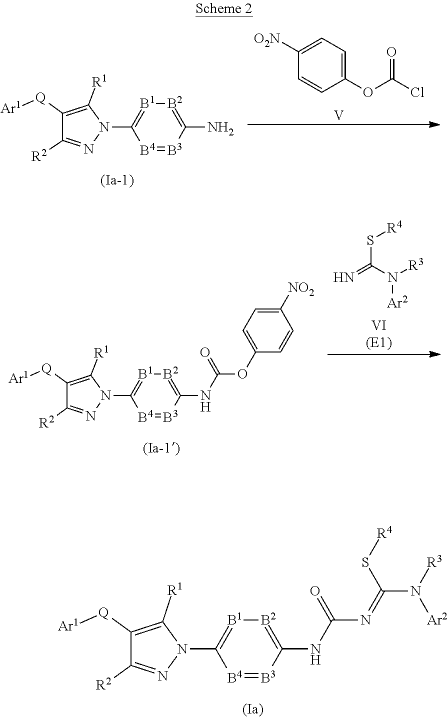

According to the method depicted in scheme 2, amine of formula (Ia-1) can be treated with an activating agent such as 4-nitrophenyl chloroformate in the presence of a polar aprotic solvent preferably tetrahydrofuran to generate an activated amine (Ia-1′), which in turn is reacted with the compound of formula (E1), in the presence of organic base such as N,N-diisopropylethylamine to form compound of formula (Ia). Compounds of formula (E1) can be prepared by analogy to the methods described in WO 2021/011722.

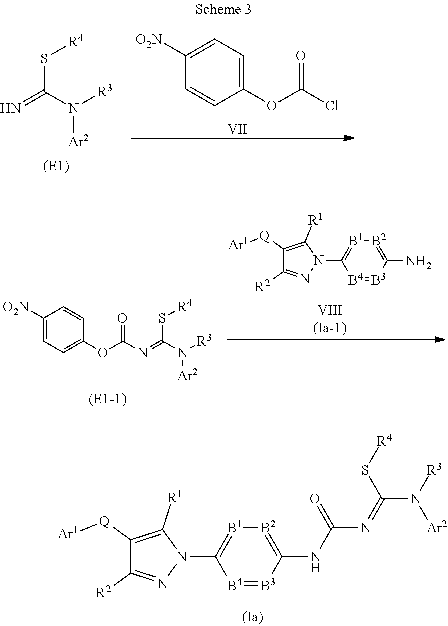

As depicted in scheme 3, compound of formula (E1) can be treated with an activating agent such as 4-nitrophenyl chloroformate in the presence of a polar aprotic solvent preferably tetrahydrofuran and an inorganic base such as cesium carbonate or potassium carbonate to generate an activated carbamate intermediate (E1-1), which in turn is reacted with amine of formula (Ia-1) in the presence of an inorganic base such as cesium carbonate or potassium carbonate to form compound of formula (Ia).

Compounds of formula (I), wherein R⁴ is H are the compounds of formula (Ia′) and can be prepared by analogy to the methods described in WO 2021/011722 or methods described in Scheme 4.

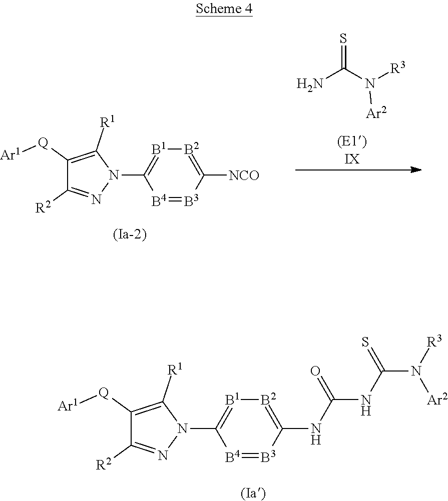

As depicted in scheme 4, compounds of the formula (Ia′) can be prepared by treating aryl thiourea of formula (E1′) with the isocyanate of formula (Ia-2) in the presence of inorganic bases such as cesium carbonate or sodium hydride in an aprotic solvent. Compounds of formula (E1′) can be prepared by analogy to the methods described in WO 2021/011722.

Compounds of formula (E1-I), (E1-II), (E1-III) and (E1-IV) can be prepared by analogy to the methods described in J. of Med. Chem. 2010, 53(10), 4198-4211 or methods described in Scheme 5.

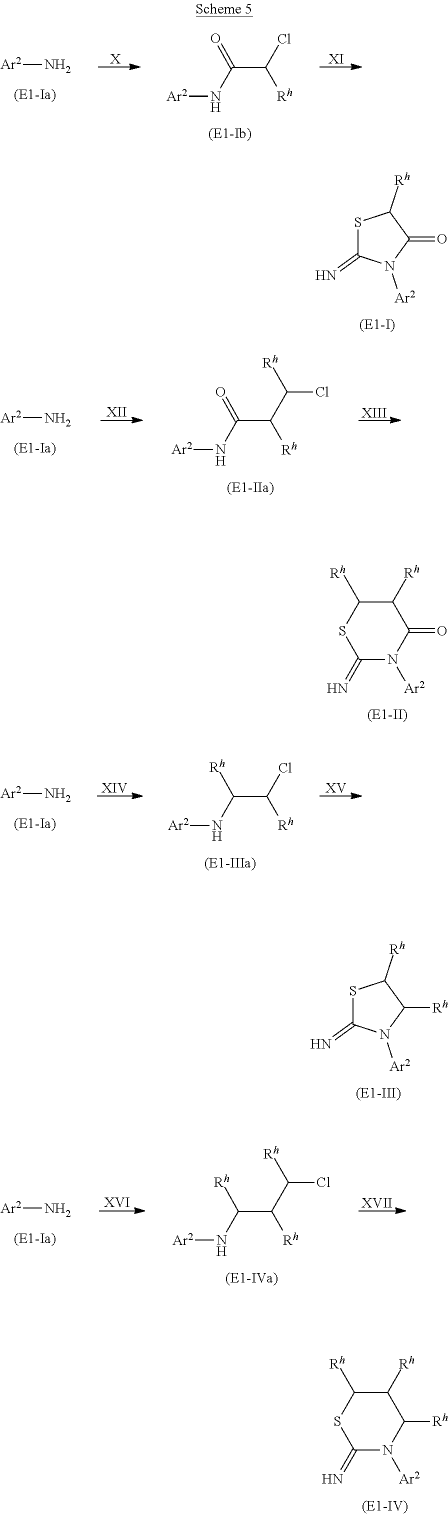

In the above reactions, compounds of formula (E1-Ia) can be converted into a variety of cyclized analogs of formula (E1-I), (E1-II), (E1-III) and (E1-IV). Compounds of formula (E1-I) and (E1-II) can be prepared by treatment of compounds of formula (E1-Ia) with unsubstituted or mono- or disubstituted 2-chloroacetylchloride and 3-chloropropanoylchloride in two steps as depicted in J. of Med. Chem. 2010, 53(10), 4198-4211. Compounds of formula (E1-IIIa) and (E1-IVa) can be prepared by treatment of compounds of formula (E1-Ia) with unsubstituted or mono- or disubstituted 2-chloroactaldehyde and 3-chloro-propanal in 2 steps as mentioned in J. of Het. Chem. 2006, 43(6), 1523-1531. Compounds of formula (E1-III) and (E1-IV) can be prepared by treatment of compounds of formula (E1-IIIa) and (E1-IVa) with potassium thiocyanate in presence of inorganic bases such as cesium carbonate in an aprotic solvent such as acetone.

Compounds of formula (Ia-I), (Ia-II), (Ia-III) and (Ia-IV) can be prepared from the compound of formula (Ia′) by analogy to the methods described in WO 2021/011722 or methods described in Scheme 6.

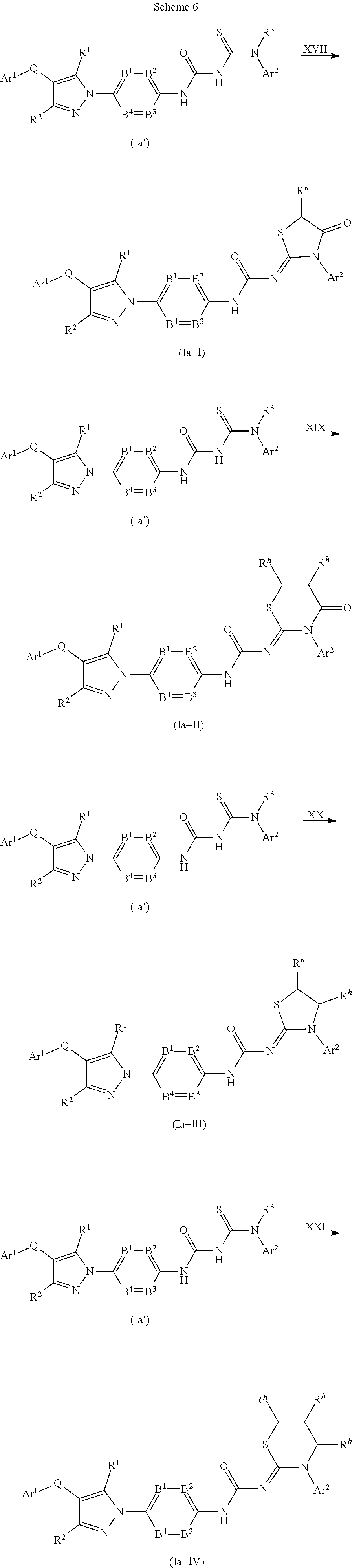

In the above reactions, compounds of formula (Ia′) can be converted into a variety of cyclized analogs of formula (Ia-I), (Ia-II), (Ia-III) and (Ia-IV). Cyclization can be achieved by treatment of compounds of formula (Ia′) with α-halo esters such as methyl bromoacetate or methyl bromo propanoate to form compounds of formula (Ia-I) and (Ia-II) unsubstituted or mono- or disubstituted with R^{h}. Compounds of formula (Ia-III) and (Ia-IV) unsubstituted or mono- or disubstituted with R^{h} can be prepared by treatment of compounds of formula (Ia′) with vicinal dihalides. For steps XVIII and XIX, use of sodium acetate in a protic solvent such as Ethanol, at temperatures ranging from about 20 °C to about 70 °C is preferred. For steps XX and XXI, use of an inorganic base such as potassium carbonate in a solvent such as acetonitrile or 2-butanone, at a temperature between about 0 °C and about 80 °C, is preferred. All the above reactions can be performed by analogy to the methods described in WO 2021/011722.

Compounds of formula (Ia-1) can be prepared by analogy to the methods described in scheme 7.

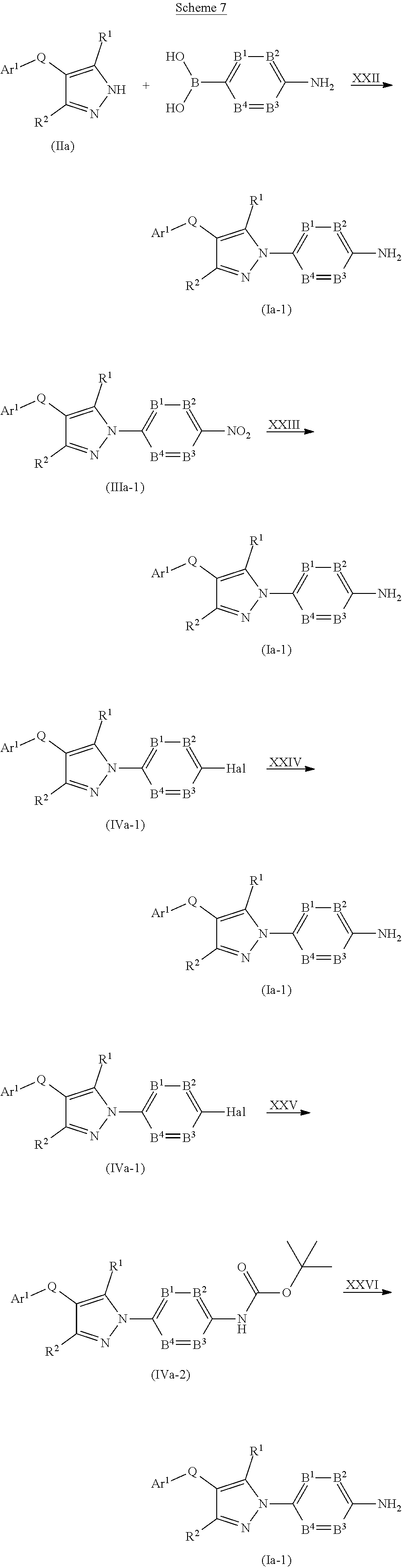

As depicted in scheme 7, compounds of formula (Ia-1) can be prepared with different synthetic routes. Step XXII can be performed via Chan-Lam coupling reaction starting from an aryl boronic acid precursor (1) as described in Chem. A Eur. J., 2017, 23(14), 3285-3290.

Alternatively, compounds of formula (Ia-1) can be prepared by reduction of nitro compounds of formula (IIIa-1) using reducing agents such as SnCl₂ in acid medium as shown in step XXIII.

Alternatively, compounds of formula (Ia-1) can also be prepared by reacting compounds of the formula (IVa-1) with ammonia in the presence of a metal catalyst or its salts, preferably copper or its salts as described in Chem. Commun., 2009, 3035-3037.

Additionally, compounds of formula (Ia-1) can also be prepared in two steps from compounds of the formula (IVa-1). Treatment of compounds of formula (IVa-1) with tert-butyl carbamate in the presence of metal catalyst or its salts, preferably palladium or its salts to form compounds of formula (IVa-2) in step XXV, followed by Boc-deprotection using trifluoroacetic acid or diluted hydrochloric acid to form the desired compound in step XXVI. All these reactions are performed as described in March's Advanced Organic Chemistry 6^{th} edition, Michael B. Smith and Jerry March.

Compounds of formula (IIIa-1) can be prepared by analogy to the methods described in scheme 8.

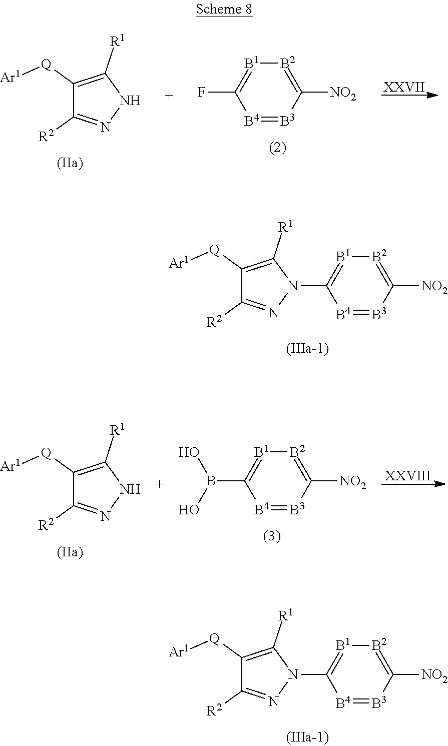

Step XXVII involves SNAr reaction between pyrazole (IIa) and p-fluoro nitroarene (2) as described in WO 2017/139274. Step XXVIII can be performed via Chan-Lam coupling reaction starting from an aryl boronic acid precursor (3) as described in Chem. A Eur. J., 2017, 23(14), 3285-3290.

Compounds of formula (IVa-1) can be prepared by analogy to the methods described in scheme 9.

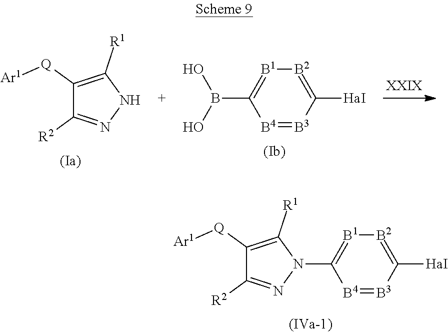

Step XXIX can be performed via Chan-Lam coupling reaction starting from an aryl boronic acid precursor (4) as described in Chem. A Eur. J., 2017, 23(14), 3285-3290. Hal as bromide is preferrable.

Compounds of formula (IVa-1) where in Q is —C(═O)—N(R⁵)—, R¹ is C₁-C₆-alkyl or C₃-C₆-cycloalkyl and R⁵ is H are the compounds of formula (IVa-1-1) can be prepared by analogy to the methods described in scheme 10.

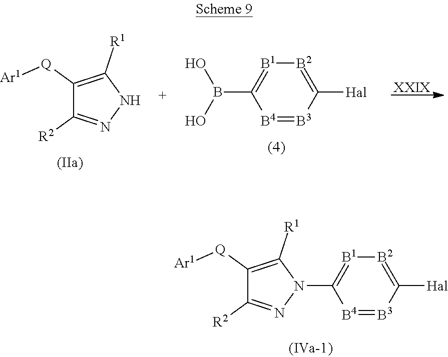

As mentioned in scheme 10, compounds of formula (IVa-1-1a) can be prepared from a commercially available 1,3 diketone derivative (5) by reacting it with substituted aryl hydrazines (ArNHNH₂, 6) as described in WO 2016/044666. Then nitration of compounds of formula (IVa-1-1a) using a mixture of nitric acid and sulfuric acid in an aprotic solvent such as dichloroethane can produce compounds of formula (IVa-1-1b). Compounds of formula (IVa-1-1b) can undergo reduction to compounds of formula (IVa-1-1c) using reducing agents such as SnCl₂ in acid medium or Fe with NH₄Cl in a mixture of Ethanol and water as shown in step XXXII. Then amide coupling reactions of compounds of formula (IVa-1-1c) with the derivatives of benzoic acids can form the compounds of formula (IVa-1-1) using suitable coupling reagents such as HATU or T₃P and bases like DIPEA, as described in March's Advanced Organic Chemistry 6^{th} edition, Michael B. Smith and Jerry March. The compounds of formula (IVa-1-1) can also be synthesized by treating compounds of formula (IVa-1-1c) with commercially available benzoyl chlorides in presence of base, as described in March's Advanced Organic Chemistry 6^{th} edition, Michael B. Smith and Jerry March. The compounds of formula (IVa-1-1) can also be synthesized by in-situ generation of benzoyl chlorides from the corresponding benzoic acids with POCl₃ or SOCl₂ then followed by treatment with the compounds of formula (IVa-1-1c) in presence of base as described in March's Advanced Organic Chemistry 6^{th} edition, Michael B. Smith and Jerry March.

Compounds of formula (IVa-1) where in Q is —N(R⁵)—C(═O)—, R¹ is C₁-C₆-alkyl or C₃-C₆-cycloalkyl and R⁵ is H are the compounds of formula (IVa-1-2) can be prepared by analogy to the methods described in scheme 11.

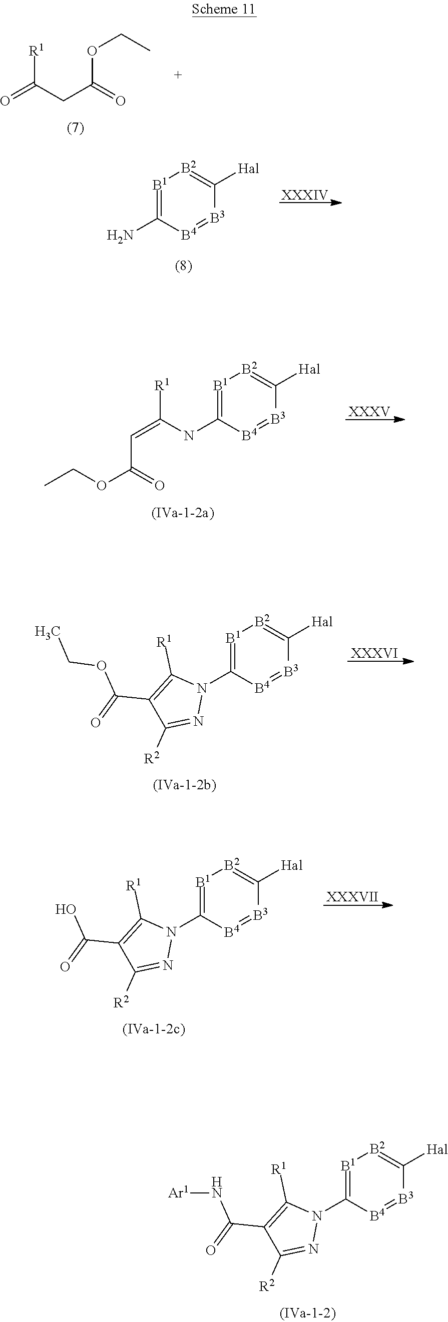

Compounds of formula (IVa-1-2b) can be prepared in two steps via intermediate of formula (IVa-1-2a), starting from commercially available beta keto ester derivative (7) as described in Synthesis, 2019, 51(6), 1473-1481. Step XXXV involves pyrazole synthesis as described in Angew., Chem., Int. Ed., 2010, 49(42), 7790-7794. Step XXXVI involves hydrolysis of ester using suitable bases like LiOH, NaOH, as mentioned in WO 2011/050245. Step XXXVII involves amide coupling reactions with aniline derivatives using suitable coupling reagents such as HATU or T₃P and bases like DIPEA, analogous to as described in March's Advanced Organic Chemistry 6^{th} edition, Michael B. Smith and Jerry March.

Compounds of formula (IVa-1) where in Q is —C(═O)—N(R⁵)—, R¹ is NR⁶R⁷ and R⁵ is H, are the compounds of formula (IVa-1-3) can be prepared by analogy to the methods described in scheme 12.

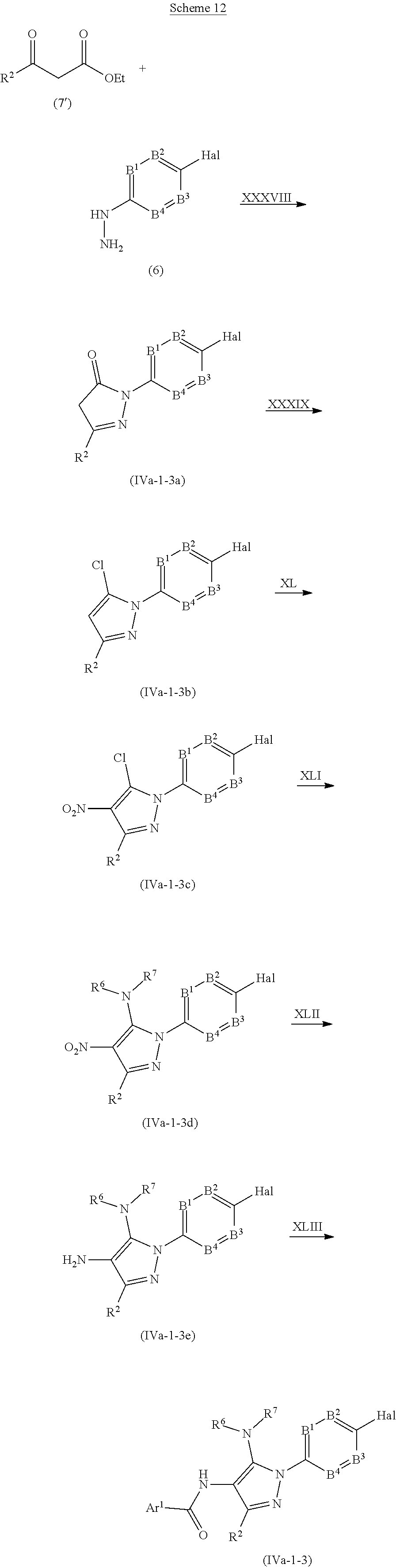

Compounds of formula (IVa-1-3a) can be prepared from a commercially available beta keto ester derivative (7′) by reacting it with substituted aryl hydrazines (ArNHNH₂, 6) as described in J. of Het. Chem. 1984, 21(6), 1747-52. Compounds of formula (IVa-1-3b) can be prepared from compounds of formula (IVa-1-3a) by treating with POCl₃. Then nitration of compounds of formula (IVa-1-3b) can be done as described in Bioorg, and Med. Chem. 2014, 22(9), 2739-2752 to produce compounds of formula (IVa-1-3c). Amination of compounds of formula (IVa-1-3c) where in R⁷ or R⁶ is C₁-C₆-alkyl or C₃-C₆-cycloalkyl or —CH₂-phenyl or —CH₂-5- or 6-membered hetaryl or 1,3-dioxolan-2-ylmethyl or 2-(methylamino)-2-oxo-ethyl, can be prepared by reacting with corresponding commercially available alkyl amines or benzyl amines in presence of bases like TEA and polar aprotic solvents like DMF to get compounds of formula (IVa-1-3d). For compounds of formula (IVa-1-3d) where in R⁷ or R⁶ is phenyl or 5- or 6-membered hetaryl can be prepared from compounds of formula (IVa-1-3c) by metal catalyzed reaction with corresponding anilines as describe in US20080036373. Compounds of formula (IVa-1-3d) where in R⁷ or R⁶ is H can be prepared from compounds of formula (IVa-1-3c) with ammonia in the presence of a metal catalyst or its salts, preferably copper or its salts as described in Chem. Commun., 2009, 3035-3037. Compounds of formula (IVa-1-3d) can be reduced to compounds of formula (IVa-1-3e) using reducing agents such as SnCl₂ in acid medium or Fe with NH₄Cl in a mixture of Ethanol and water as shown in step XLII. Finally, the compounds of formula (IVa-1-3) can be prepared by amide coupling reactions of compounds of formula (IVa-1-3e) with the derivatives of benzoic acids using suitable coupling reagents such as HATU or T₃P and bases like DIPEA, as described in March's Advanced Organic Chemistry 6^{th} edition, Michael B. Smith and Jerry March. The compounds of formula (IVa-1-3) can also be synthesized by treating compounds of formula (IVa-1-3e) with commercially available benzoyl chlorides in presence of base, as described in March's Advanced Organic Chemistry 6^{th} edition, Michael B. Smith and Jerry March. The compounds of formula (IVa-1-3) can also be synthesized by in-situ generation of benzoyl chlorides from the corresponding benzoic acids with POCl₃ or SOCl₂ then followed by treatment with the compounds of formula (IVa-1-3e) in presence of base as described in March's Advanced Organic Chemistry 6^{th} edition, Michael B. Smith and Jerry March.

Compounds of formula (IVa-1) where in Q is —N(R⁵)—C(═O)—, R¹ is NR⁶R⁷ and R⁵ is H, are the compounds of formula (IVa-1-4) can be prepared by analogy to the methods described in scheme 13.

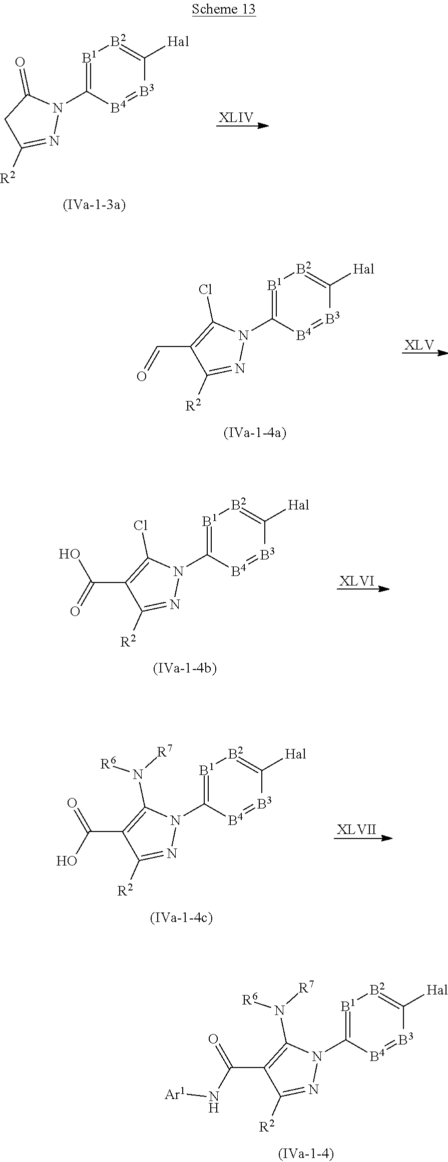

Compounds of formula (IVa-1-4a) can be prepared from compounds of formula (IVa-1-3a) analogous to the method depicted in J. of Med. Chem. 2020, 63(19), 11215-11234. Then oxidation of aldehydes to acids using oxidising agents like KMnO₄ to produce compounds of formula (IVa-1-4b), which can undergo amination to give compounds of formula (IVa-1-4c), as described under scheme 12. Finally, the amide coupling reaction to give compounds of formula (IVa-1-4) by reacting compounds of formula (IVa-1-4c) with aniline derivatives using suitable coupling reagents such as HATU or T₃P and bases like DIPEA, analogous to as described in March's Advanced Organic Chemistry 6^{th} edition, Michael B. Smith and Jerry March.

Compounds of formula (IIa) where in Q is —C(═O)—N(R⁵)—, R⁵ is H are the compounds of formula (IIa-1-1) can be prepared by analogy to the methods described in scheme 14.

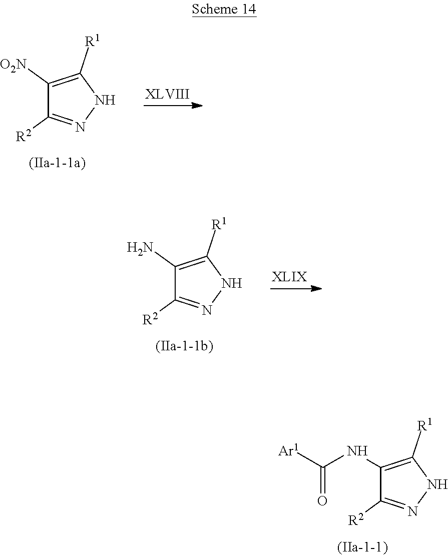

As shown in scheme 14, compounds of formula (IIa-1-1) can be prepared from compounds of formula (IIa-1-1a) in two steps. Step XLVIII involves reduction of nitro compounds of formula (IIa-1-1a) using reducing agents such as SnCl₂ in acid medium or Fe with NH₄Cl in a mixture of Ethanol and water and step XLIX involves amide coupling reactions analogous with the derivatives of benzoic acids using suitable coupling reagents such as HATU or T₃P and bases like DIPEA, as described in March's Advanced Organic Chemistry 6^{th} edition, Michael B. Smith and Jerry March.

Compounds of formula (IIa) where in Q is —N(R⁵)—C(═O)—, R⁵ is H are the compounds of formula (IIa-1-2) can be prepared by analogy to the methods described in scheme 15.

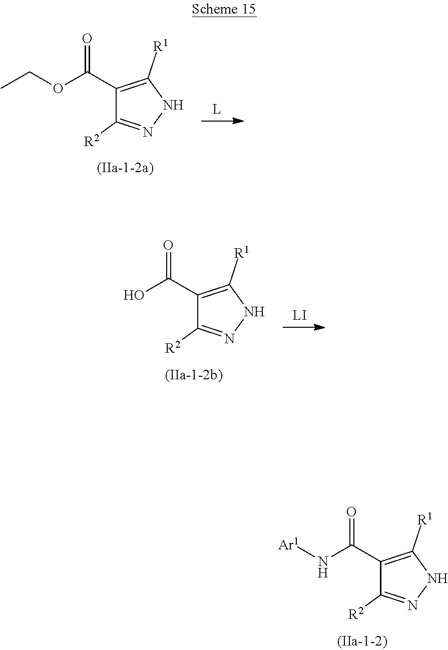

In the above reaction, compounds of formula (IIa-1-2) can be prepared from compounds of formula (IIa-1-2a) in two steps. Step L involves hydrolysis of ester using suitable base like LiOH, NaOH, as mentioned in WO 2011/050245. Step LI involves amide coupling reactions with aniline derivatives using suitable coupling reagents such as HATU or T₃P and bases like DIPEA, analogous to as described in March's Advanced Organic Chemistry 6^{th} edition, Michael B. Smith and Jerry March.

Compounds of formula (Ia-1) where in Q is —C(═O)—N(R⁵)—, R⁵ is C₁-C₆-alkyl or C₃-C₆-cycloalkyl or —CH₂-phenyl or —CH₂-5- or 6-membered hetaryl or 1,3-dioxolan-2-ylmethyl or phenyl or 5- or 6-membered hetaryl, are the compounds of formula (Ia-1-1) can be prepared by analogy to the methods described in scheme 16.

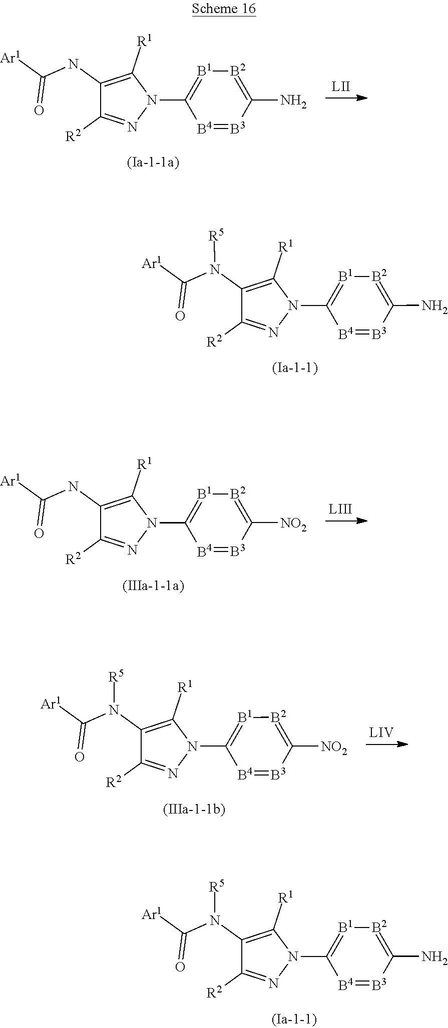

Compounds of formula (Ia-1-1) can be prepared from compounds of formula (Ia-1-1a) with R⁵ as C₁-C₆-alkyl or C₃-C₆-cycloalkyl or —CH₂-phenyl or —CH₂-5- or 6-membered hetaryl or 1,3-dioxolan-2-ylmethyl, by reacting with corresponding commercially available alkyl halides or benzyl halides preferably iodides or bromides in presence of bases like cesium carbonate and polar aprotic solvent like DMF, analogous to as described in March's Advanced Organic Chemistry 6th edition, Michael B. Smith and Jerry March. For compounds of formula (Ia-1-1) with R⁵ as phenyl or 5- or 6-membered hetaryl can be prepared from compounds of formula (Ia-1-1a) by metal catalyzed reaction with corresponding aryl halides or 5- or 6-membered hetaryl halide preferably iodides or bromides as describe in Chinese J. of Chem. 2012, 30(10), 2356-2362. Alternatively, compounds of formula (Ia-1-1) can be prepared from compounds of formula (IIIa-1-1a) in two steps. Step LIII can be performed similarly to step LII. Step LIV involves reduction using reducing agents such as SnCl₂ in acid medium or Fe with NH₄Cl in a mixture of Ethanol.

Compounds of formula (Ia-1) where in Q is —N(R⁵)—C(═O)—, R⁵ is C₁-C₆-alkyl or C₃-C₆-cycloalkyl or —CH₂-phenyl or —CH₂-5- or 6-membered hetaryl or 1,3-dioxolan-2-ylmethyl or phenyl or 5- or 6-membered hetaryl, are the compounds of formula (Ia-1-2) can be prepared by analogy to the methods described in scheme 17.

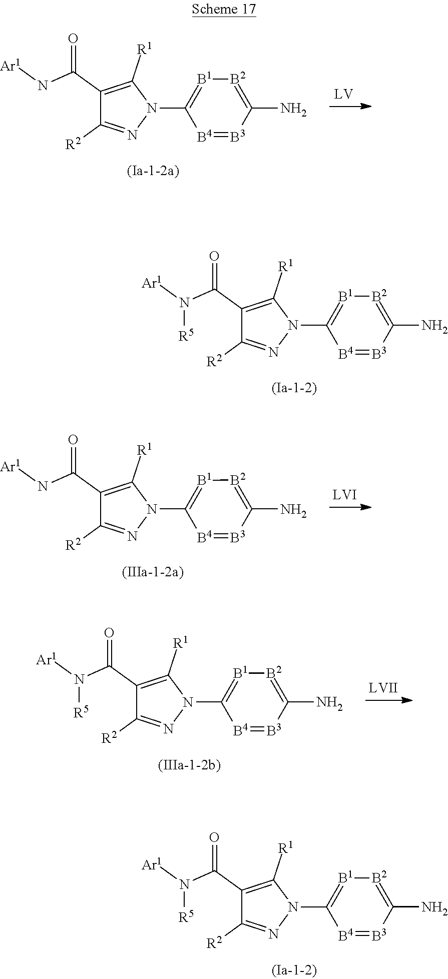

Compounds of formula (Ia-1-2) can be prepared from compounds of formula (Ia-1-2a) or from compounds of formula (IIIa-1-2a) using the reaction conditions discussed under scheme 16.

Compounds of formula (Ia-1) where in R¹ is C₁-C₆-alkyl or C₃-C₆-cycloalkyl and B² or B³ is CR^{B2} or CR^{B3} where in R^{B2} or R^{B3} is Hal, are the compounds of formula (Ia-1-3) can be prepared by analogy to the methods described in scheme 18.

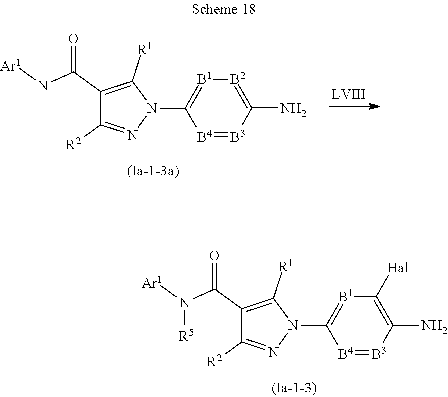

Compounds of formula (Ia-1-3) can be prepared by treating compounds of formula (Ia-1-3a) with electrophilic halogenating agent such as NCS in a polar aprotic solvent like ACN analogous to as described in March's Advanced Organic Chemistry 6^{th} edition, Michael B. Smith and Jerry March.

Compounds of formula (Ia-I) where in R¹ is Hal, are the compounds of formula (Ia-I-1) can be prepared by analogy to the methods described in scheme 19.

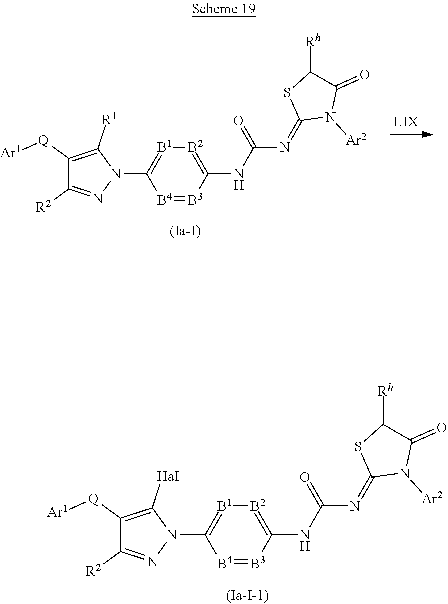

Compounds of formula (Ia-I-1) can be prepared by treating compounds of formula (Ia-I) with electrophilic halogenating agent such as Palau'Chlor in a non-polar aprotic solvent like chloroform analogous to as described in J. Am. Chem. Soc. 2014, 136, 6908-6911.

Individual compounds of formula I can also be prepared by derivatisation of other compounds of formula I or the intermediates thereof.

If the synthesis yields mixtures of isomers, a separation is generally not necessarily required since in some cases the individual isomers can be interconverted during work-up for use or during application (for example under the action of light, acids or bases). Such conversions may also take place after use, for example in the treatment of plants in the treated plant, or in the harmful fungus to be controlled.

A skilled person will readily understand that the preferences for the substituents, also in particular the ones given in the tables below for the respective substituents, given herein in connection with compounds I apply for the intermediates accordingly. Thereby, the substituents in each case have independently of each other or more preferably in combination the meanings as defined herein.

Unless otherwise indicated, the term “compound(s) according to the invention” or “compound(s) of the invention” or “compound(s) of formula (I)”, refers to the compounds of formula I.

The term “compound(s) according to the invention”, or “compounds of formula I” comprises the compound(s) as defined herein as well as a stereoisomer, salt, tautomer or N-oxide thereof. The term “compound(s) of the present invention” is to be understood as equivalent to the term “compound(s) according to the invention”, therefore also comprising a stereoisomer, salt, tautomer or N-oxide thereof.

The term “composition(s) according to the invention” or “composition(s) of the present invention” encompasses composition(s) comprising at least one compound of formula I according to the invention as defined above. The compositions of the invention are preferably agricultural or veterinary compositions.

Depending on the substitution pattern, the compounds according to the invention may have one or more centers of chirality, in which case they are present as mixtures of enantiomers or diastereomers. The invention provides both the single pure enantiomers or pure diastereomers of the compounds according to the invention, and their mixtures and the use according to the invention of the pure enantiomers or pure diastereomers of the compounds according to the invention or their mixtures. Suitable compounds according to the invention also include all possible geometrical stereoisomers (cis/trans isomers) and mixtures thereof. Cis/trans isomers may be present with respect to an alkene, carbon-nitrogen double-bond or amide group. The term “stereoisomer(s)” encompasses both optical isomers, such as enantiomers or diastereomers, the latter existing due to more than one center of chirality in the molecule, as well as geometrical isomers (cis/trans isomers). The present invention relates to every possible stereoisomer of the compounds of formula I, i.e. to single enantiomers or diastereomers, as well as to mixtures thereof.

The compounds according to the invention may be amorphous or may exist in one or more different crystalline states (polymorphs) which may have different macroscopic properties such as stability or show different biological properties such as activities. The present invention relates to amorphous and crystalline compounds according to the invention, mixtures of different crystalline states of the respective compounds according to the invention, as well as amorphous or crystalline salts thereof.

The term “tautomers” encompasses isomers, which are derived from the compounds of formula I by the shift of an H-atom involving at least one H-atom located at a nitrogen, oxygen or sulphur atom. Examples of tautomeric forms are keto-enol forms, imine-enamine forms, urea-isourea forms, thiourea-isothiourea forms, (thio)amide-(thio) imidate forms etc.

The term “stereoisomers” encompasses both optical isomers, such as enantiomers or diastereomers, the latter existing due to more than one center of chirality in the molecule, as well as geometrical isomers (cis/trans isomers).

Depending on the substitution pattern, the compounds of the formula I may have one or more centers of chirality, in which case they are present as mixtures of enantiomers or diastereomers. One center of chirality is the carbon ring atom of the isothiazoline ring carrying radical R¹. The invention provides both the pure enantiomers or diastereomers and their mixtures and the use according to the invention of the pure enantiomers or diastereomers of the compound I or its mixtures. Suitable compounds of the formula I also include all possible geometrical stereoisomers (cis/trans isomers) and mixtures thereof.

The term N-oxides relates to a form of compounds I in which at least one nitrogen atom is present in oxidized form (as NO). To be more precise, it relates to any compound of the present invention which has at least one tertiary nitrogen atom that is oxidized to an N-oxide moiety. N-oxides of compounds I can in particular be prepared by oxidizing e.g. the ring nitrogen atom of an N-heterocycle, e.g. a pyridine or pyrimidine ring present in Ar or R¹¹, or an imino-nitrogen present in central tricyclic core, with a suitable oxidizing agent, such as peroxo carboxylic acids or other peroxides. The person skilled in the art knows if and in which positions compounds of the present invention may form N-oxides.

Salts of the compounds of the formula I are preferably agriculturally and veterinarily acceptable salts. They can be formed in a customary method, e.g. by reacting the compound with an acid of the anion in question if the compound of formula I has a basic functionality or by reacting an acidic compound of formula I with a suitable base.

Suitable agriculturally or veterinarily acceptable salts are especially the salts of those cations or the acid addition salts of those acids whose cations and anions, which are known and accepted in the art for the formation of salts for agricultural or veterinary use respectively, and do not have any adverse effect on the action of the compounds according to the present invention. Suitable cations are in particular the ions of the alkali metals, preferably lithium, sodium and potassium, of the alkaline earth metals, preferably calcium, magnesium and barium, and of the transition metals, preferably manganese, copper, zinc and iron, and also ammonium (NH⁴⁺) and substituted ammonium in which one to four of the hydrogen atoms are replaced by C₁-C₄-alkyl, C₁-C₄-hydroxyalkyl, C₁-C₄-alkoxy, C₁-C₄-alkoxy-C₁-C₄-alkyl, hydroxy-C₁-C₄-alkoxy-C₁-C₄-alkyl, phenyl or —CH₂-phenyl. Examples of substituted ammonium ions comprise methylammonium, isopropylammonium, dimethylammonium, diisopropylammonium, trimethylammonium, tetramethylammonium, tetraethylammonium, tetrabutylammonium, 2-hydroxyethylammonium, 2-(2-hydroxyethoxy)ethylammonium, bis(2-hydroxyethyl) ammonium, benzyltrimethylammonium and benzyl-triethylammonium, furthermore phosphonium ions, sulfonium ions, preferably tri (C₁-C₄-alkyl) sulfonium, and sulfoxonium ions, preferably tri (C₁-C₄-alkyl) sulfoxonium. Suitable acid addition veterinarily acceptable salts, e.g. formed by compounds of formula I containing a basic nitrogen atom, e.g. an amino group, include salts with inorganic acids, for example hydrochlorides, sulphates, phosphates, and nitrates and salts of organic acids for example acetic acid, maleic acid, dimaleic acid, fumaric acid, difumaric acid, methane sulfenic acid, methane sulfonic acid, and succinic acid.

Anions of useful acid addition salts are primarily chloride, bromide, fluoride, hydrogen sulfate, sulfate, dihydrogen phosphate, hydrogen phosphate, phosphate, nitrate, hydrogen carbonate, carbonate, hexafluorosilicate, hexafluorophosphate, benzoate, and the anions of C₁-C₄-alkanoic acids, preferably formate, acetate, propionate and butyrate. They can be formed by reacting a compound of formulae I with an acid of the corresponding anion, preferably of hydrochloric acid, hydrobromic acid, sulfuric acid, phosphoric acid or nitric acid.

The term “invertebrate pest” as used herein encompasses animal populations, such as insects, arachnids and nematodes, which may attack plants, thereby causing substantial damage to the plants attacked, as well as ectoparasites which may infest animals, in particular warm blooded animals such as e.g. mammals or birds, or other higher animals such as reptiles, amphibians or fish, thereby causing substantial damage to the animals infested.

The term “plant propagation material” is to be understood to denote all the generative parts of the plant such as seeds and vegetative plant material such as cuttings and tubers (e. g. potatoes), which can be used for the multiplication of the plant. This includes seeds, roots, fruits, tubers, bulbs, rhizomes, shoots, sprouts and other parts of plants, including seedlings and young plants, which are to be transplanted after germination or after emergence from soil. The plant propagation materials may be treated prophylactically with a plant protection compound either at or before planting or transplanting. Said young plants may also be protected before transplantation by a total or partial treatment by immersion or pouring.

The term “plants” comprises any types of plants including “modified plants” and in particular “cultivated plants”.

The term “modified plants” refers to any wild type species or related species or related genera of a cultivated plant.

The term “cultivated plants” is to be understood as including plants which have been modified by breeding, mutagenesis or genetic engineering including but not limiting to agricultural biotech products on the market or in development (cf. http://www.bio.org/speeches/pubs/er/agri_products.asp). Genetically modified plants are plants, which genetic material has been so modified by the use of recombinant DNA techniques that under natural circumstances cannot readily be obtained by cross breeding, mutations or natural recombination. Typically, one or more genes have been integrated into the genetic material of a genetically modified plant in order to improve certain properties of the plant. Such genetic modifications also include but are not limited to targeted post-translational modification of protein(s), oligo- or polypeptides e. g. by glycosylation or polymer additions such as prenylated, acetylated or farnesylated moieties or PEG moieties.

Plants that have been modified by breeding, mutagenesis or genetic engineering, e. g. have been rendered tolerant to applications of specific classes of herbicides, such as auxin herbicides such as dicamba or 2,4-D; bleacher herbicides such as hydroxylphenylpyruvate dioxygenase (HPPD) inhibitors or phytoene desaturase (PDS) inhibitors; acetolactate synthase (ALS) inhibitors such as sulfonyl ureas or imidazolinones; enolpyruvylshikimate-3-phosphate synthase (EPSPS) inhibitors, such as glyphosate; glutamine synthetase (GS) inhibitors such as glufosinate; protoporphyrinogen-IX oxidase inhibitors; lipid biosynthesis inhibitors such as acetyl CoA carboxylase (ACCase) inhibitors; or oxynil (i. e. bromoxynil or ioxynil) herbicides as a result of conventional methods of breeding or genetic engineering. Furthermore, plants have been made resistant to multiple classes of herbicides through multiple genetic modifications, such as resistance to both glyphosate and glufosinate or to both glyphosate and a herbicide from another class such as ALS inhibitors, HPPD inhibitors, auxin herbicides, or ACCase inhibitors. These herbicide resistance technologies are e. g. described in Pest Managem. Sci. 61, 2005, 246; 61, 2005, 258; 61, 2005, 277; 61, 2005, 269; 61, 2005, 286; 64, 2008, 326; 64, 2008, 332; Weed Sci. 57, 2009, 108; Austral. J. Agricult. Res. 58, 2007, 708; Science 316, 2007, 1185; and references quoted therein. Several cultivated plants have been rendered tolerant to herbicides by conventional methods of breeding (mutagenesis), e. g. Clearfield® summer rape (Canola, BASF SE, Germany) being tolerant to imidazolinones, e. g. imazamox, or ExpressSun® sunflowers (DuPont, USA) being tolerant to sulfonyl ureas, e. g. tribenuron. Genetic engineering methods have been used to render cultivated plants such as soybean, cotton, corn, beets and rape, tolerant to herbicides such as glyphosate and glufosinate, some of which are commercially available under the trade names RoundupReady® (glyphosate-tolerant, Monsanto, U.S.A.), Cultivance® (imidazolinone tolerant, BASF SE, Germany) and LibertyLink® (glufosinate-tolerant, Bayer CropScience, Germany).

Furthermore, plants are also covered that are by the use of recombinant DNA techniques capable to synthesize one or more insecticidal proteins, especially those known from the bacterial genus Bacillus, particularly from Bacillus thuringiensis, such as δ-endotoxins, e. g. CrylA(b), CrylA(c), CrylF, CrylF(a2), CrylIA(b), CrylIIA, CrylIIB(b1) or Cry9c; vegetative insecticidal proteins (VIP), e. g. VIP1, VIP2, VIP3 or VIP3A; insecticidal proteins of bacteria colonizing nematodes, e. g. Photorhabdus spp, or Xenorhabdus spp.; toxins produced by animals, such as scorpion toxins, arachnid toxins, wasp toxins, or other insect-specific neurotoxins; toxins produced by fungi, such Streptomycetes toxins, plant lectins, such as pea or barley lectins; agglutinins; proteinase inhibitors, such as trypsin inhibitors, serine protease inhibitors, patatin, cystatin or papain inhibitors; ribosome-inactivating proteins (RIP), such as ricin, maize-RIP, abrin, luffin, saporin or bryodin; steroid metabolism enzymes, such as 3-hydroxysteroid oxidase, ecdysteroid-IDP-glycosyl-transferase, cholesterol oxidases, ecdysone inhibitors or HMG-COA-reductase; ion channel blockers, such as blockers of sodium or calcium channels; juvenile hormone esterase; diuretic hormone receptors (helicokinin receptors); stilben synthase, bibenzyl synthase, chitinases or glucanases. In the context of the present invention these insecticidal proteins or toxins are to be understood expressly also as pre-toxins, hybrid proteins, truncated or otherwise modified proteins. Hybrid proteins are characterized by a new combination of protein domains, (see, e. g. WO 02/015701). Further examples of such toxins or genetically modified plants capable of synthesizing such toxins are disclosed, e. g., in EP-A 374 753, WO 93/007278, WO 95/34656, EP-A 427 529, EP-A 451 878, WO 03/18810 und WO 03/52073. The methods for producing such genetically modified plants are generally known to the person skilled in the art and are described, e. g. in the publications mentioned above. These insecticidal proteins contained in the genetically modified plants impart to the plants producing these proteins tolerance to harmful pests from all taxonomic groups of athropods, especially to beetles (Coeloptera), two-winged insects (Diptera), and moths (Lepidoptera) and to nematodes (Nematoda). Genetically modified plants capable to synthesize one or more insecticidal proteins are, e. g., described in the publications mentioned above, and some of which are commercially available such as YieldGard® (corn cultivars producing the Cry1Ab toxin), YieldGard® Plus (corn cultivars producing Cry1Ab and Cry3Bb1 toxins), Starlink® (corn cultivars producing the Cry9c toxin), Herculex® RW (corn cultivars producing Cry34Ab1, Cry35Ab1 and the enzyme PhosphinothricinN-Acetyltransferase [PAT]); NuCOTN® 33B (cotton cultivars producing the CrylAc toxin), Bollgard® I (cotton cultivars producing the CrylAc toxin), Bollgard® II (cotton cultivars producing CrylAc and Cry2Ab2 toxins); VIPCOT® (cotton cultivars producing a VIP-toxin); NewLeaf® (potato cultivars producing the Cry3A toxin); Bt-Xtra®, NatureGard®, KnockOut®, BiteGard®, Protecta®, Bt11 (e. g. Agrisure® CB) and Bt176 from Syngenta Seeds SAS, France, (corn cultivars producing the Cry1Ab toxin and PAT enyzme), MIR604 from Syngenta Seeds SAS, France (corn cultivars producing a modified version of the Cry3A toxin, c.f. WO 03/018810), MON 863 from Monsanto Europe S.A., Belgium (corn cultivars producing the Cry3Bb1 toxin), IPC 531 from Monsanto Europe S.A., Belgium (cotton cultivars producing a modified version of the CrylAc toxin) and 1507 from Pioneer Overseas Corporation, Belgium (corn cultivars producing the CrylF toxin and PAT enzyme).

Furthermore, plants are also covered that are by the use of recombinant DNA techniques capable to synthesize one or more proteins to increase the resistance or tolerance of those plants to bacterial, viral or fungal pathogens. Examples of such proteins are the so-called “pathogenesis-related proteins” (PR proteins, see, e. g. EP-A 392 225), plant disease resistance genes (e. g. potato cultivars, which express resistance genes acting against Phytophthora infestans derived from the mexican wild potato Solanum bulbocastanum) or T4-lysozym (e. g. potato cultivars capable of synthesizing these proteins with increased resistance against bacteria such as Erwinia amylvora). The methods for producing such genetically modified plants are generally known to the person skilled in the art and are described, e. g. in the publications mentioned above.

Furthermore, plants are also covered that are by the use of recombinant DNA techniques capable to synthesize one or more proteins to increase the productivity (e. g. bio mass production, grain yield, starch content, oil content or protein content), tolerance to drought, salinity or other growth-limiting environmental factors or tolerance to pests and fungal, bacterial or viral pathogens of those plants.

Furthermore, plants are also covered that contain by the use of recombinant DNA techniques a modified amount of substances of content or new substances of content, specifically to improve human or animal nutrition, e. g. oil crops that produce health-promoting long-chain omega-3 fatty acids or unsaturated omega-9 fatty acids (e. g. Nexera® rape, DOW Agro Sciences, Canada). Furthermore, plants are also covered that contain by the use of recombinant DNA techniques a modified amount of substances of content or new substances of content, specifically to improve raw material production, e. g. potatoes that produce increased amounts of amylopectin (e. g. Amflora® potato, BASF SE, Germany).

The organic moieties mentioned in the above definitions of the variables are—like the term halogen—collective terms for individual listings of the individual members. The prefix Cₙ-Cₘ indicates in each case the possible number of carbon atoms in the group.

The term halogen denotes in each case F, Br, Cl or I, in particular F, Cl or Br.

The term “alkyl” as used herein and in the alkyl moieties of alkoxy, alkylthio, and the like refers to saturated straight-chain or branched hydrocarbon radicals having 1 to 2 (“C₁-C₂-alkyl”), 1 to 3 (“C₁-C₃-alkyl”), 1 to 4 (“C₁-C₄-alkyl”) or 1 to 6 (“C₁-C₆-alkyl”) carbon atoms. C₁-C₂-Alkyl is CH₃ or C₂H₅. C₁-C₃-Alkyl is additionally propyl and isopropyl, C₁-C₄-Alkyl is additionally butyl, 1-methylpropyl (sec-butyl), 2-methylpropyl (isobutyl) or 1,1-dimethylethyl (tert-butyl). C₁-C₆-Alkyl is additionally also, for example, pentyl, 1-methylbutyl, 2-methylbutyl, 3-methylbutyl, 2,2-dimethylpropyl, 1-ethylpropyl, 1,1-dimethylpropyl, 1,2-dimethylpropyl, hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, or 1-ethyl-2-methylpropyl.

The term “haloalkyl” as used herein, which is also expressed as “alkyl which is partially or fully halogenated”, refers to straight-chain or branched alkyl groups having 1 to 2 (“C₁-C₂-haloalkyl”), 1 to 3 (“C₁-C₃-haloalkyl”), 1 to 4 (“C₁-C₄-haloalkyl”) or 1 to 6 (“C₁-C₆-haloalkyl”) carbon atoms (as mentioned above), where some or all of the hydrogen atoms in these groups are replaced by halogen atoms as mentioned above: in particular C₁-C₂-haloalkyl, such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl or pentafluoroethyl. C₁-C₃-haloalkyl is additionally, for example, 1-fluoropropyl, 2-fluoropropyl, 3-fluoropropyl, 1,1-difluoropropyl, 2,2-difluoropropyl, 1,2-difluoropropyl, 3,3-difluoropropyl, 3,3,3-trifluoropropyl, heptafluoropropyl, 1,1,1-trifluoroprop-2-yl, 3-chloropropyl and the like. Examples for C₁-C₄-haloalkyl are, apart those mentioned for C₁-C₃-haloalkyl, 4-chlorobutyl and the like.

The term “alkylene” (or alkanediyl) as used herein in each case denotes an alkyl radical as defined above, wherein one hydrogen atom at any position of the carbon backbone is replaced by one further binding site, thus forming a bivalent moiety. Alkylene has preferably 1 to 6 carbon atoms (C₁-C₆-alkylene), 2 to 6 carbon atoms (C₂-C₆-alkylene), in particular 1 to 4 carbon atoms (C₁-C₄-alkylene) or 2 to 4 carbon atoms (C₂-C₄-alkylene). Examples of alkylene are methylene (CH₂), 1,1-ethandiyl, 1,2-ethandiyl, 1,3-propandiyl, 1,2-propandiyl, 2,2-propandiyl, 1,4-butandiyl, 1,2-butandiyl, 1,3-butandiyl, 2,3-butandiyl, 2,2-butandiyl, 1,5-pentandiyl, 2,2-dimethylpropan-1,3-diyl, 1,3-dimethyl-1,3-propandiyl, 1,6-hexandiyl etc.

The term “alkenyl” as used herein refers to monounsaturated straight-chain or branched hydrocarbon radicals having 2 to 3 (“C₂-C₃-alkenyl”), 2 to 4 (“C₂-C₄-alkenyl”) or 2 to 6 (“C₂-C₆-alkenyl) carbon atoms and a double bond in any position, for example C₂-C₃-alkenyl, such as ethenyl, 1-propenyl, 2-propenyl or 1-methylethenyl; C₂-C₄-alkenyl, such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl or 2-methyl-2-propenyl; C₂-C₆-alkenyl, such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl, 1-ethyl-2-methyl-2-propenyl and the like.

The term “alkynyl” as used herein refers to straight-chain or branched hydrocarbon groups having 2 to 3 (“C₂-C₃-alkynyl”), 2 to 4 (“C₂-C₄-alkynyl”) or 2 to 6 (“C₂-C₆-alkynyl”) carbon atoms and one or two triple bonds in any position, for example C₂-C₃-alkynyl, such as ethynyl, 1-propynyl or 2-propynyl; C₂-C₄-alkynyl, such as ethynyl, 1-propynyl, 2-propynyl, 1-butynyl, 2-butynyl, 3-butynyl, 1-methyl-2-propynyl and the like, C₂-C₆-alkynyl, such as ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl, 1-ethyl-1-methyl-2-propynyl and the like;

The term “cycloalkyl” as used herein refers to mono- or bi- or polycyclic saturated hydrocarbon radicals having in particular 3 to 6 (“C₃-C₆-cycloalkyl”) or 3 to 5 (“C₃-C₅-cycloalkyl”) or 3 to 4 (“C₃-C₄-cycloalkyl”) carbon atoms. Examples of monocyclic radicals having 3 to 4 carbon atoms comprise cyclopropyl and cyclobutyl. Examples of monocyclic radicals having 3 to 5 carbon atoms comprise cyclopropyl, cyclobutyl and cyclopentyl. Examples of monocyclic radicals having 3 to 6 carbon atoms comprise cyclopropyl, cyclobutyl, cyclopentyl and cyclohexyl. Examples of monocyclic radicals having 3 to 8 carbon atoms comprise cyclopropyl, cyclobutyl, cyclopentyl, cyclohexyl, cycloheptyl and cyclooctyl. Examples of bicyclic radicals having 7 or 8 carbon atoms comprise bicyclo[2.2.1]heptyl, bicyclo[3.1.1]heptyl, bicyclo[2.2.2]octyl and bicyclo[3.2.1]octyl. Preferably, the term cycloalkyl denotes a monocyclic saturated hydrocarbon radical.

The term “cycloalkoxy” as used herein refers to a cycloalkyl radical, in particular a monocyclic cycloalkyl radical, as defined above having in particular 3 to 6 (“C₃-C₆-cycloalkoxy”) or 3 to 5 (“C₃-C₅-cycloalkoxy”) or 3 to 4 (“C₃-C₄-cycloalkoxy”) carbon atoms, which is bound via an oxygen atom to the remainder of the molecule.

The term “cycloalkyl-C₁-C₄-alkyl” refers to a C₃-C₈-cycloalkyl (“C₃-C₈-cycloalkyl-C₁-C₄-alkyl”), preferably a C₃-C₆-cycloalkyl (“C₃-C₆-cycloalkyl-C₁-C₄-alkyl”), more preferably a C₃-C₄-cycloalkyl (“C₃-C₄-cycloalkyl-C₁-C₄-alkyl”) as defined above (preferably a monocyclic cycloalkyl group) which is bound to the remainder of the molecule via a C₁-C₄-alkyl group, as defined above. Examples for C₃-C₄-cycloalkyl-C₁-C₄-alkyl are cyclopropylmethyl, cyclopropylethyl, cyclopropylpropyl, cyclobutylmethyl, cyclobutylethyl and cyclobutylpropyl, Examples for C₃-C₆-cycloalkyl-C₁-C₄-alkyl, apart those mentioned for C₃-C₄-cycloalkyl-C₁-C₄-alkyl, are cyclopentylmethyl, cyclopentylethyl, cyclopentylpropyl, cyclohexylmethyl, cyclohexylethyl and cyclohexylpropyl.

The term “C₁-C₂-alkoxy” is a C₁-C₂-alkyl group, as defined above, attached via an oxygen atom. The term “C₁-C₃-alkoxy” is a C₁-C₃-alkyl group, as defined above, attached via an oxygen atom. The term “C₁-C₄-alkoxy” is a C₁-C₄-alkyl group, as defined above, attached via an oxygen atom. The term “C₁-C₆-alkoxy” is a C₁-C₆-alkyl group, as defined above, attached via an oxygen atom. The term “C₁-C₁₀-alkoxy” is a C₁-C₁₀-alkyl group, as defined above, attached via an oxygen atom. C₁-C₂-Alkoxy is OCH₃ or OC₂H₅. C₁-C₃-Alkoxy is additionally, for example, n-propoxy and 1-methylethoxy (isopropoxy). C₁-C₄-Alkoxy is additionally, for example, butoxy, 1-methylpropoxy (sec-butoxy), 2-methylpropoxy (isobutoxy) or 1,1-dimethylethoxy (tert-butoxy). C₁-C₆-Alkoxy is additionally, for example, pentoxy, 1-methylbutoxy, 2-methylbutoxy, 3-methylbutoxy, 1,1-dimethylpropoxy, 1,2-dimethylpropoxy, 2,2-dimethylpropoxy, 1-ethylpropoxy, hexoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy or 1-ethyl-2-methylpropoxy. C₁-C₈-Alkoxy is additionally, for example, heptyloxy, octyloxy, 2-ethylhexyloxy and positional isomers thereof. C₁-C₁₀-Alkoxy is additionally, for example, nonyloxy, decyloxy and positional isomers thereof.

The term “C₁-C₂-haloalkoxy” is a C₁-C₂-haloalkyl group, as defined above, attached via an oxygen atom. The term “C₁-C₃-haloalkoxy” is a C₁-C₃-haloalkyl group, as defined above, attached via an oxygen atom. The term “C₁-C₄-haloalkoxy” is a C₁-C₄-haloalkyl group, as defined above, attached via an oxygen atom. The term “C₁-C₆-haloalkoxy” is a C₁-C₆-haloalkyl group, as defined above, attached via an oxygen atom. C₁-C₂-Haloalkoxy is, for example, OCH₂F, OCHF₂, OCF₃, OCH₂Cl, OCHCl₂, OCCl₃, chlorofluoromethoxy, dichlorofluoromethoxy, chlorodifluoromethoxy, 2-fluoroethoxy, 2-chloroethoxy, 2-bromoethoxy, 2-iodoethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy or OC₂F₅. C₁-C₃-Haloalkoxy is additionally, for example, 2-fluoropropoxy, 3-fluoropropoxy, 2,2-difluoropropoxy, 2,3-difluoropropoxy, 2-chloropropoxy, 3-chloropropoxy, 2,3-dichloropropoxy, 2-bromopropoxy, 3-bromopropoxy, 3,3,3-trifluoropropoxy, 3,3,3-trichloropropoxy, OCH₂-C₂F₅, OCF₂-C₂F₅, 1-(CH₂F)-2-fluoroethoxy, 1-(CH₂Cl)-2-chloroethoxy or 1-(CH₂Br)-2-bromoethoxy. C₁-C₄-Haloalkoxy is additionally, for example, 4-fluorobutoxy, 4-chlorobutoxy, 4-bromobutoxy or nonafluorobutoxy. C₁-C₆-Haloalkoxy is additionally, for example, 5-fluoropentoxy, 5-chloropentoxy, 5-brompentoxy, 5-iodopentoxy, undecafluoropentoxy, 6-fluorohexoxy, 6-chlorohexoxy, 6-bromohexoxy, 6-iodohexoxy or dodecafluorohexoxy.

The term “C₁-C₆-alkoxy-C₁-C₄-alkyl” as used herein, refers to a straight-chain or branched alkyl having 1 to 4 carbon atoms, as defined above, where one hydrogen atom is replaced by a C₁-C₆-alkoxy group, as defined above. Examples are methoxymethyl, ethoxymethyl, propoxymethyl, isopropoxymethyl, n-butoxymethyl, sec-butoxymethyl, isobutoxymethyl, tertbutoxymethyl, 1-methoxyethyl, 1-ethoxyethyl, 1-propoxyethyl, 1-isopropoxyethyl, 1-n-butoxyethyl, 1-sec-butoxyethyl, 1-isobutoxyethyl, 1-tert-butoxyethyl, 2-methoxyethyl, 2-ethoxyethyl, 2-propoxyethyl, 2-isopropoxyethyl, 2-n-butoxyethyl, 2-sec-butoxyethyl, 2-isobutoxyethyl, 2-tert-butoxyethyl, 1-methoxypropyl, 1-ethoxypropyl, 1-propoxypropyl, 1-isopropoxypropyl, 1-n-butoxypropyl, 1-sec-butoxypropyl, 1-isobutoxypropyl, 1-tert-butoxypropyl, 2-methoxypropyl, 2-ethoxypropyl, 2-propoxypropyl, 2-isopropoxypropyl, 2-n-butoxypropyl, 2-sec-butoxypropyl, 2-isobutoxypropyl, 2-tert-butoxypropyl, 3-methoxypropyl, 3-ethoxypropyl, 3-propoxypropyl, 3-isopropoxypropyl, 3-n-butoxypropyl, 3-sec-butoxypropyl, 3-isobutoxypropyl, 3-tert-butoxypropyl and the like.

The term “alkoxyalkoxy” as used herein refers to an alkoxyalkyl radical, in particular a C₁-C₆-alkoxy-C₁-C₄-alkyl radical, as defined above, which is bound via an oxygen atom to the remainder of the molecule. Examples thereof are OCH₂—OCH₃, OCH₂—OC₂H₅, n-propoxymethoxy, OCH₂—OCH(CH₃)₂, n-butoxymethoxy, (1-methylpropoxy) methoxy, (2-methylpropoxy) methoxy, OCH₂—OC(CH₃)₃, 2-(methoxy) ethoxy, 2-(ethoxy) ethoxy, 2-(n-propoxy) ethoxy, 2-(1-methylethoxy) ethoxy, 2-(n-butoxy) ethoxy, 2-(1-methylpropoxy) ethoxy, 2-(2-methylpropoxy) ethoxy, 2-(1,1-dimethylethoxy) ethoxy, etc.

The substituent “oxo” replaces a CH₂ by a C(═O) group.

The term “aryl” relates to phenyl and bi- or polycyclic carbocycles having at least one fused phenylene ring, which is bound to the remainder of the molecule. Examples of bi- or polycyclic carbocycles having at least one phenylene ring include naphthyl, tetrahydronaphthyl, indanyl, indenyl, anthracenyl, fluorenyl etc.

The term “aryl-C₁-C₄-alkyl” relates to C₁-C₄-alkyl, as defined above, wherein one hydrogen atom has been replaced by an aryl radical, in particular a phenyl radical. Particular examples of aryl-C₁-C₄-alkyl include-CH₂-phenyl, 1-phenethyl, 2-phenetyl, 1-phenylpropyl, 2-phenylpropyl, 3-phenyl-1-propyl and 2-phenyl-2-propyl.

The term “aryloxy-C₁-C₄-alkyl” relates to C₁-C₄-alkyl, as defined above, wherein one hydrogen atom has been replaced by an aryloxy radical, in particular a phenoxy radical. Particular examples of aryloxy-C₁-C₄-alkyl include phenoxymethyl, 1-phenoxyethyl, 2-phenoxyetyl, 1-phenoxypropyl, 2-phenoxypropyl, 3-phenoxy-1-propyl and 2-phenoxy-2-propyl.

The term “aryl-C₁-C₄-carbonyl” relates to aryl as defined above, in particular a phenyl radical, which is bound by a carbonyl to the remainder of the molecule. Particular examples of arylcarbonyl include benzoyl, 1-naphthoyl and 2-naphthoyl.

The term “hetaryl” relates to aromatic heterocyclyl or heterocycles having either 5 or 6 ring atoms (5- or 6-membered hetaryl) and being monocyclic or 8, 9 or 10 ring atoms and bing bicyclic. Hetaryl will generally have at least one ring atom selected from O, S and N, which in case of N may be an imino-nitrogen or an amino-nitrogen, which carries hydrogen or a radical different from hydrogen. Hetaryl may have 1, 2, 3 or 4 further nitrogen atoms as ring members, which are imino nitrogens. Examples of 5- or 6-membered hetaryl include 2-furyl, 3-furyl, 2-thienyl, 3-thienyl, 1-pyrrolyl, 2-pyrrolyl, 3-pyrrolyl, 1-pyrazolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 1-imidazolyl, 2-imidazolyl, 4-imidazolyl, 1,3,4-triazol-1-yl, 1,3,4-triazol-2-yl, 1,3,4-oxadiazolyl-2-yl, 1,3,4-thiadiazol-2-yl, 2-pyridinyl, 3-pyridinyl, 4-pyridinyl, 3-pyridazinyl, 4-pyridazinyl, 2-pyrimidinyl, 4-pyrimidinyl, 5-pyrimidinyl, 2-pyrazinyl and 1,3,5-triazin-2-yl. Examples of 8-, 9- or 10-membered hetaryl include, for example, quinolinyl, isoquinolinyl, cinnolinyl, indolyl, indolizynyl, isoindolyl, indazolyl, benzofuryl, benzothienyl, benzo[b]thiazolyl, benzoxazolyl, benzthiazolyl, benzimidazolyl, imidazo[1,2-a]pyridine-2-yl, thieno[3,2-b]pyridine-5-yl, imidazo-[2,1-b]-thiazol-6-yl and 1,2,4-triazolo[1,5-a]pyridine-2-yl.

Examples of N-bound 5-, 6-, 7 or 8-membered saturated heterocyclyl or heterocycles include: pyrrolidin-1-yl, pyrazolidin-1-yl, imidazolidin-1-yl, oxazolidin-3-yl, isoxazolidin-2-yl, thiazolidin-3-yl, isothiazolidin-2-yl, piperidin-1-yl, piperazin-1-yl, morpholin-4-yl, thiomorpholin-4-yl, 1-oxothiomorpholin-4-yl, 1,1-dioxothiomorpholin-4-yl, azepan-1-yl and the like.

The term “hetaryl-C₁-C₄-alkyl” relates to C₁-C₄-alkyl, as defined above, wherein one hydrogen atom has been replaced by a hetaryl radical, in particular a pyridyl radical. Particular examples of hetaryl-C₁-C₄-alkyl include 2-pyridylmethyl, 3-pyridylmethyl, 4-pyridylmethyl, 1-(2-pyridyl)ethyl, 2-(2-pyridyl)ethyl, 1-(3-pyridyl)ethyl, 2-(3-pyridyl)ethyl, 1-(4-pyridyl)ethyl, 2-(4-pyridyl)ethyl etc.

The term “hetaryloxy-C₁-C₄-alkyl” relates to C₁-C₄-alkyl, as defined above, wherein one hydrogen atom has been replaced by an hetaryloxy radical, in particular a pyridyloxy radical. Particular examples of hetaryloxy-C₁-C₄-alkyl include 2-pyridyloxymethyl, 3-pyridyloxymethyl, 4-pyridyloxymethyl, 1-(2-pyridyloxy)ethyl, 2-(2-pyridyloxy)ethyl, 1-(3-pyridyloxy)ethyl, 2-(3-pyridyloxy)ethyl, 1-(4-pyridyloxy)ethyl, 2-(4-pyridyloxy)ethyl etc.

The term “hetaryl-C₁-C₄-carbonyl” relates to hetaryl as defined above, in particular a C-bound hetaryl radical, e.g. 2-, 3- or 4-pyridyl, 2- or 3-thienyl, 2- or 3-furyl, 1-, 2- or 3-pyrrolyl, 2- or 4-pyrimidinyl, pyridazinyl, 1-, 3- or 4-pyrazolyl, 1-, 2- or 4-imidazolyl radical, which is bound by a carbonyl to the remainder of the molecule.

The term “substituted” if not specified otherwise refers to substituted with 1, 2, or up to maximum possible number of substituents. If substituents as defined in compounds of formula I are more than one then they are independently from each other are same or different if not mentioned otherwise.

With respect to the variables, the embodiments of the compounds of the formula I are,

In one preferred embodiment, maximum two of B¹, B,² and B³ can be N;

In another preferred embodiment, B¹ is CR^{B1}, B² is CR^{B2}, and B³ is CR^{B3};

In another preferred embodiment, B¹ is N, B² is CR^{B2}, and B³ is CR^{B3};

In another preferred embodiment, B¹ is CR^{B1}, B² is N, and B³ is CR^{B3};

In another preferred embodiment, B¹ is CR^{B1}, B² is N, and B³ is N;

In another preferred embodiment, B¹ is N, B² is N, and B³ is CR^{B3};

In another preferred embodiment, B¹ is CR^{B1}, and B² is N or CR^{B2}, B³ is N or CR^{B3};

In another preferred embodiment, B³ is CR^{B3}, and B¹ is N or CR^{B2}, B² is N or CR^{B3}.

In one preferred embodiment, R¹ is H, halogen, C₁-C₆-alkyl, or C₃-C₆-cycloalkyl, wherein the alkyl and cycloalkyl moieties are unsubstituted or substituted with halogen or CN;

In another preferred embodiment, R¹ is H, halogen, C₁-C₆-alkyl, or C₃-C₆-cycloalkyl;

In another preferred embodiment, R¹ is H, halogen, or C₁-C₆-alkyl, wherein the alkyl moieties are unsubstituted or substituted with halogen or CN;

In another preferred embodiment, R¹ is H or C₁-C₆-alkyl, wherein the alkyl moieties are unsubstituted or substituted with halogen or CN;

In another preferred embodiment, R¹ is C₁-C₆-alkyl, which is unsubstituted or substituted with halogen or CN;

In another preferred embodiment, R¹ is C₁-C₆-alkyl, which is unsubstituted;

In another preferred embodiment, R¹ is C₁-C₆-alkyl, which is substituted with halogen or CN;

In another preferred embodiment, R¹ is C₃-C₆-cycloalkyl, which is unsubstituted or substituted with halogen or CN;

In another preferred embodiment, R¹ is C₃-C₆-cycloalkyl, which is unsubstituted;

In another preferred embodiment, R¹ is C₃-C₆-cycloalkyl, which is substituted with halogen or CN;

In another preferred embodiment, R¹ is H or halogen;

In another preferred embodiment, R¹ is H;

In another preferred embodiment, R¹ is halogen;

In another preferred embodiment, R¹ is H, Cl, CH₃, or cyclopropyl;

In another preferred embodiment, R¹ is H or NR⁶R⁷;

In another preferred embodiment, R¹ is NR⁶R⁷.

In one preferred embodiment, R⁶ and R⁷ are, identical or different, H, C₁-C₆-alkyl, phenyl, —CH₂-phenyl, 5- or 6-membered heteroaryl, —CH₂-5- or 6-membered heteroaryl, 1,3-dioxolan-2-ylmethyl, or 2-(methylamino)-2-oxo-ethyl, wherein the alkyl, phenyl and heteroaryl moieties are unsubstituted or substituted with halogen, CN, or C₁-C₆-alkyl.

In another preferred embodiment, R⁶ and R⁷ are, identical or different, H, phenyl, —CH₂-phenyl, 5- or 6-membered heteroaryl, —CH₂-5- or 6-membered heteroaryl, 1,3-dioxolan-2-ylmethyl, or 2-(methylamino)-2-oxo-ethyl, wherein the, phenyl and heteroaryl moieties are unsubstituted or substituted with halogen or CN,

In another preferred embodiment, R⁶ and R⁷ are, identical or different, H, phenyl, —CH₂-phenyl, 5- or 6-membered heteroaryl, —CH₂-5- or 6-membered heteroaryl, 1,3-dioxolan-2-ylmethyl, or 2-(methylamino)-2-oxo-ethyl, wherein the, phenyl and heteroaryl moieties are unsubstituted or substituted with halogen, CN, or C₁-C₆-alkyl;

In another preferred embodiment, R⁶ and R⁷ are, identical or different, H, —CH₂-5- or 6-membered heteroaryl, 1,3-dioxolan-2-ylmethyl, or 2-(methylamino)-2-oxo-ethyl, wherein the, phenyl and heteroaryl moieties are unsubstituted or substituted with halogen, CN, or C₁-C₆-alkyl;

In another preferred embodiment, R⁶ is H and R⁷ is —CH₂-5- or 6-membered heteroaryl, 1,3-dioxolan-2-ylmethyl, or 2-(methylamino)-2-oxo-ethyl, wherein the alkyl, phenyl and heteroaryl moieties are unsubstituted or substituted with halogen, CN, or C₁-C₆-alkyl;

In another preferred embodiment, R⁶ and R⁷ independently of each other are selected from Rx-1 to Rx-7 as shown in table Rx.

TABLE Rx

| | |
|---|---|
| 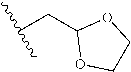 [SMILES: *CC1OCCO1] | Rx-1 |
| 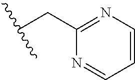 [SMILES: *Cc1ncccn1] | Rx-2 |
| 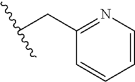 [SMILES: *Cc1ccccn1] | Rx-3 |
| 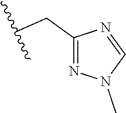 [SMILES: *Cc1ncn(C)n1] | Rx-4 |
| 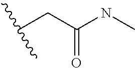 [SMILES: *CC(=O)NC] | Rx-5 |
| 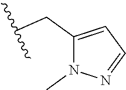 [SMILES: *Cc1ccnn1C] | Rx-6 |
| H | Rx-7 |
| 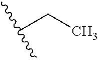 [SMILES: *CC] | Rx-8 |

In another preferred embodiment, R⁶ and R⁷ independently of each other are selected from Rx-1, Rx-2, Rx-7, and Rx-8, more preferably from Rx-1 and Rx-7.

In one preferred embodiment, R² is H, halogen, C₁-C₆-alkyl, or C₃-C₆-cycloalkyl, wherein the alkyl and cycloalkyl moieties are unsubstituted or substituted with halogen or CN;

In another preferred embodiment, R² is H, halogen, C₁-C₆-alkyl, or C₃-C₆-cycloalkyl;

In another preferred embodiment, R² is H, halogen, or C₁-C₆-alkyl, wherein the alkyl moieties are unsubstituted or substituted with halogen or CN;

In another preferred embodiment, R² is H or C₁-C₆-alkyl, wherein the alkyl moieties are unsubstituted or substituted with halogen or CN;

In another preferred embodiment, R² is C₁-C₆-alkyl, which is unsubstituted or substituted with halogen or CN;

In another preferred embodiment, R² is C₁-C₆-alkyl, which is unsubstituted;

In another preferred embodiment, R² is C₁-C₆-alkyl, which is substituted with halogen or CN;

In another preferred embodiment, R² is C₃-C₆-cycloalkyl, which is unsubstituted or substituted with halogen or CN;

In another preferred embodiment, R² is C₃-C₆-cycloalkyl, which is unsubstituted;

In another preferred embodiment, R² is C₃-C₆-cycloalkyl, which is substituted with halogen or CN;

In another preferred embodiment, R² is H or halogen;

In another preferred embodiment, R² is H;

In another preferred embodiment, R² is halogen;

In another preferred embodiment, R² is H, Cl, Br, F, CH₃, C₂H₅, n-C₃H₇, isopropyl, cyclopropyl, CH₂F, CHF₂, or CF₃.

In another preferred embodiment, R² is H, CH₃, C₂H₅, isopropyl, or cyclopropyl;

In another preferred embodiment, R² is H;

In another preferred embodiment, R² is CH₃;

In another preferred embodiment, R² is C₂H₅;

In another preferred embodiment, R² is isopropyl;

In another preferred embodiment, R² is cyclopropyl.

In one preferred embodiment, R^{B1}, R^{B2}, R^{B3}, and R^{B4} independently of each other are H, halogen, CN, C₁-C₆-alkyl, C₃-C₆-cycloalkyl, or C₁-C₆-alkoxy, wherein the alkyl, alkoxy, and cycloalkyl moieties are unsubstituted or substituted with halogen;

In another preferred embodiment, R^{B1}, R^{B2}, R^{B3}, and R^{B4} independently of each other are H, halogen, CN, C₁-C₆-alkyl, or C₁-C₆-alkoxy, wherein the alkyl, alkoxy, and cycloalkyl moieties are unsubstituted or substituted with halogen;

In another preferred embodiment, R^{B1}, R^{B2}, R^{B3}, and R^{B4} independently of each other are H, halogen, or C₁-C₆-alkyl, wherein the alkyl moieties are unsubstituted or substituted with halogen;

In another preferred embodiment, R^{B1}, R^{B2}, R^{B3}, and R^{B4} independently of each other are H, Cl, Br, F, CN, CH₃, C₂H₅, n-C₃H₇, isopropyl, cyclopropyl, CF₃, CH₂F, OCH₃, or OCHF₂.

In another preferred embodiment, R^{B1}, R^{B2}, R^{B3}, and R^{B4} independently of each other are H, Cl, Br, F, CN, CH₃, isopropyl, OCH₃, or OCHF₂.

In another preferred embodiment, R^{B1} and R^{B4} independently of each other are H, Cl, or CH₃.

In another preferred embodiment, R^{B2} and R^{B3} independently of each other are H, Cl, Br, F, CN, CH₃, isopropyl, cyclopropyl, OCH₃, or OCHF₂.

In another preferred embodiment, R^{B2} and R^{B3} independently of each other are H, Cl, F, CN, CH₃, or OCH₃;

In another preferred embodiment, R^{B2} and R^{B3} independently of each other are H or Cl.

In one preferred embodiment, Q is #—C(═O)—N(R⁵)—, wherein #denotes connection to Ar¹;

In another preferred embodiment, Q is #—N(R⁵)—C(═O)—, wherein #denotes connection to Ar¹.

In one preferred embodiment, R⁵ is H, C₁-C₆-alkyl, C₃-C₆-cycloalkyl, or C₁-C₆-alkyl-C₃-C₆-cycloalkyl, wherein the alkyl and cycloalkyl moieties are unsubstituted or substituted with halogen or CN;

In another preferred embodiment, R⁵ is H, C₁-C₆-alkyl, or C₁-C₆-alkyl-C₃-C₆-cycloalkyl, wherein the alkyl and cycloalkyl moieties are unsubstituted or substituted with halogen or CN;

In another preferred embodiment, R⁵ is H, C₁-C₆-alkyl, or C₁-C₆-alkyl-C₃-C₆-cycloalkyl, wherein the alkyl and cycloalkyl moieties are unsubstituted;

In another preferred embodiment, R⁵ is H, C₁-C₆-alkyl, or C₁-C₆-alkyl-C₃-C₆-cycloalkyl, wherein the alkyl and cycloalkyl moieties are substituted with halogen or CN;

In another preferred embodiment, R⁵ is H, CH₃, C₂H₅, or —CH₂-cyclopropyl;

In one preferred embodiment, D is DA;

In one preferred embodiment, R³ is H, C₁-C₆-alkyl, or C₃-C₆-cycloalkyl, wherein the alkyl and cycloalkyl moieties are unsubstituted or substituted with halogen or CN;

In another preferred embodiment, R³ is H or C₁-C₆-alkyl, wherein the alkyl moieties are unsubstituted or substituted with halogen or CN;

In another preferred embodiment, R³ is H or C₃-C₆-cycloalkyl, wherein the cycloalkyl moieties are unsubstituted or substituted with halogen or CN;

In another preferred embodiment, R³ is H;

In another preferred embodiment, R³ is H and DA exists in any of below tautomeric forms D3 and D4;

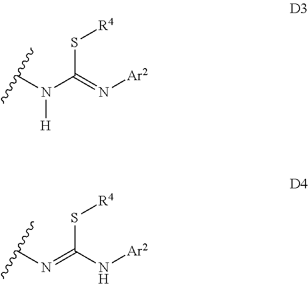

In one preferred embodiment, R⁴ is H, C₁-C₆-alkyl, or C₃-C₆-cycloalkyl, wherein the alkyl and cycloalkyl moieties are unsubstituted or substituted with halogen, —O—(C═O)—C₁-C₆-alkyl, —O(C═O)—C₁-C₆-alkoxy, or CN;

In another preferred embodiment, R⁴ is H or C₁-C₆-alkyl, wherein the alkyl moieties are unsubstituted or substituted with halogen, —O—(C═O)—C₁-C₆-alkyl, —O—(C═O)—C₁-C₆-alkoxy, or CN;

In another preferred embodiment, R⁴ is H or C₃-C₆-cycloalkyl, wherein the cycloalkyl moieties are unsubstituted or substituted with halogen or CN;

In another preferred embodiment, R⁴ is H;

In another preferred embodiment, R⁴ is H and DA exists in any of below tautomeric forms D1 and D2;

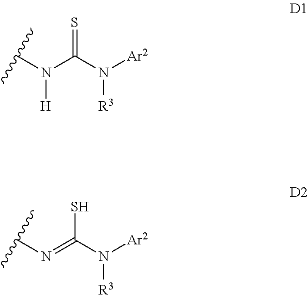

In another preferred embodiment, D is DB wherein B is a 5- or 6-membered carbocyclic group, wherein 1 or 2 CH₂ moieties of the carbocyclic group may be replaced by a carbonyl group, wherein the carbocyclic group is unsubstituted or substituted with R^{h};

In another preferred embodiment, D is DB wherein B is a 5- or 6-membered carbocyclic group, wherein one CH₂ moiety of the carbocyclic group may be replaced by a carbonyl group, wherein the carbocyclic group is unsubstituted or substituted with R^{h};

In another preferred embodiment, D is DB wherein B is a 5- or 6-membered carbocyclic group, wherein one CH₂ moiety of the carbocyclic group may be replaced by a carbonyl group;

In another preferred embodiment, D is DB wherein B is a 5- or 6-membered carbocyclic group, wherein one CH₂ moiety of the carbocyclic group is replaced by a carbonyl group;

In another preferred embodiment, D is DB which is selected from D5 to D7, and wherein the carbocyclic group of D5 to D7 moeities are unsubstituted or substituted with 1 or 2 substituents R^{h},

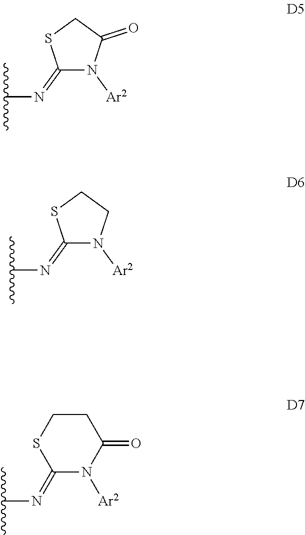

In another preferred embodiment, D is a group selected from of D5 to D7 moeities, which are unsubstituted or substituted with 1 or 2 substituents R^{h},

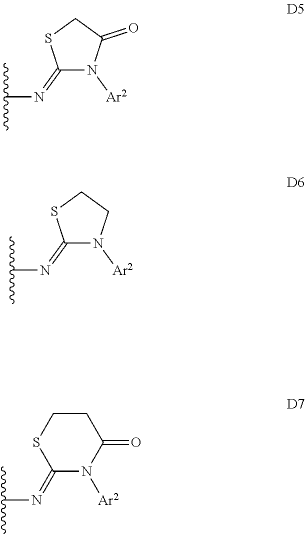

In another preferred embodiment, D is DB which is selected from D5 to D7, and wherein the carbocyclic group of D5 to D7 moeities are unsubstituted;

In another preferred embodiment, D is DB which is selected from D5 to D7, and wherein wherein the carbocyclic group of D5 to D7 moeities are substituted with 1 or 2 substituents R^{h};

In another preferred embodiment, D is selected from DA, D5, D6, and D7 as defined herein;

In another preferred embodiment, D is selected from DA, D5, D6, and D7, wherein the carbocyclic group of D5, D6, and D7 moeities are unsubstituted or substituted with 1 or 2 substituents R^{h};

In another preferred embodiment, D is DA or D5, wherein D5 is unsubstituted or substituted with 1 substituent R^{h};

In another preferred embodiment, D is selected from DA or D5, wherein the carbocyclic group of D5 is substituted with 1 substituent R^{h};

In another preferred embodiment, D is selected from DA or D5, wherein the carbocyclic group of D5 is unsubstituted;

In another preferred embodiment, D is D5, wherein the carbocyclic group of D5 is unsubstituted or substituted with 1 substituent R^{h};

In one preferred embodiment, Ar¹ is phenyl which is unsubstituted or substituted with R^{Ar1}.

In another preferred embodiment, Ar¹ is 5- or 6-membered hetaryl, which is unsubstituted or substituted with R^{Ar1};

In more preferred embodiment, Ar¹ is phenyl, pyrimidinyl, pyridazinyl, thiophenyl, thiazolyl, or pyridyl, which are unsubstituted or substituted with R^{Ar1}.

In one preferred embodiment, R^{Ar1} is halogen, SF₅, NO₂, OH, CN, C₁-C₆-alkyl, C₁-C₆-alkoxy, C₃-C₆-cycloalkyl, C₃-C₆-heterocyclyl, C₃-C₆-cycloalkoxy, C₂-C₆-alkenyl, C₂-C₆-alkynyl wherein the alkyl, alkoxy, alkenyl, alkynyl, cycloalkyl, C₃-C₆-heterocyclyl, and cycloalkoxy moieties are unsubstituted or substituted with R^{f} In another preferred embodiment, Ar¹ is 5- or 6-membered hetaryl, which is unsubstituted or substituted with R^{Ar1};
C(═O)—OR^{a}, NR^{b}R^{c}, C₁-C₆-alkylene-CN, C(═O)—NR^{b}R^{c}, C(═O)—R^{d}, NHS(═O)ₘR^{e}, —N═S(═O)—(C₁-C₆-alkyl)₂, SO₂NR^{b}R^{c}, or S(═O)ₘR^{e};

In another preferred embodiment, R^{Ar1} is halogen, SF₅, NO₂, CN, C₁-C₆-alkyl, C₁-C₆-alkoxy, C₃-C₆-cycloalkyl, C₃-C₆-heterocyclyl, C₂-C₆-alkenyl, C₂-C₆-alkynyl wherein the alkyl, alkoxy, alkenyl, alkynyl, cycloalkyl, C₃-C₆-heterocyclyl, and cycloalkoxy moieties are unsubstituted or substituted with R^{f};
NR^{b}R^{c}, C(═O)—NR^{b}R^{c}, C(═O)—R^{d}, NHS(═O)ₘR^{e}, —N═S(═O)—(C₁-C₆-alkyl)₂, or S(═O)ₘR^{e};

In another preferred embodiment, R^{Ar1} is halogen, SF₅, CN, C₁-C₆-alkyl, C₁-C₆-alkoxy, C₂-C₆-alkenyl, C₂-C₆-alkynyl wherein the alkyl, alkoxy, alkenyl, and alkynyl moieties are unsubstituted or substituted with R^{f};
or S(═O)ₘR^{e};

In another preferred embodiment, R^{Ar1} is halogen, CN, C₁-C₆-alkyl, C₁-C₆-alkoxy, wherein the alkyl and alkoxy moieties are unsubstituted or substituted with R^{f},

In another preferred embodiment of compound of formula I, wherein
Ar¹ is phenyl, pyrimidinyl, pyridazinyl, thiophenyl, thiazolyl, or pyridyl, which are unsubstituted or substituted with R^{Ar1};
R^{Ar1} is halogen, SF₅, NO₂, OH, CN, C₁-C₆-alkyl, C₁-C₆-alkoxy, C₃-C₆-cycloalkyl, C₃-C₆-heterocyclyl, C₃-C₆-cycloalkoxy, C₂-C₆-alkenyl, C₂-C₆-alkynyl wherein the alkyl, alkoxy, alkenyl, alkynyl, cycloalkyl, C₃-C₆-heterocyclyl, and cycloalkoxy moieties are unsubstituted or substituted with R^{f};
C(═O)—OR^{a}, NR^{b}R^{c}, C₁-C₆-alkylene-CN, C(═O)—NR^{b}R^{c}, C(═O)—R^{d}, NHS(═O)ₘR^{e}, —N═S(═O)—(C₁-C₆-alkyl)₂, SO₂NR^{b}R^{c}, or S(═O)ₘR^{e};
R^{a}, R^{b} and R^{c} identical or different, are H, C₁-C₆-alkyl, which are unsubstituted or substituted with halogen;
R^{d} is H or C₁-C₆-alkyl;
R^{e} is C₁-C₆-alkyl or C₁-C₆-haloalkyl;
R^{f} is halogen, OH, CN, C₁-C₆-alkyl, C₁-C₆-alkoxy, C₂-C₆-alkenyl, C₂-C₆-alkynyl, C₃-C₆-cycloalkyl, C₃-C₆-cycloalkoxy, which are unsubstituted or substituted with halogen;
m is 0, 1, or 2.

In another preferred embodiment of compound of formula I, wherein
Ar¹ is phenyl, pyrimidinyl, pyridazinyl, thiophenyl, thiazolyl, or pyridyl, which are unsubstituted or substituted with R^{Ar1};
R^{Ar1} is halogen, NO₂, CN, C₁-C₆-alkyl, C₁-C₆-alkoxy, C₃-C₆-cycloalkyl, C₃-C₆-heterocyclyl, C₂-C₆-alkenyl, C₂-C₆-alkynyl wherein the alkyl, alkoxy, alkenyl, alkynyl, cycloalkyl, and C₃-C₆-heterocyclyl moieties are unsubstituted or substituted with R^{f};
NR^{b}R^{c}, C(═O)—NR^{b}R^{c}, C(═O)—R^{d}, NHS(═O)ₘR^{e}, —N═S(═O)—(C₁-C₆-alkyl)₂, or S(═O)ₘR^{e};
R^{a}, R^{b} and R^{c} identical or different, are H, C₁-C₆-alkyl, which are unsubstituted or substituted with halogen;
R^{d} is H or C₁-C₆-alkyl;
R^{e} is C₁-C₆-alkyl or C₁-C₆-haloalkyl;
R^{f} is halogen, OH, CN, C₁-C₆-alkyl, C₁-C₆-alkoxy, C₂-C₆-alkenyl, C₂-C₆-alkynyl, C₃-C₆-cycloalkyl, C₃-C₆-cycloalkoxy, which are unsubstituted or substituted with halogen;
m is 0, 1, or 2.

In another preferred embodiment, Ar¹ is selected from Ar¹-1 to Ar¹-26 as shown in Table Ar¹,

TABLE Ar1

| Ar¹ | Structure |
|---|---|
| Ar¹-1 | 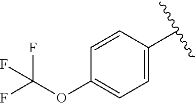 [SMILES: *c1ccc(OC(F)(F)F)cc1] |
| Ar¹-2 | 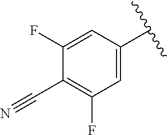 [SMILES: *c1cc(F)c(C#N)c(F)c1] |
| Ar¹-3 | 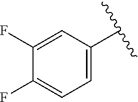 [SMILES: *c1ccc(F)c(F)c1] |
| Ar¹-4 | 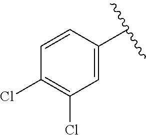 [SMILES: *c1ccc(Cl)c(Cl)c1] |
| Ar¹-5 | 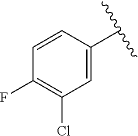 [SMILES: *c1ccc(F)c(Cl)c1] |
| Ar¹-6 | 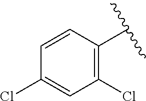 [SMILES: *c1ccc(Cl)cc1Cl] |
| Ar¹-7 | 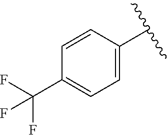 [SMILES: *c1ccc(C(F)(F)F)cc1] |
| Ar¹-8 | 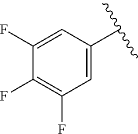 [SMILES: *c1cc(F)c(F)c(F)c1] |
| Ar¹-9 | 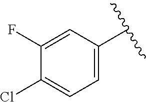 [SMILES: *c1ccc(Cl)c(F)c1] |
| Ar¹-10 | 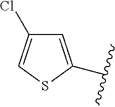 [SMILES: *c1cc(Cl)cs1] |
| Ar¹-11 | 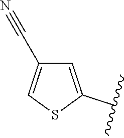 [SMILES: *c1cc(C#N)cs1] |
| Ar¹-12 | 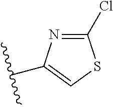 [SMILES: *c1csc(Cl)n1] |
| Ar¹-13 | 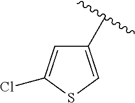 [SMILES: *c1csc(Cl)c1] |
| Ar¹-14 | 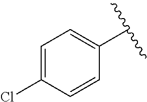 [SMILES: *c1ccc(Cl)cc1] |
| Ar¹-15 | 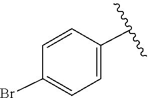 [SMILES: *c1ccc(Br)cc1] |
| Ar¹-16 | 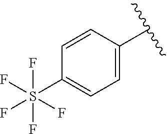 [SMILES: *c1ccc(S(F)(F)(F)(F)F)cc1] |
| Ar¹-17 | 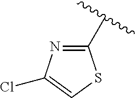 [SMILES: *c1nc(Cl)cs1] |
| Ar¹-18 | 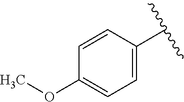 [SMILES: *c1ccc(OC)cc1] |
| Ar¹-19 | 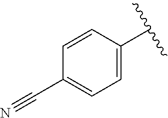 [SMILES: *c1ccc(C#N)cc1] |
| Ar¹-20 | 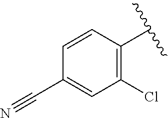 [SMILES: *c1ccc(C#N)cc1Cl] |
| Ar¹-21 | 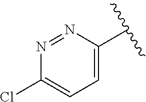 [SMILES: *c1ccc(Cl)nn1] |
| Ar¹-22 | 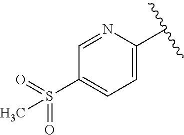 [SMILES: *c1ccc(S(C)(=O)=O)cn1] |
| Ar¹-23 | 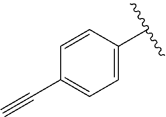 [SMILES: *c1ccc(C#C)cc1] |
| Ar¹-24 | 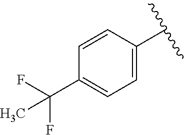 [SMILES: *c1ccc(C(C)(F)F)cc1] |
| Ar¹-25 | 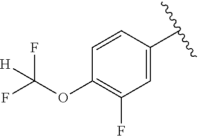 [SMILES: *c1ccc(OC(F)F)c(F)c1] |
| Ar¹-26 | 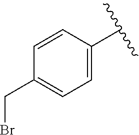 [SMILES: *c1ccc(CBr)cc1] |
| Ar¹-27 | 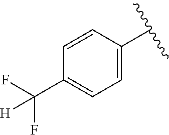 [SMILES: *c1ccc(C(F)F)cc1] |

In another preferred embodiment, Ar¹ is selected from Ar¹-1 to Ar¹-17;

In another preferred embodiment, Ar¹ is selected from Ar¹-1 to Ar¹-9;

In another preferred embodiment, Ar¹ is selected from Ar¹-1 to Ar¹-8;

In another preferred embodiment, Ar¹ is selected from Ar¹-1 to Ar¹-3;

In one preferred embodiment, Ar² is phenyl which is unsubstituted or substituted with R^{Ar2};

In another preferred embodiment, Ar² is 5- or 6-membered hetaryl, which is unsubstituted or substituted with R^{Ar2};

In another preferred embodiment, Ar² is phenyl, pyrimidinyl, thiophenyl, thiazolyl, or pyridyl, which are unsubstituted or substituted with R^{Ar2}.

In one preferred embodiment, R^{Ar2} is halogen, CN, —SCN, —SF₅, C₁-C₆-alkyl, C₁-C₆-alkoxy, C₂-C₆-alkenyl, C₂-C₆-alkynyl, C₁-C₆-alkoxy-C₁-C₄-alkyl, C₁-C₆-alkoxy-C₁-C₄-alkoxy, C₃-C₆-cycloalkyl, C₃-C₆-cycloalkoxy, C₃-C₆-cycloalkyl-C₁-C₄-alkyl, C₃-C₆-cycloalkoxy-C₁-C₄-alkyl, wherein the alkyl, alkoxy, alkenyl, alkynyl, cycloalkyl and cycloalkoxy moieties are unsubstituted or substituted with halogen;
C(═O)—OR^{a}, NR^{b}R^{c}, or C₁-C₆-alkylene-CN;

In another preferred embodiment, R^{Ar2} is halogen, CN, C₁-C₆-alkyl, C₁-C₆-alkoxy, C₁-C₆-alkoxy-C₁-C₄-alkyl, C₃-C₆-cycloalkyl, wherein the alkyl, alkoxy, and cycloalkyl moieties are unsubstituted or substituted with halogen;
or NR^{b}R^{c};

In another preferred embodiment, R^{Ar2} is halogen, CN, NR^{b}R^{c}, C₁-C₆-alkyl, C₁-C₆-alkoxy, C₁-C₆-alkoxy-C₁-C₄-alkyl, C₃-C₆-cycloalkyl, wherein the alkyl, alkoxy, and cycloalkyl moieties are unsubstituted or substituted with halogen;

In another preferred embodiment, R^{Ar2} is NR^{b}R^{c};

In another preferred embodiment, R^{Ar2} is halogen, CN, C₁-C₆-alkyl, wherein the alkyl, moieties are unsubstituted or substituted with halogen;

In another preferred embodiment, R^{Ar2} is halogen or C₁-C₆-alkyl, wherein the alkyl moieties are unsubstituted or substituted with halogen;

In another preferred embodiment, R^{Ar2} is halogen or C₁-C₆-alkyl;

In another preferred embodiment, R^{Ar2} is halogen;

In another preferred embodiment, R^{Ar2} is halogen or CN;

In another preferred embodiment, Ar² is selected from Ar²-1 to Ar²-20 as shown in Table Ar²,

TABLE Ar2

| Ar² | Structure |
|---|---|
| Ar²-1 | 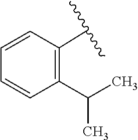 [SMILES: *c1ccccc1C(C)C] |
| Ar²-2 | 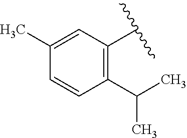 [SMILES: *c1cc(C)ccc1C(C)C] |
| Ar²-3 | 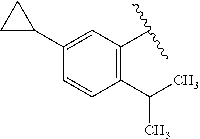 [SMILES: *c1cc(C2CC2)ccc1C(C)C] |
| Ar²-4 | 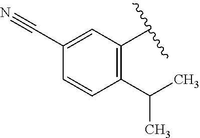 [SMILES: *c1cc(C#N)ccc1C(C)C] |
| Ar²-5 | 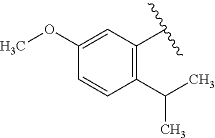 [SMILES: *c1cc(OC)ccc1C(C)C] |
| Ar²-6 | 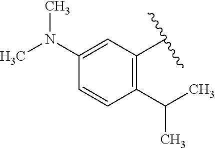 [SMILES: *c1cc(N(C)C)ccc1C(C)C] |
| Ar²-7 | 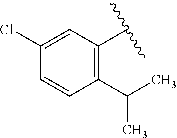 [SMILES: *c1cc(Cl)ccc1C(C)C] |
| Ar²-8 | 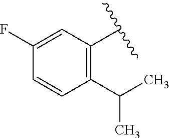 [SMILES: *c1cc(F)ccc1C(C)C] |
| Ar²-9 | 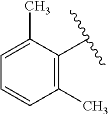 [SMILES: *c1c(C)cccc1C] |
| Ar²-10 | 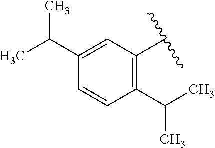 [SMILES: *c1cc(C(C)C)ccc1C(C)C] |
| Ar²-11 | 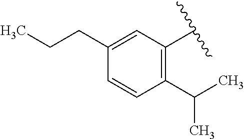 [SMILES: *c1cc(CCC)ccc1C(C)C] |
| Ar²-12 | 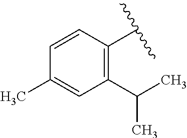 [SMILES: *c1ccc(C)cc1C(C)C] |
| Ar²-13 | 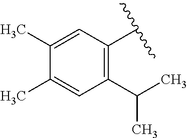 [SMILES: *c1cc(C)c(C)cc1C(C)C] |
| Ar²-14 | 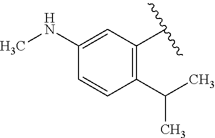 [SMILES: *c1cc(NC)ccc1C(C)C] |
| Ar²-15 | 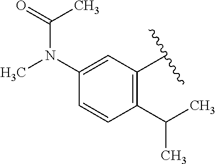 [SMILES: *c1cc(N(C)C(C)=O)ccc1C(C)C] |
| Ar²-16 | 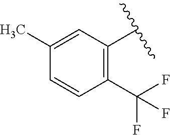 [SMILES: *c1cc(C)ccc1C(F)(F)F] |
| Ar²-17 | 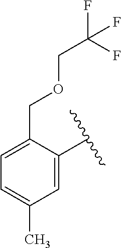 [SMILES: *c1cc(C)ccc1COCC(F)(F)F] |
| Ar²-18 | 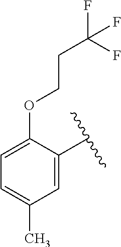 [SMILES: *c1cc(C)ccc1OCCC(F)(F)F] |
| Ar²-19 | 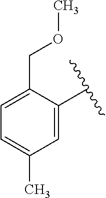 [SMILES: *c1cc(C)ccc1COC] |
| Ar²-20 | 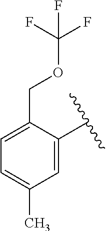 [SMILES: *c1cc(C)ccc1COC(F)(F)F] |

In another preferred embodiment, Ar² is selected from Ar²-1 to Ar²-13;

In another preferred embodiment, Ar² is selected from Ar²-1 to Ar²-3;

In one embodiment, R^{a}, R^{b} and R^{c} identical or different, are H, C₁-C₆-alkyl, C₂-C₆-alkenyl, which are unsubstituted or substituted with halogen;

In another preferred embodiment, R^{a}, R^{b} and R^{c} identical or different, are H, C₁-C₆-alkyl which is unsubstituted or substituted with halogen;

In one preferred embodiment, R^{d} is H;

In another preferred embodiment, R^{d} is C₁-C₆-alkyl.

In one preferred embodiment, R^{e} is C₁-C₆-alkyl, C₁-C₆-haloalkyl, C₃-C₆-cycloalkyl, or C₃-C₆-halocycloalkyl;

In another preferred embodiment, R^{e} is C₁-C₆-alkyl or C₁-C₆-haloalkyl;

In one preferred embodiment, R^{f} is halogen, OH, CN, C₁-C₆-alkyl, C₁-C₆-alkoxy, C₂-C₆-alkenyl, C₂-C₆-alkynyl, C₃-C₆-cycloalkyl, or C₃-C₆-cycloalkoxy, which are unsubstituted or substituted with halogen;

In another preferred embodiment, R^{f} is halogen, OH, CN, or C₁-C₆-alkyl.

In a preferred embodiment, R^{h} is halogen or C₁-C₆-alkyl;

In one preferred embodiment, m is 0;

In another preferred embodiment, m is 1;

In another preferred embodiment, m is 2;

In another preferred embodiment, m is 0 or 1;

In another preferred embodiment, m is 1 or 2.

In another preferred embodiment of compound of formula I, wherein
Q is —C(═O)—N(R⁵)—, or —N(R⁵)—C(═O)—;
D is DA, D5, D6, or D7, preferably D5;
B¹ is N or CR^{B1}, B² is CR^{B2}, and B³ is CR^{B3};
R^{B1}, R^{B2}, R^{B3}, and R^{B4} independently of each other are H, halogen, CN, C₁-C₆-alkyl, or C₁-C₆-alkoxy, wherein the alkyl and alkoxymoieties are unsubstituted or substituted with halogen;
R⁵ is H, C₁-C₆-alkyl, or C₁-C₆-alkyl-C₃-C₆-cycloalkyl;
R¹ is H, halogen, C₁-C₆-alkyl, or C₃-C₆-cycloalkyl;
R² is H or C₁-C₆-alkyl;
R³ is H or C₁-C₆-alkyl;
R⁴ is H or C₁-C₆-alkyl;
Ar¹ is phenyl, pyrimidinyl, pyridazinyl, thiophenyl, thiazolyl, or pyridyl, which are unsubstituted or substituted with R^{Ar1};
R^{Ar1} is halogen, SF₅, NO₂, OH, CN, C₁-C₆-alkyl, C₁-C₆-alkoxy, C₃-C₆-cycloalkyl, C₃-C₆-heterocyclyl, C₃-C₆-cycloalkoxy, C₂-C₆-alkenyl, C₂-C₆-alkynyl wherein the alkyl, alkoxy, alkenyl, alkynyl, cycloalkyl, C₃-C₆-heterocyclyl, and cycloalkoxy moieties are unsubstituted or substituted with R^{f}; C(═O)—OR^{a}, NR^{b}R^{c}, C₁-C₆-alkylene-CN, C(═O)—NR^{b}R^{c}, C(═O)—R^{d}, NHS(═O)ₘR^{e}, N═S(═O)—(C₁-C₆-alkyl)₂, SO₂NR^{b}R^{c}, or S(═O)ₘR^{e};
R^{a}, R^{b} and R^{c} identical or different, are H, C₁-C₆-alkyl, which are unsubstituted or substituted with halogen;
R^{d} is H or C₁-C₆-alkyl;
R^{e} is C₁-C₆-alkyl or C₁-C₆-haloalkyl;
R^{f} is halogen, OH, CN, C₁-C₆-alkyl, C₁-C₆-alkoxy, C₂-C₆-alkenyl, C₂-C₆-alkynyl, C₃-C₆-cycloalkyl, C₃-C₆-cycloalkoxy, which are unsubstituted or substituted with halogen;
m is 0, 1, or 2.

In another preferred embodiment of compound of formula I, wherein
Q is —C(═O)—N(R⁵)—;
D is DB, preferably D5;
B¹ is N or CR^{B1}, B² is CR^{B2}, and B³ is CR^{B3};
R^{B1}, R^{B2}, R^{B3}, and R^{B4} independently of each other are H, halogen, CN, C₁-C₆-alkyl, or C₁-C₆-alkoxy, wherein the alkyl, alkoxy, and cycloalkyl moieties are unsubstituted or substituted with halogen;
R⁵ is H, C₁-C₆-alkyl, or C₁-C₆-alkyl-C₃-C₆-cycloalkyl;
R¹ is H, halogen, C₁-C₆-alkyl, or C₃-C₆-cycloalkyl;
R² is C₁-C₆-alkyl;
Ar¹ is phenyl, pyrimidinyl, pyridazinyl, thiophenyl, thiazolyl, or pyridyl, which are unsubstituted or substituted with R^{Ar1};
R^{Ar1} is halogen, NO₂, CN, C₁-C₆-alkyl, C₁-C₆-alkoxy, C₃-C₆-cycloalkyl, C₃-C₆-heterocyclyl, C₂-C₆-alkenyl, C₂-C₆-alkynyl wherein the alkyl, alkoxy, alkenyl, alkynyl, cycloalkyl, and C₃-C₆-heterocyclyl moieties are unsubstituted or substituted with R^{f};
NR^{b}R^{c}, C(═O)—NR^{b}R^{c}, C(═O)—R^{d}, NHS(═O)ₘR^{e}, —N═S(═O)—(C₁-C₆-alkyl)₂, or S(═O)ₘR^{e};
Ar² is phenyl which is unsubstituted or substituted with R^{Ar2};
R^{Ar2} is halogen, CN, C₁-C₆-alkyl, C₁-C₆-alkoxy, C₃-C₆-cycloalkyl, wherein the alkyl, alkoxy, and cycloalkyl moieties are unsubstituted or substituted with halogen;
or NR^{b}R^{c};
R^{a}, R^{b} and R^{c} identical or different, are H, C₁-C₆-alkyl, which are unsubstituted or substituted with halogen;
R^{d} is H or C₁-C₆-alkyl;
R^{e} is C₁-C₆-alkyl or C₁-C₆-haloalkyl;
R^{f} is halogen, OH, CN, C₁-C₆-alkyl, C₁-C₆-alkoxy, C₂-C₆-alkenyl, C₂-C₆-alkynyl, C₃-C₆-cycloalkyl, C₃-C₆-cycloalkoxy, which are unsubstituted or substituted with halogen;
m is 0, 1, or 2.

In another preferred embodiment, compounds of formula I are selected from compounds of formulae A.1 to A.10,

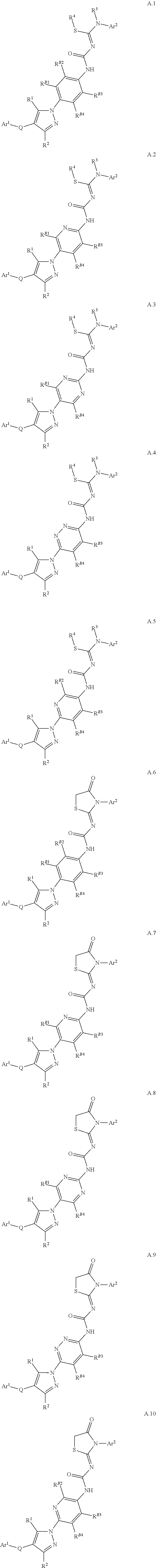

In another preferred embodiment compounds of formula I are selected from compounds of formula A.1 to A.5

In another preferred embodiment compounds of formula I are selected from compounds of formula A.6 to A.10

In another preferred embodiment compounds of formula I are selected from compounds of formula A.1 and A.6;

In another preferred embodiment compounds of formula I are selected from compounds of formula A.2 and A.7;

In another preferred embodiment compounds of formula I are selected from compounds of formula A.3 and A.8;

In another preferred embodiment compounds of formula I are selected from compounds of formula A.4 and A.9;

In another preferred embodiment compounds of formula I are selected from compounds of formula A.5 and A.10;

In another preferred embodiment compounds of formula I is A.1;

In another preferred embodiment compounds of formula I is A.2;

In another preferred embodiment compounds of formula I is A.3;

In another preferred embodiment compounds of formula I is A.4;

In another preferred embodiment compounds of formula I is A.5;

In another preferred embodiment compounds of formula I is A.6;

In another preferred embodiment compounds of formula I is A.7;

In another preferred embodiment compounds of formula I is A.8;

In another preferred embodiment compounds of formula I is A.9;

In another preferred embodiment compounds of formula I is A.10;

In another preferred embodiment compounds of formula I are selected from compounds of formula A.1 to A.5, wherein
Q is —C(═O)—N(R⁵)—, or —N(R⁵)—C(═O)—, preferably —C(═O)—N(R⁵)—;
R⁵ is H, C₁-C₆-alkyl, or C₁-C₆-alkyl-C₃-C₆-cycloalkyl, preferably H, CH₃, C₂H₅, or —CH₂-cyclopropyl, more preferably H or CH₃;
R¹ is H or C₁-C₆-alkyl, preferably H or CH₃;
R² is H or C₁-C₆-alkyl, preferably CH₃;
R^{B1}, R^{B2}, R^{B3}, and R^{B4} independently of each other are H, halogen, CN, C₁-C₆-alkyl, C₃-C₆-cycloalkyl, or C₁-C₆-alkoxy, wherein the alkyl, alkoxy, and cycloalkyl moieties are unsubstituted or substituted with halogen;
R³ is H or C₁-C₆-alkyl;
R⁴ is H or C₁-C₆-alkyl;
Ar¹ is selected from Ar¹-1 to Ar¹-17, preferably from Ar¹-1 to Ar¹-8, more preferably from Ar¹-1 to Ar¹-3;
Ar² is selected from Ar²-1 to Ar²-13, preferably from Ar²-1 to Ar²-3;

In another preferred embodiment compounds of formula I are selected from compounds of formula A.6 to A.10, wherein
Q is —C(═O)—N(R⁵)—, or —N(R⁵)—C(═O)—, preferably —C(═O)—N(R⁵)—;
R⁵ is H, C₁-C₆-alkyl, or C₁-C₆-alkyl-C₃-C₆-cycloalkyl, preferably H, CH₃, C₂H₅, or —CH₂-cyclopropyl, more preferably H or CH₃;
R¹ is H or C₁-C₆-alkyl, preferably H or CH₃;
R² is H or C₁-C₆-alkyl, preferably CH₃;
R^{B1}, R^{B2}, R^{B3}, and R^{B4} independently of each other are H, halogen, CN, C₁-C₆-alkyl, C₃-C₆-cycloalkyl, or C₁-C₆-alkoxy, wherein the alkyl, alkoxy, and cycloalkyl moieties are unsubstituted or substituted with halogen;
Ar¹ is selected from Ar¹-1 to Ar¹-17, preferably from Ar¹-1 to Ar¹-8, more preferably from Ar¹-1 to Ar¹-3;
Ar² is selected from Ar²-1 to Ar²-13, preferably from Ar²-1 to Ar²-3;

In another preferred embodiment compounds of formula I are selected from compounds of formula A.1 and A.6, Wherein
Q is —C(═O)—N(R⁵)—, or —N(R⁵)—C(═O)—, preferably —C(═O)—N(R⁵)—;
R⁵ is H, C₁-C₆-alkyl, or C₁-C₆-alkyl-C₃-C₆-cycloalkyl, preferably H, CH₃, C₂H₅, or —CH₂-cyclopropyl, more preferably H or CH₃;
R¹ is H or C₁-C₆-alkyl, preferably H or CH₃;
R² is H or C₁-C₆-alkyl, preferably CH₃;
R^{B1}, R^{B2}, R^{B3}, and R^{B4} independently of each other are H, halogen, CN, C₁-C₆-alkyl, C₃-C₆-cycloalkyl, or C₁-C₆-alkoxy, wherein the alkyl, alkoxy, and cycloalkyl moieties are unsubstituted or substituted with halogen;
R³ is H or C₁-C₆-alkyl;
R⁴ is H or C₁-C₆-alkyl;
Ar¹ is selected from Ar¹-1 to Ar¹-17, preferably from Ar¹-1 to Ar¹-8, more preferably from Ar¹-1 to Ar¹-3;
Ar² is selected from Ar²-1 to Ar²-13, preferably from Ar²-1 to Ar²-3;

In another preferred embodiment compounds of formula I are selected from compounds of formula A.2 and A.7, wherein
Q is —C(═O)—N(R⁵)—, or —N(R⁵)—C(═O)—, preferably —C(═O)—N(R⁵)—;
R⁵ is H, C₁-C₆-alkyl, or C₁-C₆-alkyl-C₃-C₆-cycloalkyl, preferably H, CH₃, C₂H₅, or —CH₂-cyclopropyl, more preferably H or CH₃;
R¹ is H or C₁-C₆-alkyl, preferably H or CH₃;
R² is H or C₁-C₆-alkyl, preferably CH₃;
R^{B1}, R^{B2}, R^{B3}, and R^{B4} independently of each other are H, halogen, CN, C₁-C₆-alkyl, C₃-C₆-cycloalkyl, or C₁-C₆-alkoxy, wherein the alkyl, alkoxy, and cycloalkyl moieties are unsubstituted or substituted with halogen;
R³ is H or C₁-C₆-alkyl;
R⁴ is H or C₁-C₆-alkyl;
Ar¹ is selected from Ar¹-1 to Ar¹-17, preferably from Ar¹-1 to Ar¹-8, more preferably from Ar¹-1 to Ar¹-3;
Ar² is selected from Ar²-1 to Ar²-13, preferably from Ar²-1 to Ar²-3;

In another preferred embodiment compounds of formula I are selected from compounds of formula A.3 and A.8, wherein
Q is —C(═O)—N(R⁵)—, or —N(R⁵)—C(═O)—, preferably —C(═O)—N(R⁵)—;
R⁵ is H, C₁-C₆-alkyl, or C₁-C₆-alkyl-C₃-C₆-cycloalkyl, preferably H, CH₃, C₂H₅, or —CH₂-cyclopropyl, more preferably H or CH₃;
R¹ is H or C₁-C₆-alkyl, preferably H or CH₃;
R² is H or C₁-C₆-alkyl, preferably CH₃;
R^{B1}, R^{B2}, R^{B3}, and R^{B4} independently of each other are H, halogen, CN, C₁-C₆-alkyl, C₃-C₆-cycloalkyl, or C₁-C₆-alkoxy, wherein the alkyl, alkoxy, and cycloalkyl moieties are unsubstituted or substituted with halogen;
R³ is H or C₁-C₆-alkyl;
R⁴ is H or C₁-C₆-alkyl;
Ar¹ is selected from Ar¹-1 to Ar¹-17, preferably from Ar¹-1 to Ar¹-8, more preferably from Ar¹-1 to Ar¹-3;
Ar² is selected from Ar²-1 to Ar²-13, preferably from Ar²-1 to Ar²-3;

In another preferred embodiment compounds of formula I are selected from compounds of formula A.4 and A.9, wherein
Q is —C(═O)—N(R⁵)—, or —N(R⁵)—C(═O)—, preferably —C(═O)—N(R⁵)—;
R⁵ is H, C₁-C₆-alkyl, or C₁-C₆-alkyl-C₃-C₆-cycloalkyl, preferably H, CH₃, C₂H₅, or —CH₂-cyclopropyl, more preferably H or CH₃;
R¹ is H or C₁-C₆-alkyl, preferably H or CH₃;
R² is H or C₁-C₆-alkyl, preferably CH₃;
R^{B1}, R^{B2}, R^{B3}, and R^{B4} independently of each other are H, halogen, CN, C₁-C₆-alkyl, C₃-C₆-cycloalkyl, or C₁-C₆-alkoxy, wherein the alkyl, alkoxy, and cycloalkyl moieties are unsubstituted or substituted with halogen;
R³ is H or C₁-C₆-alkyl;
R⁴ is H or C₁-C₆-alkyl;
Ar¹ is selected from Ar¹-1 to Ar¹-17, preferably from Ar¹-1 to Ar¹-8, more preferably from Ar¹-1 to Ar¹-3;
Ar² is selected from Ar²-1 to Ar²-13, preferably from Ar²-1 to Ar²-3;

In another preferred embodiment compounds of formula I are selected from compounds of formula A.5 and A.10, wherein
Q is —C(═O)—N(R⁵)—, or —N(R⁵)—C(═O)—, preferably —C(═O)—N(R⁵)—;
R⁵ is H, C₁-C₆-alkyl, or C₁-C₆-alkyl-C₃-C₆-cycloalkyl, preferably H, CH₃, C₂H₅, or —CH₂-cyclopropyl, more preferably H or CH₃;
R¹ is H or C₁-C₆-alkyl, preferably H or CH₃;
R² is H or C₁-C₆-alkyl, preferably CH₃;
R^{B1}, R^{B2}, R^{B3}, and R^{B4} independently of each other are H, halogen, CN, C₁-C₆-alkyl, C₃-C₆-cycloalkyl, or C₁-C₆-alkoxy, wherein the alkyl, alkoxy, and cycloalkyl moieties are unsubstituted or substituted with halogen;
R³ is H or C₁-C₆-alkyl;
R⁴ is H or C₁-C₆-alkyl;
Ar¹ is selected from Ar¹-1 to Ar¹-17, preferably from Ar¹-1 to Ar¹-8, more preferably from Ar¹-1 to Ar¹-3;
Ar² is selected from Ar²-1 to Ar²-13, preferably from Ar²-1 to Ar²-3;

In another preferred embodiment compounds of formula I is A.1, wherein
Q is —C(═O)—N(R⁵)—, or —N(R⁵)—C(═O)—, preferably —C(═O)—N(R⁵)—;
R⁵ is H, C₁-C₆-alkyl, or C₁-C₆-alkyl-C₃-C₆-cycloalkyl, preferably H, CH₃, C₂H₅, or —CH₂-cyclopropyl, more preferably H or CH₃;
R¹ is H or C₁-C₆-alkyl, preferably H or CH₃;
R² is H or C₁-C₆-alkyl, preferably CH₃;
R^{B1}, R^{B2}, R^{B3}, and R^{B4} independently of each other are H, halogen, CN, C₁-C₆-alkyl, C₃-C₆-cycloalkyl, or C₁-C₆-alkoxy, wherein the alkyl, alkoxy, and cycloalkyl moieties are unsubstituted or substituted with halogen;
R³ is H or C₁-C₆-alkyl;
R⁴ is H or C₁-C₆-alkyl;
Ar¹ is selected from Ar¹-1 to Ar¹-17, preferably from Ar¹-1 to Ar¹-8, more preferably from Ar¹-1 to Ar¹-3;
Ar² is selected from Ar²-1 to Ar²-13, preferably from Ar²-1 to Ar²-3;

In another preferred embodiment compounds of formula I is A.2, wherein
Q is —C(═O)—N(R⁵)—, or —N(R⁵)—C(═O)—, preferably —C(═O)—N(R⁵)—;
R⁵ is H, C₁-C₆-alkyl, or C₁-C₆-alkyl-C₃-C₆-cycloalkyl, preferably H, CH₃, C₂H₅, or —CH₂-cyclopropyl, more preferably H or CH₃;
R¹ is H or C₁-C₆-alkyl, preferably H or CH₃;
R² is H or C₁-C₆-alkyl, preferably CH₃;
R^{B1}, R^{B2}, R^{B3}, and R^{B4} independently of each other are H, halogen, CN, C₁-C₆-alkyl, C₃-C₆-cycloalkyl, or C₁-C₆-alkoxy, wherein the alkyl, alkoxy, and cycloalkyl moieties are unsubstituted or substituted with halogen;
R³ is H or C₁-C₆-alkyl;
R⁴ is H or C₁-C₆-alkyl;
Ar¹ is selected from Ar¹-1 to Ar¹-17, preferably from Ar¹-1 to Ar¹-8, more preferably from Ar¹-1 to Ar¹-3;
Ar² is selected from Ar²-1 to Ar²-13, preferably from Ar²-1 to Ar²-3;

In another preferred embodiment compounds of formula I is A.3, wherein
Q is —C(═O)—N(R⁵)—, or —N(R⁵)—C(═O)—, preferably —C(═O)—N(R⁵)—;
R⁵ is H, C₁-C₆-alkyl, or C₁-C₆-alkyl-C₃-C₆-cycloalkyl, preferably H, CH₃, C₂H₅, or —CH₂-cyclopropyl, more preferably H or CH₃;
R¹ is H or C₁-C₆-alkyl, preferably H or CH₃;
R² is H or C₁-C₆-alkyl, preferably CH₃;
R^{B1}, R^{B2}, R^{B3}, and R^{B4} independently of each other are H, halogen, CN, C₁-C₆-alkyl, C₃-C₆-cycloalkyl, or C₁-C₆-alkoxy, wherein the alkyl, alkoxy, and cycloalkyl moieties are unsubstituted or substituted with halogen;
R³ is H or C₁-C₆-alkyl;
R⁴ is H or C₁-C₆-alkyl;
Ar¹ is selected from Ar¹-1 to Ar¹-17, preferably from Ar¹-1 to Ar¹-8, more preferably from Ar¹-1 to Ar¹-3;
Ar² is selected from Ar²-1 to Ar²-13, preferably from Ar²-1 to Ar²-3;

In another preferred embodiment compounds of formula I is A.4, wherein
Q is —C(═O)—N(R⁵)—, or —N(R⁵)—C(═O)—, preferably —C(═O)—N(R⁵)—;
R⁵ is H, C₁-C₆-alkyl, or C₁-C₆-alkyl-C₃-C₆-cycloalkyl, preferably H, CH₃, C₂H₅, or —CH₂-cyclopropyl, more preferably H or CH₃;
R¹ is H or C₁-C₆-alkyl, preferably H or CH₃;
R² is H or C₁-C₆-alkyl, preferably CH₃;
R^{B1}, R^{B2}, R^{B3}, and R^{B4} independently of each other are H, halogen, CN, C₁-C₆-alkyl, C₃-C₆-cycloalkyl, or C₁-C₆-alkoxy, wherein the alkyl, alkoxy, and cycloalkyl moieties are unsubstituted or substituted with halogen;
R³ is H or C₁-C₆-alkyl;
R⁴ is H or C₁-C₆-alkyl;
Ar¹ is selected from Ar¹-1 to Ar¹-17, preferably from Ar¹-1 to Ar¹-8, more preferably from Ar¹-1 to Ar¹-3;
Ar² is selected from Ar²-1 to Ar²-13, preferably from Ar²-1 to Ar²-3;

In another preferred embodiment compounds of formula I is A.5, wherein
Q is —C(═O)—N(R⁵)—, or —N(R⁵)—C(═O)—, preferably —C(═O)—N(R⁵)—;
R⁵ is H, C₁-C₆-alkyl, or C₁-C₆-alkyl-C₃-C₆-cycloalkyl, preferably H, CH₃, C₂H₅, or —CH₂-cyclopropyl, more preferably H or CH₃;
R¹ is H or C₁-C₆-alkyl, preferably H or CH₃;
R² is H or C₁-C₆-alkyl, preferably CH₃;
R^{B1}, R^{B2}, R^{B3}, and R^{B4} independently of each other are H, halogen, CN, C₁-C₆-alkyl, C₃-C₆-cycloalkyl, or C₁-C₆-alkoxy, wherein the alkyl, alkoxy, and cycloalkyl moieties are unsubstituted or substituted with halogen;
R³ is H or C₁-C₆-alkyl;
R⁴ is H or C₁-C₆-alkyl;
Ar¹ is selected from Ar¹-1 to Ar¹-17, preferably from Ar¹-1 to Ar¹-8, more preferably from Ar¹-1 to Ar¹-3;
Ar² is selected from Ar²-1 to Ar²-13, preferably from Ar²-1 to Ar²-3;

In another preferred embodiment compounds of formula I is A.6, wherein
Q is —C(═O)—N(R⁵)—, or —N(R⁵)—C(═O)—, preferably —C(═O)—N(R⁵)—;
R⁵ is H, C₁-C₆-alkyl, or C₁-C₆-alkyl-C₃-C₆-cycloalkyl, preferably H, CH₃, C₂H₅, or —CH₂-cyclopropyl, more preferably H or CH₃;
R¹ is H or C₁-C₆-alkyl, preferably H or CH₃;
R² is H or C₁-C₆-alkyl, preferably CH₃;
R^{B1}, R^{B2}, R^{B3}, and R^{B4} independently of each other are H, halogen, CN, C₁-C₆-alkyl, C₃-C₆-cycloalkyl, or C₁-C₆-alkoxy, wherein the alkyl, alkoxy, and cycloalkyl moieties are unsubstituted or substituted with halogen;
Ar¹ is selected from Ar¹-1 to Ar¹-17, preferably from Ar¹-1 to Ar¹-8, more preferably from Ar¹-1 to Ar¹-3;
Ar² is selected from Ar²-1 to Ar²-13, preferably from Ar²-1 to Ar²-3;

In another preferred embodiment compounds of formula I is A.7, wherein
Q is —C(═O)—N(R⁵)—, or —N(R⁵)—C(═O)—, preferably —C(═O)—N(R⁵)—;
R⁵ is H, C₁-C₆-alkyl, or C₁-C₆-alkyl-C₃-C₆-cycloalkyl, preferably H, CH₃, C₂H₅, or —CH₂-cyclopropyl, more preferably H or CH₃;
R¹ is H or C₁-C₆-alkyl, preferably H or CH₃;
R² is H or C₁-C₆-alkyl, preferably CH₃;
R^{B1}, R^{B2}, R^{B3}, and R^{B4} independently of each other are H, halogen, CN, C₁-C₆-alkyl, C₃-C₆-cycloalkyl, or C₁-C₆-alkoxy, wherein the alkyl, alkoxy, and cycloalkyl moieties are unsubstituted or substituted with halogen;
Ar¹ is selected from Ar¹-1 to Ar¹-17, preferably from Ar¹-1 to Ar¹-8, more preferably from Ar¹-1 to Ar¹-3;
Ar² is selected from Ar²-1 to Ar²-13, preferably from Ar²-1 to Ar²-3;

In another preferred embodiment compounds of formula I is A.8, wherein
Q is —C(═O)—N(R⁵)—, or —N(R⁵)—C(═O)—, preferably —C(═O)—N(R⁵)—;
R⁵ is H, C₁-C₆-alkyl, or C₁-C₆-alkyl-C₃-C₆-cycloalkyl, preferably H, CH₃, C₂H₅, or —CH₂-cyclopropyl, more preferably H or CH₃;
R¹ is H or C₁-C₆-alkyl, preferably H or CH₃;
R² is H or C₁-C₆-alkyl, preferably CH₃;
R^{B1}, R^{B2}, R^{B3}, and R^{B4} independently of each other are H, halogen, CN, C₁-C₆-alkyl, C₃-C₆-cycloalkyl, or C₁-C₆-alkoxy, wherein the alkyl, alkoxy, and cycloalkyl moieties are unsubstituted or substituted with halogen;
Ar¹ is selected from Ar¹-1 to Ar¹-17, preferably from Ar¹-1 to Ar¹-8, more preferably from Ar¹-1 to Ar¹-3;
Ar² is selected from Ar²-1 to Ar²-13, preferably from Ar²-1 to Ar²-3;

In another preferred embodiment compounds of formula I is A.9, wherein
Q is —C(═O)—N(R⁵)—, or —N(R⁵)—C(═O)—, preferably —C(═O)—N(R⁵)—;
R⁵ is H, C₁-C₆-alkyl, or C₁-C₆-alkyl-C₃-C₆-cycloalkyl, preferably H, CH₃, C₂H₅, or —CH₂-cyclopropyl, more preferably H or CH₃;
R¹ is H or C₁-C₆-alkyl, preferably H or CH₃;
R² is H or C₁-C₆-alkyl, preferably CH₃;
R^{B1}, R^{B2}, R^{B3}, and R^{B4} independently of each other are H, halogen, CN, C₁-C₆-alkyl, C₃-C₆-cycloalkyl, or C₁-C₆-alkoxy, wherein the alkyl, alkoxy, and cycloalkyl moieties are unsubstituted or substituted with halogen;
Ar¹ is selected from Ar¹-1 to Ar¹-17, preferably from Ar¹-1 to Ar¹-8, more preferably from Ar¹-1 to Ar¹-3;
Ar² is selected from Ar²-1 to Ar²-13, preferably from Ar²-1 to Ar²-3;

In another preferred embodiment compounds of formula I is A.10, wherein
Q is —C(═O)—N(R⁵)—, or —N(R⁵)—C(═O)—, preferably —C(═O)—N(R⁵)—;
R⁵ is H, C₁-C₆-alkyl, or C₁-C₆-alkyl-C₃-C₆-cycloalkyl, preferably H, CH₃, C₂H₅, or —CH₂-cyclopropyl, more preferably H or CH₃;
R¹ is H or C₁-C₆-alkyl, preferably H or CH₃;
R² is H or C₁-C₆-alkyl, preferably CH₃;
R^{B1}, R^{B2}, R^{B3}, and R^{B4} independently of each other are H, halogen, CN, C₁-C₆-alkyl, C₃-C₆-cycloalkyl, or C₁-C₆-alkoxy, wherein the alkyl, alkoxy, and cycloalkyl moieties are unsubstituted or substituted with halogen;
Ar¹ is selected from Ar¹-1 to Ar¹-17, preferably from Ar¹-1 to Ar¹-8, more preferably from Ar¹-1 to Ar¹-3;
Ar² is selected from Ar²-1 to Ar²-13, preferably from Ar²-1 to Ar²-3;

In another preferred embodiment, the compound of formula I is selected from the compounds of formulae I.1 to I.4,

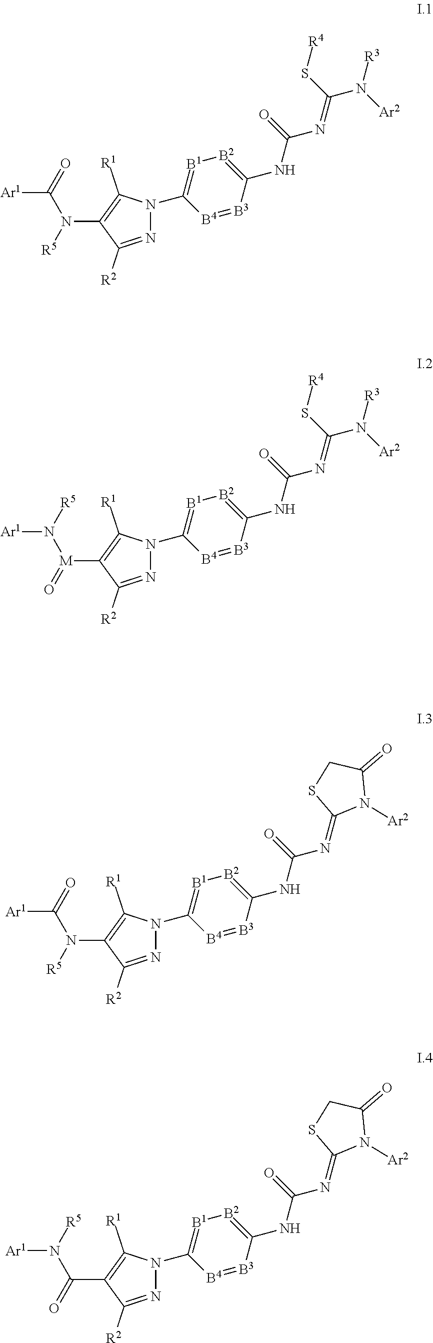

In another preferred embodiment, the compound of formula I is selected from the compounds of formulae I.1 and I.2;

In another preferred embodiment, the compound of formula I is selected from the compounds of formulae I.1 and I.3;

In another preferred embodiment, the compound of formula I is selected from the compounds of formulae I.3 and I.4;

In another preferred embodiment, the compound of formula I is selected from the compounds of formulae I.2 and I.4;

In another preferred embodiment, the compound of formula I is compound of formula I.1;

In another preferred embodiment, the compound of formula I is compound of formula I.2;

In another preferred embodiment, the compound of formula I is compound of formula I.3;

In another preferred embodiment, the compound of formula I is compound of formula I.4;

In another preferred embodiment, the compound of formula I is selected from the compounds of formulae I.1 to I.4, wherein
B¹ is N or CR^{B1}, B² is CR^{B2}, and B³ is CR^{B3};
R^{B1}, R^{B2}, R^{B3}, and R^{B4} independently of each other are H, halogen, CN, C₁-C₆-alkyl, or C₁-C₆-alkoxy, wherein the alkyl, alkoxy, and cycloalkyl moieties are unsubstituted or substituted with halogen;
R⁵ is H, C₁-C₆-alkyl, or C₁-C₆-alkyl-C₃-C₆-cycloalkyl;
R¹ is H, halogen, C₁-C₆-alkyl, or C₃-C₆-cycloalkyl;
R² is C₁-C₆-alkyl;
R³ is H or C₁-C₆-alkyl;
R⁴ is H or C₁-C₆-alkyl;
Ar¹ is phenyl, pyrimidinyl, pyridazinyl, thiophenyl, thiazolyl, or pyridyl, which are unsubstituted or substituted with R^{Ar1};
R^{Ar1} is halogen, NO₂, CN, C₁-C₆-alkyl, C₁-C₆-alkoxy, C₃-C₆-cycloalkyl, C₃-C₆-heterocyclyl, C₂-C₆-alkenyl, C₂-C₆-alkynyl wherein the alkyl, alkoxy, alkenyl, alkynyl, cycloalkyl, and C₃-C₆-heterocyclyl moieties are unsubstituted or substituted with R^{f};
NR^{b}R^{c}, C(═O)—NR^{b}R^{c}, C(═O)—R^{d}, NHS(═O)ₘR^{e}, —N═S(═O)—(C₁-C₆-alkyl)₂, or S(═O)ₘR^{e};
Ar² is phenyl which is unsubstituted or substituted with R^{Ar2};
R^{Ar2} is halogen, CN, C₁-C₆-alkyl, C₁-C₆-alkoxy, C₃-C₆-cycloalkyl, wherein the alkyl, alkoxy, and cycloalkyl moieties are unsubstituted or substituted with halogen;
or NR^{b}R^{c};
R^{a}, R^{b} and R^{c} identical or different, are H, C₁-C₆-alkyl, which are unsubstituted or substituted with halogen;
R^{d} is H or C₁-C₆-alkyl;
R^{e} is C₁-C₆-alkyl or C₁-C₆-haloalkyl;
R^{f} is halogen, OH, CN, C₁-C₆-alkyl, C₁-C₆-alkoxy, C₂-C₆-alkenyl, C₂-C₆-alkynyl, C₃-C₆-cycloalkyl, C₃-C₆-cycloalkoxy, which are unsubstituted or substituted with halogen;
m is 0, 1, or 2.

In another preferred embodiment, the compound of formula I is selected from the compounds of formulae I.1 to I.4, wherein
R⁵ is H, C₁-C₆-alkyl, or C₁-C₆-alkyl-C₃-C₆-cycloalkyl, preferably H, CH₃, C₂H₅, or —CH₂-cyclopropyl, more preferably H or CH₃;
R¹ is H, halogen, C₁-C₆-alkyl, or C₃-C₆-cycloalkyl, preferably H or CH₃;
R² is H or C₁-C₆-alkyl, preferably CH₃;
B¹ is N or CR^{B1};
B² is N or CR^{B2};
B³ is N or CR^{B3};
B⁴ is CR^{B4};
R^{B1}, R^{B2}, R^{B3}, and R^{B4} independently of each other are H, halogen, CN, C₁-C₆-alkyl, C₃-C₆-cycloalkyl, or C₁-C₆-alkoxy, wherein the alkyl, alkoxy, and cycloalkyl moieties are unsubstituted or substituted with halogen;
R³ is H or C₁-C₆-alkyl;
R⁴ is H or C₁-C₆-alkyl;
Ar¹ is selected from Ar¹-1 to Ar¹-17, preferably from Ar¹-1 to Ar¹-8, more preferably from Ar¹-1 to Ar¹-3;
Ar² is selected from Ar²-1 to Ar²-13, preferably from Ar²-1 to Ar²-3;

In another preferred embodiment, the compound of formula I is selected from the compounds of formulae I.1 to I.4, wherein
R⁵ is H, C₁-C₆-alkyl, or C₁-C₆-alkyl-C₃-C₆-cycloalkyl, preferably H, CH₃, C₂H₅, or —CH₂-cyclopropyl, more preferably H or CH₃;
R¹ is H or C₁-C₆-alkyl, preferably H or CH₃;
R² is H or C₁-C₆-alkyl, preferably CH₃;
B¹ is N or CR^{B1};
B² is N or CR^{B2};
B³ is N or CR^{B3};
B⁴ is CR^{B4};
R^{B1}, R^{B2}, R^{B3}, and R^{B4} independently of each other are H, halogen, CN, C₁-C₆-alkyl, C₃-C₆-cycloalkyl, or C₁-C₆-alkoxy, wherein the alkyl, alkoxy, and cycloalkyl moieties are unsubstituted or substituted with halogen;
R³ is H or C₁-C₆-alkyl;
R⁴ is H or C₁-C₆-alkyl;
Ar¹ is selected from Ar¹-1 to Ar¹-17, preferably from Ar¹-1 to Ar¹-8, more preferably from Ar¹-1 to Ar¹-3;
Ar² is selected from Ar²-1 to Ar²-13, preferably from Ar²-1 to Ar²-3;

In another preferred embodiment, the compound of formula I is selected from the compounds of formulae I.1 and I.2, wherein
R⁵ is H, C₁-C₆-alkyl, or C₁-C₆-alkyl-C₃-C₆-cycloalkyl, preferably H, CH₃, C₂H₅, or —CH₂-cyclopropyl, more preferably H or CH₃;
R¹ is H or C₁-C₆-alkyl, preferably H or CH₃;
R² is H or C₁-C₆-alkyl, preferably CH₃;
B¹ is N or CR^{B1};
B² is N or CR^{B2};
B³ is N or CR^{B3};
B⁴ is CR^{B4};
R^{B1}, R^{B2}, R^{B3}, and R^{B4} independently of each other are H, halogen, CN, C₁-C₆-alkyl, C₃-C₆-cycloalkyl, or C₁-C₆-alkoxy, wherein the alkyl, alkoxy, and cycloalkyl moieties are unsubstituted or substituted with halogen;
R³ is H or C₁-C₆-alkyl;
R⁴ is H or C₁-C₆-alkyl;
Ar¹ is selected from Ar¹-1 to Ar¹-17, preferably from Ar¹-1 to Ar¹-8, more preferably from Ar¹-1 to Ar¹-3;
Ar² is selected from Ar²-1 to Ar²-13, preferably from Ar²-1 to Ar²-3;

In another preferred embodiment, the compound of formula I is selected from the compounds of formulae I.1 and I.3, wherein
R⁵ is H, C₁-C₆-alkyl, or C₁-C₆-alkyl-C₃-C₆-cycloalkyl, preferably H, CH₃, C₂H₅, or —CH₂-cyclopropyl, more preferably H or CH₃;
R¹ is H or C₁-C₆-alkyl, preferably H or CH₃;
R² is H or C₁-C₆-alkyl, preferably CH₃;
B¹ is N or CR^{B1};
B² is N or CR^{B2};
B³ is N or CR^{B3};
B⁴ is CR^{B4};
R^{B1}, R^{B2}, R^{B3}, and R^{B4} independently of each other are H, halogen, CN, C₁-C₆-alkyl, C₃-C₆-cycloalkyl, or C₁-C₆-alkoxy, wherein the alkyl, alkoxy, and cycloalkyl moieties are unsubstituted or substituted with halogen;
R³ is H or C₁-C₆-alkyl;
R⁴ is H or C₁-C₆-alkyl;
Ar¹ is selected from Ar¹-1 to Ar¹-17, preferably from Ar¹-1 to Ar¹-8, more preferably from Ar¹-1 to Ar¹-3;
Ar² is selected from Ar²-1 to Ar²-13, preferably from Ar²-1 to Ar²-3;

In another preferred embodiment, the compound of formula I is selected from the compounds of formulae I.3 and I.4, wherein
R⁵ is H, C₁-C₆-alkyl, or C₁-C₆-alkyl-C₃-C₆-cycloalkyl, preferably H, CH₃, C₂H₅, or —CH₂-cyclopropyl, more preferably H or CH₃;
R¹ is H or C₁-C₆-alkyl, preferably H or CH₃;
R² is H or C₁-C₆-alkyl, preferably CH₃;
B¹ is N or CR^{B1};
B² is N or CR^{B2};
B³ is N or CR^{B3};
B⁴ is CR^{B4};
R^{B1}, R^{B2}, R^{B3}, and R^{B4} independently of each other are H, halogen, CN, C₁-C₆-alkyl, C₃-C₆-cycloalkyl, or C₁-C₆-alkoxy, wherein the alkyl, alkoxy, and cycloalkyl moieties are unsubstituted or substituted with halogen;
Ar¹ is selected from Ar¹-1 to Ar¹-17, preferably from Ar¹-1 to Ar¹-8, more preferably from Ar¹-1 to Ar¹-3;
Ar² is selected from Ar²-1 to Ar²-13, preferably from Ar²-1 to Ar²-3;

In another preferred embodiment, the compound of formula I is selected from the compounds of formulae I.2 and I.4, wherein
R⁵ is H, C₁-C₆-alkyl, or C₁-C₆-alkyl-C₃-C₆-cycloalkyl, preferably H, CH₃, C₂H₅, or —CH₂-cyclopropyl, more preferably H or CH₃;
R¹ is H or C₁-C₆-alkyl, preferably H or CH₃;
R² is H or C₁-C₆-alkyl, preferably CH₃;
B¹ is N or CR^{B1};
B² is N or CR^{B2};
B³ is N or CR^{B3};
B⁴ is CR^{B4};
R^{B1}, R^{B2}, R^{B3}, and R^{B4} independently of each other are H, halogen, CN, C₁-C₆-alkyl, C₃-C₆-cycloalkyl, or C₁-C₆-alkoxy, wherein the alkyl, alkoxy, and cycloalkyl moieties are unsubstituted or substituted with halogen;
R³ is H or C₁-C₆-alkyl;
R⁴ is H or C₁-C₆-alkyl;
Ar¹ is selected from Ar¹-1 to Ar¹-17, preferably from Ar¹-1 to Ar¹-8, more preferably from Ar¹-1 to Ar¹-3;
Ar² is selected from Ar²-1 to Ar²-13, preferably from Ar²-1 to Ar²-3;

In another preferred embodiment, the compound of formula I is compound of formula I.1, wherein
R⁵ is H, C₁-C₆-alkyl, or C₁-C₆-alkyl-C₃-C₆-cycloalkyl, preferably H, CH₃, C₂H₅, or —CH₂-cyclopropyl, more preferably H or CH₃;
R¹ is H or C₁-C₆-alkyl, preferably H or CH₃;
R² is H or C₁-C₆-alkyl, preferably CH₃;
B¹ is N or CR^{B1};
B² is N or CR^{B2};
B³ is N or CR^{B3};
B⁴ is CR^{B4};
R^{B1}, R^{B2}, R^{B3}, and R^{B4} independently of each other are H, halogen, CN, C₁-C₆-alkyl, C₃-C₆-cycloalkyl, or C₁-C₆-alkoxy, wherein the alkyl, alkoxy, and cycloalkyl moieties are unsubstituted or substituted with halogen;
R³ is H or C₁-C₆-alkyl;
R⁴ is H or C₁-C₆-alkyl;
Ar¹ is selected from Ar¹-1 to Ar¹-17, preferably from Ar¹-1 to Ar¹-8, more preferably from Ar¹-1 to Ar¹-3;
Ar² is selected from Ar²-1 to Ar²-13, preferably from Ar²-1 to Ar²-3;

In another preferred embodiment, the compound of formula I is compound of formula I.2, wherein
R⁵ is H, C₁-C₆-alkyl, or C₁-C₆-alkyl-C₃-C₆-cycloalkyl, preferably H, CH₃, C₂H₅, or —CH₂-cyclopropyl, more preferably H or CH₃;
R¹ is H or C₁-C₆-alkyl, preferably H or CH₃;
R² is H or C₁-C₆-alkyl, preferably CH₃;
B¹ is N or CR^{B1};
B² is N or CR^{B2};
B³ is N or CR^{B3};
B⁴ is CR^{B4};
R^{B1}, R^{B2}, R^{B3}, and R^{B4} independently of each other are H, halogen, CN, C₁-C₆-alkyl, C₃-C₆-cycloalkyl, or C₁-C₆-alkoxy, wherein the alkyl, alkoxy, and cycloalkyl moieties are unsubstituted or substituted with halogen;
R³ is H or C₁-C₆-alkyl;
R⁴ is H or C₁-C₆-alkyl;
Ar¹ is selected from Ar¹-1 to Ar¹-17, preferably from Ar¹-1 to Ar¹-8, more preferably from Ar¹-1 to Ar¹-3;
Ar² is selected from Ar²-1 to Ar²-13, preferably from Ar²-1 to Ar²-3;

In another preferred embodiment, the compound of formula I is compounds of formula I.3, wherein
B¹ is N or CR^{B1}, B² is CR^{B2}, and B³ is CR^{B3};
R^{B1}, R^{B2}, R^{B3}, and R^{B4} independently of each other are H, halogen, CN, C₁-C₆-alkyl, or C₁-C₆-alkoxy, wherein the alkyl, alkoxy, and cycloalkyl moieties are unsubstituted or substituted with halogen;
R⁵ is H, C₁-C₆-alkyl, or C₁-C₆-alkyl-C₃-C₆-cycloalkyl;
R¹ is H, halogen, C₁-C₆-alkyl, or C₃-C₆-cycloalkyl;
R² is C₁-C₆-alkyl;
Ar¹ is phenyl, pyrimidinyl, pyridazinyl, thiophenyl, thiazolyl, or pyridyl, which are unsubstituted or substituted with R^{Ar1};
R^{Ar1} is halogen, NO₂, CN, C₁-C₆-alkyl, C₁-C₆-alkoxy, C₃-C₆-cycloalkyl, C₃-C₆-heterocyclyl, C₂-C₆-alkenyl, C₂-C₆-alkynyl wherein the alkyl, alkoxy, alkenyl, alkynyl, cycloalkyl, and C₃-C₆-heterocyclyl moieties are unsubstituted or substituted with R^{f};
NR^{b}R^{c}, C(═O)—NR^{b}R^{c}, C(═O)—R^{d}, NHS(═O)ₘR^{e}, —N═S(═O)—(C₁-C₆-alkyl)₂, or S(═O)ₘR^{e};
Ar² is phenyl which is unsubstituted or substituted with R^{Ar2};
R^{Ar2} is halogen, CN, C₁-C₆-alkyl, C₁-C₆-alkoxy, C₃-C₆-cycloalkyl, wherein the alkyl, alkoxy, and cycloalkyl moieties are unsubstituted or substituted with halogen;
or NR^{b}R^{c};
R^{a}, R^{b} and R^{c} identical or different, are H, C₁-C₆-alkyl, which are unsubstituted or substituted with halogen;
R^{d} is H or C₁-C₆-alkyl;
R^{e} is C₁-C₆-alkyl or C₁-C₆-haloalkyl;
R^{f} is halogen, OH, CN, C₁-C₆-alkyl, C₁-C₆-alkoxy, C₂-C₆-alkenyl, C₂-C₆-alkynyl, C₃-C₆-cycloalkyl, C₃-C₆-cycloalkoxy, which are unsubstituted or substituted with halogen;
m is 0, 1, or 2.

In another preferred embodiment, the compound of formula I is compound of formula I.3, wherein
R⁵ is H, C₁-C₆-alkyl, or C₁-C₆-alkyl-C₃-C₆-cycloalkyl, preferably H, CH₃, C₂H₅, or —CH₂-cyclopropyl, more preferably H or CH₃;
R¹ is H or C₁-C₆-alkyl, preferably H or CH₃;
R² is H or C₁-C₆-alkyl, preferably CH₃;
B¹ is N or CR^{B1};
B² is N or CR^{B2};
B³ is N or CR^{B3};
B⁴ is CR^{B4};
R^{B1}, R^{B2}, R^{B3}, and R^{B4} independently of each other are H, halogen, CN, C₁-C₆-alkyl, C₃-C₆-cycloalkyl, or C₁-C₆-alkoxy, wherein the alkyl, alkoxy, and cycloalkyl moieties are unsubstituted or substituted with halogen;
Ar¹ is selected from Ar¹-1 to Ar¹-17, preferably from Ar¹-1 to Ar¹-8, more preferably from Ar¹-1 to Ar¹-3;
Ar² is selected from Ar²-1 to Ar²-13, preferably from Ar²-1 to Ar²-3;

In another preferred embodiment, the compound of formula I is compound of formula I.4, wherein
R⁵ is H, C₁-C₆-alkyl, or C₁-C₆-alkyl-C₃-C₆-cycloalkyl, preferably H, CH₃, C₂H₅, or —CH₂-cyclopropyl, more preferably H or CH₃;
R¹ is H or C₁-C₆-alkyl, preferably H or CH₃;
R² is H or C₁-C₆-alkyl, preferably CH₃;
B¹ is N or CR^{B1};
B² is N or CR^{B2};
B³ is N or CR^{B3};
B⁴ is CR^{B4};
R^{B1}, R^{B2}, R^{B3}, and R^{B4} independently of each other are H, halogen, CN, C₁-C₆-alkyl, C₃-C₆-cycloalkyl, or C₁-C₆-alkoxy, wherein the alkyl, alkoxy, and cycloalkyl moieties are unsubstituted or substituted with halogen;
Ar¹ is selected from Ar¹-1 to Ar¹-17, preferably from Ar¹-1 to Ar¹-8, more preferably from Ar¹-1 to Ar¹-3;
Ar² is selected from Ar²-1 to Ar²-13, preferably from Ar²-1 to Ar²-3;

Particular compounds of formula I are the compounds of the formulae I. 1 to I.4 that are compiled in the following tables 1 to 48, wherein the combination of variables B¹, B², B³, and B⁴ for each compound of tables 1 to 48 corresponds to each line of Table B. Each of the groups mentioned for a substituent in the tables is furthermore per se, independently of the combination in which it is mentioned, a particularly preferred aspect of the substituent in question.
Table 1. Compounds of formula I.3 where R¹ is H, R² is H, R⁵ is H, Ar¹ is Ar¹-1, Ar² is Ar²-1.
Table 2. Compounds of formula I.3 where R¹ is H, R² is H, R⁵ is H, Ar¹ is Ar¹-1, Ar² is Ar²-2.
Table 3. Compounds of formula I.3 where R¹ is H, R² is H, R⁵ is H, Ar¹ is Ar¹-2, Ar² is Ar²-1.
Table 4. Compounds of formula I.3 where R¹ is H, R² is H, R⁵ is H, Ar¹ is Ar¹-2, Ar² is Ar²-2.
Table 5. Compounds of formula I.3 where R¹ is H, R² is H, R⁵ is H, Ar¹ is Ar¹-3, Ar² is Ar²-1.
Table 6. Compounds of formula I.3 where R¹ is H, R² is H, R⁵ is H, Ar¹ is Ar¹-3, Ar² is Ar²-2.
Table 7. Compounds of formula I.3 where R¹ is H, R² is H, R⁵ is CH₃, Ar¹ is Ar¹-1, Ar² is Ar²-1.
Table 8. Compounds of formula I.3 where R¹ is H, R² is H, R⁵ is CH₃, Ar¹ is Ar¹-1, Ar² is Ar²-2.
Table 9. Compounds of formula I.3 where R¹ is H, R² is H, R⁵ is CH₃, Ar¹ is Ar¹-2, Ar² is Ar²-1.
Table 10. Compounds of formula I.3 where R¹ is H, R² is H, R⁵ is CH₃, Ar¹ is Ar¹-2, Ar² is Ar²-2.
Table 11. Compounds of formula I.3 where R¹ is H, R² is H, R⁵ is CH₃, Ar¹ is Ar¹-3, Ar² is Ar²-1.
Table 12. Compounds of formula I.3 where R¹ is H, R² is H, R⁵ is CH₃, Ar¹ is Ar¹-3, Ar² is Ar²-2.
Table 13. Compounds of formula I.3 where R¹ is H, R² is CH₃, R⁵ is H, Ar¹ is Ar¹-1, Ar² is Ar²-1.
Table 14. Compounds of formula I.3 where R¹ is H, R² is CH₃, R⁵ is H, Ar¹ is Ar¹-1, Ar² is Ar²-2.
Table 15. Compounds of formula I.3 where R¹ is H, R² is CH₃, R⁵ is H, Ar¹ is Ar¹-2, Ar² is Ar²-1.
Table 16. Compounds of formula I.3 where R¹ is H, R² is CH₃, R⁵ is H, Ar¹ is Ar¹-2, Ar² is Ar²-2.
Table 17. Compounds of formula I.3 where R¹ is H, R² is CH₃, R⁵ is H, Ar¹ is Ar¹-3, Ar² is Ar²-1.
Table 18. Compounds of formula I.3 where R¹ is H, R² is CH₃, R⁵ is H, Ar¹ is Ar¹-3, Ar² is Ar²-2.
Table 19. Compounds of formula I.3 where R¹ is H, R² is CH₃, R⁵ is CH₃, Ar¹ is Ar¹-1, Ar² is Ar²-1.
Table 20. Compounds of formula I.3 where R¹ is H, R² is CH₃, R⁵ is CH₃, Ar¹ is Ar¹-1, Ar² is Ar²-2.
Table 21. Compounds of formula I.3 where R¹ is H, R² is CH₃, R⁵ is CH₃, Ar¹ is Ar¹-2, Ar² is Ar²-1.
Table 22. Compounds of formula I.3 where R¹ is H, R² is CH₃, R⁵ is CH₃, Ar¹ is Ar¹-2, Ar² is Ar²-2.
Table 23. Compounds of formula I.3 where R¹ is H, R² is CH₃, R⁵ is CH₃, Ar¹ is Ar¹-3, Ar² is Ar²-1.
Table 24. Compounds of formula I.3 where R¹ is H, R² is CH₃, R⁵ is CH₃, Ar¹ is Ar¹-3, Ar² is Ar²-2.
Table 25. Compounds of formula I.3 where R¹ is CH₃, R² is H, R⁵ is H, Ar¹ is Ar¹-1, Ar² is Ar²-1.
Table 26. Compounds of formula I.3 where R¹ is CH₃, R² is H, R⁵ is H, Ar¹ is Ar¹-1, Ar² is Ar²-2.
Table 27. Compounds of formula I.3 where R¹ is CH₃, R² is H, R⁵ is H, Ar¹ is Ar¹-2, Ar² is Ar²-1.
Table 28. Compounds of formula I.3 where R¹ is CH₃, R² is H, R⁵ is H, Ar¹ is Ar¹-2, Ar² is Ar²-2.
Table 29. Compounds of formula I.3 where R¹ is CH₃, R² is H, R⁵ is H, Ar¹ is Ar¹-3, Ar² is Ar²-1.
Table 30. Compounds of formula I.3 where R¹ is CH₃, R² is H, R⁵ is H, Ar¹ is Ar¹-3, Ar² is Ar²-2.
Table 31. Compounds of formula I.3 where R¹ is CH₃, R² is H, R⁵ is CH₃, Ar¹ is Ar¹-1, Ar² is Ar²-1.
Table 32. Compounds of formula I.3 where R¹ is CH₃, R² is H, R⁵ is CH₃, Ar¹ is Ar¹-1, Ar² is Ar²-2.
Table 33. Compounds of formula I.3 where R¹ is CH₃, R² is H, R⁵ is CH₃, Ar¹ is Ar¹-2, Ar² is Ar²-1.
Table 34. Compounds of formula I.3 where R¹ is CH₃, R² is H, R⁵ is CH₃, Ar¹ is Ar¹-2, Ar² is Ar²-2.
Table 35. Compounds of formula I.3 where R¹ is CH₃, R² is H, R⁵ is CH₃, Ar¹ is Ar¹-3, Ar² is Ar²-1.
Table 36. Compounds of formula I.3 where R¹ is CH₃, R² is H, R⁵ is CH₃, Ar¹ is Ar¹-3, Ar² is Ar²-2.
Table 37. Compounds of formula I.3 where R¹ is CH₃, R² is CH₃, R⁵ is H, Ar¹ is Ar¹-1, Ar² is Ar²-1.
Table 38. Compounds of formula I.3 where R¹ is CH₃, R² is CH₃, R⁵ is H, Ar¹ is Ar¹-1, Ar² is Ar²-2.
Table 39. Compounds of formula I.3 where R¹ is CH₃, R² is CH₃, R⁵ is H, Ar¹ is Ar¹-2, Ar² is Ar²-1.
Table 40. Compounds of formula I.3 where R¹ is CH₃, R² is CH₃, R⁵ is H, Ar¹ is Ar¹-2, Ar² is Ar²-2.
Table 41. Compounds of formula I.3 where R¹ is CH₃, R² is CH₃, R⁵ is H, Ar¹ is Ar¹-3, Ar² is Ar²-1.
Table 42. Compounds of formula I.3 where R¹ is CH₃, R² is CH₃, R⁵ is H, Ar¹ is Ar¹-3, Ar² is Ar²-2.
Table 43. Compounds of formula I.3 where R¹ is CH₃, R² is CH₃, R⁵ is CH₃, Ar¹ is Ar¹-1, Ar² is Ar²-1.
Table 44. Compounds of formula I.3 where R¹ is CH₃, R² is CH₃, R⁵ is CH₃, Ar¹ is Ar¹-1, Ar² is Ar²-2.
Table 45. Compounds of formula I.3 where R¹ is CH₃, R² is CH₃, R⁵ is CH₃, Ar¹ is Ar¹-2, Ar² is Ar²-1.
Table 46. Compounds of formula I.3 where R¹ is CH₃, R² is CH₃, R⁵ is CH₃, Ar¹ is Ar¹-2, Ar² is Ar²-2.
Table 47. Compounds of formula I.3 where R¹ is CH₃, R² is CH₃, R⁵ is CH₃, Ar¹ is Ar¹-3, Ar² is Ar²-1.
Table 48. Compounds of formula I.3 where R¹ is CH₃, R² is CH₃, R⁵ is CH₃, Ar¹ is Ar¹-3, Ar² is Ar²-2.

TABLE B

| Line | B¹ | B² | B³ | B⁴ |
|---|---|---|---|---|
| 1. | N | N | CH | CH |
| 2. | N | N | CH | C—Cl |
| 3. | N | N | CH | C—F |
| 4. | N | N | CH | C—Br |
| 5. | N | N | CH | C—CH₃ |
| 6. | N | N | C—Cl | CH |
| 7. | N | N | C—Cl | C—Cl |
| 8. | N | N | C—Cl | C—F |
| 9. | N | N | C—Cl | C—Br |
| 10. | N | N | C—Cl | C—CH₃ |
| 11. | N | N | C—F | CH |
| 12. | N | N | C—F | C—Cl |
| 13. | N | N | C—F | C—F |
| 14. | N | N | C—F | C—Br |
| 15. | N | N | C—F | C—CH₃ |
| 16. | N | N | C—Br | CH |
| 17. | N | N | C—Br | C—Cl |
| 18. | N | N | C—Br | C—F |
| 19. | N | N | C—Br | C—Br |
| 20. | N | N | C—Br | C—CH₃ |
| 21. | N | N | C—CH₃ | CH |
| 22. | N | N | C—CH₃ | C—Cl |
| 23. | N | N | C—CH₃ | C—F |
| 24. | N | N | C—CH₃ | C—Br |
| 25. | N | N | C—CH₃ | C—CH₃ |
| 26. | N | CH | N | CH |
| 27. | N | CH | N | C—Cl |
| 28. | N | CH | N | C—F |
| 29. | N | CH | N | C—Br |
| 30. | N | CH | N | C—CH₃ |
| 31. | N | CH | CH | CH |
| 32. | N | CH | CH | C—Cl |
| 33. | N | CH | CH | C—F |
| 34. | N | CH | CH | C—Br |
| 35. | N | CH | CH | C—CH₃ |
| 36. | N | CH | C—Cl | CH |
| 37. | N | CH | C—Cl | C—Cl |
| 38. | N | CH | C—Cl | C—F |
| 39. | N | CH | C—Cl | C—Br |
| 40. | N | CH | C—Cl | C—CH₃ |
| 41. | N | CH | C—F | CH |
| 42. | N | CH | C—F | C—Cl |
| 43. | N | CH | C—F | C—F |
| 44. | N | CH | C—F | C—Br |
| 45. | N | CH | C—F | C—CH₃ |
| 46. | N | CH | C—Br | CH |
| 47. | N | CH | C—Br | C—Cl |
| 48. | N | CH | C—Br | C—F |
| 49. | N | CH | C—Br | C—Br |
| 50. | N | CH | C—Br | C—CH₃ |
| 51. | N | CH | C—CH₃ | CH |
| 52. | N | CH | C—CH₃ | C—Cl |
| 53. | N | CH | C—CH₃ | C—F |
| 54. | N | CH | C—CH₃ | C—Br |
| 55. | N | CH | C—CH₃ | C—CH₃ |
| 56. | N | C—Cl | N | CH |
| 57. | N | C—Cl | N | C—Cl |
| 58. | N | C—Cl | N | C—F |
| 59. | N | C—Cl | N | C—Br |
| 60. | N | C—Cl | N | C—CH₃ |
| 61. | N | C—Cl | CH | CH |
| 62. | N | C—Cl | CH | C—Cl |
| 63. | N | C—Cl | CH | C—F |
| 64. | N | C—Cl | CH | C—Br |
| 65. | N | C—Cl | CH | C—CH₃ |
| 66. | N | C—Cl | C—Cl | CH |
| 67. | N | C—Cl | C—Cl | C—Cl |
| 68. | N | C—Cl | C—Cl | C—F |
| 69. | N | C—Cl | C—Cl | C—Br |
| 70. | N | C—Cl | C—Cl | C—CH₃ |
| 71. | N | C—Cl | C—F | CH |
| 72. | N | C—Cl | C—F | C—Cl |
| 73. | N | C—Cl | C—F | C—F |
| 74. | N | C—Cl | C—F | C—Br |
| 75. | N | C—Cl | C—F | C—CH₃ |
| 76. | N | C—Cl | C—Br | CH |
| 77. | N | C—Cl | C—Br | C—Cl |
| 78. | N | C—Cl | C—Br | C—F |
| 79. | N | C—Cl | C—Br | C—Br |
| 80. | N | C—Cl | C—Br | C—CH₃ |
| 81. | N | C—Cl | C—CH₃ | CH |
| 82. | N | C—Cl | C—CH₃ | C—Cl |
| 83. | N | C—Cl | C—CH₃ | C—F |
| 84. | N | C—Cl | C—CH₃ | C—Br |
| 85. | N | C—Cl | C—CH₃ | C—CH₃ |
| 86. | N | C—F | N | CH |
| 87. | N | C—F | N | C—Cl |
| 88. | N | C—F | N | C—F |
| 89. | N | C—F | N | C—Br |
| 90. | N | C—F | N | C—CH₃ |
| 91. | N | C—F | CH | CH |
| 92. | N | C—F | CH | C—Cl |
| 93. | N | C—F | CH | C—F |
| 94. | N | C—F | CH | C—Br |
| 95. | N | C—F | CH | C—CH₃ |
| 96. | N | C—F | C—Cl | CH |
| 97. | N | C—F | C—Cl | C—Cl |
| 98. | N | C—F | C—Cl | C—F |
| 99. | N | C—F | C—Cl | C—Br |
| 100. | N | C—F | C—Cl | C—CH₃ |
| 101. | N | C—F | C—F | CH |
| 102. | N | C—F | C—F | C—Cl |
| 103. | N | C—F | C—F | C—F |
| 104. | N | C—F | C—F | C—Br |
| 105. | N | C—F | C—F | C—CH₃ |
| 106. | N | C—F | C—Br | CH |
| 107. | N | C—F | C—Br | C—Cl |
| 108. | N | C—F | C—Br | C—F |
| 109. | N | C—F | C—Br | C—Br |
| 110. | N | C—F | C—Br | C—CH₃ |
| 111. | N | C—F | C—CH₃ | CH |
| 112. | N | C—F | C—CH₃ | C—Cl |
| 113. | N | C—F | C—CH₃ | C—F |
| 114. | N | C—F | C—CH₃ | C—Br |
| 115. | N | C—F | C—CH₃ | C—CH₃ |
| 116. | N | C—Br | N | CH |
| 117. | N | C—Br | N | C—Cl |
| 118. | N | C—Br | N | C—F |
| 119. | N | C—Br | N | C—Br |
| 120. | N | C—Br | N | C—CH₃ |
| 121. | N | C—Br | CH | CH |
| 122. | N | C—Br | CH | C—Cl |
| 123. | N | C—Br | CH | C—F |
| 124. | N | C—Br | CH | C—Br |
| 125. | N | C—Br | CH | C—CH₃ |
| 126. | N | C—Br | C—Cl | CH |
| 127. | N | C—Br | C—Cl | C—Cl |
| 128. | N | C—Br | C—Cl | C—F |
| 129. | N | C—Br | C—Cl | C—Br |
| 130. | N | C—Br | C—Cl | C—CH₃ |
| 131. | N | C—Br | C—F | CH |
| 132. | N | C—Br | C—F | C—Cl |
| 133. | N | C—Br | C—F | C—F |
| 134. | N | C—Br | C—F | C—Br |
| 135. | N | C—Br | C—F | C—CH₃ |
| 136. | N | C—Br | C—Br | CH |
| 137. | N | C—Br | C—Br | C—Cl |
| 138. | N | C—Br | C—Br | C—F |
| 139. | N | C—Br | C—Br | C—Br |
| 140. | N | C—Br | C—Br | C—CH₃ |
| 141. | N | C—Br | C—CH₃ | CH |
| 142. | N | C—Br | C—CH₃ | C—Cl |
| 143. | N | C—Br | C—CH₃ | C—F |
| 144. | N | C—Br | C—CH₃ | C—Br |
| 145. | N | C—Br | C—CH₃ | C—CH₃ |
| 146. | N | C—CH₃ | N | CH |
| 147. | N | C—CH₃ | N | C—Cl |
| 148. | N | C—CH₃ | N | C—F |
| 149. | N | C—CH₃ | N | C—Br |
| 150. | N | C—CH₃ | N | C—CH₃ |
| 151. | N | C—CH₃ | CH | CH |
| 152. | N | C—CH₃ | CH | C—Cl |
| 153. | N | C—CH₃ | CH | C—F |
| 154. | N | C—CH₃ | CH | C—Br |
| 155. | N | C—CH₃ | CH | C—CH₃ |
| 156. | N | C—CH₃ | C—Cl | CH |
| 157. | N | C—CH₃ | C—Cl | C—Cl |
| 158. | N | C—CH₃ | C—Cl | C—F |
| 159. | N | C—CH₃ | C—Cl | C—Br |
| 160. | N | C—CH₃ | C—Cl | C—CH₃ |
| 161. | N | C—CH₃ | C—F | CH |
| 162. | N | C—CH₃ | C—F | C—Cl |
| 163. | N | C—CH₃ | C—F | C—F |
| 164 | N | C—CH₃ | C—F | C—Br |
| 165. | N | C—CH₃ | C—F | C—CH₃ |
| 166. | N | C—CH₃ | C—Br | CH |
| 167. | N | C—CH₃ | C—Br | C—Cl |
| 168. | N | C—CH₃ | C—Br | C—F |
| 169. | N | C—CH₃ | C—Br | C—Br |
| 170. | N | C—CH₃ | C—Br | C—CH₃ |
| 171. | N | C—CH₃ | C—CH₃ | CH |
| 172. | N | C—CH₃ | C—CH₃ | C—Cl |
| 173. | N | C—CH₃ | C—CH₃ | C—F |
| 174. | N | C—CH₃ | C—CH₃ | C—Br |
| 175. | N | C—CH₃ | C—CH₃ | C—CH₃ |
| 176. | CH | N | N | CH |
| 177. | CH | N | N | C—Cl |
| 178. | CH | N | N | C—F |
| 179. | CH | N | N | C—Br |
| 180. | CH | N | N | C—CH₃ |
| 181. | CH | N | CH | CH |
| 182. | CH | N | CH | C—Cl |
| 183. | CH | N | CH | C—F |
| 184. | CH | N | CH | C—Br |
| 185. | CH | N | CH | C—CH₃ |
| 186. | CH | N | C—Cl | CH |
| 187. | CH | N | C—Cl | C—Cl |
| 188. | CH | N | C—Cl | C—F |
| 189. | CH | N | C—Cl | C—Br |
| 190. | CH | N | C—Cl | C—CH₃ |
| 191. | CH | N | C—F | CH |
| 192. | CH | N | C—F | C—Cl |
| 193. | CH | N | C—F | C—F |
| 194. | CH | N | C—F | C—Br |
| 195. | CH | N | C—F | C—CH₃ |
| 196. | CH | N | C—Br | CH |
| 197. | CH | N | C—Br | C—Cl |
| 198. | CH | N | C—Br | C—F |
| 199. | CH | N | C—Br | C—Br |
| 200. | CH | N | C—Br | C—CH₃ |
| 201. | CH | N | C—CH₃ | CH |
| 202. | CH | N | C—CH₃ | C—Cl |
| 203. | CH | N | C—CH₃ | C—F |
| 204. | CH | N | C—CH₃ | C—Br |
| 205. | CH | N | C—CH₃ | C—CH₃ |
| 206. | CH | CH | N | CH |
| 207. | CH | CH | N | C—Cl |
| 208. | CH | CH | N | C—F |
| 209. | CH | CH | N | C—Br |
| 210. | CH | CH | N | C—CH₃ |
| 211. | CH | CH | CH | CH |
| 212. | CH | CH | CH | C—Cl |
| 213. | CH | CH | CH | C—F |
| 214. | CH | CH | CH | C—Br |
| 215. | CH | CH | CH | C—CH₃ |
| 216. | CH | CH | C—Cl | CH |
| 217. | CH | CH | C—Cl | C—Cl |
| 218. | CH | CH | C—Cl | C—F |
| 219. | CH | CH | C—Cl | C—Br |
| 220. | CH | CH | C—Cl | C—CH₃ |
| 221. | CH | CH | C—F | CH |
| 222. | CH | CH | C—F | C—Cl |
| 223. | CH | CH | C—F | C—F |
| 224. | CH | CH | C—F | C—Br |
| 225. | CH | CH | C—F | C—CH₃ |
| 226. | CH | CH | C—Br | CH |
| 227. | CH | CH | C—Br | C—Cl |
| 228. | CH | CH | C—Br | C—F |
| 229. | CH | CH | C—Br | C—Br |
| 230. | CH | CH | C—Br | C—CH₃ |
| 231. | CH | CH | C—CH₃ | CH |
| 232. | CH | CH | C—CH₃ | C—Cl |
| 233. | CH | CH | C—CH₃ | C—F |
| 234. | CH | CH | C—CH₃ | C—Br |
| 235. | CH | CH | C—CH₃ | C—CH₃ |
| 236. | CH | C—Cl | N | CH |
| 237. | CH | C—Cl | N | C—Cl |
| 238. | CH | C—Cl | N | C—F |
| 239. | CH | C—Cl | N | C—Br |
| 240. | CH | C—Cl | N | C—CH₃ |
| 241. | CH | C—Cl | CH | CH |
| 242. | CH | C—Cl | CH | C—Cl |
| 243. | CH | C—Cl | CH | C—F |
| 244. | CH | C—Cl | CH | C—Br |
| 245. | CH | C—Cl | CH | C—CH₃ |
| 246. | CH | C—Cl | C—Cl | CH |
| 247. | CH | C—Cl | C—Cl | C—Cl |
| 248. | CH | C—Cl | C—Cl | C—F |
| 249. | CH | C—Cl | C—Cl | C—Br |
| 250. | CH | C—Cl | C—Cl | C—CH₃ |
| 251. | CH | C—Cl | C—F | CH |
| 252. | CH | C—Cl | C—F | C—Cl |
| 253. | CH | C—Cl | C—F | C—F |
| 254. | CH | C—Cl | C—F | C—Br |
| 255. | CH | C—Cl | C—F | C—CH₃ |
| 256. | CH | C—Cl | C—Br | CH |
| 257. | CH | C—Cl | C—Br | C—Cl |
| 258. | CH | C—Cl | C—Br | C—F |
| 259. | CH | C—Cl | C—Br | C—Br |
| 260. | CH | C—Cl | C—Br | C—CH₃ |
| 261. | CH | C—Cl | C—CH₃ | CH |
| 262. | CH | C—Cl | C—CH₃ | C—Cl |
| 263. | CH | C—Cl | C—CH₃ | C—F |
| 264. | CH | C—Cl | C—CH₃ | C—Br |
| 265. | CH | C—Cl | C—CH₃ | C—CH₃ |
| 266. | CH | C—F | N | CH |
| 267. | CH | C—F | N | C—Cl |
| 268. | CH | C—F | N | C—F |
| 269. | CH | C—F | N | C—Br |
| 270. | CH | C—F | N | C—CH₃ |
| 271. | CH | C—F | CH | CH |
| 272. | CH | C—F | CH | C—Cl |
| 273. | CH | C—F | CH | C—F |
| 274. | CH | C—F | CH | C—Br |
| 275. | CH | C—F | CH | C—CH₃ |
| 276. | CH | C—F | C—Cl | CH |
| 277. | CH | C—F | C—Cl | C—Cl |
| 278. | CH | C—F | C—Cl | C—F |
| 279. | CH | C—F | C—Cl | C—Br |
| 280. | CH | C—F | C—Cl | C—CH₃ |
| 281. | CH | C—F | C—F | CH |
| 282. | CH | C—F | C—F | C—Cl |
| 283. | CH | C—F | C—F | C—F |
| 284. | CH | C—F | C—F | C—Br |
| 285. | CH | C—F | C—F | C—CH₃ |
| 286. | CH | C—F | C—Br | CH |
| 287. | CH | C—F | C—Br | C—Cl |
| 288. | CH | C—F | C—Br | C—F |
| 289. | CH | C—F | C—Br | C—Br |
| 290. | CH | C—F | C—Br | C—CH₃ |
| 291. | CH | C—F | C—CH₃ | CH |
| 292. | CH | C—F | C—CH₃ | C—Cl |
| 293. | CH | C—F | C—CH₃ | C—F |
| 294. | CH | C—F | C—CH₃ | C—Br |
| 295. | CH | C—F | C—CH₃ | C—CH₃ |
| 296. | CH | C—Br | N | CH |
| 297. | CH | C—Br | N | C—Cl |
| 298. | CH | C—Br | N | C—F |
| 299. | CH | C—Br | N | C—Br |
| 300. | CH | C—Br | N | C—CH₃ |
| 301. | CH | C—Br | CH | CH |
| 302. | CH | C—Br | CH | C—Cl |
| 303. | CH | C—Br | CH | C—F |
| 304. | CH | C—Br | CH | C—Br |
| 305. | CH | C—Br | CH | C—CH₃ |
| 306. | CH | C—Br | C—Cl | CH |
| 307. | CH | C—Br | C—Cl | C—Cl |
| 308. | CH | C—Br | C—Cl | C—F |
| 309. | CH | C—Br | C—Cl | C—Br |
| 310. | CH | C—Br | C—Cl | C—CH₃ |
| 311. | CH | C—Br | C—F | CH |
| 312. | CH | C—Br | C—F | C—Cl |
| 313. | CH | C—Br | C—F | C—F |
| 314. | CH | C—Br | C—F | C—Br |
| 315. | CH | C—Br | C—F | C—CH₃ |
| 316. | CH | C—Br | C—Br | CH |
| 317. | CH | C—Br | C—Br | C—Cl |
| 318. | CH | C—Br | C—Br | C—F |
| 319. | CH | C—Br | C—Br | C—Br |
| 320. | CH | C—Br | C—Br | C—CH₃ |
| 321. | CH | C—Br | C—CH₃ | CH |
| 322. | CH | C—Br | C—CH₃ | C—Cl |
| 323. | CH | C—Br | C—CH₃ | C—F |
| 324. | CH | C—Br | C—CH₃ | C—Br |
| 325. | CH | C—Br | C—CH₃ | C—CH₃ |
| 326. | CH | C—CH₃ | N | CH |
| 327. | CH | C—CH₃ | N | C—Cl |
| 328. | CH | C—CH₃ | N | C—F |
| 329. | CH | C—CH₃ | N | C—Br |
| 330. | CH | C—CH₃ | N | C—CH₃ |
| 331. | CH | C—CH₃ | CH | CH |
| 332. | CH | C—CH₃ | CH | C—Cl |
| 333. | CH | C—CH₃ | CH | C—F |
| 334. | CH | C—CH₃ | CH | C—Br |
| 335. | CH | C—CH₃ | CH | C—CH₃ |
| 336. | CH | C—CH₃ | C—Cl | CH |
| 337. | CH | C—CH₃ | C—Cl | C—Cl |
| 338. | CH | C—CH₃ | C—Cl | C—F |
| 339. | CH | C—CH₃ | C—Cl | C—Br |
| 340. | CH | C—CH₃ | C—Cl | C—CH₃ |
| 341. | CH | C—CH₃ | C—F | CH |
| 342. | CH | C—CH₃ | C—F | C—Cl |
| 343. | CH | C—CH₃ | C—F | C—F |
| 344. | CH | C—CH₃ | C—F | C—Br |
| 345. | CH | C—CH₃ | C—F | C—CH₃ |
| 346. | CH | C—CH₃ | C—Br | CH |
| 347. | CH | C—CH₃ | C—Br | C—Cl |
| 348. | CH | C—CH₃ | C—Br | C—F |
| 349. | CH | C—CH₃ | C—Br | C—Br |
| 350. | CH | C—CH₃ | C—Br | C—CH₃ |
| 351. | CH | C—CH₃ | C—CH₃ | CH |
| 352. | CH | C—CH₃ | C—CH₃ | C—Cl |
| 353. | CH | C—CH₃ | C—CH₃ | C—F |
| 354. | CH | C—CH₃ | C—CH₃ | C—Br |
| 355. | CH | C—CH₃ | C—CH₃ | C—CH₃ |
| 356. | C—Cl | N | N | CH |
| 357. | C—Cl | N | N | C—Cl |
| 358. | C—Cl | N | N | C—F |
| 359. | C—Cl | N | N | C—Br |
| 360. | C—Cl | N | N | C—CH₃ |
| 361. | C—Cl | N | CH | CH |
| 362. | C—Cl | N | CH | C—Cl |
| 363. | C—Cl | N | CH | C—F |
| 364. | C—Cl | N | CH | C—Br |
| 365. | C—Cl | N | CH | C—CH₃ |
| 366. | C—Cl | N | C—Cl | CH |
| 367. | C—Cl | N | C—Cl | C—Cl |
| 368. | C—Cl | N | C—Cl | C—F |
| 369. | C—Cl | N | C—Cl | C—Br |
| 370. | C—Cl | N | C—Cl | C—CH₃ |
| 371. | C—Cl | N | C—F | CH |
| 372. | C—Cl | N | C—F | C—Cl |
| 373. | C—Cl | N | C—F | C—F |
| 374. | C—Cl | N | C—F | C—Br |
| 375. | C—Cl | N | C—F | C—CH₃ |
| 376. | C—Cl | N | C—Br | CH |
| 377. | C—Cl | N | C—Br | C—Cl |
| 378. | C—Cl | N | C—Br | C—F |
| 379. | C—Cl | N | C—Br | C—Br |
| 380. | C—Cl | N | C—Br | C—CH₃ |
| 381. | C—Cl | N | C—CH₃ | CH |
| 382. | C—Cl | N | C—CH₃ | C—Cl |
| 383. | C—Cl | N | C—CH₃ | C—F |
| 384. | C—Cl | N | C—CH₃ | C—Br |
| 385. | C—Cl | N | C—CH₃ | C—CH₃ |
| 386. | C—Cl | CH | N | CH |
| 387. | C—Cl | CH | N | C—Cl |
| 388. | C—Cl | CH | N | C—F |
| 389. | C—Cl | CH | N | C—Br |
| 390. | C—Cl | CH | N | C—CH₃ |
| 391. | C—Cl | CH | CH | CH |
| 392. | C—Cl | CH | CH | C—Cl |
| 393. | C—Cl | CH | CH | C—F |
| 394. | C—Cl | CH | CH | C—Br |
| 395. | C—Cl | CH | CH | C—CH₃ |
| 396. | C—Cl | CH | C—Cl | CH |
| 397. | C—Cl | CH | C—Cl | C—Cl |
| 398. | C—Cl | CH | C—Cl | C—F |
| 399. | C—Cl | CH | C—Cl | C—Br |
| 400. | C—Cl | CH | C—Cl | C—CH₃ |
| 401. | C—Cl | CH | C—F | CH |
| 402. | C—Cl | CH | C—F | C—Cl |
| 403. | C—Cl | CH | C—F | C—F |
| 404. | C—Cl | CH | C—F | C—Br |
| 405. | C—Cl | CH | C—F | C—CH₃ |
| 406. | C—Cl | CH | C—Br | CH |
| 407. | C—Cl | CH | C—Br | C—Cl |
| 408. | C—Cl | CH | C—Br | C—F |
| 409. | C—Cl | CH | C—Br | C—Br |
| 410. | C—Cl | CH | C—Br | C—CH₃ |
| 411. | C—Cl | CH | C—CH₃ | CH |
| 412. | C—Cl | CH | C—CH₃ | C—Cl |
| 413. | C—Cl | CH | C—CH₃ | C—F |
| 414. | C—Cl | CH | C—CH₃ | C—Br |
| 415. | C—Cl | CH | C—CH₃ | C—CH₃ |
| 416. | C—Cl | C—Cl | N | CH |
| 417. | C—Cl | C—Cl | N | C—Cl |
| 418. | C—Cl | C—Cl | N | C—F |
| 419. | C—Cl | C—Cl | N | C—Br |
| 420. | C—Cl | C—Cl | N | C—CH₃ |
| 421. | C—Cl | C—Cl | CH | CH |
| 422. | C—Cl | C—Cl | CH | C—Cl |
| 423. | C—Cl | C—Cl | CH | C—F |
| 424. | C—Cl | C—Cl | CH | C—Br |
| 425. | C—Cl | C—Cl | CH | C—CH₃ |
| 426. | C—Cl | C—Cl | C—Cl | CH |
| 427. | C—Cl | C—Cl | C—Cl | C—Cl |
| 428. | C—Cl | C—Cl | C—Cl | C—F |
| 429. | C—Cl | C—Cl | C—Cl | C—Br |
| 430. | C—Cl | C—Cl | C—Cl | C—CH₃ |
| 431. | C—Cl | C—Cl | C—F | CH |
| 432. | C—Cl | C—Cl | C—F | C—Cl |
| 433. | C—Cl | C—Cl | C—F | C—F |
| 434. | C—Cl | C—Cl | C—F | C—Br |
| 435. | C—Cl | C—Cl | C—F | C—CH₃ |
| 436. | C—Cl | C—Cl | C—Br | CH |
| 437. | C—Cl | C—Cl | C—Br | C—Cl |
| 438. | C—Cl | C—Cl | C—Br | C—F |
| 439. | C—Cl | C—Cl | C—Br | C—Br |
| 440. | C—Cl | C—Cl | C—Br | C—CH₃ |
| 441. | C—Cl | C—Cl | C—CH₃ | CH |
| 442. | C—Cl | C—Cl | C—CH₃ | C—Cl |
| 443. | C—Cl | C—Cl | C—CH₃ | C—F |
| 444. | C—Cl | C—Cl | C—CH₃ | C—Br |
| 445. | C—Cl | C—Cl | C—CH₃ | C—CH₃ |
| 446. | C—Cl | C—F | N | CH |
| 447. | C—Cl | C—F | N | C—Cl |
| 448. | C—Cl | C—F | N | C—F |
| 449. | C—Cl | C—F | N | C—Br |
| 450. | C—Cl | C—F | N | C—CH₃ |
| 451. | C—Cl | C—F | CH | CH |
| 452. | C—Cl | C—F | CH | C—Cl |
| 453. | C—Cl | C—F | CH | C—F |
| 454. | C—Cl | C—F | CH | C—Br |
| 455. | C—Cl | C—F | CH | C—CH₃ |
| 456. | C—Cl | C—F | C—Cl | CH |
| 457. | C—Cl | C—F | C—Cl | C—Cl |
| 458. | C—Cl | C—F | C—Cl | C—F |
| 459. | C—Cl | C—F | C—Cl | C—Br |
| 460. | C—Cl | C—F | C—Cl | C—CH₃ |
| 461. | C—Cl | C—F | C—F | CH |
| 462. | C—Cl | C—F | C—F | C—Cl |
| 463. | C—Cl | C—F | C—F | C—F |
| 464. | C—Cl | C—F | C—F | C—Br |
| 465. | C—Cl | C—F | C—F | C—CH₃ |
| 466. | C—Cl | C—F | C—Br | CH |
| 467. | C—Cl | C—F | C—Br | C—Cl |
| 468. | C—Cl | C—F | C—Br | C—F |
| 469. | C—Cl | C—F | C—Br | C—Br |
| 470. | C—Cl | C—F | C—Br | C—CH₃ |
| 471. | C—Cl | C—F | C—CH₃ | CH |
| 472. | C—Cl | C—F | C—CH₃ | C—Cl |
| 473. | C—Cl | C—F | C—CH₃ | C—F |
| 474. | C—Cl | C—F | C—CH₃ | C—Br |
| 475. | C—Cl | C—F | C—CH₃ | C—CH₃ |
| 476. | C—Cl | C—Br | N | CH |
| 477. | C—Cl | C—Br | N | C—Cl |
| 478. | C—Cl | C—Br | N | C—F |
| 479. | C—Cl | C—Br | N | C—Br |
| 480. | C—Cl | C—Br | N | C—CH₃ |
| 481. | C—Cl | C—Br | CH | CH |
| 482. | C—Cl | C—Br | CH | C—Cl |
| 483. | C—Cl | C—Br | CH | C—F |
| 484. | C—Cl | C—Br | CH | C—Br |
| 485. | C—Cl | C—Br | CH | C—CH₃ |
| 486. | C—Cl | C—Br | C—Cl | CH |
| 487. | C—Cl | C—Br | C—Cl | C—Cl |
| 488. | C—Cl | C—Br | C—Cl | C—F |
| 489. | C—Cl | C—Br | C—Cl | C—Br |
| 490. | C—Cl | C—Br | C—Cl | C—CH₃ |
| 491. | C—Cl | C—Br | C—F | CH |
| 492. | C—Cl | C—Br | C—F | C—Cl |
| 493. | C—Cl | C—Br | C—F | C—F |
| 494. | C—Cl | C—Br | C—F | C—Br |
| 495. | C—Cl | C—Br | C—F | C—CH₃ |
| 496. | C—Cl | C—Br | C—Br | CH |
| 497. | C—Cl | C—Br | C—Br | C—Cl |
| 498. | C—Cl | C—Br | C—Br | C—F |
| 499. | C—Cl | C—Br | C—Br | C—Br |
| 500. | C—Cl | C—Br | C—Br | C—CH₃ |
| 501. | C—Cl | C—Br | C—CH₃ | CH |
| 502. | C—Cl | C—Br | C—CH₃ | C—Cl |
| 503. | C—Cl | C—Br | C—CH₃ | C—F |
| 504. | C—Cl | C—Br | C—CH₃ | C—Br |
| 505. | C—Cl | C—Br | C—CH₃ | C—CH₃ |
| 506. | C—Cl | C—CH₃ | N | CH |
| 507. | C—Cl | C—CH₃ | N | C—Cl |
| 508. | C—Cl | C—CH₃ | N | C—F |
| 509. | C—Cl | C—CH₃ | N | C—Br |
| 510. | C—Cl | C—CH₃ | N | C—CH₃ |
| 511. | C—Cl | C—CH₃ | CH | CH |
| 512. | C—Cl | C—CH₃ | CH | C—Cl |
| 513. | C—Cl | C—CH₃ | CH | C—F |
| 514. | C—Cl | C—CH₃ | CH | C—Br |
| 515. | C—Cl | C—CH₃ | CH | C—CH₃ |
| 516. | C—Cl | C—CH₃ | C—Cl | CH |
| 517. | C—Cl | C—CH₃ | C—Cl | C—Cl |
| 518. | C—Cl | C—CH₃ | C—Cl | C—F |
| 519. | C—Cl | C—CH₃ | C—Cl | C—Br |
| 520. | C—Cl | C—CH₃ | C—Cl | C—CH₃ |
| 521. | C—Cl | C—CH₃ | C—F | CH |
| 522. | C—Cl | C—CH₃ | C—F | C—Cl |
| 523. | C—Cl | C—CH₃ | C—F | C—F |
| 524. | C—Cl | C—CH₃ | C—F | C—Br |
| 525. | C—Cl | C—CH₃ | C—F | C—CH₃ |
| 526. | C—Cl | C—CH₃ | C—Br | CH |
| 527. | C—Cl | C—CH₃ | C—Br | C—Cl |
| 528. | C—Cl | C—CH₃ | C—Br | C—F |
| 529. | C—Cl | C—CH₃ | C—Br | C—Br |
| 530. | C—Cl | C—CH₃ | C—Br | C—CH₃ |
| 531. | C—Cl | C—CH₃ | C—CH₃ | CH |
| 532. | C—Cl | C—CH₃ | C—CH₃ | C—Cl |
| 533. | C—Cl | C—CH₃ | C—CH₃ | C—F |
| 534. | C—Cl | C—CH₃ | C—CH₃ | C—Br |
| 535. | C—Cl | C—CH₃ | C—CH₃ | C—CH₃ |
| 536. | C—F | N | N | CH |
| 537. | C—F | N | N | C—Cl |
| 538. | C—F | N | N | C—F |
| 539. | C—F | N | N | C—Br |
| 540. | C—F | N | N | C—CH₃ |
| 541. | C—F | N | CH | CH |
| 542. | C—F | N | CH | C—Cl |
| 543. | C—F | N | CH | C—F |
| 544. | C—F | N | CH | C—Br |
| 545. | C—F | N | CH | C—CH₃ |
| 546. | C—F | N | C—Cl | CH |
| 547. | C—F | N | C—Cl | C—Cl |
| 548. | C—F | N | C—Cl | C—F |
| 549. | C—F | N | C—Cl | C—Br |
| 550. | C—F | N | C—Cl | C—CH₃ |
| 551. | C—F | N | C—F | CH |
| 552. | C—F | N | C—F | C—Cl |
| 553. | C—F | N | C—F | C—F |
| 554. | C—F | N | C—F | C—Br |
| 555. | C—F | N | C—F | C—CH₃ |
| 556. | C—F | N | C—Br | CH |
| 557. | C—F | N | C—Br | C—Cl |
| 558. | C—F | N | C—Br | C—F |
| 559. | C—F | N | C—Br | C—Br |
| 560. | C—F | N | C—Br | C—CH₃ |
| 561. | C—F | N | C—CH₃ | CH |
| 562. | C—F | N | C—CH₃ | C—Cl |
| 563. | C—F | N | C—CH₃ | C—F |
| 564. | C—F | N | C—CH₃ | C—Br |
| 565. | C—F | N | C—CH₃ | C—CH₃ |
| 566. | C—F | CH | N | CH |
| 567. | C—F | CH | N | C—Cl |
| 568. | C—F | CH | N | C—F |
| 569. | C—F | CH | N | C—Br |
| 570. | C—F | CH | N | C—CH₃ |
| 571. | C—F | CH | CH | CH |
| 572. | C—F | CH | CH | C—Cl |
| 573. | C—F | CH | CH | C—F |
| 574. | C—F | CH | CH | C—Br |
| 575. | C—F | CH | CH | C—CH₃ |
| 576. | C—F | CH | C—Cl | CH |
| 577. | C—F | CH | C—Cl | C—Cl |
| 578. | C—F | CH | C—Cl | C—F |
| 579. | C—F | CH | C—Cl | C—Br |
| 580. | C—F | CH | C—Cl | C—CH₃ |
| 581. | C—F | CH | C—F | CH |
| 582. | C—F | CH | C—F | C—Cl |
| 583. | C—F | CH | C—F | C—F |
| 584. | C—F | CH | C—F | C—Br |
| 585. | C—F | CH | C—F | C—CH₃ |
| 586. | C—F | CH | C—Br | CH |
| 587. | C—F | CH | C—Br | C—Cl |
| 588. | C—F | CH | C—Br | C—F |
| 589. | C—F | CH | C—Br | C—Br |
| 590. | C—F | CH | C—Br | C—CH₃ |
| 591. | C—F | CH | C—CH₃ | CH |
| 592. | C—F | CH | C—CH₃ | C—Cl |
| 593. | C—F | CH | C—CH₃ | C—F |
| 594. | C—F | CH | C—CH₃ | C—Br |
| 595. | C—F | CH | C—CH₃ | C—CH₃ |
| 596. | C—F | C—Cl | N | CH |
| 597. | C—F | C—Cl | N | C—Cl |
| 598. | C—F | C—Cl | N | C—F |
| 599. | C—F | C—Cl | N | C—Br |
| 600. | C—F | C—Cl | N | C—CH₃ |
| 601. | C—F | C—Cl | CH | CH |
| 602. | C—F | C—Cl | CH | C—Cl |
| 603. | C—F | C—Cl | CH | C—F |
| 604. | C—F | C—Cl | CH | C—Br |
| 605. | C—F | C—Cl | CH | C—CH₃ |
| 606. | C—F | C—Cl | C—Cl | CH |
| 607. | C—F | C—Cl | C—Cl | C—Cl |
| 608. | C—F | C—Cl | C—Cl | C—F |
| 609. | C—F | C—Cl | C—Cl | C—Br |
| 610. | C—F | C—Cl | C—Cl | C—CH₃ |
| 611. | C—F | C—Cl | C—F | CH |
| 612. | C—F | C—Cl | C—F | C—Cl |
| 613. | C—F | C—Cl | C—F | C—F |
| 614. | C—F | C—Cl | C—F | C—Br |
| 615. | C—F | C—Cl | C—F | C—CH₃ |
| 616. | C—F | C—Cl | C—Br | CH |
| 617. | C—F | C—Cl | C—Br | C—Cl |
| 618. | C—F | C—Cl | C—Br | C—F |
| 619. | C—F | C—Cl | C—Br | C—Br |
| 620. | C—F | C—Cl | C—Br | C—CH₃ |
| 621. | C—F | C—Cl | C—CH₃ | CH |
| 622. | C—F | C—Cl | C—CH₃ | C—Cl |
| 623. | C—F | C—Cl | C—CH₃ | C—F |
| 624. | C—F | C—Cl | C—CH₃ | C—Br |
| 625. | C—F | C—Cl | C—CH₃ | C—CH₃ |
| 626. | C—F | C—F | N | CH |
| 627. | C—F | C—F | N | C—Cl |
| 628. | C—F | C—F | N | C—F |
| 629. | C—F | C—F | N | C—Br |
| 630. | C—F | C—F | N | C—CH₃ |
| 631. | C—F | C—F | CH | CH |
| 632. | C—F | C—F | CH | C—Cl |
| 633. | C—F | C—F | CH | C—F |
| 634. | C—F | C—F | CH | C—Br |
| 635. | C—F | C—F | CH | C—CH₃ |
| 636. | C—F | C—F | C—Cl | CH |
| 637. | C—F | C—F | C—Cl | C—Cl |
| 638. | C—F | C—F | C—Cl | C—F |
| 639. | C—F | C—F | C—Cl | C—Br |
| 640. | C—F | C—F | C—Cl | C—CH₃ |
| 641. | C—F | C—F | C—F | CH |
| 642. | C—F | C—F | C—F | C—Cl |
| 643. | C—F | C—F | C—F | C—F |
| 644. | C—F | C—F | C—F | C—Br |
| 645. | C—F | C—F | C—F | C—CH₃ |
| 646. | C—F | C—F | C—Br | CH |
| 647. | C—F | C—F | C—Br | C—Cl |
| 648. | C—F | C—F | C—Br | C—F |
| 649. | C—F | C—F | C—Br | C—Br |
| 650. | C—F | C—F | C—Br | C—CH₃ |
| 651. | C—F | C—F | C—CH₃ | CH |
| 652. | C—F | C—F | C—CH₃ | C—Cl |
| 653. | C—F | C—F | C—CH₃ | C—F |
| 654. | C—F | C—F | C—CH₃ | C—Br |
| 655. | C—F | C—F | C—CH₃ | C—CH₃ |
| 656. | C—F | C—Br | N | CH |
| 657. | C—F | C—Br | N | C—Cl |
| 658. | C—F | C—Br | N | C—F |
| 659. | C—F | C—Br | N | C—Br |
| 660. | C—F | C—Br | N | C—CH₃ |
| 661. | C—F | C—Br | CH | CH |
| 662. | C—F | C—Br | CH | C—Cl |
| 663. | C—F | C—Br | CH | C—F |
| 664. | C—F | C—Br | CH | C—Br |
| 665. | C—F | C—Br | CH | C—CH₃ |
| 666. | C—F | C—Br | C—Cl | CH |
| 667. | C—F | C—Br | C—Cl | C—Cl |
| 668. | C—F | C—Br | C—Cl | C—F |
| 669. | C—F | C—Br | C—Cl | C—Br |
| 670. | C—F | C—Br | C—Cl | C—CH₃ |
| 671. | C—F | C—Br | C—F | CH |
| 672. | C—F | C—Br | C—F | C—Cl |
| 673. | C—F | C—Br | C—F | C—F |
| 674. | C—F | C—Br | C—F | C—Br |
| 675. | C—F | C—Br | C—F | C—CH₃ |
| 676. | C—F | C—Br | C—Br | CH |
| 677. | C—F | C—Br | C—Br | C—Cl |
| 678. | C—F | C—Br | C—Br | C—F |
| 679. | C—F | C—Br | C—Br | C—Br |
| 680. | C—F | C—Br | C—Br | C—CH₃ |
| 681. | C—F | C—Br | C—CH₃ | CH |
| 682. | C—F | C—Br | C—CH₃ | C—Cl |
| 683. | C—F | C—Br | C—CH₃ | C—F |
| 684. | C—F | C—Br | C—CH₃ | C—Br |
| 685. | C—F | C—Br | C—CH₃ | C—CH₃ |
| 686. | C—F | C—CH₃ | N | CH |
| 687. | C—F | C—CH₃ | N | C—Cl |
| 688. | C—F | C—CH₃ | N | C—F |
| 689. | C—F | C—CH₃ | N | C—Br |
| 690. | C—F | C—CH₃ | N | C—CH₃ |
| 691. | C—F | C—CH₃ | CH | CH |
| 692. | C—F | C—CH₃ | CH | C—Cl |
| 693. | C—F | C—CH₃ | CH | C—F |
| 694. | C—F | C—CH₃ | CH | C—Br |
| 695. | C—F | C—CH₃ | CH | C—CH₃ |
| 696. | C—F | C—CH₃ | C—Cl | CH |
| 697. | C—F | C—CH₃ | C—Cl | C—Cl |
| 698. | C—F | C—CH₃ | C—Cl | C—F |
| 699. | C—F | C—CH₃ | C—Cl | C—Br |
| 700. | C—F | C—CH₃ | C—Cl | C—CH₃ |
| 701. | C—F | C—CH₃ | C—F | CH |
| 702. | C—F | C—CH₃ | C—F | C—Cl |
| 703. | C—F | C—CH₃ | C—F | C—F |
| 704. | C—F | C—CH₃ | C—F | C—Br |
| 705. | C—F | C—CH₃ | C—F | C—CH₃ |
| 706. | C—F | C—CH₃ | C—Br | CH |
| 707. | C—F | C—CH₃ | C—Br | C—Cl |
| 708. | C—F | C—CH₃ | C—Br | C—F |
| 709. | C—F | C—CH₃ | C—Br | C—Br |
| 710. | C—F | C—CH₃ | C—Br | C—CH₃ |
| 711. | C—F | C—CH₃ | C—CH₃ | CH |
| 712. | C—F | C—CH₃ | C—CH₃ | C—Cl |
| 713. | C—F | C—CH₃ | C—CH₃ | C—F |
| 714. | C—F | C—CH₃ | C—CH₃ | C—Br |
| 715. | C—F | C—CH₃ | C—CH₃ | C—CH₃ |
| 716. | C—Br | N | N | CH |
| 717. | C—Br | N | N | C—Cl |
| 718. | C—Br | N | N | C—F |
| 719. | C—Br | N | N | C—Br |
| 720. | C—Br | N | N | C—CH₃ |
| 721. | C—Br | N | CH | CH |
| 722. | C—Br | N | CH | C—Cl |
| 723. | C—Br | N | CH | C—F |
| 724. | C—Br | N | CH | C—Br |
| 725. | C—Br | N | CH | C—CH₃ |
| 726. | C—Br | N | C—Cl | CH |
| 727. | C—Br | N | C—Cl | C—Cl |
| 728. | C—Br | N | C—Cl | C—F |
| 729. | C—Br | N | C—Cl | C—Br |
| 730. | C—Br | N | C—Cl | C—CH₃ |
| 731. | C—Br | N | C—F | CH |
| 732. | C—Br | N | C—F | C—Cl |
| 733. | C—Br | N | C—F | C—F |
| 734. | C—Br | N | C—F | C—Br |
| 735. | C—Br | N | C—F | C—CH₃ |
| 736. | C—Br | N | C—Br | CH |
| 737. | C—Br | N | C—Br | C—Cl |
| 738. | C—Br | N | C—Br | C—F |
| 739. | C—Br | N | C—Br | C—Br |
| 740. | C—Br | N | C—Br | C—CH₃ |
| 741. | C—Br | N | C—CH₃ | CH |
| 742. | C—Br | N | C—CH₃ | C—Cl |
| 743. | C—Br | N | C—CH₃ | C—F |
| 744. | C—Br | N | C—CH₃ | C—Br |
| 745. | C—Br | N | C—CH₃ | C—CH₃ |
| 746. | C—Br | CH | N | CH |
| 747. | C—Br | CH | N | C—Cl |
| 748. | C—Br | CH | N | C—F |
| 749. | C—Br | CH | N | C—Br |
| 750. | C—Br | CH | N | C—CH₃ |
| 751. | C—Br | CH | CH | CH |
| 752. | C—Br | CH | CH | C—Cl |
| 753. | C—Br | CH | CH | C—F |
| 754. | C—Br | CH | CH | C—Br |
| 755. | C—Br | CH | CH | C—CH₃ |
| 756. | C—Br | CH | C—Cl | CH |
| 757. | C—Br | CH | C—Cl | C—Cl |
| 758. | C—Br | CH | C—Cl | C—F |
| 759. | C—Br | CH | C—Cl | C—Br |
| 760. | C—Br | CH | C—Cl | C—CH₃ |
| 761. | C—Br | CH | C—F | CH |
| 762. | C—Br | CH | C—F | C—Cl |
| 763. | C—Br | CH | C—F | C—F |
| 764. | C—Br | CH | C—F | C—Br |
| 765. | C—Br | CH | C—F | C—CH₃ |
| 766. | C—Br | CH | C—Br | CH |
| 767. | C—Br | CH | C—Br | C—Cl |
| 768. | C—Br | CH | C—Br | C—F |
| 769. | C—Br | CH | C—Br | C—Br |
| 770. | C—Br | CH | C—Br | C—CH₃ |
| 771. | C—Br | CH | C—CH₃ | CH |
| 772. | C—Br | CH | C—CH₃ | C—Cl |
| 773. | C—Br | CH | C—CH₃ | C—F |
| 774. | C—Br | CH | C—CH₃ | C—Br |
| 775. | C—Br | CH | C—CH₃ | C—CH₃ |
| 776. | C—Br | C—Cl | N | CH |
| 777. | C—Br | C—Cl | N | C—Cl |
| 778. | C—Br | C—Cl | N | C—F |
| 779. | C—Br | C—Cl | N | C—Br |
| 780. | C—Br | C—Cl | N | C—CH₃ |
| 781. | C—Br | C—Cl | CH | CH |
| 782. | C—Br | C—Cl | CH | C—Cl |
| 783. | C—Br | C—Cl | CH | C—F |
| 784. | C—Br | C—Cl | CH | C—Br |
| 785. | C—Br | C—Cl | CH | C—CH₃ |
| 786. | C—Br | C—Cl | C—Cl | CH |
| 787. | C—Br | C—Cl | C—Cl | C—Cl |
| 788. | C—Br | C—Cl | C—Cl | C—F |
| 789. | C—Br | C—Cl | C—Cl | C—Br |
| 790. | C—Br | C—Cl | C—Cl | C—CH₃ |
| 791. | C—Br | C—Cl | C—F | CH |
| 792. | C—Br | C—Cl | C—F | C—Cl |
| 793. | C—Br | C—Cl | C—F | C—F |
| 794. | C—Br | C—Cl | C—F | C—Br |
| 795. | C—Br | C—Cl | C—F | C—CH₃ |
| 796. | C—Br | C—Cl | C—Br | CH |
| 797. | C—Br | C—Cl | C—Br | C—Cl |
| 798. | C—Br | C—Cl | C—Br | C—F |
| 799. | C—Br | C—Cl | C—Br | C—Br |
| 800. | C—Br | C—Cl | C—Br | C—CH₃ |
| 801. | C—Br | C—Cl | C—CH₃ | CH |
| 802. | C—Br | C—Cl | C—CH₃ | C—Cl |
| 803. | C—Br | C—Cl | C—CH₃ | C—F |
| 804. | C—Br | C—Cl | C—CH₃ | C—Br |
| 805. | C—Br | C—Cl | C—CH₃ | C—CH₃ |
| 806. | C—Br | C—F | N | CH |
| 807. | C—Br | C—F | N | C—Cl |
| 808. | C—Br | C—F | N | C—F |
| 809. | C—Br | C—F | N | C—Br |
| 810. | C—Br | C—F | N | C—CH₃ |
| 811. | C—Br | C—F | CH | CH |
| 812. | C—Br | C—F | CH | C—Cl |
| 813. | C—Br | C—F | CH | C—F |
| 814. | C—Br | C—F | CH | C—Br |
| 815. | C—Br | C—F | CH | C—CH₃ |
| 816. | C—Br | C—F | C—Cl | CH |
| 817. | C—Br | C—F | C—Cl | C—Cl |
| 818. | C—Br | C—F | C—Cl | C—F |
| 819. | C—Br | C—F | C—Cl | C—Br |
| 820. | C—Br | C—F | C—Cl | C—CH₃ |
| 821. | C—Br | C—F | C—F | CH |
| 822. | C—Br | C—F | C—F | C—Cl |
| 823. | C—Br | C—F | C—F | C—F |
| 824. | C—Br | C—F | C—F | C—Br |
| 825. | C—Br | C—F | C—F | C—CH₃ |
| 826. | C—Br | C—F | C—Br | CH |
| 827. | C—Br | C—F | C—Br | C—Cl |
| 828. | C—Br | C—F | C—Br | C—F |
| 829. | C—Br | C—F | C—Br | C—Br |
| 830. | C—Br | C—F | C—Br | C—CH₃ |
| 831. | C—Br | C—F | C—CH₃ | CH |
| 832. | C—Br | C—F | C—CH₃ | C—Cl |
| 833. | C—Br | C—F | C—CH₃ | C—F |
| 834. | C—Br | C—F | C—CH₃ | C—Br |
| 835. | C—Br | C—F | C—CH₃ | C—CH₃ |
| 836. | C—Br | C—Br | N | CH |
| 837. | C—Br | C—Br | N | C—Cl |
| 838. | C—Br | C—Br | N | C—F |
| 839. | C—Br | C—Br | N | C—Br |
| 840. | C—Br | C—Br | N | C—CH₃ |
| 841. | C—Br | C—Br | CH | CH |
| 842. | C—Br | C—Br | CH | C—Cl |
| 843. | C—Br | C—Br | CH | C—F |
| 844. | C—Br | C—Br | CH | C—Br |
| 845. | C—Br | C—Br | CH | C—CH₃ |
| 846. | C—Br | C—Br | C—Cl | CH |
| 847. | C—Br | C—Br | C—Cl | C—Cl |
| 848. | C—Br | C—Br | C—Cl | C—F |
| 849. | C—Br | C—Br | C—Cl | C—Br |
| 850. | C—Br | C—Br | C—Cl | C—CH₃ |
| 851. | C—Br | C—Br | C—F | CH |
| 852. | C—Br | C—Br | C—F | C—Cl |
| 853. | C—Br | C—Br | C—F | C—F |
| 854. | C—Br | C—Br | C—F | C—Br |
| 855. | C—Br | C—Br | C—F | C—CH₃ |
| 856. | C—Br | C—Br | C—Br | CH |
| 857. | C—Br | C—Br | C—Br | C—Cl |
| 858. | C—Br | C—Br | C—Br | C—F |
| 859. | C—Br | C—Br | C—Br | C—Br |
| 860. | C—Br | C—Br | C—Br | C—CH₃ |
| 861. | C—Br | C—Br | C—CH₃ | CH |
| 862. | C—Br | C—Br | C—CH₃ | C—Cl |
| 863. | C—Br | C—Br | C—CH₃ | C—F |
| 864. | C—Br | C—Br | C—CH₃ | C—Br |
| 865. | C—Br | C—Br | C—CH₃ | C—CH₃ |
| 866. | C—Br | C—CH₃ | N | CH |
| 867. | C—Br | C—CH₃ | N | C—Cl |
| 868. | C—Br | C—CH₃ | N | C—F |
| 869. | C—Br | C—CH₃ | N | C—Br |
| 870. | C—Br | C—CH₃ | N | C—CH₃ |
| 871. | C—Br | C—CH₃ | CH | CH |
| 872. | C—Br | C—CH₃ | CH | C—Cl |
| 873. | C—Br | C—CH₃ | CH | C—F |
| 874. | C—Br | C—CH₃ | CH | C—Br |
| 875. | C—Br | C—CH₃ | CH | C—CH₃ |
| 876. | C—Br | C—CH₃ | C—Cl | CH |
| 877. | C—Br | C—CH₃ | C—Cl | C—Cl |
| 878. | C—Br | C—CH₃ | C—Cl | C—F |
| 879. | C—Br | C—CH₃ | C—Cl | C—Br |
| 880. | C—Br | C—CH₃ | C—Cl | C—CH₃ |
| 881. | C—Br | C—CH₃ | C—F | CH |
| 882. | C—Br | C—CH₃ | C—F | C—Cl |
| 883. | C—Br | C—CH₃ | C—F | C—F |
| 884. | C—Br | C—CH₃ | C—F | C—Br |
| 885. | C—Br | C—CH₃ | C—F | C—CH₃ |
| 886. | C—Br | C—CH₃ | C—Br | CH |
| 887. | C—Br | C—CH₃ | C—Br | C—Cl |
| 888. | C—Br | C—CH₃ | C—Br | C—F |
| 889. | C—Br | C—CH₃ | C—Br | C—Br |
| 890. | C—Br | C—CH₃ | C—Br | C—CH₃ |
| 891. | C—Br | C—CH₃ | C—CH₃ | CH |
| 892. | C—Br | C—CH₃ | C—CH₃ | C—Cl |
| 893. | C—Br | C—CH₃ | C—CH₃ | C—F |
| 894. | C—Br | C—CH₃ | C—CH₃ | C—Br |
| 895. | C—Br | C—CH₃ | C—CH₃ | C—CH₃ |
| 896. | C—CH₃ | N | N | CH |
| 897. | C—CH₃ | N | N | C—Cl |
| 898. | C—CH₃ | N | N | C—F |
| 899. | C—CH₃ | N | N | C—Br |
| 900. | C—CH₃ | N | N | C—CH₃ |
| 901. | C—CH₃ | N | CH | CH |
| 902. | C—CH₃ | N | CH | C—Cl |
| 903. | C—CH₃ | N | CH | C—F |
| 904. | C—CH₃ | N | CH | C—Br |
| 905. | C—CH₃ | N | CH | C—CH₃ |
| 906. | C—CH₃ | N | C—Cl | CH |
| 907. | C—CH₃ | N | C—Cl | C—Cl |
| 908. | C—CH₃ | N | C—Cl | C—F |
| 909. | C—CH₃ | N | C—Cl | C—Br |
| 910. | C—CH₃ | N | C—Cl | C—CH₃ |
| 911. | C—CH₃ | N | C—F | CH |
| 912. | C—CH₃ | N | C—F | C—Cl |
| 913. | C—CH₃ | N | C—F | C—F |
| 914. | C—CH₃ | N | C—F | C—Br |
| 915. | C—CH₃ | N | C—F | C—CH₃ |
| 916. | C—CH₃ | N | C—Br | CH |
| 917. | C—CH₃ | N | C—Br | C—Cl |
| 918. | C—CH₃ | N | C—Br | C—F |
| 919. | C—CH₃ | N | C—Br | C—Br |
| 920. | C—CH₃ | N | C—Br | C—CH₃ |
| 921. | C—CH₃ | N | C—CH₃ | CH |
| 922. | C—CH₃ | N | C—CH₃ | C—Cl |
| 923. | C—CH₃ | N | C—CH₃ | C—F |
| 924. | C—CH₃ | N | C—CH₃ | C—Br |
| 925. | C—CH₃ | N | C—CH₃ | C—CH₃ |
| 926. | C—CH₃ | CH | N | CH |
| 927. | C—CH₃ | CH | N | C—Cl |
| 928. | C—CH₃ | CH | N | C—F |
| 929. | C—CH₃ | CH | N | C—Br |
| 930. | C—CH₃ | CH | N | C—CH₃ |
| 931. | C—CH₃ | CH | CH | CH |
| 932. | C—CH₃ | CH | CH | C—Cl |
| 933. | C—CH₃ | CH | CH | C—F |
| 934. | C—CH₃ | CH | CH | C—Br |
| 935. | C—CH₃ | CH | CH | C—CH₃ |
| 936. | C—CH₃ | CH | C—Cl | CH |
| 937. | C—CH₃ | CH | C—Cl | C—Cl |
| 938. | C—CH₃ | CH | C—Cl | C—F |
| 939. | C—CH₃ | CH | C—Cl | C—Br |
| 940. | C—CH₃ | CH | C—Cl | C—CH₃ |
| 941. | C—CH₃ | CH | C—F | CH |
| 942. | C—CH₃ | CH | C—F | C—Cl |
| 943. | C—CH₃ | CH | C—F | C—F |
| 944. | C—CH₃ | CH | C—F | C—Br |
| 945. | C—CH₃ | CH | C—F | C—CH₃ |
| 946. | C—CH₃ | CH | C—Br | CH |
| 947. | C—CH₃ | CH | C—Br | C—Cl |
| 948. | C—CH₃ | CH | C—Br | C—F |
| 949. | C—CH₃ | CH | C—Br | C—Br |
| 950. | C—CH₃ | CH | C—Br | C—CH₃ |
| 951. | C—CH₃ | CH | C—CH₃ | CH |
| 952. | C—CH₃ | CH | C—CH₃ | C—Cl |
| 953. | C—CH₃ | CH | C—CH₃ | C—F |
| 954. | C—CH₃ | CH | C—CH₃ | C—Br |
| 955. | C—CH₃ | CH | C—CH₃ | C—CH₃ |
| 956. | C—CH₃ | C—Cl | N | CH |
| 957. | C—CH₃ | C—Cl | N | C—Cl |
| 958. | C—CH₃ | C—Cl | N | C—F |
| 959. | C—CH₃ | C—Cl | N | C—Br |
| 960. | C—CH₃ | C—Cl | N | C—CH₃ |
| 961. | C—CH₃ | C—Cl | CH | CH |
| 962. | C—CH₃ | C—Cl | CH | C—Cl |
| 963. | C—CH₃ | C—Cl | CH | C—F |
| 964. | C—CH₃ | C—Cl | CH | C—Br |
| 965. | C—CH₃ | C—Cl | CH | C—CH₃ |
| 966. | C—CH₃ | C—Cl | C—Cl | CH |
| 967. | C—CH₃ | C—Cl | C—Cl | C—Cl |
| 968. | C—CH₃ | C—Cl | C—Cl | C—F |
| 969. | C—CH₃ | C—Cl | C—Cl | C—Br |
| 970. | C—CH₃ | C—Cl | C—Cl | C—CH₃ |
| 971. | C—CH₃ | C—Cl | C—F | CH |
| 972. | C—CH₃ | C—Cl | C—F | C—Cl |
| 973. | C—CH₃ | C—Cl | C—F | C—F |
| 974. | C—CH₃ | C—Cl | C—F | C—Br |
| 975. | C—CH₃ | C—Cl | C—F | C—CH₃ |
| 976. | C—CH₃ | C—Cl | C—Br | CH |
| 977. | C—CH₃ | C—Cl | C—Br | C—Cl |
| 978. | C—CH₃ | C—Cl | C—Br | C—F |
| 979. | C—CH₃ | C—Cl | C—Br | C—Br |
| 980. | C—CH₃ | C—Cl | C—Br | C—CH₃ |
| 981. | C—CH₃ | C—Cl | C—CH₃ | CH |
| 982. | C—CH₃ | C—Cl | C—CH₃ | C—Cl |
| 983. | C—CH₃ | C—Cl | C—CH₃ | C—F |
| 984. | C—CH₃ | C—Cl | C—CH₃ | C—Br |
| 985. | C—CH₃ | C—Cl | C—CH₃ | C—CH₃ |
| 986. | C—CH₃ | C—F | N | CH |
| 987. | C—CH₃ | C—F | N | C—Cl |
| 988. | C—CH₃ | C—F | N | C—F |
| 989. | C—CH₃ | C—F | N | C—Br |
| 990. | C—CH₃ | C—F | N | C—CH₃ |
| 991. | C—CH₃ | C—F | CH | CH |
| 992. | C—CH₃ | C—F | CH | C—Cl |
| 993. | C—CH₃ | C—F | CH | C—F |
| 994. | C—CH₃ | C—F | CH | C—Br |
| 995. | C—CH₃ | C—F | CH | C—CH₃ |
| 996. | C—CH₃ | C—F | C—Cl | CH |
| 997. | C—CH₃ | C—F | C—Cl | C—Cl |
| 998. | C—CH₃ | C—F | C—Cl | C—F |
| 999. | C—CH₃ | C—F | C—Cl | C—Br |
| 1000. | C—CH₃ | C—F | C—Cl | C—CH₃ |
| 1001. | C—CH₃ | C—F | C—F | CH |
| 1002. | C—CH₃ | C—F | C—F | C—Cl |
| 1003. | C—CH₃ | C—F | C—F | C—F |
| 1004. | C—CH₃ | C—F | C—F | C—Br |
| 1005. | C—CH₃ | C—F | C—F | C—CH₃ |
| 1006. | C—CH₃ | C—F | C—Br | CH |
| 1007. | C—CH₃ | C—F | C—Br | C—Cl |
| 1008. | C—CH₃ | C—F | C—Br | C—F |
| 1009. | C—CH₃ | C—F | C—Br | C—Br |
| 1010. | C—CH₃ | C—F | C—Br | C—CH₃ |
| 1011. | C—CH₃ | C—F | C—CH₃ | CH |
| 1012. | C—CH₃ | C—F | C—CH₃ | C—Cl |
| 1013. | C—CH₃ | C—F | C—CH₃ | C—F |
| 1014. | C—CH₃ | C—F | C—CH₃ | C—Br |
| 1015. | C—CH₃ | C—F | C—CH₃ | C—CH₃ |
| 1016. | C—CH₃ | C—Br | N | CH |
| 1017. | C—CH₃ | C—Br | N | C—Cl |
| 1018. | C—CH₃ | C—Br | N | C—F |
| 1019. | C—CH₃ | C—Br | N | C—Br |
| 1020. | C—CH₃ | C—Br | N | C—CH₃ |
| 1021. | C—CH₃ | C—Br | CH | CH |
| 1022. | C—CH₃ | C—Br | CH | C—Cl |
| 1023. | C—CH₃ | C—Br | CH | C—F |
| 1024. | C—CH₃ | C—Br | CH | C—Br |
| 1025. | C—CH₃ | C—Br | CH | C—CH₃ |
| 1026. | C—CH₃ | C—Br | C—Cl | CH |
| 1027. | C—CH₃ | C—Br | C—Cl | C—Cl |
| 1028. | C—CH₃ | C—Br | C—Cl | C—F |
| 1029. | C—CH₃ | C—Br | C—Cl | C—Br |
| 1030. | C—CH₃ | C—Br | C—Cl | C—CH₃ |
| 1031. | C—CH₃ | C—Br | C—F | CH |
| 1032. | C—CH₃ | C—Br | C—F | C—Cl |
| 1033. | C—CH₃ | C—Br | C—F | C—F |
| 1034. | C—CH₃ | C—Br | C—F | C—Br |
| 1035. | C—CH₃ | C—Br | C—F | C—CH₃ |
| 1036. | C—CH₃ | C—Br | C—Br | CH |
| 1037. | C—CH₃ | C—Br | C—Br | C—Cl |
| 1038. | C—CH₃ | C—Br | C—Br | C—F |
| 1039. | C—CH₃ | C—Br | C—Br | C—Br |
| 1040. | C—CH₃ | C—Br | C—Br | C—CH₃ |
| 1041. | C—CH₃ | C—Br | C—CH₃ | CH |
| 1042. | C—CH₃ | C—Br | C—CH₃ | C—Cl |
| 1043. | C—CH₃ | C—Br | C—CH₃ | C—F |
| 1044. | C—CH₃ | C—Br | C—CH₃ | C—Br |
| 1045. | C—CH₃ | C—Br | C—CH₃ | C—CH₃ |
| 1046. | C—CH₃ | C—CH₃ | N | CH |
| 1047. | C—CH₃ | C—CH₃ | N | C—Cl |
| 1048. | C—CH₃ | C—CH₃ | N | C—F |
| 1049. | C—CH₃ | C—CH₃ | N | C—Br |
| 1050. | C—CH₃ | C—CH₃ | N | C—CH₃ |
| 1051. | C—CH₃ | C—CH₃ | CH | CH |
| 1052. | C—CH₃ | C—CH₃ | CH | C—Cl |
| 1053. | C—CH₃ | C—CH₃ | CH | C—F |
| 1054. | C—CH₃ | C—CH₃ | CH | C—Br |
| 1055. | C—CH₃ | C—CH₃ | CH | C—CH₃ |
| 1056. | C—CH₃ | C—CH₃ | C—Cl | CH |
| 1057. | C—CH₃ | C—CH₃ | C—Cl | C—Cl |
| 1058. | C—CH₃ | C—CH₃ | C—Cl | C—F |
| 1059. | C—CH₃ | C—CH₃ | C—Cl | C—Br |
| 1060. | C—CH₃ | C—CH₃ | C—Cl | C—CH₃ |
| 1061. | C—CH₃ | C—CH₃ | C—F | CH |
| 1062. | C—CH₃ | C—CH₃ | C—F | C—Cl |
| 1063. | C—CH₃ | C—CH₃ | C—F | C—F |
| 1064. | C—CH₃ | C—CH₃ | C—F | C—Br |
| 1065. | C—CH₃ | C—CH₃ | C—F | C—CH₃ |
| 1066. | C—CH₃ | C—CH₃ | C—Br | CH |
| 1067. | C—CH₃ | C—CH₃ | C—Br | C—Cl |
| 1068. | C—CH₃ | C—CH₃ | C—Br | C—F |
| 1069. | C—CH₃ | C—CH₃ | C—Br | C—Br |
| 1070. | C—CH₃ | C—CH₃ | C—Br | C—CH₃ |
| 1071. | C—CH₃ | C—CH₃ | C—CH₃ | CH |
| 1072. | C—CH₃ | C—CH₃ | C—CH₃ | C—Cl |
| 1073. | C—CH₃ | C—CH₃ | C—CH₃ | C—F |
| 1074. | C—CH₃ | C—CH₃ | C—CH₃ | C—Br |
| 1075. | C—CH₃ | C—CH₃ | C—CH₃ | C—CH₃ |

Table 49 to Table 96: Includes all the compounds as disclosed in Table 1 to Table 48 respectively wherein compound of formula I.3 is replaced by compound of formula I.4;

As used herein, the term “compound(s) of the present invention” or “compound(s) according to the invention” refers to the compound(s) of formula (I) as defined above, which are also referred to as “compound(s) of formula I” or “compound(s) I” or “formula I compound(s)”, and includes their salts, tautomers, stereoisomers, and N-oxides.

The present invention also relates to a mixture of at least one compound of the invention with at least one mixing partner as defined herein. Preferred are binary mixtures of one compound of the invention as component I with one mixing partner as defined herein as component II. Preferred weight ratios for such binary mixtures are from 5000:1 to 1:5000, preferably from 1000:1 to 1:1000, more preferably from 100:1 to 1:100, particularly from 10:1 to 1:10. In such binary mixtures, components I and II may be used in equal amounts, or an excess of component I, or an excess of component II may be used.

Mixing partners can be selected from pesticides, in particular insecticides, nematicides, and acaricides, fungicides, herbicides, plant growth regulators, fertilizers. Preferred mixing partners are insecticides, nematicides and fungicides.

The following list M of pesticides, grouped and numbered according the Mode of Action Classification of the Insecticide Resistance Action Committee (IRAC), together with which the compounds of the invention can be used and with which potential synergistic effects might be produced, is intended to illustrate the possible combinations, but not to impose any limitation:
M.1 Acetylcholine esterase (AChE) inhibitors: M.1A carbamates, e.g. aldicarb, alanycarb, bendiocarb, benfuracarb, butocarboxim, butoxycarboxim, carbaryl, carbofuran, carbosulfan, ethiofencarb, fenobucarb, formetanate, furathiocarb, isoprocarb, methiocarb, methomyl, metolcarb, oxamyl, pirimicarb, propoxur, thiodicarb, thiofanox, trimethacarb, XMC, xylylcarb and triazamate; or M.1B organophosphates, e.g. acephate, azamethiphos, azinphos-ethyl, azinphosmethyl, cadusafos, chlorethoxyfos, chlorfenvinphos, chlormephos, chlorpyrifos, chlorpyrifos-methyl, coumaphos, cyanophos, demeton-S-methyl, diazinon, dichlorvos/DDVP, dicrotophos, dimethoate, dimethylvinphos, disulfoton, EPN, ethion, ethoprophos, famphur, fenamiphos, fenitrothion, fenthion, fosthiazate, heptenophos, imicyafos, isofenphos, isopropyl O-(methoxyaminothio-phosphoryl) salicylate, isoxathion, malathion, mecarbam, methamidophos, methidathion, mevinphos, monocrotophos, naled, omethoate, oxydemeton-methyl, parathion, parathion-methyl, phenthoate, phorate, phosalone, phosmet, phosphamidon, phoxim, pirimiphosmethyl, profenofos, propetamphos, prothiofos, pyraclofos, pyridaphenthion, quinalphos, sulfotep, tebupirimfos, temephos, terbufos, tetrachlorvinphos, thiometon, triazophos, trichlorfon, and vamidothion;
M.2. GABA-gated chloride channel antagonists: M.2A cyclodiene organochlorine compounds, e.g. endosulfan or chlordane; or M.2B fiproles (phenylpyrazoles), e.g. ethiprole, fipronil, flufiprole, pyrafluprole, and pyriprole;
M.3 Sodium channel modulators from the class of M.3A pyrethroids, e.g. acrinathrin, allethrin, d-cis-trans allethrin, d-trans allethrin, bifenthrin, kappa-bifenthrin, bioallethrin, bioallethrin Scylclopentenyl, bioresmethrin, cycloprothrin, cyfluthrin, beta-cyfluthrin, cyhalothrin, lambdacyhalothrin, gamma-cyhalothrin, cypermethrin, alpha-cypermethrin, beta-cypermethrin, thetacypermethrin, zeta-cypermethrin, cyphenothrin, deltamethrin, empenthrin, esfenvalerate, etofenprox, fenpropathrin, fenvalerate, flucythrinate, flumethrin, tau-fluvalinate, halfenprox, heptafluthrin, imiprothrin, meperfluthrin, metofluthrin, momfluorothrin, epsilon-momfluorothrin, permethrin, phenothrin, prallethrin, profluthrin, pyrethrin (pyrethrum), resmethrin, silafluofen, tefluthrin, kappa-tefluthrin, tetramethylfluthrin, tetramethrin, tralomethrin, and transfluthrin; or M.3B sodium channel modulators such as DDT or methoxychlor;
M.4 Nicotinic acetylcholine receptor agonists (nAChR): M.4A neonicotinoids, e.g. acetamiprid, clothianidin, cycloxaprid, dinotefuran, imidacloprid, nitenpyram, thiacloprid and thiamethoxam; or the compounds M.4A.1 4,5-Dihydro-N-nitro-1-(2-oxiranylmethyl)-1H-imidazol-2-amine, M.4A.2: (2E-)-1-[(6-Chloropyridin-3-yl)methyl]-N′-nitro-2-pentylidenehydrazinecarboximidamide; or M4.A.3: 1-[(6-Chloropyridin-3-yl)methyl]-7-methyl-8-nitro-5-propoxy-1,2,3,5,6,7-hexahydroimidazo[1,2-a]pyridine; or M.4B nicotine; M.4C sulfoxaflor; M.4D flupyradifurone; M.4E triflumezopyrim, M.4E.1a) (3R)-3-(2-chlorothiazol-5-yl)-8-methyl-5-oxo-6-phenyl-2,3-dihydrothiazolo[3,2-a]pyrimidin-8-ium-7-olate, M.4E.1b) (3S)-3-(6-chloro-3-pyridyl)-8-methyl-5-oxo-6-phenyl-2,3-dihydrothiazolo[3,2-a]pyrimidin-8-ium-7-olate, M.4E.1c) (3S)-8-methyl-5-oxo-6-phenyl-3-pyrimidin-5-yl-2,3-dihydrothiazolo[3,2-a]pyrimidin-8-ium-7-olate, M.4E.1d) (3R)-3-(2-chlorothiazol-5-yl)-8-methyl-5-oxo-6-[3-(trifluoromethyl)phenyl]-2,3-dihydrothiazolo[3,2-a]pyrimidin-8-ium-7-olate; M.4E.1e) (3R)-3-(2-chlorothiazol-5-yl)-6-(3,5-dichlorophenyl)-8-methyl-5-oxo-2,3-dihydrothiazolo[3,2-a]pyrimidin-8-ium-7-olate, M.4E.1f) (3R)-3-(2-chlorothiazol-5-yl)-8-ethyl-5-oxo-6-phenyl-2,3-dihydrothiazolo[3,2-a]pyrimidin-8-ium-7-olate;
M.5 Nicotinic acetylcholine receptor allosteric activators: spinosyns, e.g. spinosad or spinetoram;
M.6 Chloride channel activators from the class of avermectins and milbemycins, e.g. abamectin, emamectin benzoate, ivermectin, lepimectin, or milbemectin;
M.7 Juvenile hormone mimics, such as M.7A juvenile hormone analogues hydroprene, kinoprene, and methoprene; or M.7B fenoxycarb, or M.7C pyriproxyfen;
M.8 miscellaneous non-specific (multi-site) inhibitors, e.g. M.8A alkyl halides as methyl bromide and other alkyl halides, M.8B chloropicrin, M.8C sulfuryl fluoride, M.8D borax, or M.8E tartar emetic;
M.9 Chordotonal organ TRPV channel modulators, e.g. M.9B pymetrozine; pyrifluquinazon;
M.10 Mite growth inhibitors, e.g. M.10A clofentezine, hexythiazox, and diflovidazin, or M.10B etoxazole;
M.11 Microbial disruptors of insect midgut membranes, e.g. Bacillus thuringiensis or Bacillus sphaericus and the insecticidal proteins they produce such as Bacillus thuringiensis subsp. israelensis, Bacillus sphaericus, Bacillus thuringiensis subsp. aizawai, Bacillus thuringiensis subsp. kurstaki and Bacillus thuringiensis subsp. tenebrionis, or the Bt crop proteins: Cry1Ab, Cry1Ac, Cry1Fa, Cry2Ab, mCry3A, Cry3Ab, Cry3Bb, and Cry34/35Ab1;
M.12 Inhibitors of mitochondrial ATP synthase, e.g. M.12A diafenthiuron, or M.12B organotin miticides such as azocyclotin, cyhexatin, or fenbutatin oxide, M.12C propargite, or M.12D tetradifon;
M.13 Uncouplers of oxidative phosphorylation via disruption of the proton gradient, e.g. chlorfenapyr, DNOC, or sulfluramid;
M.14 Nicotinic acetylcholine receptor (nAChR) channel blockers, e.g. nereistoxin analogues bensultap, cartap hydrochloride, thiocyclam, or thiosultap sodium;
M.15 Inhibitors of the chitin biosynthesis type 0, such as benzoylureas e.g. bistrifluron, chlorfluazuron, diflubenzuron, flucycloxuron, flufenoxuron, hexaflumuron, lufenuron, novaluron, noviflumuron, teflubenzuron, or triflumuron;
M.16 Inhibitors of the chitin biosynthesis type 1, e.g. buprofezin;
M.17 Moulting disruptors, Dipteran, e.g. cyromazine;
M.18 Ecdyson receptor agonists such as diacylhydrazines, e.g. methoxyfenozide, tebufenozide, halofenozide, fufenozide, or chromafenozide;
M.19 Octopamin receptor agonists, e.g. amitraz;
M.20 Mitochondrial complex III electron transport inhibitors, e.g. M.20A hydramethylnon, M.20B acequinocyl, M.20C fluacrypyrim; or M.20D bifenazate;
M.21 Mitochondrial complex I electron transport inhibitors, e.g. M.21A METI acaricides and insecticides such as fenazaquin, fenpyroximate, pyrimidifen, pyridaben, tebufenpyrad or tolfenpyrad, or M.21B rotenone;
M.22 Voltage-dependent sodium channel blockers, e.g. M.22A indoxacarb, M.22B metaflumizone, or M.22B.1: 2-[2-(4-Cyanophenyl)-1-[3-(trifluoromethyl)phenyl]ethylidene]-N-[4-(difluoromethoxy)phenyl]-hydrazinecarboxamide or M.22B.2: N-(3-Chloro-2-methylphenyl)-2-[(4-chlorophenyl) [4-[methyl(methylsulfonyl)amino]phenyl]methylene]-hydrazinecarboxamide;
M.23 Inhibitors of the of acetyl CoA carboxylase, such as Tetronic and Tetramic acid derivatives, e.g. spirodiclofen, spiromesifen, or spirotetramat; M.23.1 spiropidion;
M.24 Mitochondrial complex IV electron transport inhibitors, e.g. M.24A phosphine such as aluminium phosphide, calcium phosphide, phosphine or zinc phosphide, or M.24B cyanide;
M.25 Mitochondrial complex II electron transport inhibitors, such as beta-ketonitrile derivatives, e.g. cyenopyrafen or cyflumetofen;
M.28 Ryanodine receptor-modulators from the class of diamides, e.g. flubendiamide, chlorantraniliprole, cyantraniliprole, tetraniliprole, M.28.1: (R)-3-Chlor-N1-{2-methyl-4-[1,2,2,2-tetrafluoro-1-(trifluoromethyl)ethyl]phenyl}-N2-(1-methyl-2-methylsulfonylethyl)phthalamid, M.28.2: (S)-3-Chloro-N1-{2-methyl-4-[1,2,2,2-tetrafluoro-1-(trifluoromethyl)ethyl]phenyl}-N2-(1-methyl-2-methylsulfonylethyl) phthalamid, M.28.3: cyclaniliprole, or M.28.4: methyl-2-[3,5-dibromo-2-({[3-bromo-1-(3-chlorpyridin-2-yl)-1H-pyrazol-5-yl]carbonyl}amino)benzoyl]-1,2-dimethylhydrazinecarboxylate; M.28.5i) N-[2-(5-Amino-1,3,4-thiadiazol-2-yl)-4-chloro-6-methylphenyl]-3-bromo-1-(3-chloro-2-pyridinyl)-1H-pyrazole-5-carboxamide; M.28.5j) 3-Chloro-1-(3-chloro-2-pyridinyl)-N-[2,4-dichloro-6-[[(1-cyano-1-methylethyl)amino]carbonyl]phenyl]-1H-pyrazole-5-carboxamide; M.28.5k) tetrachlorantraniliprole; M.28.51) N-[4-Chloro-2-[[(1,1-dimethylethyl)amino]carbonyl]-6-methylphenyl]-1-(3-chloro-2-pyridinyl)-3-(fluoromethoxy)-1H-pyrazole-5-carboxamide; or
M.28.6: cyhalodiamide; or
M.29: Chordotonal organ Modulators-undefined target site, e.g. flonicamid;
M.UN. insecticidal active compounds of unknown or uncertain mode of action, e.g. afidopyropen, afoxolaner, azadirachtin, amidoflumet, benzoximate, broflanilide, bromopropylate, chinomethionat, cryolite, dicloromezotiaz, dicofol, flufenerim, flometoquin, fluensulfone, fluhexafon, fluopyram, fluralaner, metaldehyde, metoxadiazone, piperonyl butoxide, pyflubumide, pyridalyl, tioxazafen, M.UN.3: 11-(4-chloro-2,6-dimethylphenyl)-12-hydroxy-1,4-dioxa-9-azadispiro[4.2.4.2]-tetradec-11-en-10-one,
M.UN.4: 3-(4′-fluoro-2,4-dimethylbiphenyl-3-yl)-4-hydroxy-8-oxa-1-azaspiro[4.5]dec-3-en-2-one,
M.UN.5: 1-[2-fluoro-4-methyl-5-[(2,2,2-trifluoroethyl) sulfinyl]phenyl]-3-(trifluoromethyl)-1H-1,2,4-triazole-5-amine, or actives on basis of Bacillus firmus (Votivo, I-1582);
M.UN.6: flupyrimin;
M.UN.8: fluazaindolizine; M.UN.9.a): 4-[5-(3,5-dichlorophenyl)-5-(trifluoromethyl)-4H-isoxazol-3-yl]-2-methyl-N-(1-oxothietan-3-yl)benzamide; M.UN.9.b): fluxametamide; M.UN.10: 5-[3-[2,6-dichloro-4-(3,3-dichloroallyloxy)phenoxy]propoxy]-1H-pyrazole;
M.UN.11.i) 4-cyano-N-[2-cyano-5-[[2,6-dibromo-4-[1,2,2,3,3,3-hexafluoro-1-(trifluoromethyl)propyl]phenyl]carbamoyl]phenyl]-2-methyl-benzamide; M.UN.11.j) 4-cyano-3-[(4-cyano-2-methyl-benzoyl)amino]-N-[2,6-dichloro-4-[1,2,2,3,3,3-hexafluoro-1-(trifluoromethyl)propyl]phenyl]-2-fluoro-benzamide; M.UN.11.k) N-[5-[[2-chloro-6-cyano-4-[1,2,2,3,3,3-hexafluoro-1-(trifluoromethyl) propyl]phenyl]carbamoyl]-2-cyano-phenyl]-4-cyano-2-methyl-benzamide; M.UN.11.l) N-[5-[[2-bromo-6-chloro-4-[2,2,2-trifluoro-1-hydroxy-1-(trifluoromethyl)ethyl]phenyl]carbamoyl]-2-cyano-phenyl]-4-cyano-2-methyl-benzamide; M.UN.11.m) N-[5-[[2-bromo-6-chloro-4-[1,2,2,3,3,3-hexafluoro-1-(trifluoromethyl)propyl]phenyl]carbamoyl]-2-cyano-phenyl]-4-cyano-2-methyl-benzamide; M.UN.11.n) 4-cyano-N-[2-cyano-5-[[2,6-dichloro-4-[1,2,2,3,3,3-hexafluoro-1-(trifluoromethyl)propyl]phenyl]carbamoyl]phenyl]-2-methyl-benzamide; M.UN.11.0) 4-cyano-N-[2-cyano-5-[[2,6-dichloro-4-[1,2,2,2-tetrafluoro-1-(trifluoromethyl)ethyl]phenyl]carbamoyl]phenyl]-2-methyl-benzamide; M.UN.11.p) N-[5-[[2-bromo-6-chloro-4-[1,2,2,2-tetrafluoro-1-(trifluoromethyl)ethyl]phenyl]carbamoyl]-2-cyano-phenyl]-4-cyano-2-methyl-benzamide; or
M.UN.12.a) 2-(1,3-Dioxan-2-yl)-6-[2-(3-pyridinyl)-5-thiazolyl]-pyridine; M.UN.12.b) 2-[6-[2-(5-Fluoro-3-pyridinyl)-5-thiazolyl]-2-pyridinyl]-pyrimidine; M.UN.12.c) 2-[6-[2-(3-Pyridinyl)-5-thiazolyl]-2-pyridinyl]-pyrimidine; M.UN.12.d) N-Methylsulfonyl-6-[2-(3-pyridyl) thiazol-5-yl]pyridine-2-carboxamide; M.UN.12.e) N-Methylsulfonyl-6-[2-(3-pyridyl) thiazol-5-yl]pyridine-2-carboxamide;
M.UN.14a) 1-[(6-Chloro-3-pyridinyl)methyl]-1,2,3,5,6,7-hexahydro-5-methoxy-7-methyl-8-nitroimidazo[1,2-a]pyridine; or M.UN.14b) 1-[(6-Chloropyridin-3-yl)methyl]-7-methyl-8-nitro-1,2,3,5,6,7-hexahydroimidazo[1,2-a]pyridin-5-ol;
M.UN.16a) 1-isopropyl-N,5-dimethyl-N-pyridazin-4-yl-pyrazole-4-carboxamide; or M.UN.16b) 1-(1,2-dimethylpropyl)-N-ethyl-5-methyl-N-pyridazin-4-yl-pyrazole-4-carboxamide; M.UN.16c) N,5-dimethyl-N-pyridazin-4-yl-1-(2,2,2-trifluoro-1-methyl-ethyl) pyrazole-4-carboxamide; M.UN.16d) 1-[1-(1-cyanocyclopropyl)ethyl]-N-ethyl-5-methyl-N-pyridazin-4-yl-pyrazole-4-carboxamide; M.UN.16e) N-ethyl-1-(2-fluoro-1-methyl-propyl)-5-methyl-N-pyridazin-4-yl-pyrazole-4-carboxamide; M.UN.16f) 1-(1,2-dimethylpropyl)-N,5-dimethyl-N-pyridazin-4-yl-pyrazole-4-carboxamide; M.UN.16g) 1-[1-(1-cyanocyclopropyl)ethyl]-N,5-dimethyl-N-pyridazin-4-yl-pyrazole-4-carboxamide; M.UN.16h) N-methyl-1-(2-fluoro-1-methyl-propyl]-5-methyl-N-pyridazin-4-yl-pyrazole-4-carboxamide; M.UN.16i) 1-(4,4-difluorocyclohexyl)-N-ethyl-5-methyl-N-pyridazin-4-yl-pyrazole-4-carboxamide; or M.UN.16j) 1-(4,4-difluorocyclohexyl)-N,5-dimethyl-N-pyridazin-4-yl-pyrazole-4-carboxamide,
M.UN.17a) N-(1-methylethyl)-2-(3-pyridinyl)-2H-indazole-4-carboxamide; M.UN.17b) N-cyclopropyl-2-(3-pyridinyl)-2H-indazole-4-carboxamide; M.UN.17c) N-cyclohexyl-2-(3-pyridinyl)-2H-indazole-4-carboxamide; M.UN.17d) 2-(3-pyridinyl)-N-(2,2,2-trifluoroethyl)-2H-indazole-4-carboxamide; M.UN.17e) 2-(3-pyridinyl)-N-[(tetrahydro-2-furanyl)methyl]-2H-indazole-5-carboxamide; M.UN.17f) methyl 2-[[2-(3-pyridinyl)-2H-indazol-5-yl]carbonyl]hydrazinecarboxylate; M.UN.17g) N-[(2,2-difluorocyclopropyl)methyl]-2-(3-pyridinyl)-2H-indazole-5-carboxamide; M.UN.17h) N-(2,2-difluoropropyl)-2-(3-pyridinyl)-2H-indazole-5-carboxamide; M.UN.17i) 2-(3-pyridinyl)-N-(2-pyrimidinylmethyl)-2H-indazole-5-carboxamide; M.UN.17j) N-[(5-methyl-2-pyrazinyl)methyl]-2-(3-pyridinyl)-2H-indazole-5-carboxamide,
M.UN.18. tyclopyrazoflor;
M.UN.19 sarolaner, M.UN.20 lotilaner;
M.UN.21 N-[4-Chloro-3-[[(phenylmethyl)amino]carbonyl]phenyl]-1-methyl-3-(1,1,2,2,2-pentafluoroethyl)-4-(trifluoromethyl)-1H-pyrazole-5-carboxamide; M.UN.22a 2-(3-ethylsulfonyl-2-pyridyl)-3-methyl-6-(trifluoromethyl) imidazo[4,5-b]pyridine, or M.UN.22b 2-[3-ethylsulfonyl-5-(trifluoromethyl)-2-pyridyl]-3-methyl-6-(trifluoromethyl) imidazo[4,5-b]pyridine;
M.UN.23 Isocycloseram;
M.UN.24a) N-[4-chloro-3-(cyclopropylcarbamoyl)phenyl]-2-methyl-5-(1,1,2,2,2-pentafluoroethyl)-4-(trifluoromethyl) pyrazole-3-carboxamide or M.UN.24b) N-[4-chloro-3-[(1-cyanocyclopropyl) carbamoyl]phenyl]-2-methyl-5-(1,1,2,2,2-pentafluoroethyl)-4-(trifluoromethyl) pyrazole-3-carboxamide; M.UN.25 acynonapyr; M.UN.26 benzpyrimoxan; M.UN.27 tigolaner; M.UN.28 Oxazosulfyl;
M.UN.29a) [(2S,3R,4R,5S,6S)-3,5-dimethoxy-6-methyl-4-propoxy-tetrahydropyran-2-yl] N-[4-[1-[4-(trifluoromethoxy)phenyl]-1,2,4-triazol-3-yl]phenyl]carbamate; M.UN.29b) [(2S,3R,4R,5S,6S)-3,4,5-trimethoxy-6-methyl-tetrahydropyran-2-yl] N-[4-[1-[4-(trifluoromethoxy)phenyl]-1,2,4-triazol-3-yl]phenyl]carbamate; M.UN.29c) [(2S,3R,4R,5S,6S)-3,5-dimethoxy-6-methyl-4-propoxy-tetrahydropyran-2-yl] N-[4-[1-[4-(1,1,2,2,2-pentafluoroethoxy)phenyl]-1,2,4-triazol-3-yl]phenyl]carbamate; M.UN.29d) [(2S,3R,4R,5S,6S)-3,4,5-trimethoxy-6-methyl-tetrahydropyran-2-yl] N-[4-[1-[4-(1,1,2,2,2-pentafluoroethoxy)phenyl]-1,2,4-triazol-3-yl]phenyl]carbamate; M.UN.29.e) (2Z)-3-(2-isopropylphenyl)-2-[(E)-[4-[1-[4-(trifluoromethoxy)phenyl]-1,2,4-triazol-3-yl]phenyl]methylenehydrazono]thiazolidin-4-one or M.UN.29f) (2Z)-3-(2-isopropylphenyl)-2-[(E)-[4-[1-[4-(1,1,2,2,2-pentafluoroethoxy)phenyl]-1,2,4-triazol-3-yl]phenyl]methylenehydrazono]thiazolidin-4-one;
M.UN.30a) 2-(6-chloro-3-ethylsulfonyl-imidazo[1,2-a]pyridin-2-yl)-3-methyl-6-(trifluoromethyl) imidazo[4,5-b]pyridine, M.UN.30b) 2-(6-bromo-3-ethylsulfonyl-imidazo[1,2-a]pyridin-2-yl)-3-methyl-6-(trifluoromethyl) imidazo[4,5-b]pyridine, M.UN.30c) 2-(3-ethylsulfonyl-6-iodo-imidazo[1,2-a]pyridin-2-yl)-3-methyl-6-(trifluoromethyl) imidazo[4,5-b]pyridine, M.UN.30d) 2-[3-ethylsulfonyl-6-(trifluoromethyl) imidazo[1,2-a]pyridin-2-yl]-3-methyl-6-(trifluoromethyl) imidazo[4,5-b]pyridine, M.UN.30e) 2-(7-chloro-3-ethylsulfonyl-imidazo[1,2-a]pyridin-2-yl)-3-methyl-6-(trifluoromethyl) imidazo[4,5-b]pyridine, M.UN.30f) 2-(3-ethylsulfonyl-7-iodo-imidazo[1,2-a]pyridin-2-yl)-3-methyl-6-(trifluoromethyl) imidazo[4,5-b]pyridine, M.UN.30g) 3-ethylsulfonyl-6-iodo-2-[3-methyl-6-(trifluoromethyl) imidazo[4,5-b]pyridin-2-yl]imidazo[1,2-a]pyridine-8-carbonitrile, M.UN.30h) 2-[3-ethylsulfonyl-8-fluoro-6-(trifluoromethyl) imidazo[1,2-a]pyridin-2-yl]-3-methyl-6-(trifluoromethyl) imidazo[4,5-b]pyridine, M.UN.30i) 2-[3-ethylsulfonyl-7-(trifluoromethyl) imidazo[1,2-a]pyridin-2-yl]-3-methyl-6-(trifluoromethylsulfinyl) imidazo[4,5-b]pyridine, M.UN.30j) 2-[3-ethylsulfonyl-7-(trifluoromethyl) imidazo[1,2-a]pyridin-2-yl]-3-methyl-6-(trifluoromethyl) imidazo[4,5-c]pyridine, M.UN.30k) 2-(6-bromo-3-ethylsulfonyl-imidazo[1,2-a]pyridin-2-yl)-6-(trifluoromethyl) pyrazolo[4,3-c]pyridine.

The commercially available compounds of the group M listed above may be found in The Pesticide Manual, 17th Edition, C. MacBean, British Crop Protection Council (2015) among other publications. The online Pesticide Manual is updated regularly and is accessible through http://bcpcdata.com/pesticide-manual.html.

Another online data base for pesticides providing the ISO common names is http://www.alanwood.net/pesticides.

The M.4 cycloxaprid is known from WO2010/069266 and WO2011/069456. M.4A.1 is known from CN 103814937; CN105367557, CN 105481839. M.4A.2, guadipyr, is known from WO 2013/003977, and M.4A.3 (approved as paichongding in China) is known from WO 2007/101369. M.4E.1a) to M.4E.1f) are known from WO2018177970. M.22B.1 is described in CN10171577 and M.22B.2 in CN102126994. Spiropidion M.23.1 is known from WO 2014/191271. M.28.1 and M.28.2 are known from WO2007/101540. M.28.3 is described in WO2005/077934. M.28.4 is described in WO2007/043677. M.28.5a) to M.28.5d) and M.28.5h) are described in WO 2007/006670, WO2013/024009 and WO 2013/024010, M.28.5i) is described in WO2011/085575, M.28.5j) in WO2008/134969, M.28.5k) in US2011/046186 and M.28.5l) in WO2012/034403. M.28.6 can be found in WO2012/034472. M.UN.3 is known from WO2006/089633 and M.UN.4 from WO2008/067911. M.UN.5 is described in WO2006/043635, and biological control agents on the basis of Bacillus firmus are described in WO2009/124707. Flupyrimin is described in WO2012/029672. M.UN.8 is known from WO2013/055584. M.UN.9.a) is described in WO2013/050317. M.UN.9.b) is described in WO2014/126208. M.UN.10 is known from WO2010/060379. Broflanilide and M.UN.11.b) to M.UN.11.h) are described in WO2010/018714, and M.UN.11i) to M.UN.11.p) in WO 2010/127926. M.UN.12.a) to M.UN.12.c) are known from WO2010/006713, M.UN.12.d) and M.UN.12.e) are known from WO2012/000896. M.UN.14a) and M.UN.14b) are known from WO2007/101369. M.UN.16.a) to M.UN.16h) are described in WO2010/034737, WO2012/084670, and WO2012/143317, resp., and M.UN.16i) and M.UN.16j) are described in WO2015/055497. M.UN.17a) to M.UN.17.j) are described in WO2015/038503. M.UN.18 Tycloprazoflor is described in US2014/0213448. M.UN.19 is described in WO2014/036056. M.UN.20 is known from WO2014/090918. M.UN.21 is known from EP2910126. M.UN.22a and M.UN.22b are known from WO2015/059039 and WO2015/190316. M.UN.23a and M.UN.23b are known from WO2013/050302. M.UN.24a) and M.UN.24b) are known from WO2012/126766. Acynonapyr M.UN.25 is known from WO 2011/105506. Benzpyrimoxan M.UN.26 is known from WO2016/104516. M.UN.27 is known from WO2016/174049. M.UN.28 Oxazosulfyl is known from WO2017/104592. M.UN.29a) to M.UN.29f) are known from WO2009/102736 or WO2013116053. M.UN.30 is known from WO2013/050302. M.UN.30a) to M.UN.30k) are known from WO2018/052136.

The following list of fungicides, in conjunction with which the compounds of the present invention can be used, is intended to illustrate the possible combinations but does not limit them:
A) Respiration inhibitors
   Inhibitors of complex III at Qₒ site: azoxystrobin (A.1.1), coumethoxystrobin (A.1.2), coumoxystrobin (A.1.3), dimoxystrobin (A.1.4), enestroburin (A.1.5), fenaminstrobin (A.1.6), fenoxystrobin/flufenoxystrobin (A.1.7), fluoxastrobin (A.1.8), kresoxim-methyl (A.1.9), mandestrobin (A.1.10), metominostrobin (A.1.11), orysastrobin (A.1.12), picoxystrobin (A.1.13), pyraclostrobin (A.1.14), pyrametostrobin (A.1.15), pyraoxystrobin (A.1.16), trifloxystrobin (A.1.17), 2-(2-(3-(2,6-dichlorophenyl)-1-methyl-allylideneaminooxymethyl)-phenyl)-2-methoxyimino-N-methyl-acetamide (A.1.18), pyribencarb (A.1.19), triclopyricarb/chlorodincarb (A.1.20), famoxadone (A.1.21), fenamidone (A.1.21), methyl-N-[2-[(1,4-dimethyl-5-phenylpyrazol-3-yl)oxylmethyl]phenyl]-N-methoxy-carbamate (A.1.22), metyltetrapole (A.1.25), (Z,2E)-5-[1-(2,4-dichlorophenyl) pyrazol-3-yl]-oxy-2-methoxyimino-N,3-dimethyl-pent-3-enamide (A.1.34), (Z,2E)-5-[1-(4-chlorophenyl) pyrazol-3-yl]oxy-2-methoxyimino-N,3-dimethyl-pent-3-enamide (A.1.35), pyriminostrobin (A.1.36), bifujunzhi (A.1.37), 2-(ortho-((2,5-dimethylphenyloxymethylen)phenyl)-3-methoxy-acrylic acid methylester (A.1.38);
   inhibitors of complex III at Qᵢ site: cyazofamid (A.2.1), amisulbrom (A.2.2), [(6S,7R,8R)-8-benzyl-3-[(3-hydroxy-4-methoxy-pyridine-2-carbonyl)amino]-6-methyl-4,9-dioxo-1,5-dioxonan-7-yl] 2-methylpropanoate (A.2.3), fenpicoxamid (A.2.4), florylpicoxamid (A.2.5);
   inhibitors of complex II: benodanil (A.3.1), benzovindiflupyr (A.3.2), bixafen (A.3.3), boscalid (A.3.4), carboxin (A.3.5), fenfuram (A.3.6), fluopyram (A.3.7), flutolanil (A.3.8), fluxapyroxad (A.3.9), furametpyr (A.3.10), isofetamid (A.3.11), isopyrazam (A.3.12), mepronil (A.3.13), oxycarboxin (A.3.14), penflufen (A.3.15), penthiopyrad (A.3.16), pydiflumetofen (A.3.17), pyraziflumid (A.3.18), sedaxane (A.3.19), tecloftalam (A.3.20), thifluzamide (A.3.21), inpyrfluxam (A.3.22), pyrapropoyne (A.3.23), fluindapyr (A.3.28), N-[2-[2-chloro-4-(trifluoromethyl) phenoxy]phenyl]-3-(difluoromethyl)-5-fluoro-1-methyl-pyrazole-4-carboxamide (A.3.29), methyl (E)-2-[2-[(5-cyano-2-methyl-phenoxy)methyl]phenyl]-3-methoxy-prop-2-enoate (A.3.30), isoflucypram (A.3.31), 2-(difluoromethyl)-N-(1,1,3-trimethyl-indan-4-yl)pyridine-3-carboxamide (A.3.32), 2-(difluoromethyl)-N-[(3R)-1,1,3-trimethylindan-4-yl]pyridine-3-carboxamide (A.3.33), 2-(difluoromethyl)-N-(3-ethyl-1,1-dimethyl-indan-4-yl)pyridine-3-carboxamide (A.3.34), 2-(difluoromethyl)-N-[(3R)-3-ethyl-1,1-dimethyl-indan-4-yl]pyridine-3-carboxamide (A.3.35), 2-(difluoromethyl)-N-(1,1-dimethyl-3-propyl-indan-4-yl)pyridine-3-carboxamide (A.3.36), 2-(difluoromethyl)-N-[(3R)-1,1-dimethyl-3-propyl-indan-4-yl]pyridine-3-carboxamide (A.3.37), 2-(difluoromethyl)-N-(3-isobutyl-1,1-dimethyl-indan-4-yl)pyridine-3-carboxamide (A.3.38), 2-(difluoromethyl)-N-[(3R)-3-isobutyl-1,1-dimethyl-indan-4-yl]pyridine-3-carboxamide (A.3.39);
   other respiration inhibitors: diflumetorim (A.4.1); nitrophenyl derivates: binapacryl (A.4.2), dinobuton (A.4.3), dinocap (A.4.4), fluazinam (A.4.5), meptyldinocap (A.4.6), ferimzone (A.4.7); organometal compounds: fentin salts, e. g. fentin-acetate (A.4.8), fentin chloride (A.4.9) or fentin hydroxide (A.4.10); ametoctradin (A.4.11); silthiofam (A.4.12);
B) Sterol biosynthesis inhibitors (SBI fungicides)
   C14 demethylase inhibitors: triazoles: azaconazole (B.1.1), bitertanol (B.1.2), bromuconazole (B.1.3), cyproconazole (B.1.4), difenoconazole (B.1.5), diniconazole (B.1.6), diniconazole-M (B.1.7), epoxiconazole (B.1.8), fenbuconazole (B.1.9), fluquinconazole (B.1.10), flusilazole (B.1.11), flutriafol (B.1.12), hexaconazole (B.1.13), imibenconazole (B.1.14), ipconazole (B.1.15), metconazole (B.1.17), myclobutanil (B.1.18), oxpoconazole (B.1.19), paclobutrazole (B.1.20), penconazole (B.1.21), propiconazole (B.1.22), prothioconazole (B.1.23), simeconazole (B.1.24), tebuconazole (B.1.25), tetraconazole (B.1.26), triadimefon (B.1.27), triadimenol (B.1.28), triticonazole (B.1.29), uniconazole (B.1.30), 2-(2,4-difluorophenyl)-1,1-difluoro-3-(tetrazol-1-yl)-1-[5-[4-(2,2,2-trifluoroethoxy)phenyl]-2-pyridyl]propan-2-ol (B.1.31), 2-(2,4-difluorophenyl)-1,1-difluoro-3-(tetrazol-1-yl)-1-[5-[4-(trifluoromethoxy)phenyl]-2-pyridyl]propan-2-ol (B.1.32), ipfentrifluconazole (B.1.37), mefentrifluconazole (B.1.38), 2-(chloromethyl)-2-methyl-5-(p-tolylmethyl)-1-(1,2,4-triazol-1-ylmethyl)cyclopentanol (B.1.43); imidazoles: imazalil (B.1.44), pefurazoate (B.1.45), prochloraz (B.1.46), triflumizol (B.1.47); pyrimidines, pyridines, piperazines: fenarimol (B.1.49), pyrifenox (B.1.50), triforine (B.1.51), [3-(4-chloro-2-fluoro-phenyl)-5-(2,4-difluorophenyl) isoxazol-4-yl]-(3-pyridyl) methanol (B.1.52);
   Delta14-reductase inhibitors: aldimorph (B.2.1), dodemorph (B.2.2), dodemorphacetate (B.2.3), fenpropimorph (B.2.4), tridemorph (B.2.5), fenpropidin (B.2.6), piperalin (B.2.7), spiroxamine (B.2.8);
   Inhibitors of 3-keto reductase: fenhexamid (B.3.1);
   Other Sterol biosynthesis inhibitors: chlorphenomizole (B.4.1);
C) Nucleic acid synthesis inhibitors
   phenylamides or acyl amino acid fungicides: benalaxyl (C.1.1), benalaxyl-M (C.1.2), kiralaxyl (C.1.3), metalaxyl (C.1.4), metalaxyl-M (C.1.5), ofurace (C.1.6), oxadixyl (C.1.7);
   other nucleic acid synthesis inhibitors: hymexazole (C.2.1), octhilinone (C.2.2), oxolinic acid (C.2.3), bupirimate (C.2.4), 5-fluorocytosine (C.2.5), 5-fluoro-2-(p-tolylmethoxy)pyrimidin-4-amine (C.2.6), 5-fluoro-2-(4-fluorophenylmethoxy)pyrimidin-4-amine (C.2.7), 5-fluoro-2-(4-chlorophenylmethoxy)pyrimidin-4 amine (C.2.8);
D) Inhibitors of cell division and cytoskeleton
   tubulin inhibitors: benomyl (D.1.1), carbendazim (D.1.2), fuberidazole (D1.3), thiabendazole (D.1.4), thiophanate-methyl (D.1.5), pyridachlometyl (D.1.6), N-ethyl-2-[(3-ethynyl-8-methyl-6-quinolyl)oxy]butanamide (D.1.8), N-ethyl-2-[(3-ethynyl-8-methyl-6-quinolyl)oxy]-2-methylsulfanyl-acetamide (D.1.9), 2-[(3-ethynyl-8-methyl-6-quinolyl)oxy]-N-(2-fluoroethyl) butanamide (D.1.10), 2-[(3-ethynyl-8-methyl-6-quinolyl)oxy]-N-(2-fluoroethyl)-2-methoxy-acetamide (D.1.11), 2-[(3-ethynyl-8-methyl-6-quinolyl)oxy]-N-propyl-butanamide (D.1.12), 2-[(3-ethynyl-8-methyl-6-quinolyl)oxy]-2-methoxy-N-propyl-acetamide (D.1.13), 2-[(3-ethynyl-8-methyl-6-quinolyl)oxy]-2-methylsulfanyl-N-propyl-acetamide (D.1.14), 2-[(3-ethynyl-8-methyl-6-quinolyl)oxy]-N-(2-fluoroethyl)-2-methylsulfanyl-acetamide (D.1.15), 4-(2-bromo-4-fluoro-phenyl)-N-(2-chloro-6-fluoro-phenyl)-2,5-dimethyl-pyrazol-3-amine (D.1.16);
   other cell division inhibitors: diethofencarb (D.2.1), ethaboxam (D.2.2), pencycuron (D.2.3), fluopicolide (D.2.4), zoxamide (D.2.5), metrafenone (D.2.6), pyriofenone (D.2.7);
E) Inhibitors of amino acid and protein synthesis
   methionine synthesis inhibitors: cyprodinil (E.1.1), mepanipyrim (E.1.2), pyrimethanil (E.1.3);
   protein synthesis inhibitors: blasticidin-S (E.2.1), kasugamycin (E.2.2), kasugamycin hydrochloride-hydrate (E.2.3), mildiomycin (E.2.4), streptomycin (E.2.5), oxytetracyclin (E.2.6);
F) Signal transduction inhibitors
   MAP/histidine kinase inhibitors: fluoroimid (F.1.1), iprodione (F.1.2), procymidone (F.1.3), vinclozolin (F.1.4), fludioxonil (F.1.5);
   G protein inhibitors: quinoxyfen (F.2.1);
G) Lipid and membrane synthesis inhibitors
   Phospholipid biosynthesis inhibitors: edifenphos (G.1.1), iprobenfos (G.1.2), pyrazophos (G.1.3), isoprothiolane (G.1.4);
   lipid peroxidation: dicloran (G.2.1), quintozene (G.2.2), tecnazene (G.2.3), tolclofos-methyl (G.2.4), biphenyl (G.2.5), chloroneb (G.2.6), etridiazole (G.2.7);
   phospholipid biosynthesis and cell wall deposition: dimethomorph (G.3.1), flumorph (G.3.2), mandipropamid (G.3.3), pyrimorph (G.3.4), benthiavalicarb (G.3.5), iprovalicarb (G.3.6), valifenalate (G.3.7);
   compounds affecting cell membrane permeability and fatty acides: propamocarb (G.4.1);
   inhibitors of oxysterol binding protein: oxathiapiprolin (G.5.1), fluoxapiprolin (G.5.3), 4-[1-[2-[3-(difluoromethyl)-5-methyl-pyrazol-1-yl]acetyl]-4-piperidyl]-N-tetralin-1-ylpyridine-2-carboxamide (G.5.4), 4-[1-[2-[3,5-bis(difluoromethyl) pyrazol-1-yl]acetyl]-4-piperidyl]-N-tetralin-1-yl-pyridine-2-carboxamide (G.5.5), 4-[1-[2-[3-(difluoromethyl)-5-(trifluoromethyl) pyrazol-1-yl]acetyl]-4-piperidyl]-N-tetralin-1-yl-pyridine-2-carboxamide (G.5.6), 4-[1-[2-[5-cyclopropyl-3-(difluoromethyl) pyrazol-1-yl]acetyl]-4-piperidyl]-N-tetralin-1-yl-pyridine-2-carboxamide (G.5.7), 4-[1-[2-[5-methyl-3-(trifluoromethyl) pyrazol-1-yl]acetyl]-4-piperidyl]-N-tetralin-1-yl-pyridine-2-carboxamide (G.5.8), 4-[1-[2-[5-(difluoromethyl)-3-(trifluoromethyl) pyrazol-1-yl]acetyl]-4-piperidyl]-N-tetralin-1-yl-pyridine-2-carboxamide (G.5.9), 4-[1-[2-[3,5-bis(trifluoromethyl) pyrazol-1-yl]acetyl]-4-piperidyl]-N-tetralin-1-yl-pyridine-2-carboxamide (G.5.10), (4-[1-[2-[5-cyclopropyl-3-(trifluoromethyl) pyrazol-1-yl]acetyl]-4-piperidyl]-N-tetralin-1-ylpyridine-2-carboxamide (G.5.11);
H) Inhibitors with Multi Site Action
   inorganic active substances: Bordeaux mixture (H.1.1), copper (H.1.2), copper acetate (H.1.3), copper hydroxide (H.1.4), copper oxychloride (H.1.5), basic copper sulfate (H.1.6), sulfur (H.1.7);
   thio- and dithiocarbamates: ferbam (H.2.1), mancozeb (H.2.2), maneb (H.2.3), metam (H.2.4), metiram (H.2.5), propineb (H.2.6), thiram (H.2.7), zineb (H.2.8), ziram (H.2.9);
   organochlorine compounds: anilazine (H.3.1), chlorothalonil (H.3.2), captafol (H.3.3), captan (H.3.4), folpet (H.3.5), dichlofluanid (H.3.6), dichlorophen (H.3.7), hexachlorobenzene (H.3.8), pentachlorphenole (H.3.9) and its salts, phthalide (H.3.10), tolylfluanid (H.3.11);
   guanidines and others: guanidine (H.4.1), dodine (H.4.2), dodine free base (H.4.3), guazatine (H.4.4), guazatine-acetate (H.4.5), iminoctadine (H.4.6), iminoctadine-triacetate (H.4.7), iminoctadine-tris(albesilate) (H.4.8), dithianon (H.4.9), 2,6-dimethyl-1H,5H-[1,4]dithiino[2,3-c:5,6-c′]dipyrrole-1,3,5,7(2H,6H)-tetraone (H.4.10);
I) Cell wall synthesis inhibitors
   inhibitors of glucan synthesis: validamycin (I.1.1), polyoxin B (1.1.2);
   melanin synthesis inhibitors: pyroquilon (1.2.1), tricyclazole (1.2.2), carpropamid (1.2.3), dicyclomet (1.2.4), fenoxanil (1.2.5);
J) Plant defence inducers
   acibenzolar-S-methyl (J.1.1), probenazole (J.1.2), isotianil (J.1.3), tiadinil (J.1.4), prohexadione-calcium (J.1.5); phosphonates: fosetyl (J.1.6), fosetyl-aluminum (J.1.7), phosphorous acid and its salts (J.1.8), calcium phosphonate (J.1.11), potassium phosphonate (J.1.12), potassium or sodium bicarbonate (J.1.9), 4-cyclopropyl-N-(2,4-dimethoxy-phenyl) thiadiazole-5-carboxamide (J.1.10);
K) Unknown mode of action
   bronopol (K.1.1), chinomethionat (K.1.2), cyflufenamid (K.1.3), cymoxanil (K.1.4), dazomet (K.1.5), debacarb (K.1.6), diclocymet (K.1.7), diclomezine (K.1.8), difenzoquat (K.1.9), difenzoquat-methylsulfate (K.1.10), diphenylamin (K.1.11), fenitropan (K.1.12), fenpyrazamine (K.1.13), flumetover (K.1.14), flusulfamide (K.1.15), flutianil (K.1.16), harpin (K.1.17), methasulfocarb (K.1.18), nitrapyrin (K.1.19), nitrothal-isopropyl (K.1.20), tolprocarb (K.1.21), oxin-copper (K.1.22), proquinazid (K.1.23), tebufloquin (K.1.24), tecloftalam (K.1.25), triazoxide (K.1.26), N′-(4-(4-chloro-3-trifluoromethyl-phenoxy)-2,5-dimethyl-phenyl)-N-ethyl-N-methyl formamidine (K.1.27), N′-(4-(4-fluoro-3-trifluoromethyl-phenoxy)-2,5-dimethyl-phenyl)-N-ethyl-N-methyl formamidine (K.1.28), N′-[4-[[3-[(4-chlorophenyl)methyl]-1,2,4-thiadiazol-5-yl]oxy]-2,5-dimethyl-phenyl]-N-ethyl-N-methyl-formamidine (K.1.29), N′-(5-bromo-6-indan-2-yloxy-2-methyl-3-pyridyl)-N-ethyl-N-methyl-formamidine (K.1.30), N′-[5-bromo-6-[1-(3,5-difluorophenyl) ethoxy]-2-methyl-3-pyridyl]-N-ethyl-N-methyl-formamidine (K.1.31), N′-[5-bromo-6-(4-isopropylcyclohexoxy)-2-methyl-3-pyridyl]-N-ethyl-N-methyl-formamidine (K.1.32), N′-[5-bromo-2-methyl-6-(1-phenylethoxy)-3-pyridyl]-N-ethyl-N-methyl-formamidine (K.1.33), N′-(2-methyl-5-trifluoromethyl-4-(3-trimethylsilanyl-propoxy)-phenyl)-N-ethyl-N-methyl formamidine (K.1.34), N′-(5-difluoromethyl-2-methyl-4-(3-trimethylsilanyl-propoxy)-phenyl)-N-ethyl-N-methyl formamidine (K.1.35), 2-(4-chloro-phenyl)-N-[4-(3,4-dimethoxy-phenyl)-isoxazol-5-yl]-2-prop-2-ynyloxy-acetamide (K.1.36), 3-[5-(4-chloro-phenyl)-2,3-dimethyl-isoxazolidin-3-yl]-pyridine (pyrisoxazole) (K.1.37), 3-[5-(4-methylphenyl)-2,3-dimethyl-isoxazolidin-3 yl]-pyridine (K.1.38), 5-chloro-1-(4,6-dimethoxy-pyrimidin-2-yl)-2-methyl-1H-benzoimidazole (K.1.39), ethyl(Z)-3-amino-2-cyano-3-phenyl-prop-2-enoate (K.1.40), picarbutrazox (K.1.41), pentyl N-[6-[[(Z)-[(1-methyltetrazol-5-yl)phenyl-methylene]amino]oxymethyl]-2-pyridyl]carbamate (K.1.42), but-3-ynyl N-[6-[[(Z)-[(1-methyltetrazol-5-yl)-phenyl-methylene]amino]oxymethyl]-2-pyridyl]carbamate (K.1.43), ipflufenoquin (K.1.44), quinofumelin (K.1.47), 2-(6-benzyl-2-pyridyl) quinazoline (K.1.50), 2-[6-(3-fluoro-4-methoxy-phenyl)-5-methyl-2-pyridyl]quinazoline (K.1.51), dichlobentiazox (K.1.52), N′-(2,5-dimethyl-4-phenoxy-phenyl)-N-ethyl-N-methyl-formamidine (K.1.53), pyrifenamine (K.1.54).

The fungicides described by common names, their preparation and their activity e.g. against harmful fungi is known (cf.: http://www.alanwood.net/pesticides/); these substances are commercially available.

The active substances referred to as component 2, their preparation and their activity e. g. against harmful fungi is known (cf.: http://www.alanwood.net/pesticides/); these substances are commercially available. The compounds described by IUPAC nomenclature, their preparation and their pesticidal activity are also known (cf. Can. J. Plant Sci. 48(6), 587-94, 1968; EP-A 141 317; EP-A 152 031; EP-A 226 917; EP-A 243 970; EP-A 256 503; EP-A 428 941; EP-A 532 022; EPA 1 028 125; EP-A 1 035 122; EP-A 1 201 648; EP-A 1 122 244, JP 2002316902; DE 19650197; DE 10021412; DE 102005009458; U.S. Pat. Nos. 3,296,272; 3,325,503; WO 98/46608; WO 99/14187; WO 99/24413; WO 99/27783; WO 00/29404; WO 00/46148; WO 00/65913; WO 01/54501; WO 01/56358; WO 02/22583; WO 02/40431; WO 03/10149; WO 03/11853; WO 03/14103; WO 03/16286; WO 03/53145; WO 03/61388; WO 03/66609; WO 03/74491; WO 04/49804; WO 04/83193; WO 05/120234; WO 05/123689; WO 05/123690; WO 05/63721; WO 05/87772; WO 05/87773; WO 06/15866; WO 06/87325; WO 06/87343; WO 07/82098; WO 07/90624, WO 10/139271, WO 11/028657, WO 12/168188, WO 07/006670, WO 11/77514; WO 13/047749, WO 10/069882, WO 13/047441, WO 03/16303, WO 09/90181, WO 13/007767, WO 13/010862, WO 13/127704, WO 13/024009, WO 13/24010, WO 13/047441, WO 13/162072, WO 13/092224, WO 11/135833, CN 1907024, CN 1456054, CN 103387541, CN 1309897, WO 12/84812, CN 1907024, WO 09094442, WO 14/60177, WO 13/116251, WO 08/013622, WO 15/65922, WO 94/01546, EP 2865265, WO 07/129454, WO 12/165511, WO 11/081174, WO 13/47441). Some compounds are identified by their CAS Registry Number which is separated by hyphens into three parts, the first consisting from two up to seven digits, the second consisting of two digits, and the third consisting of a single digit.

### Biopesticides

Suitable mixing partners for the compounds of the present invention also include biopesticides.

Biopesticides have been defined as a form of pesticides based on micro-organisms (bacteria, fungi, viruses, nematodes, etc.) or natural products (compounds, such as metabolites, proteins, or extracts from biological or other natural sources) (U.S. Environmental Protection Agency: http://www.epa.gov/pesticides/biopesticides/). Biopesticides fall into two major classes, microbial and biochemical pesticides:
(1) Microbial pesticides consist of bacteria, fungi or viruses (and often include the metabolites that bacteria and fungi produce). Entomopathogenic nematodes are also classified as microbial pesticides, even though they are multi-cellular.
(2) Biochemical pesticides are naturally occurring substances or or structurally-similar and functionally identical to a naturally-occurring substance and extracts from biological sources that control pests or provide other crop protection uses as defined below, but have non-toxic mode of actions (such as growth or developmental regulation, attractents, repellents or defence activators (e.g. induced resistance) and are relatively non-toxic to mammals.

Biopesticides for use against crop diseases have already established themselves on a variety of crops. For example, biopesticides already play an important role in controlling downy mildew diseases. Their benefits include: a 0-Day Pre-Harvest Interval, the ability to use under moderate to severe disease pressure, and the ability to use in mixture or in a rotational program with other registered pesticides.

A major growth area for biopesticides is in the area of seed treatments and soil amendments. Biopesticidal seed treatments are e.g. used to control soil borne fungal pathogens that cause seed rots, damping-off, root rot and seedling blights. They can also be used to control internal seed borne fungal pathogens as well as fungal pathogens that are on the surface of the seed. Many biopesticidal products also show capacities to stimulate plant host defenses and other physiological processes that can make treated crops more resistant to a variety of biotic and abiotic stresses or can regulate plant growth. Many biopesticidal products also show capacities to stimulate plant health, plant growth and/or yield enhancing activity.

The following list of biopesticides, in conjunction with which the compounds of the present invention can be used, is intended to illustrate the possible combinations but does not limit them:
L) Biopesticides
L1) Microbial pesticides with fungicidal, bactericidal, viricidal and/or plant defense activator activity: Ampelomyces quisqualis, Aspergillus flavus, Aureobasidium pullulans, Bacillus altitudinis, B. amyloliquefaciens, B. amyloliquefaciens ssp. plantarum (also referred to as B. velezensis), B. megaterium, B. mojavensis, B. mycoides, B. pumilus, B. simplex, B. solisalsi, B. subtilis, B. subtilis var. amyloliquefaciens, B. velezensis, Candida oleophila, C. saitoana, Clavibacter michiganensis (bacteriophages), Coniothyrium minitans, Cryphonectria parasitica, Cryptococcus albidus, Dilophosphora alopecuri, Fusarium oxysporum, Clonostachys rosea f. catenulate (also named Gliocladium catenulatum), Gliocladium roseum, Lysobacter antibioticus, L. enzymogenes, Metschnikowia fructicola, Microdochium dimerum, Microsphaeropsis ochracea, Muscodor albus, Paenibacillus alvei, Paenibacillus epiphyticus, P. polymyxa, Pantoea vagans, Penicillium bilaiae, Phlebiopsis gigantea, Pseudomonas sp., Pseudomonas chloraphis, Pseudozyma flocculosa, Pichia anomala, Pythium oligandrum, Sphaerodes mycoparasitica, Streptomyces griseoviridis, S. lydicus, S. violaceusniger, Talaromyces flavus, Trichoderma asperelloides, T. asperellum, T. atroviride, T. fertile, T. gamsii, T. harmatum, T. harzianum, T. polysporum, T. stromaticum, T. virens, T. viride, Typhula phacorrhiza, Ulocladium oudemansii, Verticillium dahlia, zucchini yellow mosaic virus (avirulent strain);
L2) Biochemical pesticides with fungicidal, bactericidal, viricidal and/or plant defense activator activity: harpin protein, Reynoutria sachalinensis extract;
L3) Microbial pesticides with insecticidal, acaricidal, molluscidal and/or nematicidal activity: Agrobacterium radiobacter, Bacillus cereus, B. firmus, B. thuringiensis, B. thuringiensis ssp. aizawai, B. t. ssp. israelensis, B. t. ssp. galleriae, B. t. ssp. kurstaki, B. t. ssp. tenebrionis, Beauveria bassiana, B. brongniartii, Burkholderia spp., Chromobacterium subtsugae, Cydia pomonella granulovirus (CpGV), Cryptophlebia leucotreta granulovirus (CrleGV), Flavobacterium spp., Helicoverpa armigera nucleopolyhedrovirus (HearNPV), Helicoverpa zea nucleopolyhedrovirus (HzNPV), Helicoverpa zea single capsid nucleopolyhedrovirus (HzSNPV), Heterorhabditis bacteriophora, Isaria fumosorosea, Lecanicillium longisporum, L. muscarium, Metarhizium anisopliae, M. anisopliae var. anisopliae, M. anisopliae var. acridum, Nomuraea rileyi, Paecilomyces fumosoroseus, P. lilacinus, Paenibacillus popilliae, Pasteuria spp., P. nishizawae, P. penetrans, P. ramosa, P. thornea, P. usgae, Pseudomonas fluorescens, Spodoptera littoralis nucleopolyhedrovirus (SpliNPV), Steinernema carpocapsae, S. feltiae, S. kraussei, Streptomyces galbus, S. microflavus;
L4) Biochemical pesticides with insecticidal, acaricidal, molluscidal, pheromone and/or nematicidal activity: L-carvone, citral, (E,Z)-7,9-dodecadien-1-yl acetate, ethyl formate, (E,Z)-2,4-ethyl decadienoate (pear ester), (Z,Z,E)-7,11,13-hexadecatrienal, heptyl butyrate, isopropyl myristate, lavanulyl senecioate, cis-jasmone, 2-methyl 1-butanol, methyl eugenol, methyl jasmonate, (E,Z)-2,13-octadecadien-1-ol, (E,Z)-2,13-octadecadien-1-ol acetate, (E,Z)-3,13-octadecadien-1-ol, (R)-1-octen-3-ol, pentatermanone, (E,Z,Z)-3,8,11-tetradecatrienyl acetate, (Z,E)-9,12-tetradecadien-1-yl acetate, (Z)-7-tetradecen-2-one, (Z)-9-tetradecen-1-yl acetate, (Z)-11-tetradecenal, (Z)-11-tetradecen-1-ol, extract of Chenopodium ambrosiodes, Neem oil, Quillay extract;
L5) Microbial pesticides with plant stress reducing, plant growth regulator, plant growth promoting and/or yield enhancing activity: Azospirillum amazonense, A. brasilense, A. lipoferum, A. irakense, A. halopraeferens, Bradyrhizobium spp., B. elkanii, B. japonicum, B. liaoningense, B. lupini, Delftia acidovorans, Glomus intraradices, Mesorhizobium spp., Rhizobium leguminosarum bv. phaseoli, R. I. bv. trifolii, R. I. bv. viciae, R. tropici, Sinorhizobium meliloti.

The biopesticides from group L1) and/or L2) may also have insecticidal, acaricidal, molluscidal, pheromone, nematicidal, plant stress reducing, plant growth regulator, plant growth promoting and/or yield enhancing activity. The biopesticides from group L3) and/or L4) may also have fungicidal, bactericidal, viricidal, plant defense activator, plant stress reducing, plant growth regulator, plant growth promoting and/or yield enhancing activity. The biopesticides from group L5) may also have fungicidal, bactericidal, viricidal, plant defense activator, insecticidal, acaricidal, molluscidal, pheromone and/or nematicidal activity.

Many of these biopesticides have been deposited under deposition numbers mentioned herein (the prefices such as ATCC or DSM refer to the acronym of the respective culture collection, for details see e. g. here: http://www.wfcc.info/ccinfo/collection/by_acronym/), are referred to in literature, registered and/or are commercially available: mixtures of Aureobasidium pullulans DSM 14940 and DSM 14941 isolated in 1989 in Konstanz, Germany (e. g. blastospores in BlossomProtect® from bio-ferm GmbH, Austria), Azospirillum brasilense Sp245 originally isolated in wheat reagion of South Brazil (Passo Fundo) at least prior to 1980 (BR 11005; e. g. GELFIX® Gramíneas from BASF Agricultural Specialties Ltd., Brazil), A. brasilense strains Ab-V5 and AbV6 (e. g. in AzoMax from Novozymes BioAg Produtos papra Agricultura Ltda., Quattro Barras, Brazil or Simbiose-Maíz® from Simbiose-Agro, Brazil; Plant Soil 331, 413-425, 2010), Bacillus amyloliquefaciens strain AP-188 (NRRL B-50615 and B-50331; U.S. Pat. No. 8,445,255); B. amyloliquefaciens ssp. plantarum strains formerly also sometimes referred to as B. subtilis, recently together with B. methylotrophicus, and B. velezensis classified as B. velezensis (Int. J. Syst. Evol. Microbiol. 66, 1212-1217, 2016): B. a. ssp. plantarum or B. velezensis D747 isolated from air in Kikugawa-shi, Japan (US20130236522 A1; FERM BP-8234; e. g. Double Nickel™ 55 WDG from Certis LLC, USA), B. a. ssp. plantarum or B. velezensis FZB24 isolated from soil in Brandenburg, Germany (also called SB3615; DSM 96-2; J. Plant Dis. Prot. 105, 181-197, 1998; e. g. Taegro® from Novozyme Biologicals, Inc., USA), B. a. ssp. plantarum or B. velezensis FZB42 isolated from soil in Brandenburg, Germany (DSM 23117; J. Plant Dis. Prot. 105, 181-197, 1998; e. g. RhizoVital® 42 from AbiTEP GmbH, Germany), B. a. ssp. plantarum or B. velezensis MBI600 isolated from faba bean in Sutton Bonington, Nottinghamshire, U.K. at least before 1988 (also called 1430; NRRL B-50595; US 2012/0149571 A1; e. g. Integral® from BASF Corp., USA), B. a. ssp. plantarum or B. velezensis QST-713 isolated from peach orchard in 1995 in California, U.S.A. (NRRL B-21661; e. g. Serenade® MAX from Bayer Crop Science LP, USA), B. a. ssp. plantarum or B. velezensis TJ1000 isolated in 1992 in South Dakoda, U.S.A. (also called 1BE; ATCC BAA-390; CA 2471555 A1; e. g. QuickRoots™ from TJ Technologies, Watertown, SD, USA); B. firmus CNCM I-1582, a variant of parental strain EIP-N1 (CNCM I-1556) isolated from soil of central plain area of Israel (WO 2009/126473, U.S. Pat. No. 6,406,690; e. g. Votivo® from Bayer CropScience LP, USA), B. pumilus GHA 180 isolated from apple tree rhizosphere in Mexico (IDAC 260707-01; e. g. PRO-MIX® BX from Premier Horticulture, Quebec, Canada), B. pumilus INR-7 otherwise referred to as BU-F22 and BU-F33 isolated at least before 1993 from cucumber infested by Erwinia tracheiphila (NRRL B-50185, NRRL B-50153; U.S. Pat. No. 8,445,255), B. pumilus KFP9F isolated from the rhizosphere of grasses in South Africa at least before 2008 (NRRL B-50754; WO 2014/029697; e. g. BAC-UP or FUSION-P from BASF Agricultural Specialities (Pty) Ltd., South Africa), B. pumilus QST 2808 was isolated from soil collected in Pohnpei, Federated States of Micronesia, in 1998 (NRRL B-30087; e. g. Sonata® or Ballad® Plus from Bayer Crop Science LP, USA), B. simplex ABU 288 (NRRL B-50304; U.S. Pat. No. 8,445,255), B. subtilis FB17 also called UD 1022 or UD10-22 isolated from red beet roots in North America (ATCC PTA-11857; System. Appl. Microbiol. 27, 372-379, 2004; US 2010/0260735; WO 2011/109395); B. thuringiensis ssp. aizawai ABTS-1857 isolated from soil taken from a lawn in Ephraim, Wisconsin, U.S.A., in 1987 (also called ABG-6346; ATCC SD-1372; e. g. XenTari® from BioFa AG, Münsingen, Germany), B. t. ssp. kurstaki ABTS-351 identical to HD-1 isolated in 1967 from diseased Pink Bollworm black larvae in Brownsville, Texas, U.S.A. (ATCC SD-1275; e. g. Dipel® DF from Valent BioSciences, IL, USA), B. t. ssp. kurstaki SB4 isolated from E. saccharina larval cadavers (NRRL B-50753; e. g. Beta Pro® from BASF Agricultural Specialities (Pty) Ltd., South Africa), B. t. ssp. tenebrionis NB-176-1, a mutant of strain NB-125, a wild type strain isolated in 1982 from a dead pupa of the beetle Tenebrio molitor (DSM 5480; EP 585 215 B1; e. g. Novodor® from Valent BioSciences, Switzerland), Beauveria bassiana GHA (ATCC 74250; e. g. BotaniGard® 22WGP from Laverlam Int. Corp., USA), B. bassiana JW-1 (ATCC 74040; e. g. Naturalis® from CBC (Europe) S.r.l., Italy), B. bassiana PPRI 5339 isolated from the larva of the tortoise beetle Conchyloctenia punctata (NRRL 50757; e. g. BroadBand® from BASF Agricultural Specialities (Pty) Ltd., South Africa), Bradyrhizobium elkanii strains SEMIA 5019 (also called 29W) isolated in Rio de Janeiro, Brazil and SEMIA 587 isolated in 1967 in the State of Rio Grande do Sul, from an area previously inoculated with a North American isolate, and used in commercial inoculants since 1968 (Appl. Environ. Microbiol. 73(8), 2635, 2007; e. g. GELFIX 5 from BASF Agricultural Specialties Ltd., Brazil), B. japonicum 532c isolated from Wisconsin field in U.S.A. (Nitragin 61A152; Can. J. Plant. Sci. 70, 661-666, 1990; e. g. in Rhizoflo®, Histick®, Hicoat® Super from BASF Agricultural Specialties Ltd., Canada), B. japonicum E-109 variant of strain USDA 138 (INTA E109, SEMIA 5085; Eur. J. Soil Biol. 45, 28-35, 2009; Biol. Fertil. Soils 47, 81-89, 2011); B. japonicum strains deposited at SEMIA known from Appl. Environ. Microbiol. 73(8), 2635, 2007: SEMIA 5079 isolated from soil in Cerrados region, Brazil by Embrapa-Cerrados used in commercial inoculants since 1992 (CPAC 15; e. g. GELFIX 5 or ADHERE 60 from BASF Agricultural Specialties Ltd., Brazil), B. japonicum SEMIA 5080 obtained under lab condtions by Embrapa-Cerrados in Brazil and used in commercial inoculants since 1992, being a natural variant of SEMIA 586 (CB1809) originally isolated in U.S.A. (CPAC 7; e. g. GELFIX 5 or ADHERE 60 from BASF Agricultural Specialties Ltd., Brazil); Burkholderia sp. A396 isolated from soil in Nikko, Japan, in 2008 (NRRL B-50319; WO 2013/032693; Marrone Bio Innovations, Inc., USA), Coniothyrium minitans CON/M/91-08 isolated from oilseed rape (WO 1996/021358; DSM 9660; e. g. Contans® WG, Intercept® WG from Bayer CropScience AG, Germany), harpin (alphabeta) protein (Science 257, 85-88, 1992; e. g. Messenger™ or HARP-N-Tek from Plant Health Care plc, U.K.), Helicoverpa armigera nucleopolyhedrovirus (HearNPV) (J. Invertebrate Pathol. 107, 112-126, 2011; e. g. Helicovex® from Adermatt Biocontrol, Switzerland; Diplomata® from Koppert, Brazil; Vivus® Max from AgBiTech Pty Ltd., Queensland, Australia), Helicoverpa zea single capsid nucleopolyhedrovirus (HzSNPV) (e. g. Gemstar® from Certis LLC, USA), Helicoverpa zea nucleopolyhedrovirus ABA-NPV-U (e. g. Heligen® from AgBiTech Pty Ltd., Queensland, Australia), Heterorhabditis bacteriophora (e. g. Nemasys® G from BASF Agricultural Specialities Limited, UK), Isaria fumosorosea Apopka-97 isolated from mealy bug on gynura in Apopka, Florida, U.S.A. (ATCC 20874; Biocontrol Science Technol. 22(7), 747-761, 2012; e. g. PFR-97™ or PreFeRal® from Certis LLC, USA), Metarhizium anisopliae var. anisopliae F52 also called 275 or V275 isolated from codling moth in Austria (DSM 3884, ATCC 90448; e. g. Met52® Novozymes Biologicals BioAg Group, Canada), Metschnikowia fructicola 277 isolated from grapes in the central part of Israel (U.S. Pat. No. 6,994,849; NRRL Y-30752; e. g. formerly Shemer® from Agrogreen, Israel), Paecilomyces ilacinus 251 isolated from infected nematode eggs in the Philippines (AGAL 89/030550; WO1991/02051; Crop Protection 27, 352-361, 2008; e. g. BioAct® from Bayer CropScience AG, Germany and MeloCon® from Certis, USA), Paenibacillus alvei NAS6G6 isolated from the rhizosphere of grasses in South Africa at least before 2008 (WO 2014/029697; NRRL B-50755; e.g. BAC-UP from BASF Agricultural Specialities (Pty) Ltd., South Africa), Paenibacillus strains isolated from soil samples from a variety of European locations including Germany: P. epiphyticus Lu17015 (WO 2016/020371; DSM 26971), P. polymyxa ssp. plantarum Lu16774 (WO 2016/020371; DSM 26969), P. p. ssp. plantarum strain Lu17007 (WO 2016/020371; DSM 26970); Pasteuria nishizawae Pn1 isolated from a soybean field in the mid-2000s in Illinois, U.S.A. (ATCC SD-5833; Federal Register 76(22), 5808 Feb. 2, 2011; e.g. Clariva™ PN from Syngenta Crop Protection, LLC, USA), Penicillium bilaiae (also called P. bilaii) strains ATCC 18309 (=ATCC 74319), ATCC 20851 and/or ATCC 22348 (=ATCC 74318) originally isolated from soil in Alberta, Canada (Fertilizer Res. 39, 97-103, 1994; Can. J. Plant Sci. 78(1), 91-102, 1998; U.S. Pat. No. 5,026,417, WO 1995/017806; e. g. Jump Start®, Provide® from Novozymes Biologicals BioAg Group, Canada), Reynoutria sachalinensis extract (EP 0307510 B1; e. g. Regalia® SC from Marrone BioInnovations, Davis, CA, USA or Milsana® from BioFa AG, Germany), Steinernema carpocapsae (e. g. Millenium® from BASF Agricultural Specialities Limited, UK), S. feltiae (e. g. Nemashield® from BioWorks, Inc., USA; Nemasys® from BASF Agricultural Specialities Limited, UK), Streptomyces microflavus NRRL B-50550 (WO 2014/124369; Bayer CropScience, Germany), Trichoderma asperelloides JM41R isolated in South Africa (NRRL 50759; also referred to as T. fertile; e. g. Trichoplus® from BASF Agricultural Specialities (Pty) Ltd., South Africa), T. harzianum T-22 also called KRL-AG2 (ATCC 20847; BioControl 57, 687-696, 2012; e. g. Plantshield® from BioWorks Inc., USA or SabrEx™ from Advanced Biological Marketing Inc., Van Wert, OH, USA).

According to the invention, the solid material (dry matter) of the biopesticides (with the exception of oils such as Neem oil) are considered as active components (e.g. to be obtained after drying or evaporation of the extraction or suspension medium in case of liquid formulations of the microbial pesticides).

In accordance with the present invention, the weight ratios and percentages used herein for a biological extract such as Quillay extract are based on the total weight of the dry content (solid material) of the respective extract(s).

The total weight ratios of compositions comprising at least one microbial pesticide in the form of viable microbial cells including dormant forms, can be determined using the amount of CFU of the respective microorganism to calculate the total weight of the respective active component with the following equation that 1×10¹⁰ CFU equals one gram of total weight of the respective active component. Colony forming unit is measure of viable microbial cells, in particular fungal and bacterial cells. In addition, here “CFU” may also be understood as the number of (juvenile) individual nematodes in case of (entomopathogenic) nematode biopesticides, such as Steinernema feltiae.

When mixtures comprising microbial pesticides are employed in crop protection, the application rates preferably range from about 1×10⁶ to 5×10¹⁵ (or more) CFU/ha, preferably from about 1×10⁸ to about 1×10¹³ CFU/ha, and even more preferably from about 1×10⁹ to about 1×10¹² CFU/ha. In the case of (entomopathogenic) nematodes as microbial pesticides (e. g. Steinernema feltiae), the application rates preferably range inform about 1×10⁵ to 1×10¹² (or more), more preferably from 1×10⁸ to 1×10¹¹, even more preferably from 5×10⁸ to 1×10¹⁰ individuals (e. g. in the form of eggs, juvenile or any other live stages, preferably in an infetive juvenile stage) per ha.

When mixtures comprising microbial pesticides are employed in seed treatment, the application rates with respect to plant propagation material preferably range from about 1×10⁶ to 1×10¹² (or more) CFU/seed. Preferably, the concentration is about 1×10⁶ to about 1×10⁹ CFU/seed. In the case of the microbial pesticides II, the application rates with respect to plant propagation material also preferably range from about 1×10⁷ to 1×10¹⁴ (or more) CFU per 100 kg of seed, preferably from 1×10⁹ to about 1×10¹² CFU per 100 kg of seed.

The invention also relates to agrochemical compositions comprising an auxiliary and at least one compound of the present invention or a mixture thereof.

An agrochemical composition comprises a pesticidally effective amount of a compound of the present invention or a mixture thereof. The term “pesticidally effective amount” is defined below.

The compounds of the present invention or the mixtures thereof can be converted into customary types of agro-chemical compositions, e. g. solutions, emulsions, suspensions, dusts, powders, pastes, granules, pressings, capsules, and mixtures thereof. Examples for composition types are suspensions (e.g. SC, OD, FS), emulsifiable concentrates (e.g. EC), emulsions (e.g. EW, EO, ES, ME), capsules (e.g. CS, ZC), pastes, pastilles, wettable powders or dusts (e.g. WP, SP, WS, DP, DS), pressings (e.g. BR, TB, DT), granules (e.g. WG, SG, GR, FG, GG, MG), insecticidal articles (e.g. LN), as well as gel formulations for the treatment of plant propagation materials such as seeds (e.g. GF). These and further compositions types are defined in the “Catalogue of pesticide formulation types and international coding system”, Technical Monograph No. 2, 6th Ed. May 2008, CropLife International.

The compositions are prepared in a known manner, such as described by Mollet and Grubemann, Formulation technology, Wiley VCH, Weinheim, 2001; or Knowles, New developments in crop protection product formulation, Agrow Reports DS243, T&F Informa, London, 2005.

Examples for suitable auxiliaries are solvents, liquid carriers, solid carriers or fillers, surfactants, dispersants, emulsifiers, wetters, adjuvants, solubilizers, penetration enhancers, protective colloids, adhesion agents, thickeners, humectants, repellents, attractants, feeding stimulants, compatibilizers, bactericides, anti-freezing agents, anti-foaming agents, colorants, tackifiers and binders.

Suitable solvents and liquid carriers are water and organic solvents, such as mineral oil fractions of medium to high boiling point, e.g. kerosene, diesel oil; oils of vegetable or animal origin; aliphatic, cyclic and aromatic hydrocarbons, e. g. toluene, paraffin, tetrahydronaphthalene, alkylated naphthalenes; alcohols, e.g. ethanol, propanol, butanol, benzylalcohol, cyclohexanol; glycols; DMSO; ketones, e.g. cyclohexanone; esters, e.g. lactates, carbonates, fatty acid esters, gamma-butyrolactone; fatty acids; phosphonates; amines; amides, e.g. N-methylpyrrolidone, fatty acid dimethylamides; and mixtures thereof.

Suitable solid carriers or fillers are mineral earths, e.g. silicates, silica gels, talc, kaolins, limestone, lime, chalk, clays, dolomite, diatomaceous earth, bentonite, calcium sulfate, magnesium sulfate, magnesium oxide; polysaccharide powders, e.g. cellulose, starch; fertilizers, e.g. ammonium sulfate, ammonium phosphate, ammonium nitrate, ureas; products of vegetable origin, e.g. cereal meal, tree bark meal, wood meal, nutshell meal, and mixtures thereof.

Suitable surfactants are surface-active compounds, such as anionic, cationic, nonionic and amphoteric surfactants, block polymers, polyelectrolytes, and mixtures thereof. Such surfactants can be used as emusifier, dispersant, solubilizer, wetter, penetration enhancer, protective colloid, or adjuvant. Examples of surfactants are listed in Mccutcheon's, Vol. 1: Emulsifiers & Detergents, Mccutcheon's Directories, Glen Rock, USA, 2008 (International Ed. or North American Ed.).

Suitable anionic surfactants are alkali, alkaline earth or ammonium salts of sulfonates, sulfates, phosphates, carboxylates, and mixtures thereof. Examples of sulfonates are alkylaryl-sulfonates, diphenylsulfonates, alpha-olefin sulfonates, lignine sulfonates, sulfonates of fatty acids and oils, sulfonates of ethoxylated alkylphenols, sulfonates of alkoxylated arylphenols, sulfonates of condensed naphthalenes, sulfonates of dodecyl- and tridecylbenzenes, sulfonates of naphthalenes and alkylnaphthalenes, sulfosuccinates or sulfosuccinamates. Examples of sulfates are sulfates of fatty acids and oils, of ethoxylated alkylphenols, of alcohols, of ethoxylated alcohols, or of fatty acid esters. Examples of phosphates are phosphate esters. Examples of carboxylates are alkyl carboxylates, and carboxylated alcohol or alkylphenol ethoxylates.

Suitable nonionic surfactants are alkoxylates, N-substituted fatty acid amides, amine oxides, esters, sugar-based surfactants, polymeric surfactants, and mixtures thereof. Examples of alkoxylates are compounds such as alcohols, alkylphenols, amines, amides, arylphenols, fatty acids or fatty acid esters which have been alkoxylated with 1 to 50 equivalents. Ethylene oxide and/or propylene oxide may be employed for the alkoxylation, preferably ethylene oxide. Examples of N-substituted fatty acid amides are fatty acid glucamides or fatty acid alkanolamides. Examples of esters are fatty acid esters, glycerol esters or monoglycerides. Examples of sugar-based surfactants are sorbitans, ethoxylated sorbitans, sucrose and glucose esters or alkylpolyglucosides. Examples of polymeric surfactants are homo- or copolymers of vinylpyrrolidone, vinylalcohols, or vinylacetate.

Suitable cationic surfactants are quaternary surfactants, for example quaternary ammonium compounds with one or two hydrophobic groups, or salts of long-chain primary amines. Suitable amphoteric surfactants are alkylbetains and imidazolines. Suitable block polymers are block polymers of the A-B or A-B-A type comprising blocks of polyethylene oxide and polypropylene oxide, or of the A-B-C type comprising alkanol, polyethylene oxide and polypropylene oxide. Suitable polyelectrolytes are polyacids or polybases. Examples of polyacids are alkali salts of polyacrylic acid or polyacid comb polymers. Examples of polybases are polyvinylamines or polyethyleneamines.

Suitable adjuvants are compounds, which have a neglectable or even no pesticidal activity themselves, and which improve the biological performance of the compounds of the present invention on the target. Examples are surfactants, mineral or vegetable oils, and other auxiliaries. Further examples are listed by Knowles, Adjuvants and additives, Agrow Reports DS256, T&F Informa UK, 2006, chapter 5.

Suitable thickeners are polysaccharides (e.g. xanthan gum, carboxymethylcellulose), anorganic clays (organically modified or unmodified), polycarboxylates, and silicates.

Suitable bactericides are bronopol and isothiazolinone derivatives such as alkylisothiazolinones and benzisothiazolinones.

Suitable anti-freezing agents are ethylene glycol, propylene glycol, urea and glycerin.

Suitable anti-foaming agents are silicones, long chain alcohols, and salts of fatty acids.

Suitable colorants (e.g. in red, blue, or green) are pigments of low water solubility and water-soluble dyes. Examples are inorganic colorants (e.g. iron oxide, titan oxide, iron hexacyanoferrate) and organic colorants (e.g. alizarin-, azo- and phthalocyanine colorants).

Suitable tackifiers or binders are polyvinylpyrrolidons, polyvinylacetates, polyvinyl alcohols, polyacrylates, biological or synthetic waxes, and cellulose ethers.

Examples for composition types and their preparation are:

### i) Water-Soluble Concentrates (SL, LS)

10-60 wt % of a compound I according to the invention and 5-15 wt % wetting agent (e.g. alcohol alkoxylates) are dissolved in water and/or in a water-soluble solvent (e.g. alcohols) up to 100 wt %. The active substance dissolves upon dilution with water.

### ii) Dispersible Concentrates (DC)

5-25 wt % of a compound I according to the invention and 1-10 wt % dispersant (e. g. polyvinylpyrrolidone) are dissolved in up to 100 wt % organic solvent (e.g. cyclohexanone). Dilution with water gives a dispersion.

iii) Emulsifiable Concentrates (EC)

15-70 wt % of a compound I according to the invention and 5-10 wt % emulsifiers (e.g. calcium dodecylbenzenesulfonate and castor oil ethoxylate) are dissolved in up to 100 wt % water-insoluble organic solvent (e.g. aromatic hydrocarbon). Dilution with water gives an emulsion.

### iv) Emulsions (EW, EO, ES)

5-40 wt % of a compound I according to the invention and 1-10 wt % emulsifiers (e.g. calcium dodecylbenzenesulfonate and castor oil ethoxylate) are dissolved in 20-40 wt % water-insoluble organic solvent (e.g. aromatic hydrocarbon). This mixture is introduced into up to 100 wt % water by means of an emulsifying machine and made into a homogeneous emulsion. Dilution with water gives an emulsion.

### v) Suspensions (SC, OD, FS)

In an agitated ball mill, 20-60 wt % of a compound I according to the invention are comminuted with addition of 2-10 wt % dispersants and wetting agents (e.g. sodium lignosulfonate and alcohol ethoxylate), 0.1-2 wt % thickener (e.g. xanthan gum) and up to 100 wt % water to give a fine active substance suspension. Dilution with water gives a stable suspension of the active substance. For FS type composition up to 40 wt % binder (e.g. polyvinylalcohol) is added.

### vi) Water-Dispersible Granules and Water-Soluble Granules (WG, SG)

50-80 wt % of a compound I according to the invention are ground finely with addition of up to 100 wt % dispersants and wetting agents (e.g. sodium lignosulfonate and alcohol ethoxylate) and prepared as water-dispersible or water-soluble granules by means of technical appliances (e. g. extrusion, spray tower, fluidized bed). Dilution with water gives a stable dispersion or solution of the active substance.

vii) Water-Dispersible Powders and Water-Soluble Powders (WP, SP, WS)

50-80 wt % of a compound I according to the invention are ground in a rotor-stator mill with addition of 1-5 wt % dispersants (e.g. sodium lignosulfonate), 1-3 wt % wetting agents (e.g. alcohol ethoxylate) and up to 100 wt % solid carrier, e.g. silica gel. Dilution with water gives a stable dispersion or solution of the active substance.

viii) Gel (GW, GF)

In an agitated ball mill, 5-25 wt % of a compound I according to the invention are comminuted with addition of 3-10 wt % dispersants (e.g. sodium lignosulfonate), 1-5 wt % thickener (e.g. carboxymethylcellulose) and up to 100 wt % water to give a fine suspension of the active substance. Dilution with water gives a stable suspension of the active substance.

### ix) Microemulsion (ME)

5-20 wt % of a compound I according to the invention are added to 5-30 wt % organic solvent blend (e.g. fatty acid dimethylamide and cyclohexanone), 10-25 wt % surfactant blend (e.g. alkohol ethoxylate and arylphenol ethoxylate), and water up to 100%. This mixture is stirred for 1 h to produce spontaneously a thermodynamically stable microemulsion.

### x) Microcapsules (CS)

An oil phase comprising 5-50 wt % of a compound I according to the invention, 0-40 wt % water insoluble organic solvent (e.g. aromatic hydrocarbon), 2-15 wt % acrylic monomers (e.g. methylmethacrylate, methacrylic acid and a di- or triacrylate) are dispersed into an aqueous solution of a protective colloid (e.g. polyvinyl alcohol). Radical polymerization initiated by a radical initiator results in the formation of poly(meth)acrylate microcapsules. Alternatively, an oil phase comprising 5-50 wt % of a compound I according to the invention, 0-40 wt % water insoluble organic solvent (e.g. aromatic hydrocarbon), and an isocyanate monomer (e.g. diphenylmethene-4,4′-diisocyanatae) are dispersed into an aqueous solution of a protective colloid (e.g. polyvinyl alcohol). The addition of a polyamine (e.g. hexamethylenediamine) results in the formation of a polyurea microcapsule. The monomers amount to 1-10 wt %. The wt % relate to the total CS composition.

### xi) Dustable Powders (DP, DS)

1-10 wt % of a compound I according to the invention are ground finely and mixed intimately with up to 100 wt % solid carrier, e.g. finely divided kaolin.

xii) Granules (GR, FG)

0.5-30 wt % of a compound I according to the invention is ground finely and associated with up to 100 wt % solid carrier (e.g. silicate). Granulation is achieved by extrusion, spray-drying or the fluidized bed.

xiii) Ultra-Low Volume Liquids (UL)

1-50 wt % of a compound I according to the invention are dissolved in up to 100 wt % organic solvent, e.g. aromatic hydrocarbon.

The compositions types i) to xi) may optionally comprise further auxiliaries, such as 0.1-1 wt % bactericides, 5-15 wt % anti-freezing agents, 0.1-1 wt % anti-foaming agents, and 0.1-1 wt % colorants.

The agrochemical compositions generally comprise between 0.01 and 95%, preferably between 0.1 and 90%, and most preferably between 0.5 and 75%, by weight of active substance. The active substances are employed in a purity of from 90% to 100%, preferably from 95% to 100% (according to NMR spectrum).

Various types of oils, wetters, adjuvants, fertilizer, or micronutrients, and other pesticides (e.g. herbicides, insecticides, fungicides, growth regulators, safeners) may be added to the active substances or the compositions comprising them as premix or, if appropriate not until immediately prior to use (tank mix). These agents can be admixed with the compositions according to the invention in a weight ratio of 1:100 to 100:1, preferably 1:10 to 10:1.

The user applies the composition according to the invention usually from a predosage de-vice, a knapsack sprayer, a spray tank, a spray plane, or an irrigation system. Usually, the agrochemical composition is made up with water, buffer, and/or further auxiliaries to the desired application concentration and the ready-to-use spray liquor or the agrochemical composition according to the invention is thus obtained. Usually, 20 to 2000 liters, preferably 50 to 400 liters, of the ready-to-use spray liquor are applied per hectare of agricultural useful area.

According to one embodiment, individual components of the composition according to the invention such as parts of a kit or parts of a binary or ternary mixture may be mixed by the user himself in a spray tank and further auxiliaries may be added, if appropriate.

In a further embodiment, either individual components of the composition according to the invention or partially premixed components, e. g. components comprising compounds of the present invention and/or mixing partners as defined above, may be mixed by the user in a spray tank and further auxiliaries and additives may be added, if appropriate.

In a further embodiment, either individual components of the composition according to the invention or partially premixed components, e. g. components comprising compounds of the present invention and/or mixing partners as defined above, can be applied jointly (e.g. after tank mix) or consecutively.

The compounds of the present invention are suitable for use in protecting crops, plants, plant propagation materials, such as seeds, or soil or water, in which the plants are growing, from attack or infestation by animal pests. Therefore, the present invention also relates to a plant protection method, which comprises contacting crops, plants, plant propagation materials, such as seeds, or soil or water, in which the plants are growing, to be protected from attack or infestation by animal pests, with a pesticidally effective amount of a compound of the present invention.

The compounds of the present invention are also suitable for use in combating or controlling animal pests. Therefore, the present invention also relates to a method of combating or controlling animal pests, which comprises contacting the animal pests, their habitat, breeding ground, or food supply, or the crops, plants, plant propagation materials, such as seeds, or soil, or the area, material or environment in which the animal pests are growing or may grow, with a pesticidally effective amount of a compound of the present invention.

The compounds of the present invention are effective through both contact and ingestion. Furthermore, the compounds of the present invention can be applied to any and all developmental stages, such as egg, larva, pupa, and adult.

The compounds of the present invention can be applied as such or in form of compositions comprising them as defined above. Furthermore, the compounds of the present invention can be applied together with a mixing partner as defined above or in form of compositions comprising said mixtures as defined above. The components of said mixture can be applied simultaneously, jointly or separately, or in succession, that is immediately one after another and thereby creating the mixture “in situ” on the desired location, e.g. the plant, the sequence, in the case of separate application, generally not having any effect on the result of the control measures.

The application can be carried out both before and after the infestation of the crops, plants, plant propagation materials, such as seeds, soil, or the area, material or environment by the pests.

Suitable application methods include inter alia soil treatment, seed treatment, in furrow application, and foliar application. Soil treatment methods include drenching the soil, drip irrigation (drip application onto the soil), dipping roots, tubers or bulbs, or soil injection. Seed treatment techniques include seed dressing, seed coating, seed dusting, seed soaking, and seed pelleting. In furrow applications typically include the steps of making a furrow in cultivated land, seeding the furrow with seeds, applying the pesticidally active compound to the furrow, and closing the furrow. Foliar application refers to the application of the pesticidally active compound to plant foliage, e.g. through spray equipment. For foliar applications, it can be advantageous to modify the behavior of the pests by use of pheromones in combination with the compounds of the present invention. Suitable pheromones for specific crops and pests are known to a skilled person and publicly available from databases of pheromones and semiochemicals, such as http://www.pherobase.com.

As used herein, the term “contacting” includes both direct contact (applying the compounds/compositions directly on the animal pest or plant-typically to the foliage, stem or roots of the plant) and indirect contact (applying the compounds/compositions to the locus, i.e. habitat, breeding ground, plant, seed, soil, area, material or environment in which a pest is growing or may grow, of the animal pest or plant).

The term “animal pest” includes arthropods, gastropods, and nematodes. Preferred animal pests according to the invention are arthropods, preferably insects and arachnids, in particular insects. Insects, which are of particular relevance for crops, are typically referred to as crop insect pests.

The term “crop” refers to both, growing and harvested crops.

The term “plant” includes cereals, e.g. durum and other wheat, rye, barley, triticale, oats, rice, or maize (fodder maize and sugar maize/sweet and field corn); beet, e.g. sugar beet or fodder beet; fruits, such as pomes, stone fruits or soft fruits, e.g. apples, pears, plums, peaches, nectarines, almonds, cherries, papayas, strawberries, raspberries, blackberries or gooseberries; leguminous plants, such as beans, lentils, peas, alfalfa or soybeans; oil plants, such as rapeseed (oilseed rape), turnip rape, mustard, olives, sunflowers, coconut, cocoa beans, castor oil plants, oil palms, ground nuts or soybeans; cucurbits, such as squashes, pumpkins, cucumber or melons; fiber plants, such as cotton, flax, hemp or jute; citrus fruit, such as oranges, lemons, grapefruits or mandarins; vegetables, such as eggplant, spinach, lettuce (e.g. iceberg lettuce), chicory, cabbage, asparagus, cabbages, carrots, onions, garlic, leeks, tomatoes, potatoes, cucurbits or sweet peppers; lauraceous plants, such as avocados, cinnamon or camphor; energy and raw material plants, such as corn, soybean, rapeseed, sugar cane or oil palm; tobacco; nuts, e.g. walnuts; pistachios; coffee; tea; bananas; vines (table grapes and grape juice grape vines); hop; sweet leaf (also called Stevia); natural rubber plants or ornamental and forestry plants, such as flowers (e.g. carnation, petunias, geranium/pelargoniums, pansies and impatiens), shrubs, broadleaved trees (e.g. poplar) or evergreens, e.g. conifers; eucalyptus; turf; lawn; grass such as grass for animal feed or ornamental uses. Preferred plants include potatoes sugar beets, tobacco, wheat, rye, barley, oats, rice, corn, cotton, soybeans, rapeseed, legumes, sunflowers, coffee or sugar cane; fruits; vines; ornamentals; or vegetables, such as cucumbers, tomatoes, beans or squashes.

The term “cultivated plants” is to be understood as including plants which have been modified by mutagenesis or genetic engineering in order to provide a new trait to a plant or to modify an already present trait.

Mutagenesis includes techniques of random mutagenesis using X-rays or mutagenic chemicals, but also techniques of targeted mutagenesis, in order to create mutations at a specific locus of a plant genome. Targeted mutagenesis techniques frequently use oligonucleotides or proteins like

CRISPR/Cas, zinc-finger nucleases, TALENs or meganucleases to achieve the targeting effect. Genetic engineering usually uses recombinant DNA techniques to create modifications in a plant genome which under natural circumstances cannot readily be obtained by cross breeding, mutagenesis or natural recombination. Typically, one or more genes are integrated into the genome of a plant in order to add a trait or improve a trait. These integrated genes are also referred to as transgenes in the art, while plant comprising such transgenes are referred to as transgenic plants. The process of plant transformation usually produces several transformation events, wich differ in the genomic locus in which a transgene has been integrated. Plants comprising a specific transgene on a specific genomic locus are usually described as comprising a specific “event”, which is referred to by a specific event name. Traits which have been introduced in plants or have been modified include in particular herbicide tolerance, insect resistance, increased yield and tolerance to abiotic conditions, like drought.

Herbicide tolerance has been created by using mutagenesis as well as using genetic engineering. Plants which have been rendered tolerant to acetolactate synthase (ALS) inhibitor herbicides by conventional methods of mutagenesis and breeding comprise plant varieties commercially available under the name Clearfield®. However, most of the herbicide tolerance traits have been created via the use of transgenes.

Herbicide tolerance has been created to glyphosate, glufosinate, 2,4-D, dicamba, oxynil herbicides, like bromoxynil and ioxynil, sulfonylurea herbicides, ALS inhibitor herbicides and 4-hydroxyphenylpyruvate dioxygenase (HPPD) inhibitors, like isoxaflutole and mesotrione.

Transgenes wich have been used to provide herbicide tolerance traits comprise: for tolerance to glyphosate: cp4 epsps, epsps grg23ace5, mepsps, 2mepsps, gat4601, gat4621 and goxv247, for tolerance to glufosinate: pat and bar, for tolerance to 2,4-D: aad-1 and aad-12, for tolerance to dicamba: dmo, for tolerance to oxynil herbicies: bxn, for tolerance to sulfonylurea herbicides: zm-hra, csr1-2, gm-hra, S4-HrA, for tolerance to ALS inhibitor herbicides: csr1-2, for tolerance to HPPD inhibitor herbicides: hppdPF, W336 and avhppd-03.

Transgenic corn events comprising herbicide tolerance genes are for example, but not excluding others, DAS40278, MON801, MON802, MON809, MON810, MON832, MON87411, MON87419, MON87427, MON88017, MON89034, NK603, GA21, MZHG0JG, HCEM485, VCO-Ø1981-5, 676, 678, 680, 33121, 4114, 59122, 98140, Bt10, Bt176, CBH-351, DBT418, DLL25, MS3, MS6, MZIR098, T25, TC1507 and TC6275.

Transgenic soybean events comprising herbicide tolerance genes are for example, but not excluding others, GTS 40-3-2, MON87705, MON87708, MON87712, MON87769, MON89788, A2704-12, A2704-21, A5547-127, A5547-35, DP356043, DAS44406-6, DAS68416-4, DAS81419-2, GU262, SYHTØH2, W62, W98, FG72 and CV127.

Transgenic cotton events comprising herbicide tolerance genes are for example, but not excluding others, 19-51a, 31707, 42317, 81910, 281-24-236, 3006-210-23, BXN10211, BXN10215, BXN10222, BXN10224, MON1445, MON1698, MON88701, MON88913, GHB119, GHB614, LLCotton25, T303-3 and T304-40.

Transgenic canola events comprising herbicide tolerance genes are for example, but not excluding others, MON88302, HCR-1, HCN10, HCN28, HCN92, MS1, MS8, PHY14, PHY23, PHY35, PHY36, RF1, RF2 and RF3.

Insect resistance has mainly been created by transferring bacterial genes for insecticidal proteins to plants. Transgenes which have most frequently been used are toxin genes of Bacillus spec, and synthetic variants thereof, like cry1A, cry1Ab, cry1Ab-Ac, cry1Ac, cry1A.105, cry1F, cry1Fa2, cry2Ab2, cry2Ae, mcry3A, ecry3.1Ab, cry3Bb1, cry34Ab1, cry35Ab1, cry9C, vip3A (a), vip3Aa20. However, also genes of plant origin have been transferred to other plants. In particular genes coding for protease inhibitors, like CpTI and pinII. A further approach uses transgenes in order to produce double stranded RNA in plants to target and downregulate insect genes. An example for such a transgene is dvsnf7.

Transgenic corn events comprising genes for insecticidal proteins or double stranded RNA are for example, but not excluding others, Bt10, Bt11, Bt176, MON801, MON802, MON809, MON810, MON863, MON87411, MON88017, MON89034, 33121, 4114, 5307, 59122, TC1507, TC6275, CBH-351, MIR162, DBT418 and MZIR098.

Transgenic soybean events comprising genes for insecticidal proteins are for example, but not excluding others, MON87701, MON87751 and DAS-81419.

Transgenic cotton events comprising genes for insecticidal proteins are for example, but not excluding others, SGK321, MON531, MON757, MON1076, MON15985, 31707, 31803, 31807, 31808, 42317, BNLA-601, Event1, COT67B, COT102, T303-3, T304-40, GFM CrylA, GK12, MLS 9124, 281-24-236, 3006-210-23, GHB119 and SGK321.

Increased yield has been created by increasing ear biomass using the transgene athb17, being present in corn event MON87403, or by enhancing photosynthesis using the transgene bbx32, being present in the soybean event MON87712.

Cultivated plants comprising a modified oil content have been created by using the transgenes: gm-fad2-1, Pj.D6D, Nc.Fad3, fad2-1A and fatb1-A. Soybean events comprising at least one of these genes are: 260-05, MON87705 and MON87769.

Tolerance to abiotic conditions, in particular to tolerance to drought, has been created by using the transgene cspB, comprised by the corn event MON87460 and by using the transgene Hahb-4, comprised by soybean event IND-ØØ41Ø-5.

Traits are frequently combined by combining genes in a transformation event or by combining different events during the breeding process. Preferred combination of traits are herbicide tolerance to different groups of herbicides, insect tolerance to different kind of insects, in particular tolerance to lepidopteran and coleopteran insects, herbicide tolerance with one or several types of insect resistance, herbicide tolerance with increased yield as well as a combination of herbicide tolerance and tolerance to abiotic conditions.

Plants comprising singular or stacked traits as well as the genes and events providing these traits are well known in the art. For example, detailed information as to the mutagenized or integrated genes and the respective events are available from websites of the organizations “International Service for the Acquisition of Agri-biotech Applications (ISAAA)” (http://www.isaaa.org/gmapprovaldatabase) and the “Center for Environmental Risk Assessment (CERA)” (http://cera-gmc.org/GMCropDatabase), Further information on specific events and methods to detect them can be found for canola events MS1, MS8, RF3, GT73, MON88302, KK179 in WO01/031042, WO01/041558, WO01/041558, WO02/036831, WO11/153186, WO13/003558, for cotton events MON1445, MON15985, MON531 (MON15985), LLCotton25, MON88913, COT102, 281-24-236, 3006-210-23, COT67B, GHB614, T304-40, GHB119, MON88701, 81910 in WO02/034946, WO02/100163, WO02/100163, WO03/013224, WO04/072235, WO04/039986, WO05/103266, WO05/103266, WO06/128573, WO07/017186, WO08/122406, WO08/151780, WO12/134808, WO13/112527, for corn events GA21, MON810, DLL25, TC1507, MON863, MIR604, LY038, MON88017, 3272, 59122, NK603, MIR162, MON89034, 98140, 32138, MON87460, 5307, 4114, MON87427, DAS40278, MON87411, 33121, MON87403, MON87419 in WO98/044140, U.S. Ser. No. 02/102,582, U.S. Ser. No. 03/126,634, WO04/099447, WO04/011601, WO05/103301, WO05/061720, WO05/059103, WO06/098952, WO06/039376, US2007/292854, WO07/142840, WO07/140256, WO08/112019, WO09/103049, WO09/111263, WO10/077816, WO11/084621, WO11/062904, WO11/022469, WO13/169923, WO14/116854, WO15/053998, WO15/142571, for potato events E12, F10, J3, J55, V11, X17, Y9 in WO14/178910, WO14/178913, WO14/178941, WO14/179276, WO16/183445, WO17/062831, WO17/062825, for rice events LLRICE06, LLRICE601, LLRICE62 in WO00/026345, WO00/026356, WO00/026345 for soybean events H7-1, MON89788, A2704-12, A5547-127, DP305423, DP356043, MON87701, MON87769, CV127, MON87705, DAS68416-4, MON87708, MON87712, SYHTOH2, DAS81419, DAS81419×DAS44406-6, MON87751 in WO04/074492, WO06/130436, WO06/108674, WO06/108675, WO08/054747, WO08/002872, WO09/064652, WO09/102873, WO10/080829, WO10/037016, WO11/066384, WO11/034704, WO12/051199, WO12/082548, WO13/016527, WO13/016516, WO14/201235.

The use of compositions according to the invention on cultivated plants may result in effects which are specific to a cultivated plant comprising a certain gene or event. These effects might involve changes in growth behavior or changed resistance to biotic or abiotic stress factors. Such effects may in particular comprise enhanced yield, enhanced resistance or tolerance to insects, nematodes, fungal, bacterial, mycoplasma, viral or viroid pathogens as well as early vigour, early or delayed ripening, cold or heat tolerance as well as changed amino acid or fatty acid spectrum or content.

It has surprisingly been found that the pesticidal activity of the compounds of the present invention may be enhanced by the insecticidal trait of a modified plant. Furthermore, it has been found that the compounds of the present invention are suitable for preventing insects to become resistant to the insecticidal trait or for combating pests, which already have become resistant to the insecticidal trait of a modified plant. Moreover, the compounds of the present invention are suitable for combating pests, against which the insecticidal trait is not effective, so that a complementary insecticidal activity can advantageously be used.

The term “plant propagation material” refers to all the generative parts of the plant such as seeds and vegetative plant material such as cuttings and tubers (e.g. potatoes), which can be used for the multiplication of the plant. This includes seeds, roots, fruits, tubers, bulbs, rhizomes, shoots, sprouts and other parts of plants. Seedlings and young plants, which are to be transplanted after germination or after emergence from soil, may also be included. These plant propagation materials may be treated prophylactically with a plant protection compound either at or before planting or transplanting.

The term “seed” embraces seeds and plant propagules of all kinds including but not limited to true seeds, seed pieces, suckers, corms, bulbs, fruit, tubers, grains, cuttings, cut shoots and the like, and means in a preferred embodiment true seeds.

In general, “pesticidally effective amount” means the amount of active ingredient needed to achieve an observable effect on growth, including the effects of necrosis, death, retardation, prevention, and removal, destruction, or otherwise diminishing the occurrence and activity of the target organism. The pesticidally effective amount can vary for the various compounds/compositions used in the invention. A pesticidally effective amount of the compositions will also vary according to the prevailing conditions such as desired pesticidal effect and duration, weather, target species, locus, mode of application, and the like.

In the case of soil treatment, in furrow application or of application to the pests dwelling place or nest, the quantity of active ingredient ranges from 0.0001 to 500 g per 100 m², preferably from 0.001 to 20 g per 100 m².

For use in treating crop plants, e.g. by foliar application, the rate of application of the active ingredients of this invention may be in the range of 0.0001 g to 4000 g per hectare, e.g. from 1 g to 2 kg per hectare or from 1 g to 750 g per hectare, desirably from 1 g to 100 g per hectare, more desirably from 10 g to 50 g per hectare, e.g., 10 to 20 g per hectare, 20 to 30 g per hectare, 30 to 40 g per hectare, or 40 to 50 g per hectare.

The compounds of the invention are particularly suitable for use in the treatment of seeds in order to protect the seeds from insect pests, in particular from soil-living insect pests, and the resulting seedling's roots and shoots against soil pests and foliar insects. The invention therefore also relates to a method for the protection of seeds from insects, in particular from soil insects, and of the seedling's roots and shoots from insects, in particular from soil and foliar insects, said method comprising treating the seeds before sowing and/or after pregermination with a compound of the invention. The protection of the seedling's roots and shoots is preferred. More preferred is the protection of seedling's shoots from piercing and sucking insects, chewing insects and nematodes.

The term “seed treatment” comprises all suitable seed treatment techniques known in the art, such as seed dressing, seed coating, seed dusting, seed soaking, seed pelleting, and in-furrow application methods. Preferably, the seed treatment application of the active compound is carried out by spraying or by dusting the seeds before sowing of the plants and before emergence of the plants.

The invention also comprises seeds coated with or containing the active compound. The term “coated with and/or containing” generally signifies that the active ingredient is for the most part on the surface of the propagation product at the time of application, although a greater or lesser part of the ingredient may penetrate into the propagation product, depending on the method of application. When the said propagation product is (re)planted, it may absorb the active ingredient.

Suitable seed is for example seed of cereals, root crops, oil crops, vegetables, spices, ornamentals, for example seed of durum and other wheat, barley, oats, rye, maize (fodder maize and sugar maize/sweet and field corn), soybeans, oil crops, crucifers, cotton, sunflowers, bananas, rice, oilseed rape, turnip rape, sugarbeet, fodder beet, eggplants, potatoes, grass, lawn, turf, fodder grass, tomatoes, leeks, pumpkin/squash, cabbage, iceberg lettuce, pepper, cucumbers, melons, Brassica species, melons, beans, peas, garlic, onions, carrots, tuberous plants such as potatoes, sugar cane, tobacco, grapes, petunias, geranium/pelargoniums, pansies and impatiens.

In addition, the active compound may also be used for the treatment of seeds from plants, which have been modified by mutagenisis or genetic engineering, and which e.g. tolerate the action of herbicides or fungicides or insecticides. Such modified plants have been described in detail above.

Conventional seed treatment formulations include for example flowable concentrates FS, solutions LS, suspoemulsions (SE), powders for dry treatment DS, water dispersible powders for slurry treatment WS, water-soluble powders SS and emulsion ES and EC and gel formulation GF. These formulations can be applied to the seed diluted or undiluted. Application to the seeds is carried out before sowing, either directly on the seeds or after having pregerminated the latter. Preferably, the formulations are applied such that germination is not included.

The active substance concentrations in ready-to-use formulations, which may be obtained after two-to-tenfold dilution, are preferably from 0.01 to 60% by weight, more preferably from 0.1 to 40% by weight.

In a preferred embodiment a FS formulation is used for seed treatment. Typically, a FS formulation may comprise 1-800 g/l of active ingredient, 1-200 g/l Surfactant, 0 to 200 g/l antifreezing agent, 0 to 400 g/l of binder, 0 to 200 g/l of a pigment and up to 1 liter of a solvent, preferably water.

Especially preferred FS formulations of the compounds of the invention for seed treatment usually comprise from 0.1 to 80% by weight (1 to 800 g/l) of the active ingredient, from 0.1 to 20% by weight (1 to 200 g/l) of at least one surfactant, e.g. 0.05 to 5% by weight of a wetter and from 0.5 to 15% by weight of a dispersing agent, up to 20% by weight, e.g. from 5 to 20% of an anti-freeze agent, from 0 to 15% by weight, e.g. 1 to 15% by weight of a pigment and/or a dye, from 0 to 40% by weight, e.g. 1 to 40% by weight of a binder (sticker/adhesion agent), optionally up to 5% by weight, e.g. from 0.1 to 5% by weight of a thickener, optionally from 0.1 to 2% of an anti-foam agent, and optionally a preservative such as a biocide, antioxidant or the like, e.g. in an amount from 0.01 to 1% by weight and a filler/vehicle up to 100% by weight.

In the treatment of seed, the application rates of the compounds of the invention are generally from 0.1 g to 10 kg per 100 kg of seed, preferably from 1 g to 5 kg per 100 kg of seed, more preferably from 1 g to 1000 g per 100 kg of seed and in particular from 1 g to 200 g per 100 kg of seed, e.g. from 1 g to 100 g or from 5 g to 100 g per 100 kg of seed.

The invention therefore also relates to seed comprising a compound of the invention, or an agriculturally useful salt thereof, as defined herein. The amount of the compound of the invention or the agriculturally useful salt thereof will in general vary from 0.1 g to 10 kg per 100 kg of seed, preferably from 1 g to 5 kg per 100 kg of seed, in particular from 1 g to 1000 g per 100 kg of seed. For specific crops such as lettuce the rate can be higher.

The compounds of the invention may also be used for improving the health of a plant. Therefore, the invention also relates to a method for improving plant health by treating a plant, plant propagation material and/or the locus where the plant is growing or is to grow with an effective and non-phytotoxic amount of a compound of the invention.

As used herein “an effective and non-phytotoxic amount” means that the compound is used in a quantity which allows to obtain the desired effect but which does not give rise to any phytotoxic symptom on the treated plant or on the plant grown from the treated propagule or treated soil.

The terms “plant” and “plant propagation material” are defined above.

“Plant health” is defined as a condition of the plant and/or its products which is determined by several aspects alone or in combination with each other such as yield (for example increased biomass and/or increased content of valuable ingredients), quality (for example improved content or composition of certain ingredients or shelf life), plant vigour (for example improved plant growth and/or greener leaves (“greening effect”), tolerance to abiotic (for example drought) and/or biotic stress (for example disease) and production efficiency (for example, harvesting efficiency, processability).

The above identified indicators for the health condition of a plant may be interdependent and may result from each other. Each indicator is defined in the art and can be determined by methods known to a skilled person.

The compounds of the invention are also suitable for use against non-crop insect pests. For use against said non-crop pests, compounds of the invention can be used as bait composition, gel, general insect spray, aerosol, as ultra-low volume application and bed net (impregnated or surface applied). Furthermore, drenching and rodding methods can be used.

As used herein, the term “non-crop insect pest” refers to pests, which are particularly relevant for non-crop targets, such as ants, termites, wasps, flies, ticks, mosquitoes, bed bugs, crickets, or cockroaches.

The bait can be a liquid, a solid or a semisolid preparation (e.g. a gel). The bait employed in the composition is a product, which is sufficiently attractive to incite insects such as ants, termites, wasps, flies, mosquitoes, crickets etc. or cockroaches to eat it. The attractiveness can be manipulated by using feeding stimulants or sex pheromones. Food stimulants are chosen, for example, but not exclusively, from animal and/or plant proteins (meat-, fish- or blood meal, insect parts, egg yolk), from fats and oils of animal and/or plant origin, or mono-, oligo- or polyorganosaccharides, especially from sucrose, lactose, fructose, dextrose, glucose, starch, pectin or even molasses or honey. Fresh or decaying parts of fruits, crops, plants, animals, insects or specific parts thereof can also serve as a feeding stimulant. Sex pheromones are known to be more insect specific. Specific pheromones are described in the literature (e.g. http://www.pherobase.com), and are known to those skilled in the art.

For use in bait compositions, the typical content of active ingredient is from 0.001 weight % to 15 weight %, desirably from 0.001 weight % to 5% weight % of active compound.

Formulations of the compounds of the invention as aerosols (e.g in spray cans), oil sprays or pump sprays are highly suitable for professional or non-professional users for controlling pests such as flies, fleas, ticks, bed bugs, mosquitoes or cockroaches. Aerosol recipes are preferably composed of the active compound, solvents, furthermore auxiliaries such as emulsifiers, perfume oils, if appropriate stabilizers, and, if required, propellants.

The oil spray formulations differ from the aerosol recipes in that no propellants are used.

For use in spray compositions, the content of active ingredient is from 0.001 to 80 weights %, preferably from 0.01 to 50 weight % and most preferably from 0.01 to 15 weight %.

The compounds of the invention and its respective compositions can also be used in mosquito and fumigating coils, smoke cartridges, vaporizer plates or long-term vaporizers and also in moth papers, moth pads or other heat-independent vaporizer systems.

Methods to control infectious diseases transmitted by insects (e.g. malaria, dengue and yellow fever, lymphatic filariasis, and leishmaniasis) with compounds of the invention and its respective compositions also comprise treating surfaces of huts and houses, air spraying and impregnation of curtains, tents, clothing items, bed nets, tsetse-fly trap or the like. Insecticidal compositions for application to fibers, fabric, knitgoods, nonwovens, netting material or foils and tarpaulins preferably comprise a mixture including the insecticide, optionally a repellent and at least one binder.

The compounds of the invention and its compositions can be used for protecting wooden materials such as trees, board fences, sleepers, frames, artistic artifacts, etc. and buildings, but also construction materials, furniture, leathers, fibers, vinyl articles, electric wires and cables etc. from ants, termites and/or wood or textile destroying beetles, and for controlling ants and termites from doing harm to crops or human beings (e.g. when the pests invade into houses and public facilities or nest in yards, orchards or parks).

Customary application rates in the protection of materials are, for example, from 0.001 g to 2000 g or from 0.01 g to 1000 g of active compound per m² treated material, desirably from 0.1 g to 50 g per m².

Insecticidal compositions for use in the impregnation of materials typically contain from 0.001 to 95 weight %, preferably from 0.1 to 45 weight %, and more preferably from 1 to 25 weight % of at least one repellent and/or insecticide.

The compounds of the the invention are especially suitable for efficiently combating animal pests such as arthropods, gastropods and nematodes including but not limited to:
insects from the order of Lepidoptera, for example Achroia grisella, Acleris spp, such as A. fimbriana, A. gloverana, A. variana; Acrolepiopsis assectella, Acronicta major, Adoxophyes spp. such as A. cyrtosema, A. orana; Aedia leucomelas, Agrotis spp, such as A. exclamationis, A. fucosa, A. ipsilon, A. orthogoma, A. segetum, A. subterranea; Alabama argillacea, Aleurodicus dispersus, Alsophila pometaria, Ampelophaga rubiginosa, Amyelois transitella, Anacampsis sarcitella, Anagasta kuehniella, Anarsia lineatella, Anisota senatoria, Antheraea pernyi, Anticarsia (=Thermesia) spp, such as A. gemmatalis; Apamea spp., Aproaerema modicella, Archips spp. such as A. argyrospila, A. fuscocupreanus, A. rosana, A. xyloseanus; Argyresthia conjugella, Argyroploce spp., Argyrotaenia spp, such as A. velutinana; Athetis mindara, Austroasca viridigrisea, Autographa gamma, Autographa nigrisigna, Barathra brassicae, Bedellia spp., Bonagota salubricola, Borbo cinnara, Bucculatrix thurberiella, Bupalus piniarius, Busseola spp., Cacoecia spp, such as C. murinana, C. podana; Cactoblastis cactorum, Cadra cautella, Calingo braziliensis, Caloptilis theivora, Capua reticulana, Carposina spp, such as C. niponensis, C. sasakii; Cephus spp., Chaetocnema aridula, Cheimatobia brumata, Chilo spp, such as C. Indicus, C. suppressalis, C. partellus; Choreutis pariana, Choristoneura spp, such as C. conflictana, C. fumiferana, C. longicellana, C. murinana, C. occidentalis, C. rosaceana; Chrysodeixis (=Pseudoplusia) spp, such as C. eriosoma, C. includens; Cirphis unipuncta, Clysia ambiguella, Cnaphalocerus spp., Cnaphalocrocis medinalis, Cnephasia spp., Cochylis hospes, Coleophora spp., Colias eurytheme, Conopomorpha spp., Conotrachelus spp., Copitarsia spp., Corcyra cephalonica, Crambus caliginosellus, Crambus teterrellus, Crocidosema (=Epinotia) aporema, Cydalima (=Diaphania) perspectalis, Cydia (=Carpocapsa) spp, such as C. pomonella, C. latiferreana; Dalaca noctuides, Datana integerrima, Dasychira pinicola, Dendrolimus spp. such as D. pini, D. spectabilis, D. sibiricus; Desmia funeralis, Diaphania spp, such as D. nitidalis, D. hyalinata; Diatraea grandiosella, Diatraea saccharalis, Diphthera festiva, Earias spp, such as E. insulana, E. vittella; Ecdytolopha aurantianu, Egira (=Xylomyges) curialis, Elasmopalpus lignosellus, Eldana saccharina, Endopiza viteana, Ennomos subsignaria, Eoreuma loftini, Ephestia spp, such as E. cautella, E. elutella, E. kuehniella; Epinotia aporema, Epiphyas postvittana, Erannis tiliaria, Erionota thrax, Etiella spp., Eulia spp., Eupoecilia ambiguella, Euproctis chrysorrhoea, Euxoa spp., Evetria bouliana, Faronta albilinea, Feltia spp, such as F. subterranean; Galleria mellonella, Gracillaria spp., Grapholita spp, such as G. funebrana, G. molesta, G. inopinata; Halysidota spp., Harrisina americana, Hedylepta spp., Helicoverpa spp. such as H. armigera (=Heliothis armigera), H. zea (=Heliothis zea); Heliothis spp, such as H. assulta, H. subflexa, H. virescens; Hellula spp, such as H. undalis, H. rogatalis; Helocoverpa gelotopoeon, Hemileuca oliviae, Herpetogramma licarsisalis, Hibernia defoliaria, Hofmannophila pseudospretella, Homoeosoma electellum, Homona magnanima, Hypena scabra, Hyphantria cunea, Hyponomeuta padella, Hyponomeuta malinellus, Kakivoria flavofasciata, Keiferia lycopersicella, Lambdina fiscellaria fiscellaria, Lambdina fiscellaria lugubrosa, Lamprosema indicata, Laspeyresia molesta, Leguminivora glycinivorella, Lerodea eufala, Leucinodes orbonalis, Leucoma salicis, Leucoptera spp, such as L. coffeella, L. scitella; Leuminivora lycinivorella, Lithocolletis blancardella, Lithophane antennata, Llattia octo (=Amyna axis), Lobesia botrana, Lophocampa spp., Loxagrotis albicosta, Loxostege spp, such as L. sticticalis, L. cereralis; Lymantria spp, such as L. dispar, L. monacha; Lyonetia clerkella, Lyonetia prunifoliella, Malacosoma spp, such as M. americanum, M. californicum, M. constrictum, M. neustria; Mamestra spp, such as M. brassicae, M. configurata; Mamstra brassicae, Manduca spp, such as M. quinquemaculata, M. sexta; Marasmia spp, Marmara spp., Maruca testulalis, Megalopyge lanata, Melanchra picta, Melanitis leda, Mocis spp, such as M. lapites, M. repanda; Mocis latipes, Monochroa fragariae, Mythimna separata, Nemapogon cloacella, Neoleucinodes elegantalis, Nepytia spp., Nymphula spp., Oiketicus spp., Omiodes indicata, Omphisa anastomosalis, Operophtera brumata, Orgyia pseudotsugata, Oria spp., Orthaga thyrisalis, Ostrinia spp, such as O. nubilalis; Oulema oryzae, Paleacrita vernata, Panolis flammea, Parnara spp., Papaipema nebris, Papilio cresphontes, Paramyelois transitella, Paranthrene regalis, Paysandisia archon, Pectinophora spp, such as P. gossypiella; Peridroma saucia, Perileucoptera spp., such as P. coffeella; Phalera bucephala, Phryganidia californica, Phthorimaea spp, such as P. operculella; Phyllocnistis citrella, Phyllonorycter spp, such as P. blancardella, P. crataegella, P. issikii, P. ringoniella; Pieris spp, such as P. brassicae, P. rapae, P. napi; Pilocrocis tripunctata, Plathypena scabra, Platynota spp, such as P. flavedana, P. idaeusalis, P. stultana; Platyptilia carduidactyla, Plebejus argus, Plodia interpunctella, Plusia spp, Plutella maculipennis, Plutella xylostella, Pontia protodica, Prays spp., Prodenia spp., Proxenus lepigone, Pseudaletia spp, such as P. sequax, P. unipuncta; Pyrausta nubilalis, Rachiplusia nu, Richia albicosta, Rhizobius ventralis, Rhyacionia frustrana, Sabulodes aegrotata, Schizura concinna, Schoenobius spp., Schreckensteinia festaliella, Scirpophaga spp, such as S. incertulas, S. innotata; Scotia segetum, Sesamia spp. such as S. inferens, Seudyra subflava, Sitotroga cerealella, Sparganothis pilleriana, Spilonota lechriaspis, S. ocellana, Spodoptera (=Lamphygma) spp, such as S. cosmoides, S. eridania, S. exigua, S. frugiperda, S. latisfascia, S. littoralis, S. litura, S. omithogalli; Stigmella spp., Stomopteryx subsecivella, Strymon bazochii, Sylepta derogata, Synanthedon spp, such as S. exitiosa, Tecia solanivora, Telehin licus, Thaumatopoea pityocampa, Thaumatotibia (=Cryptophlebia) leucotreta, Thaumetopoea pityocampa, Thecla spp., Theresimima ampelophaga, Thyrinteina spp, Tildenia inconspicuella, Tinea spp, such as T. cloacella, T. pellionella; Tineola bisselliella, Tortrix spp, such as T. viridana; Trichophaga tapetzella, Trichoplusia spp, such as T. ni; Tuta (=Scrobipalpula) absoluta, Udea spp, such as U. rubigalis, U. rubigalis; Virachola spp., Yponomeuta padella, and Zeiraphera canadensis;
insects from the order of Coleoptera, for example Acalymma vittatum, Acanthoscehdes obtectus, Adoretus spp., Agelastica alni, Agrilus spp, such as A. anxius, A. planipennis, A. sinuatus; Agriotes spp, such as A. fuscicollis, A. lineatus, A. obscurus; Alphitobius diaperinus, Amphimallus solstitialis, Anisandrus dispar, Anisoplia austriaca, Anobium punctatum, Anomala corpulenta, Anomala rufocuprea, Anoplophora spp, such as A. glabripennis; Anthonomus spp. such as A. eugenii, A. grandis, A. pomorum; Anthrenus spp., Aphthona euphoridae, Apion spp., Apogonia spp., Athous haemorrhoidalis, Atomaria spp, such as A. linearis; Attagenus spp., Aulacophora femoralis, Blastophagus piniperda, Blitophaga undata, Bruchidius obtectus, Bruchus spp, such as B. lentis, B. pisorum, B. rufimanus; Byctiscus betulae, Callidiellum rufipenne, Callopistria floridensis, Callosobruchus chinensis, Cameraria ohridella, Cassida nebulosa, Cerotoma trifurcata, Cetonia aurata, Ceuthorhynchus spp, such as C. assimilis, C. napi; Chaetocnema tibialis, Cleonus mendicus, Conoderus spp, such as C. vespertinus; Conotrachelus nenuphar, Cosmopolites spp., Costelytra zealandica, Crioceris asparagi, Cryptolestes ferrugineus, Cryptorhynchus lapathi, Ctenicera spp, such as C. destructor; Curculio spp., Cylindrocopturus spp., Cyclocephala spp., Dactylispa balyi, Dectes texanus, Dermestes spp., Diabrotica spp, such as D. undecimpunctata, D. speciosa, D. longicornis, D. semipunctata, D. virgifera; Diaprepes abbreviates, Dichocrocis spp., Dicladispa armigera, Diloboderus abderus, Diocalandra frumenti (Diocalandra stigmaticollis), Enaphalodes rufulus, Epilachna spp, such as E. varivestis, E. vigintioctomaculata; Epitrix spp, such as E. hirtipennis, E. similaris; Eutheola humilis, Eutinobothrus brasiliensis, Faustinus cubae, Gibbium psylloides, Gnathocerus cornutus, Hellula undalis, Heteronychus arator, Hylamorpha elegans, Hylobius abietis, Hylotrupes bajulus, Hypera spp, such as H. brunneipennis, H. postica; Hypomeces squamosus, Hypothenemus spp., Ips typographus, Lachnosterna consanguinea, Lasioderma serricorne, Latheticus oryzae, Lathridius spp., Lema spp, such as L. bilineata, L. melanopus; Leptinotarsa spp, such as L. decemlineata; Leptispa pygmaea, Limonius californicus, Lissorhoptrus oryzophilus, Lixus spp., Luperodes spp., Lyctus spp, such as L. bruneus; Liogenys fuscus, Macrodactylus spp, such as M. subspinosus; Maladera matrida, Megaplatypus mutates, Megascelis spp., Melanotus communis, Meligethes spp, such as M. aeneus; Melolontha spp, such as M. hippocastani, M. melolontha; Metamasius hemipterus, Microtheca spp., Migdolus spp, such as M. fryanus, Monochamus spp, such as M. alternatus; Naupactus xanthographus, Niptus hololeucus, Oberia brevis, Oemona hirta, Oryctes rhinoceros, Oryzaephilus surinamensis, Oryzaphagus oryzae, Otiorrhynchus sulcatus, Otiorrhynchus ovatus, Otiorrhynchus sulcatus, Oulema melanopus, Oulema oryzae, Oxycetonia jucunda, Phaedon spp, such as P. brassicae, P. cochleariae; Phoracantha recurva, Phyllobius pyri, Phyllopertha horticola, Phyllophaga spp, such as P. helleri; Phyllotreta spp, such as P. chrysocephala, P. nemorum, P. striolata, P. vittula; Phyllopertha horticola, Popillia japonica, Premnotrypes spp., Psacothea hilaris, Psylliodes chrysocephala, Prostephanus truncates, Psylliodes spp., Ptinus spp., Pulga saltona, Rhizopertha dominica, Rhynchophorus spp, such as R. billineatus, R. ferrugineus, R. palmarum, R. phoenicis, R. vulneratus; Saperda candida, Scolytus schevyrewi, Scyphophorus acupunctatus, Sitona lineatus, Sitophilus spp, such as S. granaria, S. oryzae, S. zeamais; Sphenophorus spp, such as S. levis; Stegobium paniceum, Sternechus spp, such as S. subsignatus; Strophomorphus ctenotus, Symphyletes spp., Tanymecus spp., Tenebrio molitor, Tenebrioides mauretanicus, Tribolium spp. such as T. castaneum; Trogoderma spp., Tychius spp., Xylotrechus spp, such as X. pyrrhoderus; and, Zabrus spp, such as Z. tenebrioides;
insects from the order of Diptera e.g. Aedes spp, such as A. aegypti, A. albopictus, A. vexans; Anastrepha ludens, Anopheles spp, such as A. albimanus, A. crucians, A. freeborni, A. gambiae, A. leucosphyrus, A. maculipennis, A. minimus, A. quadrimaculatus, A. sinensis; Bactrocera invadens, Bibio hortulanus, Calliphora erythrocephala, Calliphora vicina, Ceratitis capitata, Chrysomyia spp, such as C. bezziana, C. hominivorax, C. macellaria; Chrysops atlanticus, Chrysops discalis, Chrysops silacea, Cochliomyia spp, such as C. hominivorax; Contarinia spp. such as C. sorghicola; Cordylobia anthropophage, Culex spp, such as C. nigripalpus, C. pipiens, C. quinquefasciatus, C. tarsalis, C. tritaeniorhynchus; Culicoides furens, Culiseta inornata, Culiseta melanura, Cuterebra spp., Dacus cucurbitae, Dacus oleae, Dasineura brassicae, Dasineura oxycoccana, Delia spp, such as D. antique, D. coarctata, D. platura, D. radicum; Dermatobia hominis, Drosophila spp, such as D. suzukii, Fannia spp, such as F. canicularis; Gastraphilus spp, such as G. intestinalis; Geomyza tipunctata, Glossina spp, such as G. fuscipes, G. morsitans, G. palpalis, G. tachinoides; Haematobia irritans, Haplodiplosis equestris, Hippelates spp., Hylemyia spp, such as H. platura; Hypoderma spp, such as H. lineata; Hyppobosca spp., Hydrellia philippina, Leptoconops torrens, Liriomyza spp, such as L. sativae, L. trifolii; Lucilia spp, such as L. caprina, L. cuprina, L. sericata; Lycoria pectoralis, Mansonia titillanus, Mayetiola spp, such as M. destructor; Musca spp, such as M. autumnalis, M. domestica; Muscina stabulans, Oestrus spp, such as O. ovis; Opomyza florum, Oscinella spp, such as O. frit; Orseolia oryzae, Pegomya hysocyami, Phlebotomus argentipes, Phorbia spp, such as P. antiqua, P. brassicae, P. coarctata; Phytomyza gymnostoma, Prosimulium mixtum, Psila rosae, Psorophora columbiae, Psorophora discolor, Rhagoletis spp, such as R. cerasi, R. cingulate, R. indifferens, R. mendax, R. pomonella; Rivellia quadrifasciata, Sarcophaga spp, such as S. haemorrhoidalis; Simulium vittatum, Sitodiplosis mosellana, Stomoxys spp, such as S. calcitrans; Tabanus spp, such as T. atratus, T. bovinus, T. lineola, T. similis; Tannia spp., Thecodiplosis japonensis, Tipula oleracea, Tipula paludosa, and Wohlfahrtia spp;
insects from the order of Thysanoptera for example, Baliothrips biformis, Dichromothrips corbetti, Dichromothrips ssp., Echinothrips americanus, Enneothrips flavens, Frankliniella spp. such as F. fusca, F. occidentalis, F. tritici; Heliothrips spp., Hercinothrips femoralis, Kakothrips spp., Microcephalothrips abdominalis, Neohydatothrips samayunkur, Pezothrips kellyanus, Rhipiphorothrips cruentatus, Scirtothrips spp, such as S. citri, S. dorsalis, S. perseae; Stenchaetothrips spp, Taeniothrips cardamoni, Taeniothrips inconsequens, Thrips spp, such as T. imagines, T. hawaiiensis, T. oryzae, T. palmi, T. parvispinus, T. tabaci;
insects from the order of Hemiptera for example, Acizzia jamatonica, Acrosternum spp, such as A. hilare; Acyrthosipon spp, such as A. onobrychis, A. pisum; Adelges laricis, Adelges tsugae, Adelphocoris spp., such as A. rapidus, A. superbus; Aeneolamia spp., Agonoscena spp., Aulacorthum solani, Aleurocanthus woglumi, Aleurodes spp., Aleurodicus disperses, Aleurolobus barodensis, Aleurothrixus spp., Amrasca spp., Anasa tristis, Antestiopsis spp., Anuraphis cardui, Aonidiella spp., Aphanostigma piri, Aphidula nasturtii, Aphis spp, such as A. craccivora, A. fabae, A. forbesi, A. gossypii, A. grossulariae, A. maidiradicis, A. pomi, A. sambuci, A. schneideri, A. spiraecola; Arboridia apicalis, Arilus critatus, Aspidiella spp., Aspidiotus spp., Atanus spp., Aulacaspis yasumatsui, Aulacorthum solani, Bactericera cockerelli (Paratrioza cockerelli), Bemisia spp, such as B. argentifolii, B. tabaci (Aleurodes tabaci); Blissus spp, such as B. leucopterus; Brachycaudus spp, such as B. cardui, B. helichrysi, B. persicae, B. prunicola; Brachycolus spp., Brachycorynella asparagi, Brevicoryne brassicae, Cacopsylla spp, such as C. fulguralis, C. pyricola (Psylla piri); Calligypona marginata, Calocoris spp., Campylomma livida, Capitophorus horni, Carneocephala fulgida, Cavelerius spp., Ceraplastes spp., Ceratovacuna lanigera, Ceroplastes ceriferus, Cerosipha gossypii, Chaetosiphon fragaefolii, Chionaspis tegalensis, Chlorita onukii, Chromaphis juglandicola, Chrysomphalus ficus, Cicadulina mbila, Cimex spp, such as C. hemipterus, C. lectularius; Coccomytilus halli, Coccus spp, such as C. hesperidum, C. pseudomagnoliarum; Corythucha arcuata, Creontiades dilutus, Cryptomyzus ribis, Chrysomphalus aonidum, Cryptomyzus ribis, Ctenarytaina spatulata, Cyrtopeltis notatus, Dalbulus spp., Dasynus piperis, Dialeurodes spp, such as D. citrifolii; Dalbulus maidis, Diaphorina spp, such as D. citri; Diaspis spp, such as D. bromeliae; Dichelops furcatus, Diconocoris hewetti, Doralis spp., Dreyfusia nordmannianae, Dreyfusia piceae, Drosicha spp., Dysaphis spp, such as D. plantaginea, D. pyri, D. radicola; Dysaulacorthum pseudosolani, Dysdercus spp, such as D. cingulatus, D. intermedius; Dysmicoccus spp., Edessa spp., Geocoris spp., Empoasca spp. such as E. fabae, E. solana; Epidiaspis leperii, Eriosoma spp, such as E. lanigerum, E. pyricola; Erythroneura spp., Eurygaster spp, such as E. integriceps; Euscelis bilobatus, Euschistus spp. such as E. heros, E. impictiventris, E. servus; Fiorinia theae, Geococcus coffeae, Glycaspis brimblecombei, Halyomorpha spp, such as H. halys; Heliopeltis spp., Homalodisca vitripennis (=H. coagulata), Horcias nobilellus, Hyalopterus pruni, Hyperomyzus lactucae, Icerya spp. such as I. purchase; Idiocerus spp., Idioscopus spp., Laodelphax striatellus, Lecanium spp., Lecanoideus floccissimus, Lepidosaphes spp, such as L. ulmi; Leptocorisa spp., Leptoglossus phyllopus, Lipaphis erysimi, Lygus spp, such as L. hesperus, L. lineolaris, L. pratensis; Maconellicoccus hirsutus, Marchalina hellenica, Macropes excavatus, Macrosiphum spp, such as M. rosae, M. avenae, M. euphorbiae; Macrosteles quadrilineatus, Mahanarva fimbriolata, Megacopta cribraria, Megoura viciae, Melanaphis pyrarius, Melanaphis sacchari, Melanocallis (=Tinocallis) caryaefoliae, Metcafiella spp., Metopolophium dirhodum, Monellia costalis, Monelliopsis pecanis, Myzocallis coryli, Murgantia spp., Myzus spp, such as M. ascalonicus, M. cerasi, M. nicotianae, M. persicae, M. varians; Nasonovia ribis-nigri, Neotoxoptera formosana, Neomegalotomus spp, Nephotettix spp, such as N. malayanus, N. nigropictus, N. parvus, N. virescens; Nezara spp, such as N. viridula; Nilaparvata lugens, Nysius huttoni, Oebalus spp. such as O. pugnax; Oncometopia spp., Orthezia praelonga, Oxycaraenus hyalinipennis, Parabemisia myricae, Parlatoria spp., Parthenolecanium spp, such as P. corni, P. persicae; Pemphigus spp. such as P. bursarius, P. populivenae; Peregrinus maidis, Perkinsiella saccharicida, Phenacoccus spp, such as P. aceris, P. gossypii; Phloeomyzus passerinii, Phorodon humuli, Phylloxera spp. such as P. devastatrix, Piesma quadrata, Piezodorus spp, such as P. guildinii; Pinnaspis aspidistrae, Planococcus spp, such as P. citri, P. ficus; Prosapia bicincta, Protopulvinaria pyriformis, Psallus seriatus, Pseudacysta persea, Pseudaulacaspis pentagona, Pseudococcus spp, such as P. comstocki; Psylla spp, such as P. mali; Pteromalus spp., Pulvinaria amygdali, Pyrilla spp., Quadraspidiotus spp., such as Q. perniciosus; Quesada gigas, Rastrococcus spp., Reduvius senilis, Rhizoecus americanus, Rhodnius spp., Rhopalomyzus ascalonicus, Rhopalosiphum spp, such as R. pseudobrassicas, R. insertum, R. maidis, R. padi; Sagatodes spp., Sahlbergella singularis, Saissetia spp., Sappaphis mala, Sappaphis mali, Scaptocoris spp., Scaphoides titanus, Schizaphis graminum, Schizoneura lanuginosa, Scotinophora spp., Selenaspidus articulatus, Sitobion avenae, Sogata spp., Sogatella furcifera, Solubea insularis, Spissistilus festinus (=Stictocephala festina), Stephanitis nashi, Stephanitis pyrioides, Stephanitis takeyai, Tenalaphara malayensis, Tetraleurodes perseae, Therioaphis maculate, Thyanta spp. such as T. accerra, T. perditor; Tibraca spp., Tomaspis spp., Toxoptera spp, such as T. aurantii; Trialeurodes spp, such as T. abutilonea, T. ricini, T. vaporariorum; Triatoma spp., Trioza spp., Typhlocyba spp., Unaspis spp, such as U. citri, U. yanonensis; and Viteus vitifolii,
Insects from the order Hymenoptera for example Acanthomyops interjectus, Athalia rosae, Atta spp, such as A. capiguara, A. cephalotes, A. cephalotes, A. laevigata, A. robusta, A. sexdens, A. texana, Bombus spp., Brachymyrmex spp., Camponotus spp, such as C. floridanus, C. pennsylvanicus, C. modoc; Cardiocondyla nuda, Chalibion sp, Crematogaster spp., Dasymutilla occidentalis, Diprion spp., Dolichovespula maculata, Dorymyrmex spp., Dryocosmus kuriphilus, Formica spp., Hoplocampa spp, such as H. minuta, H. testudinea; Iridomyrmex humilis, Lasius spp, such as L. niger, Linepithema humile, Liometopum spp., Leptocybe invasa, Monomorium spp, such as M. pharaonis, Monomorium, Nylandria fulva, Pachycondyla chinensis, Paratrechina longicornis, Paravespula spp., such as P. germanica, P. pennsylvanica, P. vulgaris; Pheidole spp, such as P. megacephala; Pogonomyrmex spp, such as P. barbatus, P. californicus, Polistes rubiginosa, Prenolepis impairs, Pseudomyrmex gracilis, Schelipron spp., Sirex cyaneus, Solenopsis spp, such as S. geminata, S. invicta, S. molesta, S. richteri, S. xyloni, Sphecius speciosus, Sphex spp., Tapinoma spp, such as T. melanocephalum, T. sessile; Tetramorium spp. such as T. caespitum, T. bicarinatum, Vespa spp, such as V. crabro; Vespula spp, such as V. squamosal; Wasmannia auropunctata, Xylocopa sp;
Insects from the order Orthoptera for example Acheta domesticus, Calliptamus italicus, Chortoicetes terminifera, Ceuthophilus spp., Diastrammena asynamora, Dociostaurus maroccanus, Gryllotalpa spp, such as G. africana, G. gryllotalpa; Gryllus spp., Hieroglyphus daganensis, Kraussaria angulifera, Locusta spp, such as L. migratoria, L. pardalina; Melanoplus spp, such as M. bivittatus, M. femurrubrum, M. mexicanus, M. sanguinipes, M. spretus; Nomadacris septemfasciata, Oedaleus senegalensis, Scapteriscus spp., Schistocerca spp. such as S. americana, S. gregaria, Stemopelmatus spp., Tachycines asynamorus, and Zonozerus variegatus;
Pests from the Class Arachnida for example Acari, e.g. of the families Argasidae, Ixodidae and Sarcoptidae, such as Amblyomma spp. (e.g. A. americanum, A. variegatum, A. maculatum), Argas spp, such as A. persicu), Boophilus spp, such as B. annulatus, B. decoloratus, B. microplus, Dermacentor spp, such as D. silvarum, D. andersoni, D. variabilis, Hyalomma spp, such as H. truncatum, Ixodes spp, such as I. ricinus, I. rubicundus, I. scapularis, I. holocyclus, I. pacificus, Rhipicephalus sanguineus, Ornithodorus spp, such as O. moubata, O. hermsi, O. turicata, Ornithonyssus bacoti, Otobius megnini, Dermanyssus gallinae, Psoroptes spp, such as P. ovis, Rhipicephalus spp, such as R. sanguineus, R. appendiculatus, Rhipicephalus evertsi, Rhizoglyphus spp., Sarcoptes spp, such as S. Scabiei; and Family Eriophyidae including Aceria spp. such as A. sheldoni, A. anthocoptes, Acallitus spp., Aculops spp, such as A. lycopersici, A. pelekassi; Aculus spp, such as A. schlechtendali; Colomerus vitis, Epitrimerus pyri, Phyllocoptruta oleivora; Eriophytes ribis and Eriophyes spp, such as Eriophyes sheldoni; Family Tarsonemidae including Hemitarsonemus spp., Phytonemus pallidus and Polyphagotarsonemus latus, Stenotarsonemus spp. Steneotarsonemus spinki; Family Tenuipalpidae including Brevipalpus spp, such as B. phoenicis; Family Tetranychidae including Eotetranychus spp., Eutetranychus spp., Oligonychus spp., Petrobia latens, Tetranychus spp, such as T. cinnabarinus, T. evansi, T. kanzawai, T, pacificus, T. phaseulus, T. telarius and T. urticae; Bryobia praetiosa; Panonychus spp, such as P. ulmi, P. citri; Metatetranychus spp, and Oligonychus spp, such as O. pratensis, O. perseae, Vasates lycopersici; Raoiella indica, Family Carpoglyphidae including Carpoglyphus spp.; Penthaleidae spp, such as Halotydeus destructor, Family Demodicidae with species such as Demodex spp.; Family Trombicidea including Trombicula spp.; Family Macronyssidae including Ornothonyssus spp.; Family Pyemotidae including Pyemotes tritici; Tyrophagus putrescentiae; Family Acaridae including Acarus siro; Family Araneida including Latrodectus mactans, Tegenaria agrestis, Chiracanthium sp, Lycosa sp Achaearanea tepidariorum and Loxosceles reclusa;
Pests from the Phylum Nematoda, for example, plant parasitic nematodes such as root-knot nematodes, Meloidogyne spp, such as M. hapla, M. incognita, M. javanica; cyst-forming nematodes, Globodera spp, such as G. rostochiensis; Heterodera spp, such as H. avenae, H. glycines, H. schachtii, H. trifolii; Seed gall nematodes, Anguina spp.; Stem and foliar nematodes, Aphelenchoides spp, such as A. besseyi; Sting nematodes, Belonolaimus spp, such as B. longicaudatus; Pine nematodes, Bursaphelenchus spp, such as B. lignicolus, B. xylophilus; Ring nematodes, Criconema spp., Criconemella spp, such as C. xenoplax and C. ornata; and, Criconemoides spp, such as Criconemoides informis; Mesocriconema spp.; Stem and bulb nematodes, Ditylenchus spp, such as D. destructor, D. dipsaci; Awl nematodes, Dolichodorus spp.; Spiral nematodes, Heliocotylenchus multicinctus; Sheath and sheathoid nematodes, Hemicycliophora spp, and Hemicriconemoides spp.; Hirshmanniella spp.; Lance nematodes, Hoploaimus spp.; False rootknot nematodes, Nacobbus spp.; Needle nematodes, Longidorus spp, such as L. elongatus; Lesion nematodes, Pratylenchus spp, such as P. brachyurus, P. neglectus, P. penetrans, P. curvitatus, P. goodeyi; Burrowing nematodes, Radopholus spp. such as R. similis; Rhadopholus spp.; Rhodopholus spp.; Reniform nematodes, Rotylenchus spp. such as R. robustus, R. reniformis; Scutellonema spp.; Stubby-root nematode, Trichodorus spp. such as T. obtusus, T. primitivus; Paratrichodorus spp, such as P. minor; Stunt nematodes, Tylenchorhynchus spp, such as T. claytoni, T. dubius; Citrus nematodes, Tylenchulus spp. such as T. semipenetrans; Dagger nematodes, Xiphinema spp.; and other plant parasitic nematode species;
Insects from the order Blattodea for example Macrotermes spp, such as M. natalensis; Cornitermes cumulans, Procornitermes spp., Globitermes sulfureus, Neocapritermes spp. such as N. opacus, N. parvus; Odontotermes spp., Nasutitermes spp, such as N. corniger; Coptotermes spp, such as C. formosanus, C. gestroi, C. acinaciformis; Reticulitermes spp. such as R. hesperus, R. tibialis, R. speratus, R. flavipes, R. grassei, R. lucifugus, R. virginicus; Heterotermes spp, such as H. aureus, H. longiceps, H. tenuis; Cryptotermes spp, such as C. brevis, C. cavifrons; Incisitermes spp, such as I. minor, I. snyderi; Marginitermes hubbardi, Kalotermes flavicollis, Neotermes spp, such as N. castaneus, Zootermopsis spp, such as Z. angusticollis, Z. nevadensis, Mastotermes spp, such as M. darwiniensis; Blatta spp, such as B. orientalis, B. lateralis; Blattella spp, such as B. asahinae, B. germanica; Rhyparobia maderae, Panchlora nivea, Periplaneta spp, such as P. americana, P. australasiae, P. brunnea, P. fuliginosa, P. japonica; Supella longipalpa, Parcoblatta pennsylvanica, Eurycotis floridana, Pycnoscelus surinamensis,
Insects from the order Siphonoptera for example Cediopsylla simples, Ceratophyllus spp., Ctenocephalides spp, such as C. felis, C. canis, Xenopsylla cheopis, Pulex irritans, Trichodectes canis, Tunga penetrans, and Nosopsyllus fasciatus,
Insects from the order Thysanura for example Lepisma saccharina, Ctenolepisma urbana, and Thermobia domestica,
Pests from the class Chilopoda for example Geophilus spp., Scutigera spp, such as Scutigera coleoptrata;
Pests from the class Diplopoda for example Blaniulus guttulatus, Julus spp., Narceus spp.,
Pests from the class Symphyla for example Scutigerella immaculata,
Insects from the order Dermaptera, for example Forficula auricularia,
Insects from the order Collembola, for example Onychiurus spp., such as Onychiurus armatus,
Pests from the order Isopoda for example, Armadillidium vulgare, Oniscus asellus, Porcellio scaber,
Insects from the order Phthiraptera, for example Damalinia spp., Pediculus spp, such as Pediculus humanus capitis, Pediculus humanus corporis, Pediculus humanus humanus; Pthirus pubis, Haematopinus spp, such as Haematopinus eurysternus, Haematopinus suis; Linognathus spp, such as Linognathus vituli; Bovicola bovis, Menopon gallinae, Menacanthus stramineus and Solenopotes capillatus, Trichodectes spp.,

Examples of further pest species which may be controlled by compounds of formula (I) include: from the Phylum Mollusca, class Bivalvia, for example, Dreissena spp.; class Gastropoda, for example, Arion spp., Biomphalaria spp., Bulinus spp., Deroceras spp., Galba spp., Lymnaea spp., Oncomelania spp., Pomacea canaliclata, Succinea spp.; from the class of the helminths, for example, Ancylostoma duodenale, Ancylostoma ceylanicum, Acylostoma braziliensis, Ancylostoma spp., Ascaris lumbricoides, Ascaris spp., Brugia malayi, Brugia timori, Bunostomum spp., Chabertia spp., Clonorchis spp., Cooperia spp., Dicrocoelium spp., Dictyocaulus filaria, Diphyllobothrium latum, Dracunculus medinensis, Echinococcus granulosus, Echinococcus multilocularis, Enterobius vermicularis, Faciola spp., Haemonchus spp, such as Haemonchus contortus; Heterakis spp., Hymenolepis nana, Hyostrongulus spp., Loa Loa, Nematodirus spp., Oesophagostomum spp., Opisthorchis spp., Onchocerca volvulus, Ostertagia spp., Paragonimus spp., Schistosomen spp., Strongyloides fuelleborni, Strongyloides stercoralis, Stronyloides spp., Taenia saginata, Taenia solium, Trichinella spiralis, Trichinella nativa, Trichinella britovi, Trichinella nelsoni, Trichinella pseudopsiralis, Trichostrongulus spp., Trichuris trichiura, Wuchereria bancrofti.

The compounds of the invention are suitable for use in treating or protecting animals against infestation or infection by parasites. Therefore, the invention also relates to the use of a compound of the invention for the manufacture of a medicament for the treatment or protection of animals against infestation or infection by parasites. Furthermore, the invention relates to a method of treating or protecting animals against infestation and infection by parasites, which comprises orally, topically or parenterally administering or applying to the animals a parasiticidally effective amount of a compound of the invention.

The present invention also relates to the non-therapeutic use of compounds of the invention for treating or protecting animals against infestation and infection by parasites. Moreover, the invention relates to a non-therapeutic method of treating or protecting animals against infestation and infection by parasites, which comprises applying to a locus a parasiticidally effective amount of a compound of the invention.

The compounds of the invention are further suitable for use in combating or controlling parasites in and on animals. Furthermore, the invention relates to a method of combating or controlling parasites in and on animals, which comprises contacting the parasites with a parasitically effective amount of a compound of the invention.

The invention also relates to the non-therapeutic use of compounds of the invention for controlling or combating parasites. Moreover, the invention relates to a non-therapeutic method of combating or controlling parasites, which comprises applying to a locus a parasiticidally effective amount of a compound of the invention.

The compounds of the invention can be effective through both contact (via soil, glass, wall, bed net, carpet, blankets or animal parts) and ingestion (e.g. baits). Furthermore, the compounds of the invention can be applied to any and all developmental stages.

The compounds of the invention can be applied as such or in form of compositions comprising the compounds of the invention.

The compounds of the invention can also be applied together with a mixing partner, which acts against pathogenic parasites, e.g. with synthetic coccidiosis compounds, polyetherantibiotics such as Amprolium, Robenidin, Toltrazuril, Monensin, Salinomycin, Maduramicin, Lasalocid, Narasin or Semduramicin, or with other mixing partners as defined above, or in form of compositions comprising said mixtures.

The compounds of the invention and compositions comprising them can be applied orally, parenterally or topically, e.g. dermally. The compounds of the invention can be systemically or non-systemically effective.

The application can be carried out prophylactically, therapeutically or non-therapeutically. Furthermore, the application can be carried out preventively to places at which occurrence of the parasites is expected.

As used herein, the term “contacting” includes both direct contact (applying the compounds/compositions directly on the parasite, including the application directly on the animal or excluding the application directly on the animal, e.g. at it's locus for the latter) and indirect contact (applying the compounds/compositions to the locus of the parasite). The contact of the parasite through application to its locus is an example of a non-therapeutic use of the compounds of the invention.

The term “locus” means the habitat, food supply, breeding ground, area, material or environment in which a parasite is growing or may grow outside of the animal.

As used herein, the term “parasites” includes endo- and ectoparasites. In some embodiments of the invention, endoparasites can be preferred. In other embodiments, ectoparasites can be preferred. Infestations in warm-blooded animals and fish include, but are not limited to, lice, biting lice, ticks, nasal bots, keds, biting flies, muscoid flies, flies, myiasitic fly larvae, chiggers, gnats, mosquitoes and fleas.

The compounds of the invention are especially useful for combating parasites of the following orders and species, respectively:
fleas (Siphonaptera), e.g. Ctenocephalides felis, Ctenocephalides canis, Xenopsylla cheopis, Pulex irritans, Tunga penetrans, and Nosopsyllus fasciatus; cockroaches (Blattaria—Blattodea), e.g. Blattella germanica, Blattella asahinae, Periplaneta americana, Periplaneta japonica, Periplaneta brunnea, Periplaneta fuligginosa, Periplaneta australasiae, and Blatta orientalis; flies, mosquitoes (Diptera), e.g. Aedes aegypti, Aedes albopictus, Aedes vexans, Anastrepha ludens, Anopheles maculipennis, Anopheles crucians, Anopheles albimanus, Anopheles gambiae, Anopheles freeborni, Anopheles leucosphyrus, Anopheles minimus, Anopheles quadrimaculatus, Calliphora vicina, Chrysomya bezziana, Chrysomya hominivorax, Chrysomya macellaria, Chrysops discalis, Chrysops silacea, Chrysops atlanticus, Cochliomyia hominivorax, Cordylobia anthropophage, Culicoides furens, Culex pipiens, Culex nigripalpus, Culex quinquefasciatus, Culex tarsalis, Culiseta inornata, Culiseta melanura, Dermatobia hominis, Fannia canicularis, Gasterophilus intestinalis, Glossina morsitans, Glossina palpalis, Glossina fuscipes, Glossina tachinoides, Haematobia irritans, Haplodiplosis equestris, Hippelates spp., Hypoderma lineata, Leptoconops torrens, Lucilia caprina, Lucilia cuprina, Lucilia sericata, Lycoria pectoralis, Mansonia spp., Musca domestica, Muscina stabulans, Oestrus ovis, Phlebotomus argentipes, Psorophora columbiae, Psorophora discolor, Prosimulium mixtum, Sarcophaga haemorrhoidalis, Sarcophaga sp., Simulium vittatum, Stomoxys calcitrans, Tabanus bovinus, Tabanus atratus, Tabanus lineola, and Tabanus similis; lice (Phthiraptera), e.g. Pediculus humanus capitis, Pediculus humanus humanus, Pthirus pubis, Haematopinus eurysternus, Haematopinus suis, Linognathus vituli, Bovicola bovis, Menopon gallinae, Menacanthus stramineus and Solenopotes capillatus; ticks and parasitic mites (Parasitiformes): ticks (Ixodida), e.g. Ixodes scapularis, Ixodes holocyclus, Ixodes pacificus, Rhiphicephalus sanguineus, Dermacentor andersoni, Dermacentor variabilis, Amblyomma americanum, Ambryomma maculatum, Ornithodorus hermsi, Ornithodorus turicata and parasitic mites (Mesostigmata), e.g. Ornithonyssus bacoti and Dermanyssus gallinae; Actinedida (Prostigmata) und Acaridida (Astigmata), e.g. Acarapis spp., Cheyletiella spp., Ornithocheyletia spp., Myobia spp., Psorergates spp., Demodex spp., Trombicula spp., Listrophorus spp., Acarus spp., Tyrophagus spp., Caloglyphus spp., Hypodectes spp., Pterolichus spp., Psoroptes spp., Chorioptes spp., Otodectes spp., Sarcoptes spp., Notoedres spp., Knemidocoptes spp., Cytodites spp., and Laminosioptes spp; Bugs (Heteropterida): Cimex lectularius, Cimex hemipterus, Reduvius senilis, Triatoma spp., Rhodnius ssp., Panstrongylus ssp., and Arilus critatus; Anoplurida, e.g. Haematopinus spp., Linognathus spp., Pediculus spp., Phtirus spp., and Solenopotes spp.; Mallophagida (suborders Arnblycerina and Ischnocerina), e.g. Trimenopon spp., Menopon spp., Trinoton spp., Bovicola spp., Werneckiella spp., Lepikentron spp., Trichodectes spp., and Felicola spp.; Roundworms Nematoda: Wipeworms and Trichinosis (Trichosyringida), e.g. Trichinellidae (Trichinella spp.), (Trichuridae) Trichuris spp., Capillaria spp.; Rhabditida, e.g. Rhabditis spp., Strongyloides spp., Helicephalobus spp.; Strongylida, e.g. Strongylus spp., Ancylostoma spp., Necator americanus, Bunostomum spp. (Hookworm), Trichostrongylus spp., Haemonchus contortus, Ostertagia spp., Cooperia spp., Nematodirus spp., Dictyocaulus spp., Cyathostoma spp., Oesophagostomum spp., Stephanurus dentatus, Ollulanus spp., Chabertia spp., Stephanurus dentatus, Syngamus trachea, Ancylostoma spp., Uncinaria spp., Globocephalus spp., Necator spp., Metastrongylus spp., Muellerius capillaris, Protostrongylus spp., Angiostrongylus spp., Parelaphostrongylus spp., Aleurostrongylus abstrusus, and Dioctophyma renale; Intestinal roundworms (Ascaridida), e.g. Ascaris lumbricoides, Ascaris suum, Ascaridia galli, Parascaris equorum, Enterobius vermicularis (Threadworm), Toxocara canis, Toxascaris leonine, Skrjabinema spp., and Oxyuris equi; Camallanida, e.g. Dracunculus medinensis (guinea worm); Spirurida, e.g. Thelazia spp., Wuchereria spp., Brugia spp., Onchocerca spp., Dirofilari spp.a, Dipetalonema spp., Setaria spp., Elaeophora spp., Spirocerca lupi, and Habronema spp.; Thorny headed worms (Acanthocephala), e.g. Acanthocephalus spp., Macracanthorhynchus hirudinaceus and Oncicola spp.; Planarians (Plathelminthes): Flukes (Trematoda), e.g. Faciola spp., Fascioloides magna, Paragonimus spp., Dicrocoelium spp., Fasciolopsis buski, Clonorchis sinensis, Schistosoma spp., Trichobilharzia spp., Alaria alata, Paragonimus spp., and Nanocyetes spp.; Cercomeromorpha, in particular Cestoda (Tapeworms), e.g. Diphyllobothrium spp., Tenia spp., Echinococcus spp., Dipylidium caninum, Multiceps spp., Hymenolepis spp., Mesocestoides spp., Vampirolepis spp., Moniezia spp., Anoplocephala spp., Sirometra spp., Anoplocephala spp., and Hymenolepis spp.

As used herein, the term “animal” includes warm-blooded animals (including humans) and fish. Preferred are mammals, such as cattle, sheep, swine, camels, deer, horses, pigs, poultry, rabbits, goats, dogs and cats, water buffalo, donkeys, fallow deer and reindeer, and also in fur-bearing animals such as mink, chinchilla and raccoon, birds such as hens, geese, turkeys and ducks and fish such as fresh- and salt-water fish such as trout, carp and eels. Particularly preferred are domestic animals, such as dogs or cats.

In general, “parasiticidally effective amount” means the amount of active ingredient needed to achieve an observable effect on growth, including the effects of necrosis, death, retardation, prevention, and removal, destruction, or otherwise diminishing the occurrence and activity of the target organism. The parasiticidally effective amount can vary for the various compounds/compositions used in the invention. A parasiticidally effective amount of the compositions will also vary according to the prevailing conditions such as desired parasiticidal effect and duration, target species, mode of application, and the like.

Generally, it is favorable to apply the compounds of the invention in total amounts of 0.5 mg/kg to 100 mg/kg per day, preferably 1 mg/kg to 50 mg/kg per day.

For oral administration to warm-blooded animals, the formula I compounds may be formulated as animal feeds, animal feed premixes, animal feed concentrates, pills, solutions, pastes, suspensions, drenches, gels, tablets, boluses and capsules. In addition, the formula I compounds may be administered to the animals in their drinking water. For oral administration, the dosage form chosen should provide the animal with 0.01 mg/kg to 100 mg/kg of animal body weight per day of the formula I compound, preferably with 0.5 mg/kg to 100 mg/kg of animal body weight per day.

Alternatively, the formula I compounds may be administered to animals parenterally, for example, by intraruminal, intramuscular, intravenous or subcutaneous injection. The formula I compounds may be dispersed or dissolved in a physiologically acceptable carrier for subcutaneous injection. Alternatively, the formula I compounds may be formulated into an implant for subcutaneous administration. In addition the formula I compound may be transdermally administered to animals. For parenteral administration, the dosage form chosen should provide the animal with 0.01 mg/kg to 100 mg/kg of animal body weight per day of the formula I compound.

The formula I compounds may also be applied topically to the animals in the form of dips, dusts, powders, collars, medallions, sprays, shampoos, spot-on and pour-on formulations and in ointments or oil-in-water or water-in-oil emulsions. For topical application, dips and sprays usually contain 0.5 ppm to 5,000 ppm and preferably 1 ppm to 3,000 ppm of the formula I compound. In addition, the formula I compounds may be formulated as ear tags for animals, particularly quadrupeds such as cattle and sheep.

Suitable preparations are:
Solutions such as oral solutions, concentrates for oral administration after dilution, solutions for use on the skin or in body cavities, pouring-on formulations, gels;
Emulsions and suspensions for oral or dermal administration; semi-solid preparations;
Formulations in which the active compound is processed in an ointment base or in an oil-in-water or water-in-oil emulsion base;
Solid preparations such as powders, premixes or concentrates, granules, pellets, tablets, boluses, capsules; aerosols and inhalants, and active compound-containing shaped articles.

Compositions suitable for injection are prepared by dissolving the active ingredient in a suitable solvent and optionally adding further auxiliaries such as acids, bases, buffer salts, preservatives, and solubilizers. Suitable auxiliaries for injection solutions are known in the art. The solutions are filtered and filled sterile.

Oral solutions are administered directly. Concentrates are administered orally after prior dilution to the use concentration. Oral solutions and concentrates are prepared according to the state of the art and as described above for injection solutions, sterile procedures not being necessary.

Solutions for use on the skin are trickled on, spread on, rubbed in, sprinkled on or sprayed on. Solutions for use on the skin are prepared according to the state of the art and according to what is described above for injection solutions, sterile procedures not being necessary.

Gels are applied to or spread on the skin or introduced into body cavities. Gels are prepared by treating solutions which have been prepared as described in the case of the injection solutions with sufficient thickener that a clear material having an ointment-like consistency results. Suitable thickeners are known in the art.

Pour-on formulations are poured or sprayed onto limited areas of the skin, the active compound penetrating the skin and acting systemically. Pour-on formulations are prepared by dissolving, suspending or emulsifying the active compound in suitable skin-compatible solvents or solvent mixtures. If appropriate, other auxiliaries such as colorants, bioabsorption-promoting substances, antioxidants, light stabilizers, adhesives are added. Suitable such auxiliaries are known in the art.

Emulsions can be administered orally, dermally or as injections. Emulsions are either of the water-in-oil type or of the oil-in-water type. They are prepared by dissolving the active compound either in the hydrophobic or in the hydrophilic phase and homogenizing this with the solvent of the other phase with the aid of suitable emulsifiers and, if appropriate, other auxiliaries such as colorants, absorption-promoting substances, preservatives, antioxidants, light stabilizers, viscosity-enhancing substances. Suitable hydrophobic phases (oils), suitable hydrophilic phases, suitable emulsifiers, and suitable further auxiliaries for emulsions are known in the art.

Suspensions can be administered orally or topically/dermally. They are prepared by suspending the active compound in a suspending agent, if appropriate with addition of other auxiliaries such as wetting agents, colorants, bioabsorption-promoting substances, preservatives, antioxidants, light stabilizers. Suitable suspending agents, and suitable other auxiliaries for suspensions including wetting agents are known in the art.

Semi-solid preparations can be administered orally or topically/dermally. They differ from the suspensions and emulsions described above only by their higher viscosity.

For the production of solid preparations, the active compound is mixed with suitable excipients, if appropriate with addition of auxiliaries, and brought into the desired form. Suitable auxiliaries for this purpose are known in the art.

The compositions which can be used in the invention can comprise generally from about 0.001 to 95% of the compound of the invention.

Ready-to-use preparations contain the compounds acting against parasites, preferably ectoparasites, in concentrations of 10 ppm to 80 percent by weight, preferably from 0.1 to 65 percent by weight, more preferably from 1 to 50 percent by weight, most preferably from 5 to 40 percent by weight.

Preparations which are diluted before use contain the compounds acting against ectoparasites in concentrations of 0.5 to 90 percent by weight, preferably of 1 to 50 percent by weight.

Furthermore, the preparations comprise the compounds of formula I against endoparasites in concentrations of 10 ppm to 2 percent by weight, preferably of 0.05 to 0.9 percent by weight, very particularly preferably of 0.005 to 0.25 percent by weight.

Topical application may be conducted with compound-containing shaped articles such as collars, medallions, ear tags, bands for fixing at body parts, and adhesive strips and foils.

Generally it is favorable to apply solid formulations which release compounds of the invention in total amounts of 10 mg/kg to 300 mg/kg, preferably 20 mg/kg to 200 mg/kg, most preferably 25 mg/kg to 160 mg/kg body weight of the treated animal in the course of three weeks.

### EXAMPLES

With appropriate modification of the starting materials, the procedures as described in the preparation examples below were used to obtain further compounds of formula I. The compounds obtained in this manner are listed in the table C that follows, together with physical data.

Compounds can be characterized e.g., by coupled High Performance Liquid Chromatography/mass spectrometry (HPLC/MS), by ¹H-NMR and/or by their melting points.

Analytical HPLC-Method 1: Agilent Eclipse Plus C18, 50×4.6 mm, ID 5 μm; Elution: A=10 mM Amm. Formate (0.1% Formic Acid), B=Acetonitrile (0.1% Formic Acid), Flow=1.2 ml/min. at 30 °C; Gradient: 10% B to 100% B-3 min, hold for 1 min, 1 min-10% B. Run Time=5.01 min.

Analytical HPLC-Method 2: Kinetex XB C18 1.7μ 50×2.1 mm; A=Water+0.1% TFA, B=Acetonitrile, Flow=0.8 ml/min-1.0 ml/min in 1.5 min. at 60 °C; Gradient: 5% B to 100% B-1.5 min.

¹H-NMR: The signals are characterized by chemical shift (ppm, δ [delta]) vs. tetramethylsilane respectively, CDCl₃ for ¹³C-NMR, by their multiplicity and by their integral (relative number of hydrogen atoms given). The following abbreviations are used to characterize the multiplicity of the signals: m=multiplet, q=quartet, t=triplet, d=doublet and s=singlet.

Abbreviations used are: d for day(s), h for hour(s), min for minute(s), RT/room temperature for 20-25 °C, Rt for retention time; DMSO for dimethyl sulfoxide, OAc for acetate, EtOAc for ethyl acetate, IPA for isopropyl alcohol, MeOH for methanol, EtOH for ethanol, THF for tetrahydrofuran, DMF for N,N-Dimethylformamide and t-BuOH for tert-butanol.

### Preparation Examples

### Example C-1

### Synthesis of N-[1-[4-[[(Z)-[3-(2-isopropyl-5-methyl-phenyl)-4-oxo-thiazolidin-2-ylidene]carbamoyl]amino]-3-methyl-phenyl]-3-methyl-pyrazol-4-yl]-4-(trifluoromethoxy)benzamide

### Step 1: Synthesis of 3-methyl-1H-pyrazol-4-amine

To a stirred solution of 3-methyl-4-nitro pyrazole (5 g) in IPA (40 mL) and MeOH (40 mL) mixture were added bispinacolato diboron (30.0 g) and potassium tert-butoxide (5.3 g) at RT. The whole reaction mixture was heated at 110 °C for 3 h. The progress of the reaction was monitored by GCMS analysis. The reaction mixture was filtered through celite pad and the filtrate was concentrated under reduced pressure to get the desired product as brown solid (3.8 g). GC/MS: Rt: 4.07 min; m/z=97 (M).

### Step 2: Synthesis of N-(3-methyl-1H-pyrazol-4-yl)-4-(trifluoromethoxy)benzamide

To a stirred solution of 3-methyl 4-amino pyrazole (2.9 g) in dry THF (55 mL) was added triethyl amine (14.14 mL) followed by slow addition of 4-trifluoromethoxy benzoyl chloride (7.85 g) at 0 °C. The reaction mixture was stirred at RT for 3 h. The progress of the reaction was monitored by TLC. The reaction mixture was diluted with water (200 mL) then extracted with EtOAc (100 mL×2). The organic layers were dried over sodium sulphate and concentrated under reduced pressure to obtain the desired product as brown solid (8.0 g). ¹H NMR (300 MHz, DMSO-d₆) δ 12.43 (d, J=12.0 Hz, 1H), 9.81 (s, 1H), 8.15-7.99 (m, 2H), 7.88 (s, 1H), 7.61-7.40 (m, 2H), 2.18 (s, 3H).

### Step 3: Synthesis of N-[3-methyl-1-(3-methyl-4-nitro-phenyl) pyrazol-4-yl]-4-(trifluoromethoxy)benzamide

To a stirred solution of N-(3-methyl-1H-pyrazol-4-yl)-4-(trifluoromethoxy)benzamide (0.4 g) in dry DMF (10 mL) were added Cesium carbonate (0.912 g) and 4-fluoro-2-methyl-1-nitrobenzene (0.326 g) at RT. The reaction mixture was heated at 110 °C for 24 h. The progress of the reaction was monitored by TLC. The reaction mixture was diluted with water (100 mL) then extracted with EtOAc (50 mL×2). The combined organic layers were dried over sodium sulphate and concentrated under reduced pressure to get the crude product. The crude product was purified by column chromatography using EtOAc and heptane as eluent to offer the desired product as off-white solid (0.45 g). HPLC/MS (Method 1): Rt: 2.388 min; m/z=419 (M−1)⁺; ¹H NMR (300 MHz, DMSO-d₆) δ10.15 (s, 1H), 8.89 (s, 1H), 8.16 (d, J=9.0 Hz, 1H), 8.10 (d, J=8.7 Hz, 2H), 7.99 (d, J=1.9 Hz, 1H), 7.89 (dd, J=9.1, 2.5 Hz, 1H), 7.56 (d, J=7.9 Hz, 2H), 2.64 (s, 3H), 2.35 (s, 3H).

### Step 4: Synthesis of N-[1-(4-amino-3-methyl-phenyl)-3-methyl-pyrazol-4-yl]-4-(trifluoromethoxy)benzamide

To a stirred solution of N-[3-methyl-1-(3-methyl-4-nitro-phenyl) pyrazol-4-yl]-4-(trifluoromethoxy)benzamide (0.4 g) in a mixture of EtOH: EtOAc (5 mL: 5 mL) was added Tin (II) chloride dihydrate (0.854 g) at RT. The reaction mixture was heated at 80 °C for 6 h. The progress of the reaction was monitored by TLC. The reaction mixture was diluted with water (50 mL) and basified with solid sodium bicarbonate then extracted with EtOAc (25 mL×2). The combined organic layers were dried over sodium sulfate and concentrated under reduced pressure to get the crude product which was purified by n-pentane washings (2 mL×2) to afford the desired product as beige solid (0.35 g). HPLC/MS (Method 1): Rt: 2.091 min; m/z=391 (M+1)⁺. ¹H NMR (500 MHz, DMSO-d₆) 0 9.95 (s, 1H), 8.36 (s, 1H), 8.08 (d, J=8.8 Hz, 2H), 7.53 (d, J=8.2 Hz, 2H), 7.34 (d, J=2.2 Hz, 1H), 7.29-7.23 (m, 1H), 6.66 (d, J=8.5 Hz, 1H), 4.93 (s, 2H), 2.25 (s, 3H), 2.12 (s, 3H).

### Step 5: Synthesis of N-[1-[4-[[(Z)-[3-(2-isopropyl-5-methyl-phenyl)-4-oxo-thiazolidin-2-ylidene]carbamoyl]amino]-3-methyl-phenyl]-3-methyl-pyrazol-4-yl]-4-(trifluoromethoxy)benzamide

To a stirred solution of N-[1-(4-amino-3-methyl-phenyl)-3-methyl-pyrazol-4-yl]-4-(trifluoromethoxy)benzamide (0.15 g) in dry THF (5 mL) at 0 °C was added 4-nitrophenylchloroformate (0.077 g). The reaction mixture was allowed to stir at RT for 2 h. The progress of the reaction was monitored by TLC. The reaction mixture was again cooled to 0 °C and was added N,N-diisopropylethyl amine (0.122 mL) followed by 2-imino-3-(2-isopropyl-5-methylphenyl) thiazolidin-4-one (0.116 g). To the reaction mixture was added dry acetonitrile (5 mL) and Potassium phosphate tribasic (0.149 g). Reaction mixture was allowed to stir at RT for 12 h. The progress of the reaction was monitored by TLC. The reaction mixture was diluted with water (20 mL) and was extracted with EtOAc (20 mL×2). The combined organic layer was dried over sodium sulfate and concentrated under reduced pressure to get the crude product which was purified by column chromatography using EtOAc and heptane as eluent to afford the desired product as beige solid (0.12 g). HPLC/MS (Method 1): Rt: 2.464 min; m/z=665 (M+1)⁺. ¹H NMR (500 MHz, DMSO-d₆) δ 10.03 (s, 1H), 9.13 (s, 1H), 8.60 (s, 1H), 8.09 (d, J=8.7 Hz, 2H), 7.63 (s, 1H), 7.54 (m, 3H), 7.40 (d, J=7.9 Hz, 1H), 7.33 (d, J=8.5 Hz, 1H), 7.27 (d, J=6.7 Hz, 1H), 7.07 (s, 1H), 4.17 (m, 1H), 4.06 (m, 2H), 2.72-2.65 (m, 1H), 2.32 (s, 3H), 2.29 (s, 3H), 2.20 (s, 3H), 1.21 (d, J=6.9 Hz, 3H), 1.10 (d, J=6.8 Hz, 3H).

### Example C-2

### Synthesis of N-[1-[4-[[(Z)-[3-(2-isopropyl-5-methyl-phenyl)-4-oxo-thiazolidin-2-ylidene]carbamoyl]amino]phenyl]-3-methyl-pyrazol-4-yl]-N-methyl-4-(trifluoromethoxy)benzamide

### Step 1: Synthesis of N-methyl-N-[3-methyl-1-(4-nitrophenyl) pyrazol-4-yl]-4-(trifluoromethoxy)benzamide

To a stirred solution of N-[3-methyl-1-(4-nitrophenyl) pyrazol-4-yl]-4-(trifluoromethoxy)benzamide (2 g) in DMF (50 mL) were added cesium carbonate (3.2 g) and methyl iodide (0.9 g). The reaction mixture was stirred at 50 °C until the starting material has been consumed. Subsequently, DMF was evaporated, and EtOAc was added to the remaining residues. A precipitate was filtered off and purified via column chromatography to yield the desired product (2 g). ¹H NMR (400 MHz, Chloroform-d) δ 8.33-8.25 (m, 2H), 7.71 (d, J=8.3 Hz, 3H), 7.45 (d, J=8.3 Hz, 2H), 7.10 (m, 2H), 3.41 (s, 3H), 2.18 (s, 3H).

### Step 2: Synthesis of N-[1-(4-aminophenyl)-3-methyl-pyrazol-4-yl]-N-methyl-4-(trifluoromethoxy)benzamide

N-methyl-[3-methyl-1-(4-nitrophenyl) pyrazol-4-yl]-4-(trifluoromethxy)benzamide (2 g) was dissolved in a 1:1 mixture of EtOAc (16.5 mL) and EtOH (16.5 mL). To the reaction mixture tin (II) chloride dihydrate (4.7 g) was added at RT. The reaction progress was monitored by TLC and upon consumption of the starting material, the reaction was quenched by addition of saturated aqueous sodium bicarbonate solution (220 mL). The reaction mixture was filtered over a celite pad, and the organic phases extracted three times with EtOAc. The combined organic phases were dried over magnesium sulphate, and the residue was purified by flash chromatography to yield the desired product (1.8 g). ¹H NMR (400 MHz, Chloroform-d) δ 7.52-7.33 (m, 3H), 7.24 (d, J=8.3 Hz, 2H), 7.06 (d, J=8.3 Hz, 2H), 6.78-6.55 (m, 2H), 3.65 (m, 2H), 3.40 (s, 3H), 2.08 (s, 3H).

### Step 3: Syntheis of N-[1-[4-[[(Z)-[3-(2-isopropyl-5-methyl-phenyl)-4-oxo-thiazolidin-2-ylidene]carbamoyl]amino]phenyl]-3-methyl-pyrazol-4-yl]-N-methyl-4-(trifluoromethoxy)benzamide (C-2)

To a stirred solution of N-[1-(4-aminophenyl)-3-methylpyrazol-4-yl)-N-methyl-4-(trifluoromethxy)benzamide (0.4 g) in THF (12 mL) was added 4-nitrophenyl chloroformate (0.2 g) at 0 °C. The starting material was consumed after 3 h of stirring at RT as indicated by TLC. The solvent was evaporated, and the crude product was used directly in the next step. The residue was dissolved in acetonitrile (15 mL), followed by addition of 2-imino-3-(2-isopropyl-5-methylphenyl) thiazolidine-4-one (0.2 g), Diisoproyplamine (0.25 mL) and Potassium phosphate (0.3 g). The reaction mixture was stirred at RT for overnight, followed by dilution with water (40 mL). The organic phases were extracted three times with EtOAc, washed with brine and dried over magnesium sulphate. Purification by column chromatography yielded the desired product (0.36 g). ¹H NMR (400 MHz, Chloroform-d) δ 7.58-7.50 (m, 3H), 7.45-7.27 (m, 7H), 7.06 (d, J=8.0 Hz, 2H), 6.90 (s, 1H), 3.97 (s, 2H), 3.39 (s, 3H), 2.66 (m, 1H), 2.38 (s, 3H), 2.13 (s, 3H), 1.19 (m, 6H).

### Example C-3

### Synthesis of N-[5-chloro-1-[4-[[(Z)-[3-(2-isopropyl-5-methyl-phenyl)-4-oxo-thiazolidin-2-ylidene]carbamoyl]amino]phenyl]-3-methyl-pyrazol-4-yl]-N-methyl-4-(trifluoromethoxy)benzamide

### Step 1: Synthesis of N-[5-chloro-1-[4-[[(Z)-[3-(2-isopropyl-5-methyl-phenyl)-4-oxo-thiazolidin-2-ylidene]carbamoyl]amino]phenyl]-3-methyl-pyrazol-4-yl]-N-methyl-4-(trifluoromethoxy)benzamide (C-3)

Thiobiuret C-2 (160 mg) was dissolved in Chloroform (5 mL) and Methyl-N—(N′-chloro-N-methoxycarbonyl-carbamimidoyl) carbamate (Palau'Chlor®) (0.076 g) was added at RT and stirred for 22 h. The solvent was partially evaporated, the mixture was quenched with water (30 mL) and the aqueous phase was extracted three times with EtOAc. The combined organic phases were dried over magnesium sulphate. Purification via column chromatography yielded the desired product (0.093 g). ¹H NMR (400 MHz, chloroform-d) d 7.57 (d, J=8.7 Hz, 2H), 7.45-7.25 (m, 6H), 7.08 (d, J=8.3 Hz, 2H), 6.93-6.88 (s, 1H), 3.97 (d, J=3.2 Hz, 2H), 3.77 (s, 1H), 3.36 (s, 3H), 2.66 (m, 1H), 2.38 (s, 3H), 2.15 (s, 3H), 1.19 (m, 6H).

### Example C-4

### Synthesis of N-[1-[3-chloro-4-[[(Z)-[3-(2-isopropyl-5-methyl-phenyl)-4-oxo-thiazolidin-2-ylidene]carbamoyl]amino]phenyl]-3,5-dimethyl-pyrazol-4-yl]-4-(trifluoromethoxy)benzamide

### Step 1: Synthesis of 3,5-dimethyl-1H-pyrazol-4-amine

To a stirred solution of 3,5-dimethyl-4-nitro-1H-pyrazole (10 g) in a mixture 250 ml of EtOH: H₂O (4:1) was added Fe (11.872 g) and Ammonium chloride (37.9 g) at RT. The reaction mixture was heated at 80 °C for 5 h. The progress of the reaction was monitored by TLC. The reaction mixture was cooled down to RT and was filtered through celite pad, the celite pad was washed with EtOAc (100 mL). The filtrate was concentrated under reduced pressure to two-third of volume and was diluted with EtOAc (100 mL) again and water (50 mL) was added. The layers were separated and organic layers were dried over sodium sulphate and concentrated under reduced pressure to get the crude product which was purified by n-pentane washings (25 mL×2) to afford the desired product as beige solid (4 g).

### Step 2: Synthesis of N-(3,5-dimethyl-1H-pyrazol-4-yl)-4-(trifluoromethoxy)benzamide

To a stirred solution of 3-methyl 4-amino pyrazole (4 g) in Dichloromethane (60 mL) was added 4-trifluoro methoxy benzoic acid (7.4 g) followed by addition of Di-isopropyl ethylamine (25 mL) at 10 °C. The reaction mixture was cooled to 0 °C and to the reaction mixture was added 1-Propanephosphonic anhydride solution, (160.5 mL, 50% solution in EtOAc) dropwise over a period of 4 h. The progress of the reaction was monitored by HPLC. After completion of reaction, the reaction mixture was added to water dropwise (750 mL) and stirred for 60 min. White precipitate was formed which was filtered and dried under reduced pressure to obtain the desired product as white solid compound (9.8 g). HPLC/MS (Method 1): Rt: 1.75 min; m/z=300 (M+1)⁺. ¹H NMR (300 MHz, DMSO-d₆) δ 9.59 (s, 1H), 8.10 (d, J=8.3 Hz, 2H), 7.50 (d, J=8.3 Hz, 2H), 2.06 (s, 6H).

### Step 3: Synthesis of N-[3,5-dimethyl-1-(4-nitrophenyl) pyrazol-4-yl]-4-(trifluoromethoxy)benzamide

To a stirred solution of N-(3,5-dimethyl-1H-pyrazol-4-yl)-4-(trifluoromethoxy)benzamide (1 g) in dry DMF (10 mL) were added cesium carbonate (1.415 g) and 1-fluoro-4-nitrobenzene (0.707 g) at RT. The reaction mixture was heated at 70 °C for 24 h. The progress of the reaction was monitored by TLC. The reaction mixture was diluted with water (100 mL) and the precipitated collected by filtration. The solid was dried under vacuum and washed with n-pentane to afford desired product as yellow solid (1.3 g). HPLC/MS (Method 1): Rt: 1.871 min; m/z=419 (M−1)⁺; ¹H NMR (300 MHz, DMSO-d₆) δ 9.94 (s, 1H), 8.37 (d, J=9.1 Hz, 2H), 8.14 (d, J=8.7 Hz, 2H), 7.90 (d, J=9.1 Hz, 2H), 7.55 (d, J=8.3 Hz, 2H), 2.35 (s, 3H), 2.16 (s, 3H).

### Step 4: Synthesis of N-[1-(4-aminophenyl)-3,5-dimethyl-pyrazol-4-yl]-4-(trifluoromethoxy)benzamide

To a solution of N-[3,5-dimethyl-1-(4-nitrophenyl) pyrazol-4-yl]-4-(trifluoromethoxy)benzamide (20 g) in 127 mL of MeOH (300 mL) was added palladium on carbon (1.45 g). Reaction mixture was subjected to hydrogenation at 40 psi for 18 h. The progress of the reaction was monitored by TLC. The reaction mixture was filtered through celite pad, the celite pad was washed with MeOH (100 mL). The filtrate was concentrated under reduced pressure to get the crude product which was purified by column chromatography using EtOAc and heptane as eluent to afford the desired product as beige solid (7 g). HPLC/MS (Method 1): Rt: 1.697 min; m/z=391 (M+1)⁺; ¹H NMR (500 MHz, DMSO-d₆) δ 9.72 (s, 1H), 8.11 (d, J=8.8 Hz, 2H), 7.53 (d, J=8.1 Hz, 2H), 7.09 (d, J=8.6 Hz, 2H), 6.65 (d, J=8.7 Hz, 2H), 5.31 (s, 2H), 2.09 (s, 3H), 2.08 (s, 3H).

### Step 5: Synthesis of N-[1-(4-amino-3-chloro-phenyl)-3,5-dimethyl-pyrazol-4-yl]-4-(trifluoromethoxy)benzamide

To a stirred solution of N-[1-(4-aminophenyl)-3,5-dimethyl-pyrazol-4-yl]-4-(trifluoromethoxy)benzamide (0.3 g) in acetonitrile (5 mL) was added N-chlorosuccinimide (0.123 g) at RT. The reaction mixture was heated at 90 °C for 2 h. The progress of the reaction was monitored by TLC. The reaction mixture was diluted with water (20 mL) and extracted with EtOAc (20 mL×2). The combined organic layers were dried over sodium sulfate and concentrated under reduced pressure to get the crude product which was purified by column chromatography using EtOAc and heptane as eluent to afford the desired product as beige solid (0.1 g). HPLC/MS (Method 1): Rt: 1.995 min; m/z=426 (M+1)⁺. ¹H NMR (500 MHz, DMSO-d₆) δ 9.75 (s, 1H), 8.12 (d, J=8.4 Hz, 2H), 7.53 (d, J=8.3 Hz, 2H), 7.32 (s, 1H), 7.16 (d, J=10.1 Hz, 1H), 6.88 (d, J=8.6 Hz, 1H), 5.59 (s, 2H), 2.13 (s, 3H), 2.09 (s, 3H).

### Step 6: Synthesis of N-[1-[3-chloro-4-[[(Z)-[3-(2-isopropyl-5-methyl-phenyl)-4-oxo-thiazolidin-2-ylidene]carbamoyl]amino]phenyl]-3,5-dimethyl-pyrazol-4-yl]-4-(trifluoromethoxy)benzamide

To a stirred solution of N-[1-(4-amino-3-chloro-phenyl)-3,5-dimethyl-pyrazol-4-yl]-4-(trifluoromethoxy)benzamide (0.9 g) in dry THF (15 mL) was added 4-nitrophenylchloroformate (0.512 g) at 0 °C. The reaction mixture was allowed to stir at RT for 2 h. The progress of the reaction was monitored by TLC. The reaction mixture was again cooled to 0 °C and was added N,N-diisopropylethyl amine (0.531 mL) followed by 2-imino-3-(2-isopropyl-5-methyl-phenyl)thiazolidin-4-one (0.505 g). To the reaction mixture was added dry acetonitrile (20 mL) and potassium phosphate tribasic (0.648 g). Reaction mixture was allowed to stir at RT for 12 h. The progress of the reaction was monitored by TLC. The reaction mixture was diluted with water (50 mL) and was extracted with EtOAc (50 mL×2). The combined organic layers were dried over sodium sulfate and concentrated under reduced pressure to get the crude product which was purified by column chromatography using EtOAc and heptane as eluent to afford the desired product as beige solid (1 g). HPLC/MS (Method 1): Rt: 1.291 min; m/z=700 (M+1)+. ¹H NMR (500 MHz, DMSO-d₆) δ 9.83 (s, 1H), 9.17 (s, 1H), 8.12 (d, J=8.2 Hz, 2H), 7.67 (m, 2H), 7.52 (m, 3H), 7.39 (m, 1H), 7.33-7.22 (m, 1H), 7.07 (s, 1H), 4.28-4.04 (m, 2H), 2.73-2.62 (m, 1H), 2.32 (s, 3H), 2.24 (s, 3H), 2.12 (s, 3H), 1.27-1.14 (m, 3H), 1.10 (d, J=6.1 Hz, 3H).

### Example C-5

### Synthesis of N-[5-cyclopropyl-1-[4-[[(Z)-[3-(2-isopropyl-5-methyl-phenyl)-4-oxo-thiazolidin-2-ylidene]carbamoyl]amino]phenyl]-3-methyl-pyrazol-4-yl]-4-(trifluoromethoxy)benzamide

### Step 1: Synthesis of 1-cyclopropylbutane-1,3-dione

To a stirred solution of Sodium Methoxide (3.8 g) in Methyl tert-butyl ether MTBE (30 mL) was added Cyclopropyl Methyl Ketone (3 g). The reaction mass was heated to 30 °C followed by addition of EtOAc (6.3 g) at 30 °C over a period of 45 min. The reaction mixture was stirred at 30 °C for 3 h. The progress of the reaction was monitored by TLC and GCMS. After the reaction was complete, reaction mixture was added to water (30 mL) dropwise. Layers were separated and aqueous layer was acidified with 2.5 M HCl (10 mL) followed by extraction with MTBE (2×10 mL). Organic layers were dried over sodium sulphate and evaporated under reduced pressure to afford the crude product as brown liquid (4 g) which was purified by column chromatography using EtOAc and heptane as eluent to afford the desired product as colorless oily liquid (2 g). GCMS (Low Temp Volatiles): Rt: 5.41 min; m/z=126. ¹H NMR (500 MHz, DMSO) mixture of keto and enol tautomers, δ 12.01 (s, 1H), 5.86 (s, 1H), 3.82 (s, 1H), 2.22-2.18 (m, 2H), 2.06-2.02 (m, 4H), 1.99-1.94 (m, 2H), 1.86-1.80 (m, 1H), 1.03-0.96 (m, 4H).

### Step 2: Synthesis of 1-(4-bromophenyl)-5-cyclopropyl-3-methyl-pyrazole

To a stirred solution of 1-cyclopropylbutane-1,3-dione (2 g) in Acetic Acid (20 mL) was added solution of 4-Bromo phenyl hydrazine hydrochloride salt (3.5 g) in water (20 mL) dropwise maintaining temperature below 30 °C Reaction mass was stirred at 25 °C for 6 h. The progress of the reaction was monitored by TLC and LCMS. After completion, the reaction mixture was added to water (50 mL) and extracted with MTBE (2×10 mL), organic layers were washed with saturated sodium bicarbonate solution (2×30 mL), organic layers were dried over sodium sulphate and evaporated under reduced pressure to afford the desired product as off-white solid (4 g). HPLC/MS (Method 1): Rt: 1.93 min; m/z=277 (M+1)+279 (M+3)⁺ bromo pattern. ¹H NMR (500 MHz, DMSO) δ 7.67 (d, J=8.8, 2.1 Hz, 2H), 7.61-7.56 (d, 2H), 5.92 (s, J=1.9 Hz, 1H), 2.16 (s, J=2.1 Hz, 3H), 1.81 (m, J=8.3, 5.4, 2.5 Hz, 1H), 0.96-0.90 (dd, 2H), 0.68 (dd, J=5.0, 2.4 Hz, 2H).

### Step 3: Synthesis of 1-(4-bromophenyl)-5-cyclopropyl-3-methyl-4-nitro-pyrazole

To a stirred solution of 1-(4-bromophenyl)-5-cyclopropyl-3-methyl-pyrazole (1 g) in Dichloroethane (10 mL) was added Sulphuric acid (1.76 mL) dropwise maintaining temperature below 20 °C Reaction mass was cooled to 10 °C and to the reaction mass was added fuming Nitric acid (0.16 mL) dropwise maintaining the temperature below 10 °C and reaction mass was stirred for 1 h at 10 °C. The progress of the reaction was monitored by TLC and LCMS. After the reaction was complete, it was added to water (50 mL) and aqueous layer was extracted with MTBE (2×10 mL), organic layers were washed with saturated sodium bicarbonate solution (2×30 mL), dried over sodium sulphate, and evaporated under reduced pressure to obtain the crude product as pale brown solid (4 g). The crude product was purified by column chromatography using EtOAc and heptane as eluent to afford the desired product as off-white solid (0.55 g). HPLC/MS (Method 1): Rt: 2.15 min; m/z=322.3 (M+1)+324 (M+3)⁺ bromo pattern. ¹H NMR (300 MHz, DMSO) δ 7.78 (d, J=8.7 Hz, 2H), 7.66-7.58 (m, 2H), 2.26 (tt, J=8.5, 5.7 Hz, 1H), 0.94 (dd, J=8.4, 2.2 Hz, 2H), 0.37 (dd, J=5.7, 1.9 Hz, 2H).

### Step 4: Synthesis of 1-(4-bromophenyl)-5-cyclopropyl-3-methyl-pyrazol-4-amine

To a stirred solution of 1-(4-bromophenyl)-5-cyclopropyl-3-methyl-4-nitro-pyrazole (1 g) in Ethanol (10 mL) was added Iron powder (0.521 g) followed by dropwise addition of aq. Ammonium chloride solution (16.63 g in 50 ml of water) maintaining the temperature below 25 °C. Reaction mass was heated to 90 °C for 3 h. The progress of the reaction was monitored by TLC and LCMS. After the reaction was complete, the reaction mixture was filtered through celite pad and washed with EtOAc (100 mL). Filtrate was concentrated to its one-third volume and extracted with EtOAc (2×100 mL). Combined organic layers were washed with saturated sodium bicarbonate solution (2×100 mL), organic layers were dried over sodium sulphate and evaporated under reduced pressure to obtain the desired product as off-white solid (6 g). HPLC/MS (Method 1): Rt: 1.63 min; m/z=292 (M+1)+294 (M+3)⁺ bromo pattern. ¹H NMR (300 MHz, DMSO) δ 7.77-7.59 (m, 4H), 3.75 (s, 2H), 2.14 (s, 3H), 1.87 (m, 2H), 0.95 (dd, 2H), 0.41 (dd, 2H).

### Step 5: Synthesis of N-[1-(4-bromophenyl)-5-cyclopropyl-3-methyl-pyrazol-4-yl]-4-(trifluoromethoxy)benzamide

To a stirred solution of 1-(4-bromophenyl)-5-cyclopropyl-3-methyl-pyrazol-4-amine (6 g) in Dichloromethane (600 mL) was added 4-Trifluoro methoxy Benzoic acid (4.25 g) and the reaction mixture was cooled to 10 °C. Diisopropylethylamine (14.32 mL) was added maintaining temperature below 10 °C. To the reaction mixture was added 1-Propanephosphonic anhydride solution (19.65 g, 50% solution in EtOAc) dropwise over a period of 30 min. Reaction mass was stirred and temperature was raised to RT and stirred for 12 h. The progress of the reaction was monitored by TLC and LCMS. After completion of reaction, the reaction mixture was added to water (500 mL) dropwise and layer was separated. Aqueous layer was extracted with DCM (2×100 mL), organic layers were dried over sodium sulphate and evaporated under reduced pressure to obtain the desired product as off white solid (7.4 g). HPLC/MS (Method 1): Rt: 2.15 min; m/z=480 (M+1)+482 (M+3)⁺ bromo pattern. ¹H NMR (500 MHz, DMSO) δ 9.77 (s, 1H), 8.11 (dd, J=8.7, 2.1 Hz, 2H), 7.70 (dd, J=8.8, 2.1 Hz, 2H), 7.66-7.60 (m, 2H), 7.55 (d, J=8.3 Hz, 2H), 2.08 (d, J=2.0 Hz, 3H), 1.97-1.85 (m, 1H), 0.78 (dt, J=8.7, 2.4 Hz, 2H), 0.51-0.47 (m, 2H).

### Step 6: Synthesis of N-[1-(4-aminophenyl)-5-cyclopropyl-3-methyl-pyrazol-4-yl]-4-(trifluoromethoxy)benzamide

To a stirred solution of N-[1-(4-bromophenyl)-5-cyclopropyl-3-methyl-pyrazol-4-yl]-4-(trifluoromethoxy)benzamide (4.2 g) and Boc-amide (1.54 g) in 1,4-Dioxane (80 mL) was degassed with Nitrogen gas for 30 min followed by addition of Potassium tert-butoxide (3.14 g), Tris(dibenzylideneacetone) dipalladium (0.80 g), 5-(Di-tert-butylphosphino)-1′,3′,5′-triphenyl-1′H[1,4′]bipyrazole (0.44 g) at RT. Reaction mass was heated for 12 h at 90 °C. The progress of the reaction was monitored by TLC and LCMS. After the reaction was complete, the reaction mixture was filtered through celite pad and washed with EtOAc (100 mL). Filtrate was concentrated to its one-third volume and extracted with EtOAc (2×100 mL). Organic layers were dried over sodium sulphate and evaporated under reduced pressure to obtain the crude product as off-white solid (3 g). To this crude compound was added Dichloromethane (30 mL) followed by dropwise addition of Trifluoro acetic acid (3.3 mL) at 10 °C. Reaction mass was stirred for 12 h at RT. The progress of the reaction was monitored by TLC and LCMS. After the completion of the reaction, it was added to water (100 mL) dropwise under stirring, extracted with EtOAc (2×50 mL). Aqueous layer was basified with saturated sodium carbonate solution (50 mL) followed by extraction with EtOAc (4×50 mL). Combined organic layers were dried over sodium sulphate and evaporated under reduced pressure to obtain the crude product as off-white solid (2 g). The crude product was purified by column chromatography using EtOAc and heptane as eluent to afford the desired product as off-white solid (1.1g). HPLC/MS (Method 1): Rt: 1.93 min; m/z=417 (M+1)+¹H NMR (300 MHz, DMSO) δ 9.62 (s, 1H), 8.09 (d, J=8.5 Hz, 2H), 7.54 (d, J=8.3 Hz, 2H), 7.17 (d, J=8.3 Hz, 2H), 6.64 (d, J=8.4 Hz, 2H), 5.30 (s, 2H), 2.02 (s, 3H), 1.74 (ddd, J=13.7, 8.5, 5.2 Hz, 1H), 0.70 (dt, J=8.5, 3.1 Hz, 2H), 0.62-0.51 (m, 2H).

### Step 7: Synthesis of N-[5-cyclopropyl-1-[4-[[(Z)-[3-(2-isopropyl-5-methyl-phenyl)-4-oxothiazolidin-2-ylidene]carbamoyl]amino]phenyl]-3-methyl-pyrazol-4-yl]-4-(trifluoromethoxy)benzamide (C-5)

To a stirred solution of N-[1-(4-aminophenyl)-5-cyclopropyl-3-methyl-pyrazol-4-yl]-4-(trifluoromethoxy)benzamide (0.17 g) in dry THF (4 mL) at 0 °C was added 4-nitrophenylchloroformate (0.099 g). The reaction mixture was allowed to stir at RT for 2 h. The progress of the reaction was monitored by TLC. The reaction mixture was again cooled to 0 °C and was added N, N-diisopropylethyl amine (0.076 mL) followed by 2-imino-3-(2-isopropyl-5-methylphenyl) thiazolidin-4-one (0.073 g). Then to the reaction mixture was added dry acetonitrile (5 mL) and Potassium phosphate tribasic (0.093 g). Reaction mixture was allowed to stir at RT for 12 h. The progress of the reaction was monitored by TLC. The reaction mixture was diluted with water (20 mL) and was extracted with EtOAc (20 mL×2). The combined organic layers were dried over sodium sulfate and concentrated under reduced pressure to get the crude product which was purified by column chromatography using EtOAc and heptane as eluent to afford the desired product as beige solid (0.19 g). HPLC/MS (Method 1): Rt: 1.195 min; m/z=692 (M+1)+. ¹H NMR (500 MHz, DMSO-d₆) δ 9.95 (s, 1H), 9.69 (s, 1H), 8.10 (d, J=7.6 Hz, 2H), 7.78 (d, J=8.1 Hz, 2H), 7.52 (dd, J=21.3, 8.1 Hz, 4H), 7.40 (d, J=8.0 Hz, 1H), 7.28 (d, J=8.0 Hz, 1H), 7.07 (s, 1H), 4.26-4.04 (m, 2H), 2.73-2.61 (m, 1H), 2.32 (s, 3H), 2.06 (s, 3H), 1.90-1.79 (m, 1H), 1.18 (d, J=6.4 Hz, 3H), 1.10 (d, J=6.5 Hz, 3H), 0.73 (d, J=7.9 Hz, 2H), 0.50 (d, J=3.1 Hz, 2H).

All other examples enlisted in the table C are synthesized analogous to the methods mentioned in either general procedure or experimental procedure mentioned above.

### Table C:

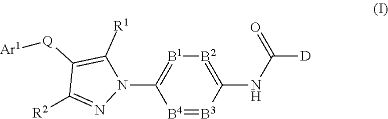

| No | Ar^{1—}Q | 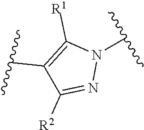 | 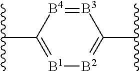 |
|---|---|---|---|
| C-1 | 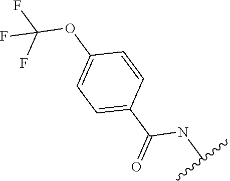 [SMILES: *NC(=O)c1ccc(OC(F)(F)F)cc1] | 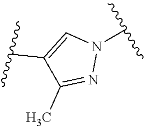 [SMILES: *c1cn(*)nc1C] | 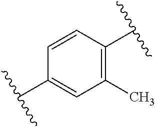 [SMILES: *c1ccc(*)c(C)c1] |
| C-2 | 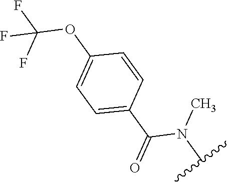 [SMILES: *N(C)C(=O)c1ccc(OC(F)(F)F)cc1] | 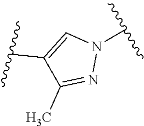 [SMILES: *c1cn(*)nc1C] | 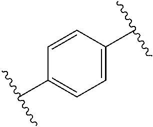 [SMILES: *c1ccc(*)cc1] |
| C-3 | 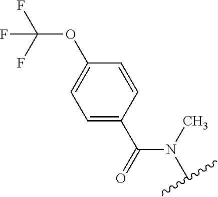 [SMILES: *N(C)C(=O)c1ccc(OC(F)(F)F)cc1] | 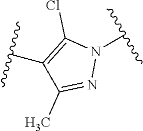 [SMILES: *c1c(C)nn(*)c1Cl] | 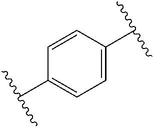 [SMILES: *c1ccc(*)cc1] |
| C-4 | 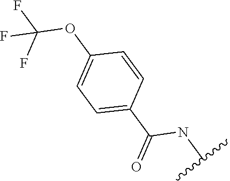 [SMILES: *NC(=O)c1ccc(OC(F)(F)F)cc1] | 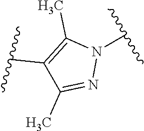 [SMILES: *c1c(C)nn(*)c1C] | 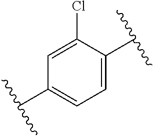 [SMILES: *c1ccc(*)c(Cl)c1] |
| C-5 | 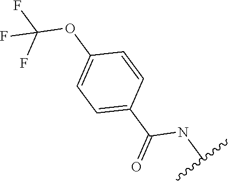 [SMILES: *NC(=O)c1ccc(OC(F)(F)F)cc1] | 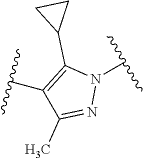 [SMILES: *c1c(C)nn(*)c1C1CC1] | 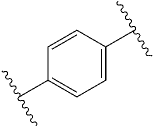 [SMILES: *c1ccc(*)cc1] |
| C-6 | 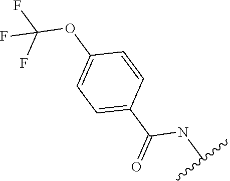 [SMILES: *NC(=O)c1ccc(OC(F)(F)F)cc1] | 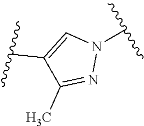 [SMILES: *c1cn(*)nc1C] | 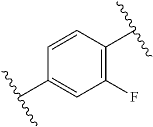 [SMILES: *c1ccc(*)c(F)c1] |
| C-7 | 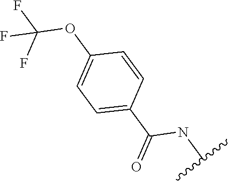 [SMILES: *NC(=O)c1ccc(OC(F)(F)F)cc1] | 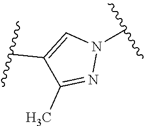 [SMILES: *c1cn(*)nc1C] | 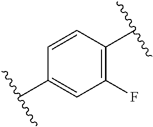 [SMILES: *c1ccc(*)c(F)c1] |
| C-8 | 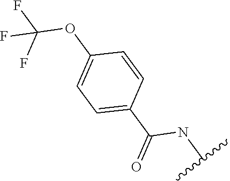 [SMILES: *NC(=O)c1ccc(OC(F)(F)F)cc1] | 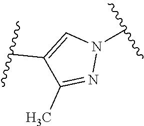 [SMILES: *c1cn(*)nc1C] | 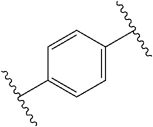 [SMILES: *c1ccc(*)cc1] |
| C-9 | 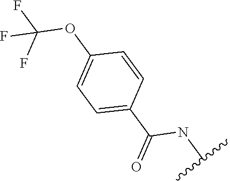 [SMILES: *NC(=O)c1ccc(OC(F)(F)F)cc1] | 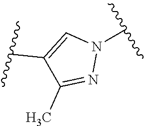 [SMILES: *c1cn(*)nc1C] | 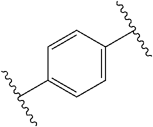 [SMILES: *c1ccc(*)cc1] |
| C-10 | 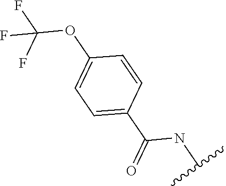 [SMILES: *NC(=O)c1ccc(OC(F)(F)F)cc1] | 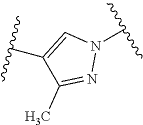 [SMILES: *c1cn(*)nc1C] | 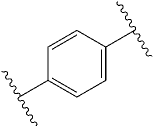 [SMILES: *c1ccc(*)cc1] |
| C-11 | 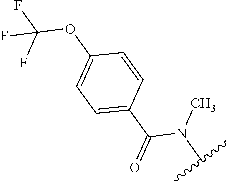 [SMILES: *N(C)C(=O)c1ccc(OC(F)(F)F)cc1] | 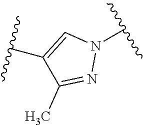 [SMILES: *c1cn(*)nc1C] | 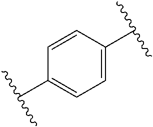 [SMILES: *c1ccc(*)cc1] |
| C-12 | 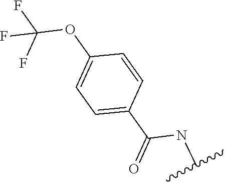 [SMILES: *NC(=O)c1ccc(OC(F)(F)F)cc1] | 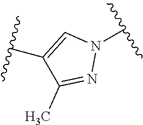 [SMILES: *c1cn(*)nc1C] | 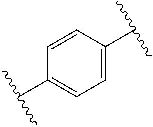 [SMILES: *c1ccc(*)cc1] |
| C-13 | 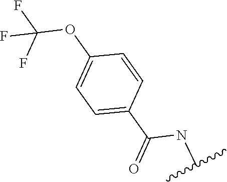 [SMILES: *NC(=O)c1ccc(OC(F)(F)F)cc1] | 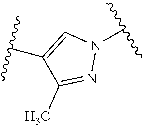 [SMILES: *c1cn(*)nc1C] | 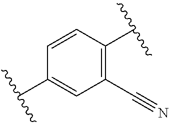 [SMILES: *c1ccc(*)c(C#N)c1] |
| C-14 | 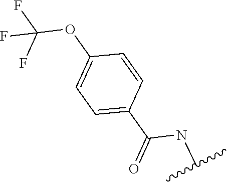 [SMILES: *NC(=O)c1ccc(OC(F)(F)F)cc1] | 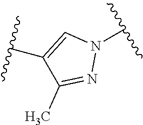 [SMILES: *c1cn(*)nc1C] | 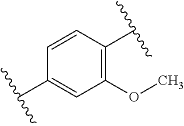 [SMILES: *c1ccc(*)c(OC)c1] |
| C-15 | 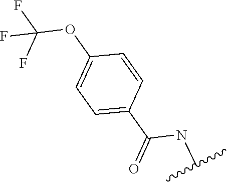 [SMILES: *NC(=O)c1ccc(OC(F)(F)F)cc1] | 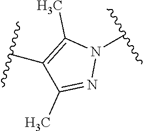 [SMILES: *c1c(C)nn(*)c1C] | 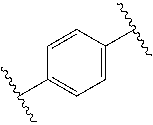 [SMILES: *c1ccc(*)cc1] |
| C-16 | 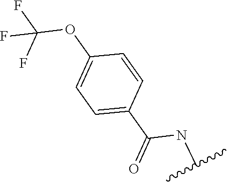 [SMILES: *NC(=O)c1ccc(OC(F)(F)F)cc1] | 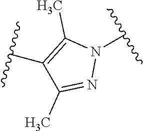 [SMILES: *c1c(C)nn(*)c1C] | 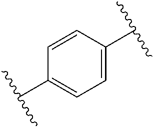 [SMILES: *c1ccc(*)cc1] |
| C-17 | 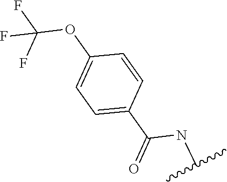 [SMILES: *NC(=O)c1ccc(OC(F)(F)F)cc1] | 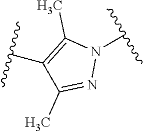 [SMILES: *c1c(C)nn(*)c1C] | 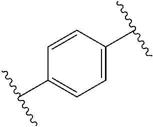 [SMILES: *c1ccc(*)cc1] |
| C-18 | 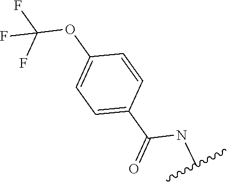 [SMILES: *NC(=O)c1ccc(OC(F)(F)F)cc1] | 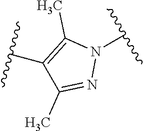 [SMILES: *c1c(C)nn(*)c1C] | 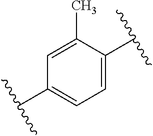 [SMILES: *c1ccc(*)c(C)c1] |
| C-19 | 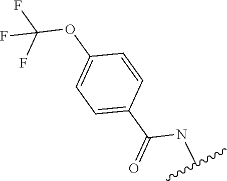 [SMILES: *NC(=O)c1ccc(OC(F)(F)F)cc1] | 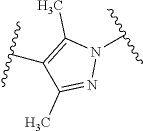 [SMILES: *c1c(C)nn(*)c1C] | 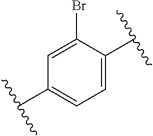 [SMILES: *c1ccc(*)c(Br)c1] |
| C-20 | 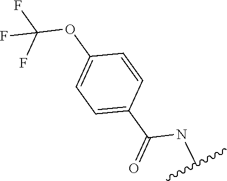 [SMILES: *NC(=O)c1ccc(OC(F)(F)F)cc1] | 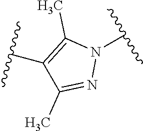 [SMILES: *c1c(C)nn(*)c1C] | 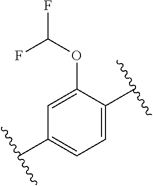 [SMILES: *c1ccc(*)c(OC(F)F)c1] |
| C-21 | 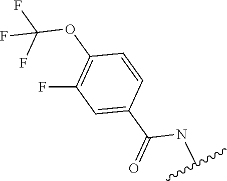 [SMILES: *NC(=O)c1ccc(OC(F)(F)F)c(F)c1] | 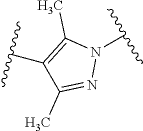 [SMILES: *c1c(C)nn(*)c1C] | 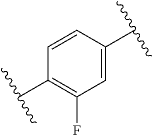 [SMILES: *c1ccc(*)c(F)c1] |
| C-22 | 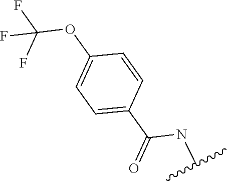 [SMILES: *NC(=O)c1ccc(OC(F)(F)F)cc1] | 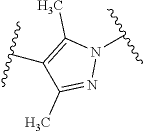 [SMILES: *c1c(C)nn(*)c1C] | 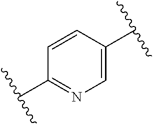 [SMILES: *c1ccc(*)nc1] |
| C-23 | 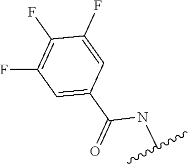 [SMILES: *NC(=O)c1cc(F)c(F)c(F)c1] | 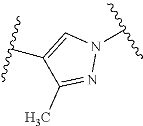 [SMILES: *c1cn(*)nc1C] | 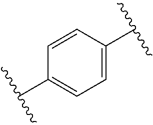 [SMILES: *c1ccc(*)cc1] |
| C-24 | 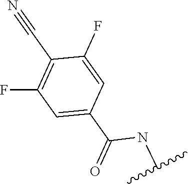 [SMILES: *NC(=O)c1cc(F)c(C#N)c(F)c1] | 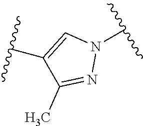 [SMILES: *c1cn(*)nc1C] | 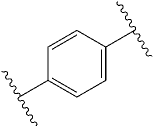 [SMILES: *c1ccc(*)cc1] |
| C-25 | 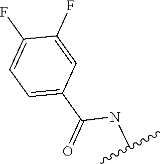 [SMILES: *NC(=O)c1ccc(F)c(F)c1] | 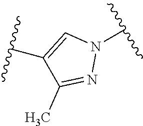 [SMILES: *c1cn(*)nc1C] | 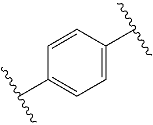 [SMILES: *c1ccc(*)cc1] |
| C-26 | 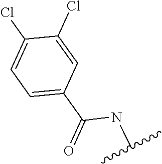 [SMILES: *NC(=O)c1ccc(Cl)c(Cl)c1] | 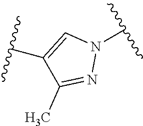 [SMILES: *c1cn(*)nc1C] | 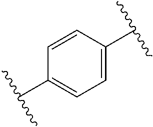 [SMILES: *c1ccc(*)cc1] |
| C-27 | 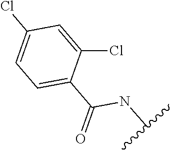 [SMILES: *NC(=O)c1ccc(Cl)cc1Cl] | 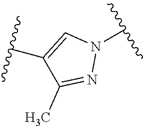 [SMILES: *c1cn(*)nc1C] | 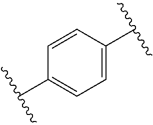 [SMILES: *c1ccc(*)cc1] |
| C-28 | 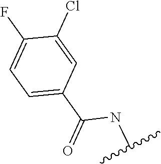 [SMILES: *NC(=O)c1ccc(F)c(Cl)c1] | 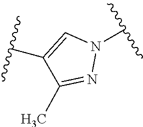 [SMILES: *c1cn(*)nc1C] | 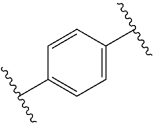 [SMILES: *c1ccc(*)cc1] |
| C-29 | 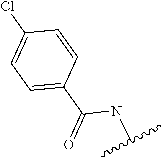 [SMILES: *NC(=O)c1ccc(Cl)cc1] | 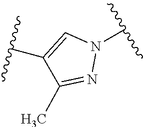 [SMILES: *c1cn(*)nc1C] | 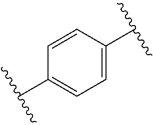 [SMILES: *c1ccc(*)cc1] |
| C-30 | 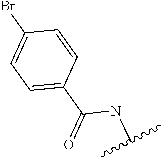 [SMILES: *NC(=O)c1ccc(Br)cc1] | 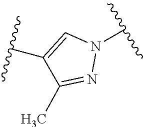 [SMILES: *c1cn(*)nc1C] | 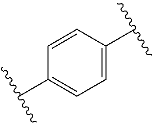 [SMILES: *c1ccc(*)cc1] |
| C-31 | 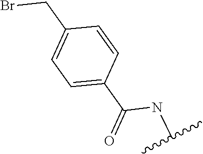 [SMILES: *NC(=O)c1ccc(CBr)cc1] | 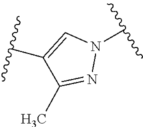 [SMILES: *c1cn(*)nc1C] | 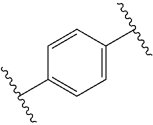 [SMILES: *c1ccc(*)cc1] |
| C-32 | 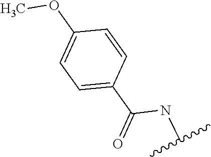 [SMILES: *NC(=O)c1ccc(OC)cc1] | 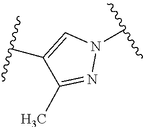 [SMILES: *c1cn(*)nc1C] | 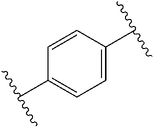 [SMILES: *c1ccc(*)cc1] |
| C-33 | 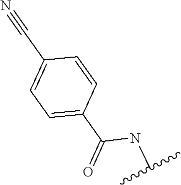 [SMILES: *NC(=O)c1ccc(C#N)cc1] | 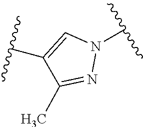 [SMILES: *c1cn(*)nc1C] | 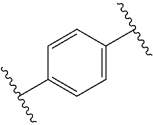 [SMILES: *c1ccc(*)cc1] |
| C-34 | 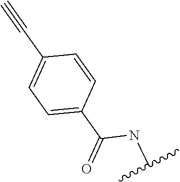 [SMILES: *NC(=O)c1ccc(C#C)cc1] | 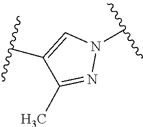 [SMILES: *c1cn(*)nc1C] | 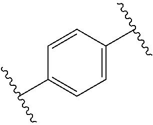 [SMILES: *c1ccc(*)cc1] |
| C-35 | 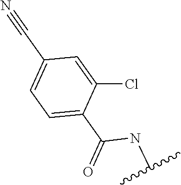 [SMILES: *NC(=O)c1ccc(C#N)cc1Cl] | 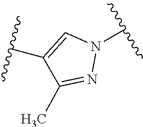 [SMILES: *c1cn(*)nc1C] | 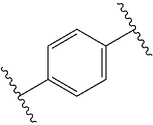 [SMILES: *c1ccc(*)cc1] |
| C-36 | 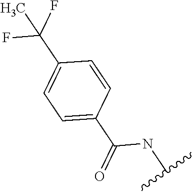 [SMILES: *NC(=O)c1ccc(C(C)(F)F)cc1] | 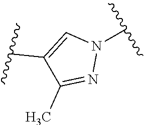 [SMILES: *c1cn(*)nc1C] | 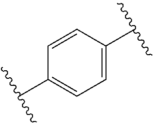 [SMILES: *c1ccc(*)cc1] |
| C-37 | 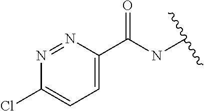 [SMILES: *NC(=O)c1ccc(Cl)nn1] | 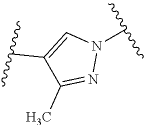 [SMILES: *c1cn(*)nc1C] | 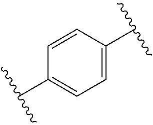 [SMILES: *c1ccc(*)cc1] |
| C-38 | 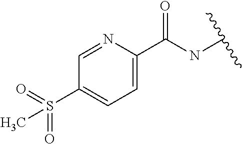 [SMILES: *NC(=O)c1ccc(S(C)(=O)=O)cn1] | 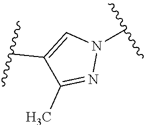 [SMILES: *c1cn(*)nc1C] | 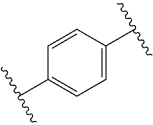 [SMILES: *c1ccc(*)cc1] |
| C-39 | 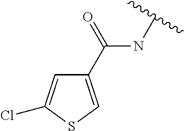 [SMILES: *NC(=O)c1csc(Cl)c1] | 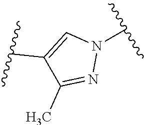 [SMILES: *c1cn(*)nc1C] | 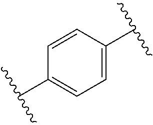 [SMILES: *c1ccc(*)cc1] |
| C-40 | 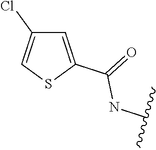 [SMILES: *NC(=O)c1cc(Cl)cs1] | 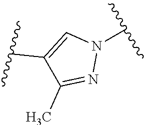 [SMILES: *c1cn(*)nc1C] | 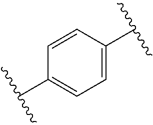 [SMILES: *c1ccc(*)cc1] |
| C-41 | 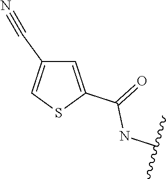 [SMILES: *NC(=O)c1cc(C#N)cs1] | 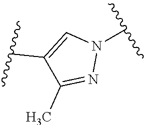 [SMILES: *c1cn(*)nc1C] | 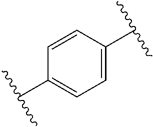 [SMILES: *c1ccc(*)cc1] |
| C-42 | 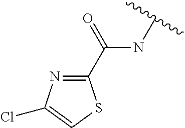 [SMILES: *NC(=O)c1nc(Cl)cs1] | 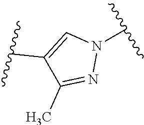 [SMILES: *c1cn(*)nc1C] | 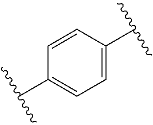 [SMILES: *c1ccc(*)cc1] |
| C-43 | 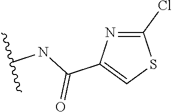 [SMILES: *NC(=O)c1csc(Cl)n1] | 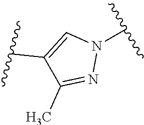 [SMILES: *c1cn(*)nc1C] | 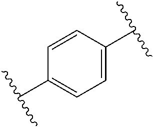 [SMILES: *c1ccc(*)cc1] |
| C-44 | 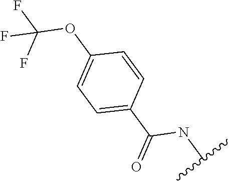 [SMILES: *NC(=O)c1ccc(OC(F)(F)F)cc1] | 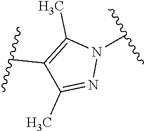 [SMILES: *c1c(C)nn(*)c1C] | 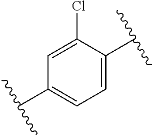 [SMILES: *c1ccc(*)c(Cl)c1] |
| C-45 | 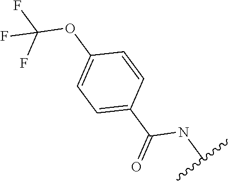 [SMILES: *NC(=O)c1ccc(OC(F)(F)F)cc1] | 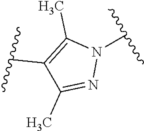 [SMILES: *c1c(C)nn(*)c1C] | 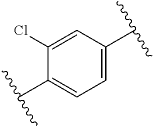 [SMILES: *c1ccc(*)c(Cl)c1] |
| C-46 | 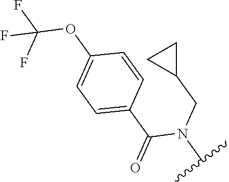 [SMILES: *N(CC1CC1)C(=O)c1ccc(OC(F)(F)F)cc1] | 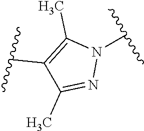 [SMILES: *c1c(C)nn(*)c1C] | 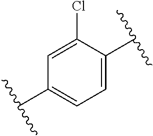 [SMILES: *c1ccc(*)c(Cl)c1] |
| C-47 | 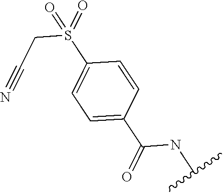 [SMILES: *NC(=O)c1ccc(S(=O)(=O)CC#N)cc1] | 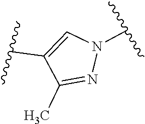 [SMILES: *c1cn(*)nc1C] | 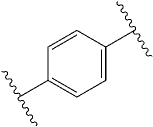 [SMILES: *c1ccc(*)cc1] |
| C-48 | 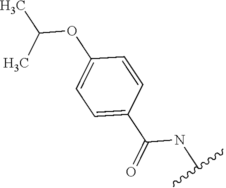 [SMILES: *NC(=O)c1ccc(OC(C)C)cc1] | 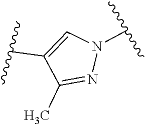 [SMILES: *c1cn(*)nc1C] | 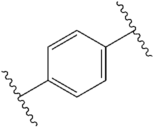 [SMILES: *c1ccc(*)cc1] |
| C-49 | 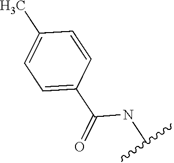 [SMILES: *NC(=O)c1ccc(C)cc1] | 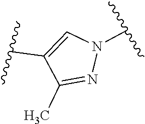 [SMILES: *c1cn(*)nc1C] | 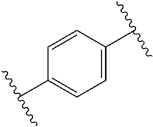 [SMILES: *c1ccc(*)cc1] |
| C-50 | 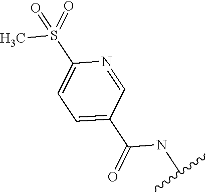 [SMILES: *NC(=O)c1ccc(S(C)(=O)=O)nc1] | 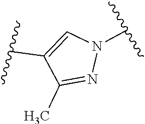 [SMILES: *c1cn(*)nc1C] | 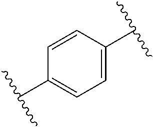 [SMILES: *c1ccc(*)cc1] |
| C-51 | 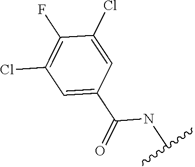 [SMILES: *NC(=O)c1cc(Cl)c(F)c(Cl)c1] | 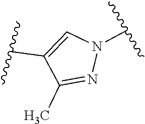 [SMILES: *c1cn(*)nc1C] | 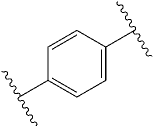 [SMILES: *c1ccc(*)cc1] |
| C-52 | 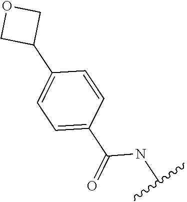 [SMILES: *NC(=O)c1ccc(C2COC2)cc1] | 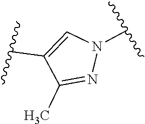 [SMILES: *c1cn(*)nc1C] | 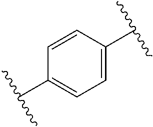 [SMILES: *c1ccc(*)cc1] |
| C-53 | 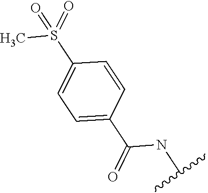 [SMILES: *NC(=O)c1ccc(S(C)(=O)=O)cc1] | 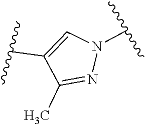 [SMILES: *c1cn(*)nc1C] | 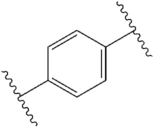 [SMILES: *c1ccc(*)cc1] |
| C-54 | 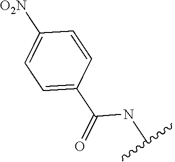 [SMILES: *NC(=O)c1ccc([N+](=O)[O-])cc1] | 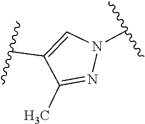 [SMILES: *c1cn(*)nc1C] | 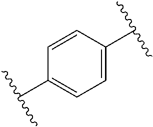 [SMILES: *c1ccc(*)cc1] |
| C-55 | 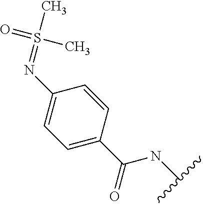 [SMILES: *NC(=O)c1ccc(N=S(C)(C)=O)cc1] | 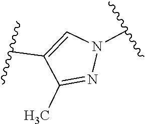 [SMILES: *c1cn(*)nc1C] | 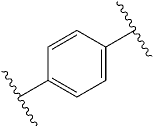 [SMILES: *c1ccc(*)cc1] |
| C-56 | 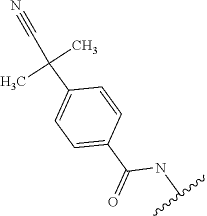 [SMILES: *NC(=O)c1ccc(C(C)(C)C#N)cc1] | 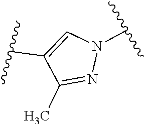 [SMILES: *c1cn(*)nc1C] | 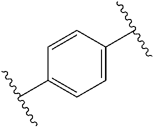 [SMILES: *c1ccc(*)cc1] |
| C-57 | 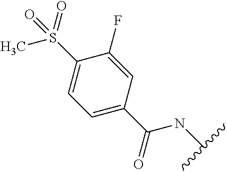 [SMILES: *NC(=O)c1ccc(S(C)(=O)=O)c(F)c1] | 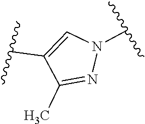 [SMILES: *c1cn(*)nc1C] | 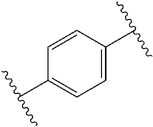 [SMILES: *c1ccc(*)cc1] |
| C-58 | 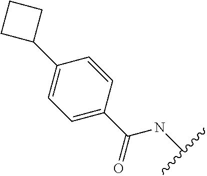 [SMILES: *NC(=O)c1ccc(C2CCC2)cc1] | 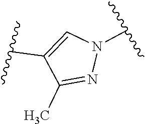 [SMILES: *c1cn(*)nc1C] | 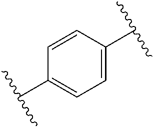 [SMILES: *c1ccc(*)cc1] |
| C-59 | 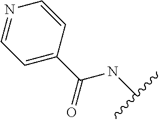 [SMILES: *NC(=O)c1ccncc1] | 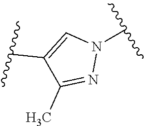 [SMILES: *c1cn(*)nc1C] | 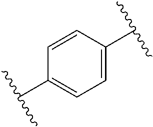 [SMILES: *c1ccc(*)cc1] |
| C-60 | 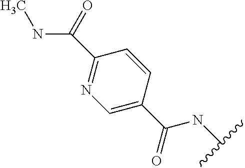 [SMILES: *NC(=O)c1ccc(C(=O)NC)nc1] | 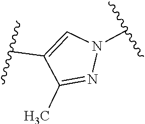 [SMILES: *c1cn(*)nc1C] | 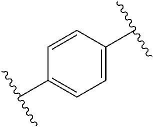 [SMILES: *c1ccc(*)cc1] |
| C-61 | 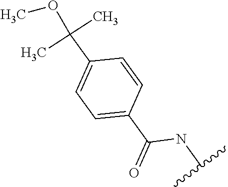 [SMILES: *NC(=O)c1ccc(C(C)(C)OC)cc1] | 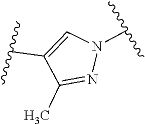 [SMILES: *c1cn(*)nc1C] | 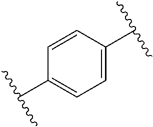 [SMILES: *c1ccc(*)cc1] |
| C-62 | 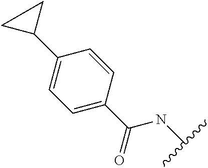 [SMILES: *NC(=O)c1ccc(C2CC2)cc1] | 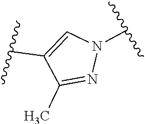 [SMILES: *c1cn(*)nc1C] | 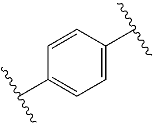 [SMILES: *c1ccc(*)cc1] |
| C-63 | 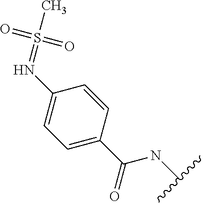 | 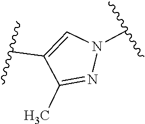 [SMILES: *c1cn(*)nc1C] | 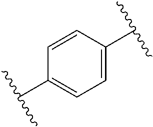 [SMILES: *c1ccc(*)cc1] |
| C-64 | 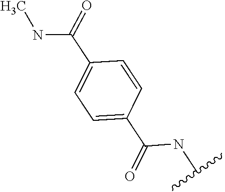 [SMILES: *NC(=O)c1ccc(C(=O)NC)cc1] | 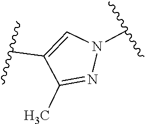 [SMILES: *c1cn(*)nc1C] | 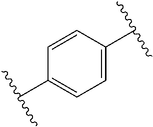 [SMILES: *c1ccc(*)cc1] |
| C-65 | 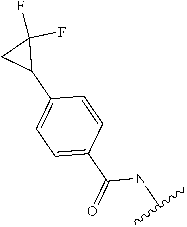 [SMILES: *NC(=O)c1ccc(C2CC2(F)F)cc1] | 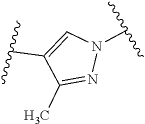 [SMILES: *c1cn(*)nc1C] | 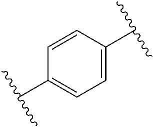 [SMILES: *c1ccc(*)cc1] |
| C-66 | 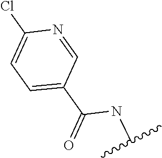 [SMILES: *NC(=O)c1ccc(Cl)nc1] | 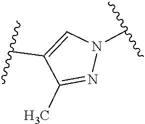 [SMILES: *c1cn(*)nc1C] | 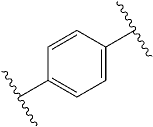 [SMILES: *c1ccc(*)cc1] |
| C-67 | 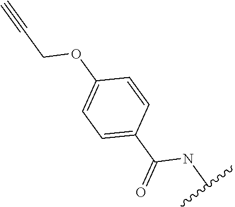 [SMILES: *NC(=O)c1ccc(OCC#C)cc1] | 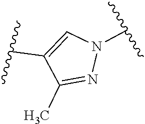 [SMILES: *c1cn(*)nc1C] | 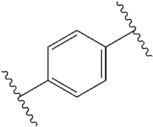 [SMILES: *c1ccc(*)cc1] |
| C-68 | 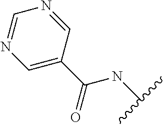 [SMILES: *NC(=O)c1cncnc1] | 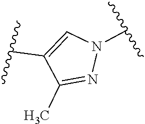 [SMILES: *c1cn(*)nc1C] | 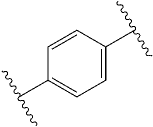 [SMILES: *c1ccc(*)cc1] |
| C-69 | 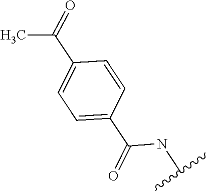 [SMILES: *NC(=O)c1ccc(C(C)=O)cc1] | 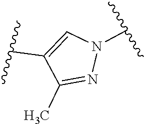 [SMILES: *c1cn(*)nc1C] | 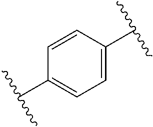 [SMILES: *c1ccc(*)cc1] |
| C-70 | 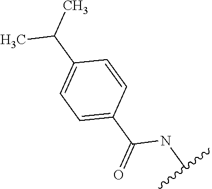 [SMILES: *NC(=O)c1ccc(C(C)C)cc1] | 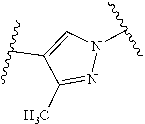 [SMILES: *c1cn(*)nc1C] | 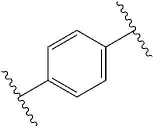 [SMILES: *c1ccc(*)cc1] |
| C-71 | 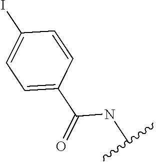 [SMILES: *NC(=O)c1ccc(I)cc1] | 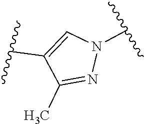 [SMILES: *c1cn(*)nc1C] | 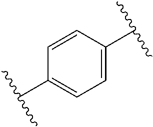 [SMILES: *c1ccc(*)cc1] |
| C-72 | 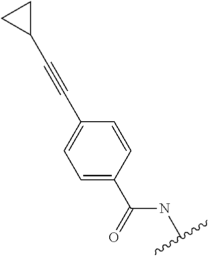 [SMILES: *NC(=O)c1ccc(C#CC2CC2)cc1] | 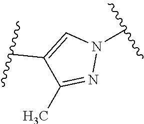 [SMILES: *c1cn(*)nc1C] | 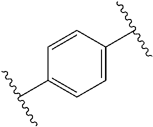 [SMILES: *c1ccc(*)cc1] |
| C-73 | 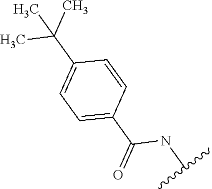 [SMILES: *NC(=O)c1ccc(C(C)(C)C)cc1] | 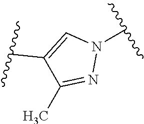 [SMILES: *c1cn(*)nc1C] | 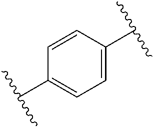 [SMILES: *c1ccc(*)cc1] |
| C-74 | 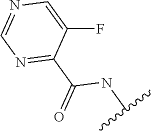 [SMILES: *NC(=O)c1ncncc1F] | 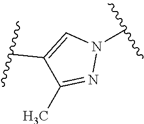 [SMILES: *c1cn(*)nc1C] | 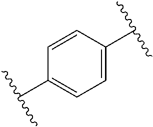 [SMILES: *c1ccc(*)cc1] |
| C-75 | 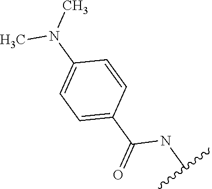 [SMILES: *NC(=O)c1ccc(N(C)C)cc1] | 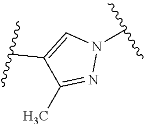 [SMILES: *c1cn(*)nc1C] | 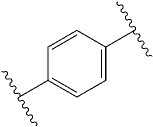 [SMILES: *c1ccc(*)cc1] |
| C-76 | 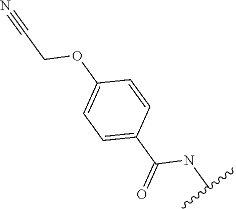 [SMILES: *NC(=O)c1ccc(OCC#N)cc1] | 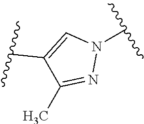 [SMILES: *c1cn(*)nc1C] | 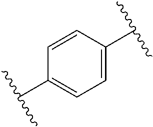 [SMILES: *c1ccc(*)cc1] |
| C-77 | 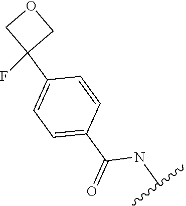 [SMILES: *NC(=O)c1ccc(C2(F)COC2)cc1] | 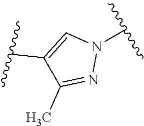 [SMILES: *c1cn(*)nc1C] | 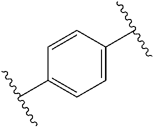 [SMILES: *c1ccc(*)cc1] |
| C-78 | 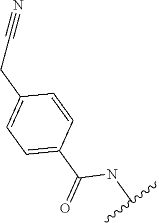 [SMILES: *NC(=O)c1ccc(CC#N)cc1] | 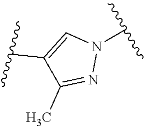 [SMILES: *c1cn(*)nc1C] | 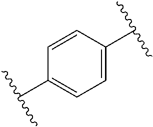 [SMILES: *c1ccc(*)cc1] |
| C-79 | 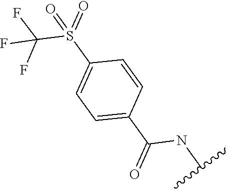 [SMILES: *NC(=O)c1ccc(S(=O)(=O)C(F)(F)F)cc1] | 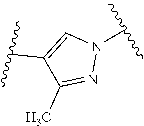 [SMILES: *c1cn(*)nc1C] | 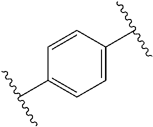 [SMILES: *c1ccc(*)cc1] |
| C-80 | 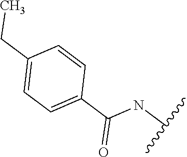 [SMILES: *NC(=O)c1ccc(CC)cc1] | 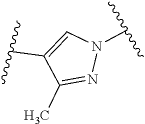 [SMILES: *c1cn(*)nc1C] | 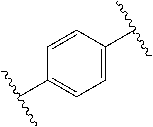 [SMILES: *c1ccc(*)cc1] |
| C-81 | 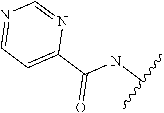 [SMILES: *NC(=O)c1ccncn1] | 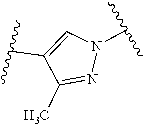 [SMILES: *c1cn(*)nc1C] | 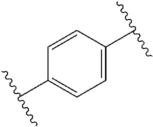 [SMILES: *c1ccc(*)cc1] |
| C-82 | 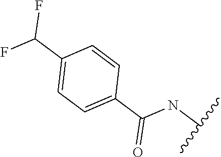 [SMILES: *NC(=O)c1ccc(C(F)F)cc1] | 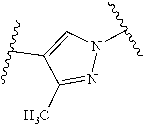 [SMILES: *c1cn(*)nc1C] | 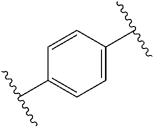 [SMILES: *c1ccc(*)cc1] |
| C-83 | 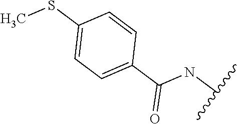 [SMILES: *NC(=O)c1ccc(SC)cc1] | 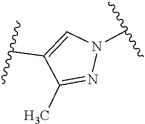 [SMILES: *c1cn(*)nc1C] | 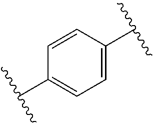 [SMILES: *c1ccc(*)cc1] |
| C-84 | 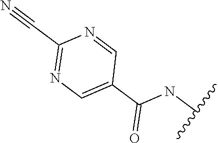 [SMILES: *NC(=O)c1cnc(C#N)nc1] | 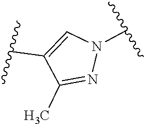 [SMILES: *c1cn(*)nc1C] | 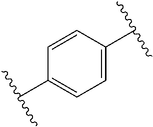 [SMILES: *c1ccc(*)cc1] |
| C-85 | 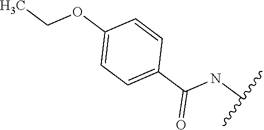 [SMILES: *NC(=O)c1ccc(OCC)cc1] | 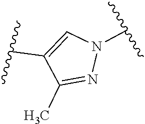 [SMILES: *c1cn(*)nc1C] | 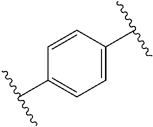 [SMILES: *c1ccc(*)cc1] |
| C-86 | 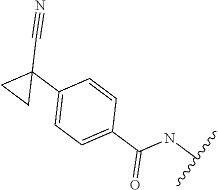 [SMILES: *NC(=O)c1ccc(C2(C#N)CC2)cc1] | 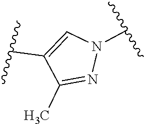 [SMILES: *c1cn(*)nc1C] | 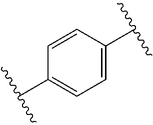 [SMILES: *c1ccc(*)cc1] |
| C-87 | 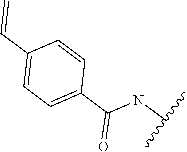 [SMILES: *NC(=O)c1ccc(C=C)cc1] | 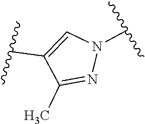 [SMILES: *c1cn(*)nc1C] | 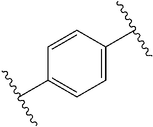 [SMILES: *c1ccc(*)cc1] |
| C-88 | 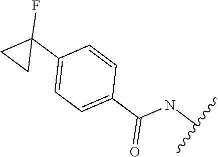 [SMILES: *NC(=O)c1ccc(C2(F)CC2)cc1] | 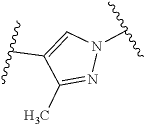 [SMILES: *c1cn(*)nc1C] | 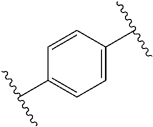 [SMILES: *c1ccc(*)cc1] |
| C-89 | 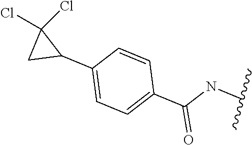 [SMILES: *NC(=O)c1ccc(C2CC2(Cl)Cl)cc1] | 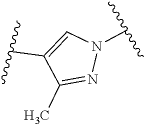 [SMILES: *c1cn(*)nc1C] | 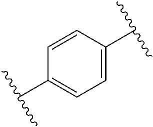 [SMILES: *c1ccc(*)cc1] |
| C-90 | 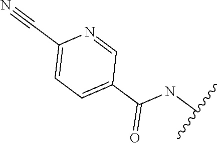 [SMILES: *NC(=O)c1ccc(C#N)nc1] | 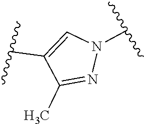 [SMILES: *c1cn(*)nc1C] | 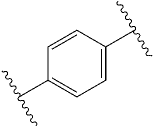 [SMILES: *c1ccc(*)cc1] |
| C-91 | 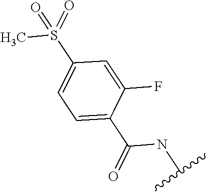 [SMILES: *NC(=O)c1ccc(S(C)(=O)=O)cc1F] | 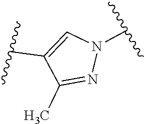 [SMILES: *c1cn(*)nc1C] | 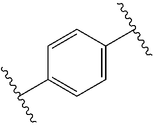 [SMILES: *c1ccc(*)cc1] |
| C-92 | 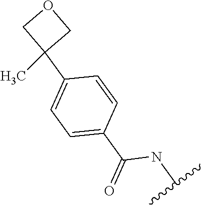 [SMILES: *NC(=O)c1ccc(C2(C)COC2)cc1] | 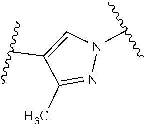 [SMILES: *c1cn(*)nc1C] | 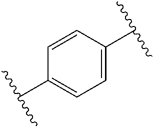 [SMILES: *c1ccc(*)cc1] |
| C-93 | 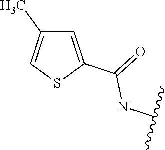 [SMILES: *NC(=O)c1cc(C)cs1] | 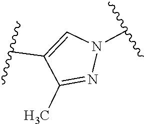 [SMILES: *c1cn(*)nc1C] | 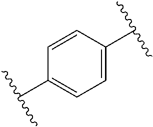 [SMILES: *c1ccc(*)cc1] |
| C-94 | 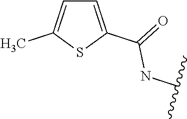 [SMILES: *NC(=O)c1ccc(C)s1] | 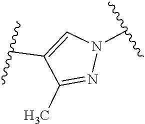 [SMILES: *c1cn(*)nc1C] | 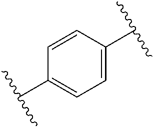 [SMILES: *c1ccc(*)cc1] |
| C-95 | 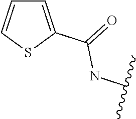 [SMILES: *NC(=O)c1cccs1] | 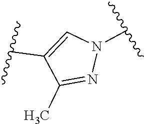 [SMILES: *c1cn(*)nc1C] | 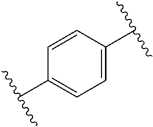 [SMILES: *c1ccc(*)cc1] |
| C-96 | 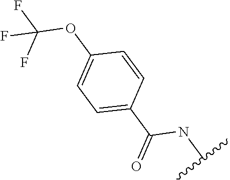 [SMILES: *NC(=O)c1ccc(OC(F)(F)F)cc1] | 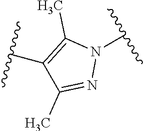 [SMILES: *c1c(C)nn(*)c1C] | 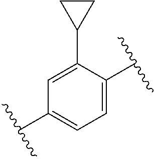 [SMILES: *c1ccc(*)c(C2CC2)c1] |
| C-97 | 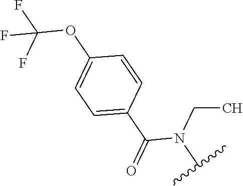 [SMILES: *N(CC)C(=O)c1ccc(OC(F)(F)F)cc1] | 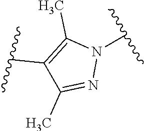 [SMILES: *c1c(C)nn(*)c1C] | 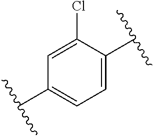 [SMILES: *c1ccc(*)c(Cl)c1] |
| C-98 | 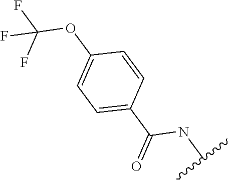 [SMILES: *NC(=O)c1ccc(OC(F)(F)F)cc1] | 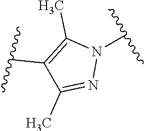 [SMILES: *c1c(C)nn(*)c1C] | 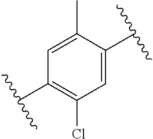 [SMILES: *c1cc(Cl)c(*)cc1C] |
| C-99 | 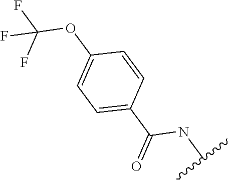 [SMILES: *NC(=O)c1ccc(OC(F)(F)F)cc1] | 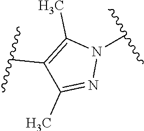 [SMILES: *c1c(C)nn(*)c1C] | 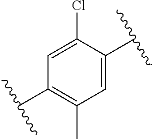 [SMILES: *c1cc(Cl)c(*)cc1C] |
| C-100 | 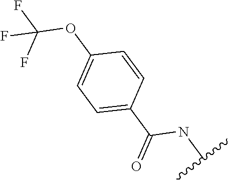 [SMILES: *NC(=O)c1ccc(OC(F)(F)F)cc1] | 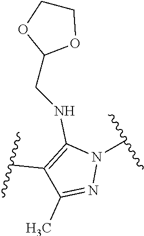 [SMILES: *c1c(C)nn(*)c1NCC1OCCO1] | 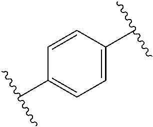 [SMILES: *c1ccc(*)cc1] |
| C-101 | 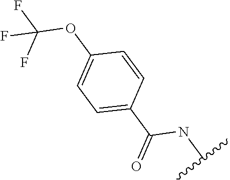 [SMILES: *NC(=O)c1ccc(OC(F)(F)F)cc1] | 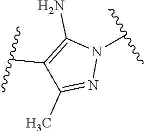 [SMILES: *c1c(C)nn(*)c1N] | 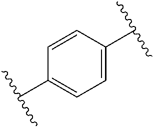 [SMILES: *c1ccc(*)cc1] |
| C-102 | 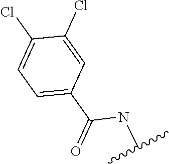 [SMILES: *NC(=O)c1ccc(Cl)c(Cl)c1] | 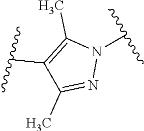 [SMILES: *c1c(C)nn(*)c1C] | 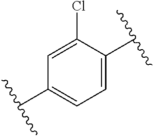 [SMILES: *c1ccc(*)c(Cl)c1] |
| C-103 | 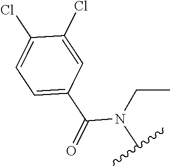 [SMILES: *N(CC)C(=O)c1ccc(Cl)c(Cl)c1] | 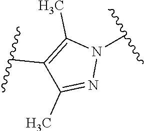 [SMILES: *c1c(C)nn(*)c1C] | 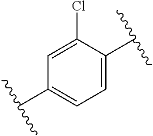 [SMILES: *c1ccc(*)c(Cl)c1] |
| C-104 | 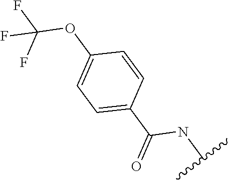 [SMILES: *NC(=O)c1ccc(OC(F)(F)F)cc1] | 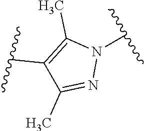 [SMILES: *c1c(C)nn(*)c1C] | 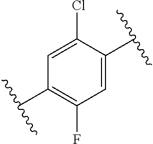 [SMILES: *c1cc(Cl)c(*)cc1F] |
| C-105 | 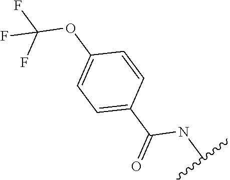 [SMILES: *NC(=O)c1ccc(OC(F)(F)F)cc1] | 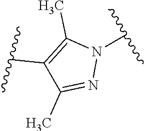 [SMILES: *c1c(C)nn(*)c1C] | 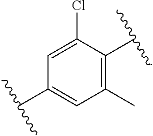 [SMILES: *c1cc(C)c(*)c(Cl)c1] |
| C-106 | 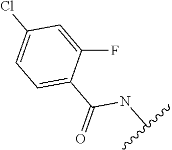 [SMILES: *NC(=O)c1ccc(Cl)cc1F] | 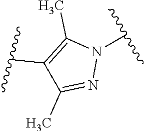 [SMILES: *c1c(C)nn(*)c1C] | 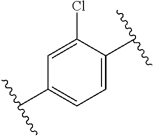 [SMILES: *c1ccc(*)c(Cl)c1] |
| C-107 | 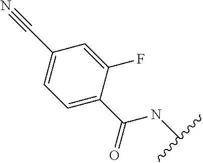 [SMILES: *NC(=O)c1ccc(C#N)cc1F] | 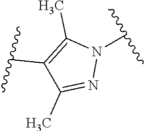 [SMILES: *c1c(C)nn(*)c1C] | 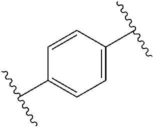 [SMILES: *c1ccc(*)cc1] |
| C-108 | 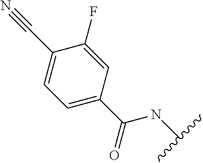 [SMILES: *NC(=O)c1ccc(C#N)c(F)c1] | 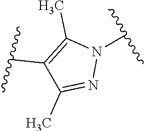 [SMILES: *c1c(C)nn(*)c1C] | 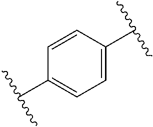 [SMILES: *c1ccc(*)cc1] |
| C-109 | 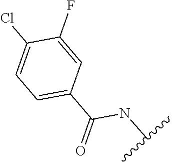 [SMILES: *NC(=O)c1ccc(Cl)c(F)c1] | 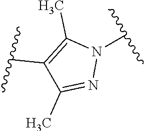 [SMILES: *c1c(C)nn(*)c1C] | 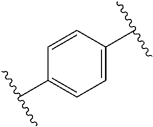 [SMILES: *c1ccc(*)cc1] |
| C-110 | 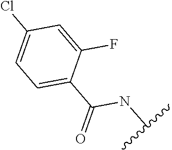 [SMILES: *NC(=O)c1ccc(Cl)cc1F] | 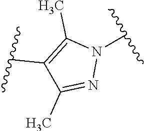 [SMILES: *c1c(C)nn(*)c1C] | 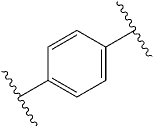 [SMILES: *c1ccc(*)cc1] |
| C-111 | 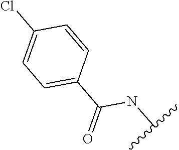 [SMILES: *NC(=O)c1ccc(Cl)cc1] | 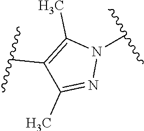 [SMILES: *c1c(C)nn(*)c1C] | 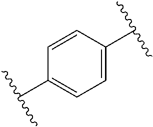 [SMILES: *c1ccc(*)cc1] |
| C-112 | 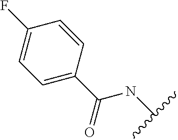 [SMILES: *NC(=O)c1ccc(F)cc1] | 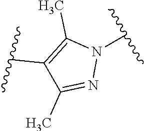 [SMILES: *c1c(C)nn(*)c1C] | 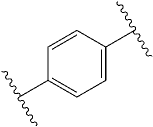 [SMILES: *c1ccc(*)cc1] |
| C-113 | 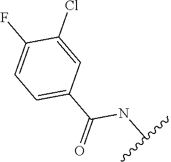 [SMILES: *NC(=O)c1ccc(F)c(Cl)c1] | 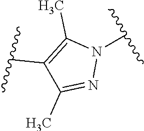 [SMILES: *c1c(C)nn(*)c1C] | 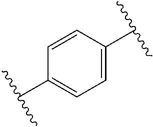 [SMILES: *c1ccc(*)cc1] |
| C-114 | 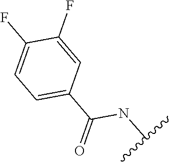 [SMILES: *NC(=O)c1ccc(F)c(F)c1] | 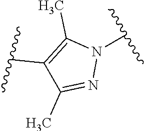 [SMILES: *c1c(C)nn(*)c1C] | 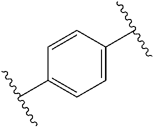 [SMILES: *c1ccc(*)cc1] |
| C-115 | 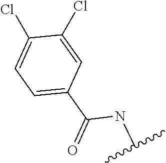 [SMILES: *NC(=O)c1ccc(Cl)c(Cl)c1] | 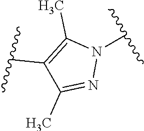 [SMILES: *c1c(C)nn(*)c1C] | 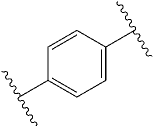 [SMILES: *c1ccc(*)cc1] |
| C-116 | 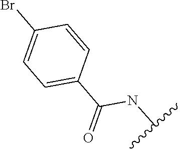 [SMILES: *NC(=O)c1ccc(Br)cc1] | 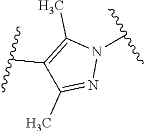 [SMILES: *c1c(C)nn(*)c1C] | 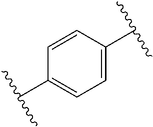 [SMILES: *c1ccc(*)cc1] |
| C-117 | 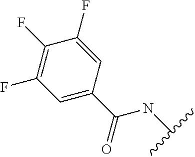 [SMILES: *NC(=O)c1cc(F)c(F)c(F)c1] | 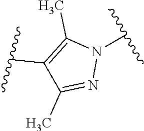 [SMILES: *c1c(C)nn(*)c1C] | 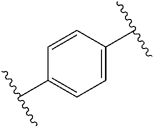 [SMILES: *c1ccc(*)cc1] |
| C-118 | 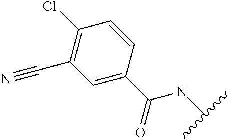 [SMILES: *NC(=O)c1ccc(Cl)c(C#N)c1] | 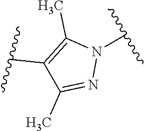 [SMILES: *c1c(C)nn(*)c1C] | 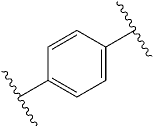 [SMILES: *c1ccc(*)cc1] |
| C-119 | 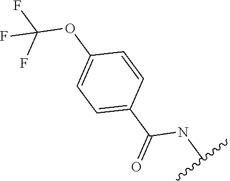 [SMILES: *NC(=O)c1ccc(OC(F)(F)F)cc1] | 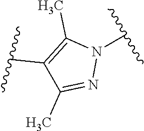 [SMILES: *c1c(C)nn(*)c1C] | 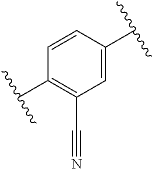 [SMILES: *c1ccc(*)c(C#N)c1] |
| C-120 | 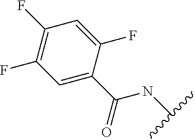 [SMILES: *NC(=O)c1cc(F)c(F)cc1F] | 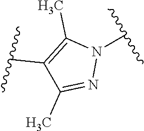 [SMILES: *c1c(C)nn(*)c1C] | 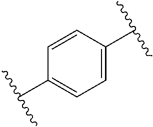 [SMILES: *c1ccc(*)cc1] |
| C-121 | 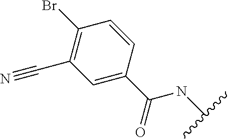 [SMILES: *NC(=O)c1ccc(Br)c(C#N)c1] | 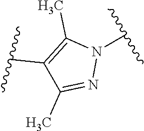 [SMILES: *c1c(C)nn(*)c1C] | 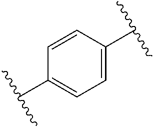 [SMILES: *c1ccc(*)cc1] |
| C-122 | 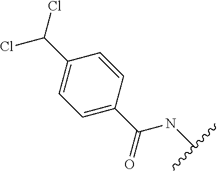 [SMILES: *NC(=O)c1ccc(C(Cl)Cl)cc1] | 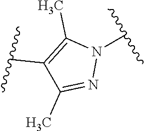 [SMILES: *c1c(C)nn(*)c1C] | 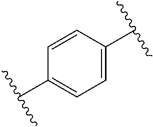 [SMILES: *c1ccc(*)cc1] |
| C-123 | 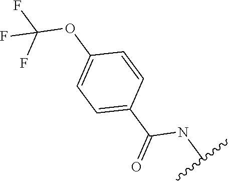 [SMILES: *NC(=O)c1ccc(OC(F)(F)F)cc1] | 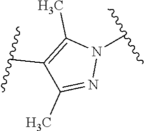 [SMILES: *c1c(C)nn(*)c1C] | 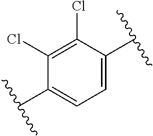 [SMILES: *c1ccc(*)c(Cl)c1Cl] |
| C-124 | 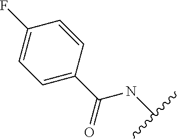 [SMILES: *NC(=O)c1ccc(F)cc1] | 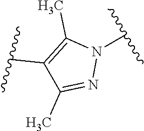 [SMILES: *c1c(C)nn(*)c1C] | 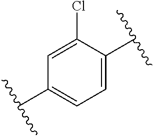 [SMILES: *c1ccc(*)c(Cl)c1] |
| C-125 | 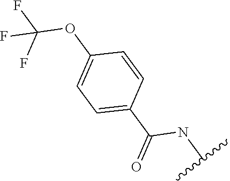 [SMILES: *NC(=O)c1ccc(OC(F)(F)F)cc1] | 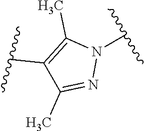 [SMILES: *c1c(C)nn(*)c1C] | 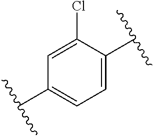 [SMILES: *c1ccc(*)c(Cl)c1] |
| C-126 | 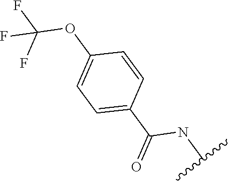 [SMILES: *NC(=O)c1ccc(OC(F)(F)F)cc1] | 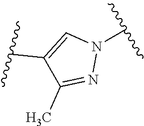 [SMILES: *c1cn(*)nc1C] | 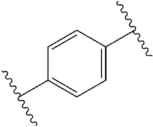 [SMILES: *c1ccc(*)cc1] |
| C-127 | 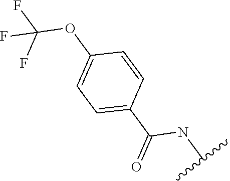 [SMILES: *NC(=O)c1ccc(OC(F)(F)F)cc1] | 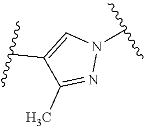 [SMILES: *c1cn(*)nc1C] | 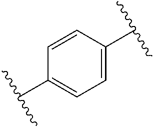 [SMILES: *c1ccc(*)cc1] |
| C-128 | 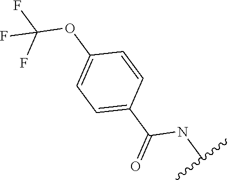 [SMILES: *NC(=O)c1ccc(OC(F)(F)F)cc1] | 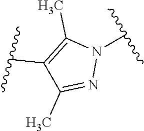 [SMILES: *c1c(C)nn(*)c1C] | 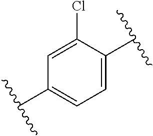 [SMILES: *c1ccc(*)c(Cl)c1] |
| C-129 | 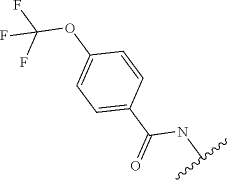 [SMILES: *NC(=O)c1ccc(OC(F)(F)F)cc1] | 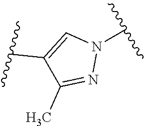 [SMILES: *c1cn(*)nc1C] | 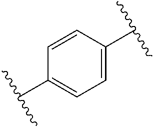 [SMILES: *c1ccc(*)cc1] |
| C-130 | 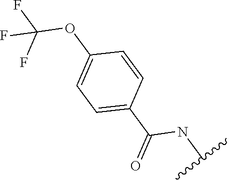 [SMILES: *NC(=O)c1ccc(OC(F)(F)F)cc1] | 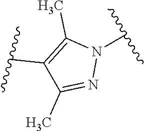 [SMILES: *c1c(C)nn(*)c1C] | 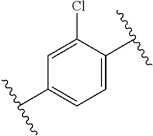 [SMILES: *c1ccc(*)c(Cl)c1] |
| C-131 | 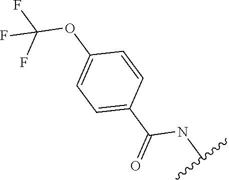 [SMILES: *NC(=O)c1ccc(OC(F)(F)F)cc1] | 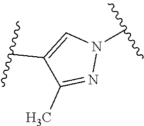 [SMILES: *c1cn(*)nc1C] | 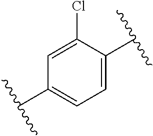 [SMILES: *c1ccc(*)c(Cl)c1] |
| C-132 | 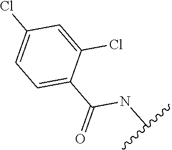 [SMILES: *NC(=O)c1ccc(Cl)cc1Cl] | 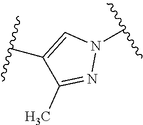 [SMILES: *c1cn(*)nc1C] | 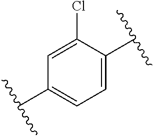 [SMILES: *c1ccc(*)c(Cl)c1] |
| C-133 | 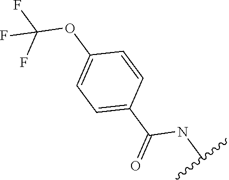 [SMILES: *NC(=O)c1ccc(OC(F)(F)F)cc1] | 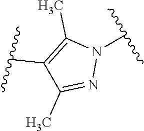 [SMILES: *c1c(C)nn(*)c1C] | 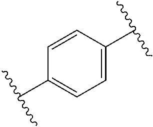 [SMILES: *c1ccc(*)cc1] |
| C-134 | 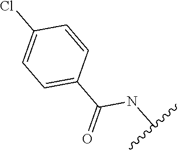 [SMILES: *NC(=O)c1ccc(Cl)cc1] | 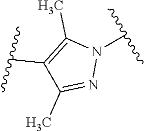 [SMILES: *c1c(C)nn(*)c1C] | 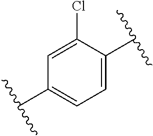 [SMILES: *c1ccc(*)c(Cl)c1] |
| No. | 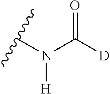 [SMILES: *NC([2H])=O] | LC MS/MZ | Rt min |
| C-1 | 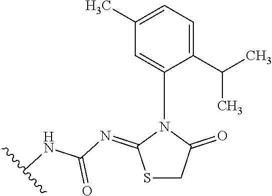 [SMILES: *NC(=O)/N=C1\SCC(=O)N1c1cc(C)ccc1C(C)C] | 665 (Method 1) | 2.464 |
| C-2 | 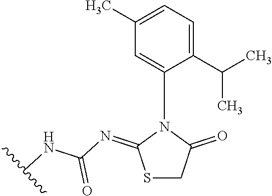 [SMILES: *NC(=O)/N=C1\SCC(=O)N1c1cc(C)ccc1C(C)C] | 665.1 (Method 2) | 1.39 |
| C-3 | 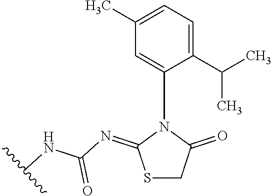 [SMILES: *NC(=O)/N=C1\SCC(=O)N1c1cc(C)ccc1C(C)C] | 699.1 (Method 1) | 1.394 |
| C-4 | 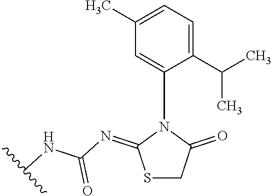 [SMILES: *NC(=O)/N=C1\SCC(=O)N1c1cc(C)ccc1C(C)C] | 699 (Method 1) | 2.507 |
| C-5 | 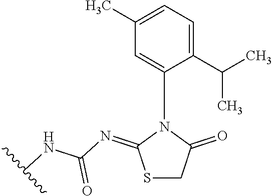 [SMILES: *NC(=O)/N=C1\SCC(=O)N1c1cc(C)ccc1C(C)C] | 690 (Method 1) | 1.195 |
| C-6 | 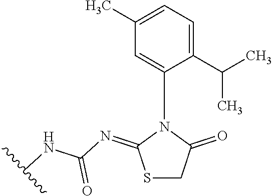 [SMILES: *NC(=O)/N=C1\SCC(=O)N1c1cc(C)ccc1C(C)C] | 669 (Method 1) | 2.528 |
| C-7 | 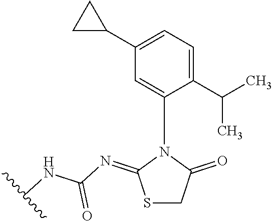 [SMILES: *NC(=O)/N=C1\SCC(=O)N1c1cc(C2CC2)ccc1C(C)C] | 695 (Method 1) | 2.667 |
| C-8 | 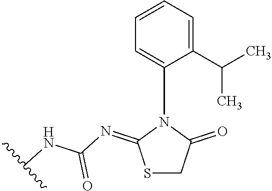 [SMILES: *NC(=O)/N=C1\SCC(=O)N1c1ccccc1C(C)C] | 635 (Method 1) | 2.069 |
| C-9 | 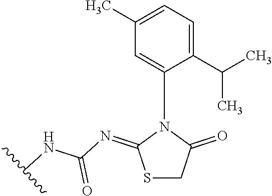 [SMILES: *NC(=O)/N=C1\SCC(=O)N1c1cc(C)ccc1C(C)C] | 651 (Method 1) | 2.101 |
| C-10 | 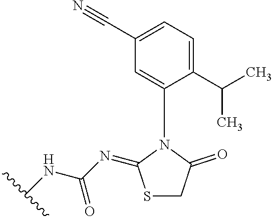 [SMILES: *NC(=O)/N=C1\SCC(=O)N1c1cc(C#N)ccc1C(C)C] | 662.2 (Method 2) | 1.307 |
| C-11 | 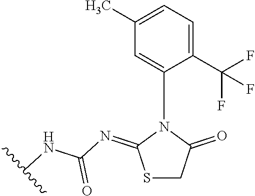 [SMILES: *NC(=O)/N=C1\SCC(=O)N1c1cc(C)ccc1C(F)(F)F] | 691 (Method 2) | 1.284 |
| C-12 | 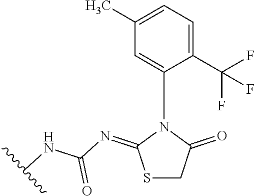 [SMILES: *NC(=O)/N=C1\SCC(=O)N1c1cc(C)ccc1C(F)(F)F] | 677 (Method 2) | 1.314 |
| C-13 | 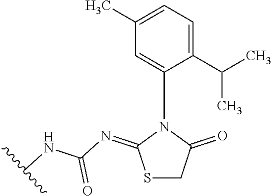 [SMILES: *NC(=O)/N=C1\SCC(=O)N1c1cc(C)ccc1C(C)C] | 674 (Method 1) | 1.301 |
| C-14 | 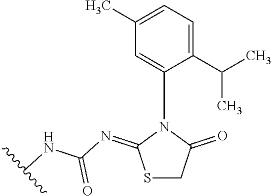 [SMILES: *NC(=O)/N=C1\SCC(=O)N1c1cc(C)ccc1C(C)C] | 682 (Method 1) | 1.312 |
| C-15 | 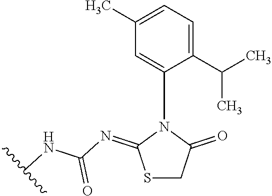 [SMILES: *NC(=O)/N=C1\SCC(=O)N1c1cc(C)ccc1C(C)C] | 665 (Method 1) | 1.998 |
| C-16 | 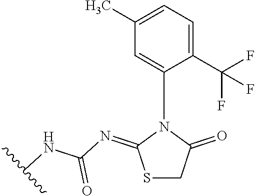 [SMILES: *NC(=O)/N=C1\SCC(=O)N1c1cc(C)ccc1C(F)(F)F] | 691 (Method 1) | 2.373 |
| C-17 | 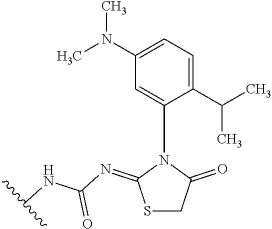 [SMILES: *NC(=O)/N=C1\SCC(=O)N1c1cc(N(C)C)ccc1C(C)C] | 694 (Method 1) | 2.219 |
| C-18 | 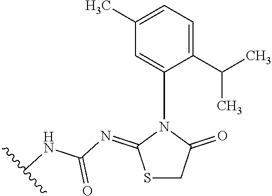 [SMILES: *NC(=O)/N=C1\SCC(=O)N1c1cc(C)ccc1C(C)C] | 680 (Method 1) | 1.088 |
| C-19 | 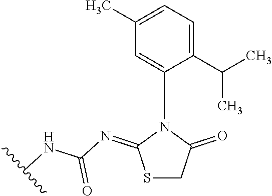 [SMILES: *NC(=O)/N=C1\SCC(=O)N1c1cc(C)ccc1C(C)C] | 743 (Method 1) | 2.26 |
| C-20 | 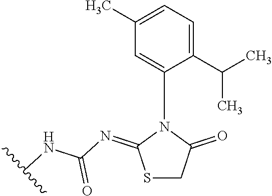 [SMILES: *NC(=O)/N=C1\SCC(=O)N1c1cc(C)ccc1C(C)C] | 729 (Method 1) | 2.528 |
| C-21 | 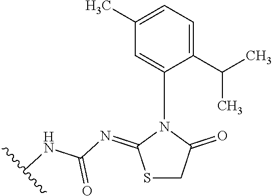 [SMILES: *NC(=O)/N=C1\SCC(=O)N1c1cc(C)ccc1C(C)C] | 699 (Method 1) | 2.53 |
| C-22 | 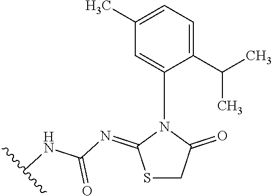 [SMILES: *NC(=O)/N=C1\SCC(=O)N1c1cc(C)ccc1C(C)C] | 666.3 (Method 1) | 2.16 |
| C-23 | 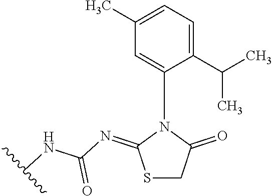 [SMILES: *NC(=O)/N=C1\SCC(=O)N1c1cc(C)ccc1C(C)C] | 621.3 (Method 2) | 1.336 |
| C-24 | 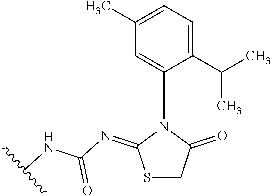 [SMILES: *NC(=O)/N=C1\SCC(=O)N1c1cc(C)ccc1C(C)C] | 628.3 (Method 2) | 1.316 |
| C-25 | 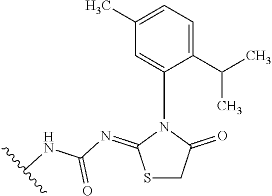 [SMILES: *NC(=O)/N=C1\SCC(=O)N1c1cc(C)ccc1C(C)C] | 603.3 (Method 2) | 1.298 |
| C-26 | 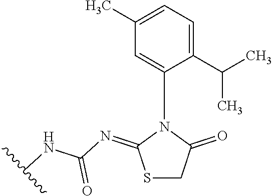 [SMILES: *NC(=O)/N=C1\SCC(=O)N1c1cc(C)ccc1C(C)C] | 635.2 (Method 2) | 1.393 |
| C-27 | 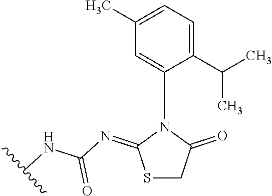 [SMILES: *NC(=O)/N=C1\SCC(=O)N1c1cc(C)ccc1C(C)C] | 635.3 (Method 2) | 1.336 |
| C-28 | 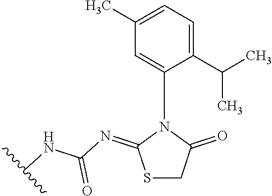 [SMILES: *NC(=O)/N=C1\SCC(=O)N1c1cc(C)ccc1C(C)C] | 619.3 (Method 2) | 1.317 |
| C-29 | 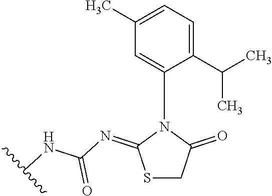 [SMILES: *NC(=O)/N=C1\SCC(=O)N1c1cc(C)ccc1C(C)C] | 601.3 (Method 2) | 1.311 |
| C-30 | 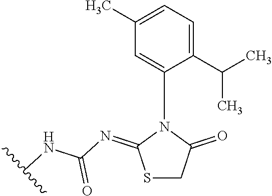 [SMILES: *NC(=O)/N=C1\SCC(=O)N1c1cc(C)ccc1C(C)C] | 647.2 (Method 2) | 1.319 |
| C-31 | 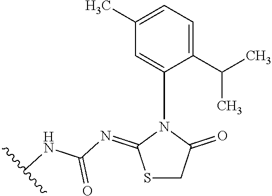 [SMILES: *NC(=O)/N=C1\SCC(=O)N1c1cc(C)ccc1C(C)C] | 661.2 (Method 2) | 1.317 |
| C-32 | 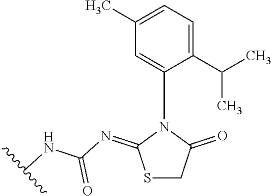 [SMILES: *NC(=O)/N=C1\SCC(=O)N1c1cc(C)ccc1C(C)C] | 597.3 (Method 2) | 1.241 |
| C-33 | 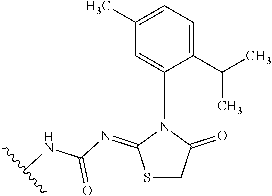 [SMILES: *NC(=O)/N=C1\SCC(=O)N1c1cc(C)ccc1C(C)C] | 592.3 (Method 2) | 1.231 |
| C-34 | 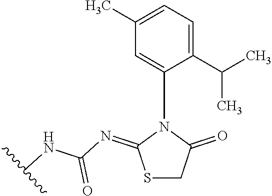 [SMILES: *NC(=O)/N=C1\SCC(=O)N1c1cc(C)ccc1C(C)C] | 591.4 (Method 2) | 1.377 |
| C-35 | 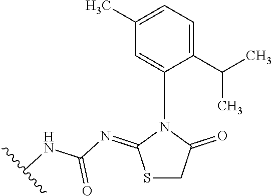 [SMILES: *NC(=O)/N=C1\SCC(=O)N1c1cc(C)ccc1C(C)C] | 626.2 (Method 2) | 1.264 |
| C-36 | 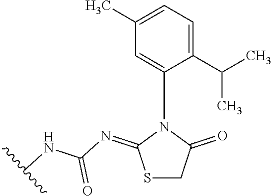 [SMILES: *NC(=O)/N=C1\SCC(=O)N1c1cc(C)ccc1C(C)C] | 631.3 (Method 2) | 1.306 |
| C-37 | 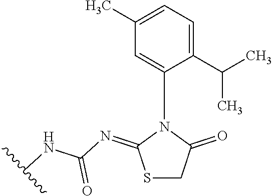 [SMILES: *NC(=O)/N=C1\SCC(=O)N1c1cc(C)ccc1C(C)C] | 603.3 (Method 2) | 1.257 |
| C-38 | 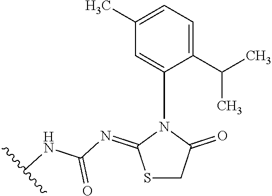 [SMILES: *NC(=O)/N=C1\SCC(=O)N1c1cc(C)ccc1C(C)C] | 646.3 (Method 2) | 1.202 |
| C-39 | 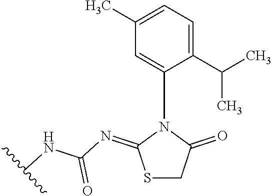 [SMILES: *NC(=O)/N=C1\SCC(=O)N1c1cc(C)ccc1C(C)C] | 607.3 (Method 2) | 1.31 |
| C-40 | 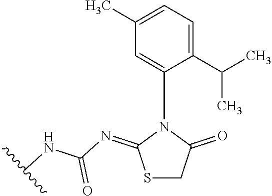 [SMILES: *NC(=O)/N=C1\SCC(=O)N1c1cc(C)ccc1C(C)C] | 607.2 (Method 2) | 1.318 |
| C-41 | 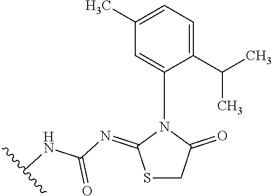 [SMILES: *NC(=O)/N=C1\SCC(=O)N1c1cc(C)ccc1C(C)C] | 598.3 (Method 2) | 1.23 |
| C-42 | 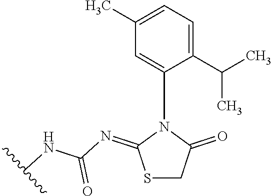 [SMILES: *NC(=O)/N=C1\SCC(=O)N1c1cc(C)ccc1C(C)C] | 608.2 (Method 2) | 1.333 |
| C-43 | 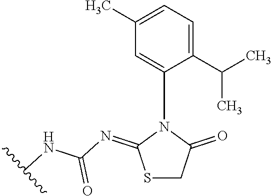 [SMILES: *NC(=O)/N=C1\SCC(=O)N1c1cc(C)ccc1C(C)C] | 608.2 (Method 2) | 1.316 |
| C-44 | 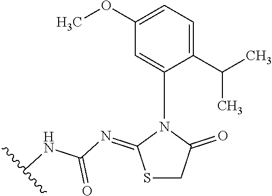 [SMILES: *NC(=O)/N=C1\SCC(=O)N1c1cc(OC)ccc1C(C)C] | 715.2 (Method 1) | 2.18 |
| C-45 | 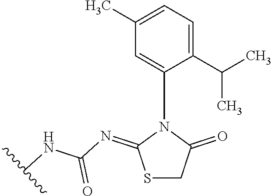 [SMILES: *NC(=O)/N=C1\SCC(=O)N1c1cc(C)ccc1C(C)C] | 699.2 (Method 1) | 2.19 |
| C-46 | 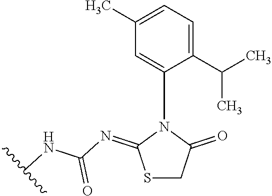 [SMILES: *NC(=O)/N=C1\SCC(=O)N1c1cc(C)ccc1C(C)C] | 753.3 (Method 1) | 2.37 |
| C-47 | 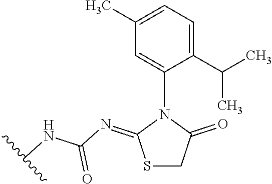 [SMILES: *NC(=O)/N=C1\SCC(=O)N1c1cc(C)ccc1C(C)C] | 670.3 (Method 2) | 1.203 |
| C-48 | 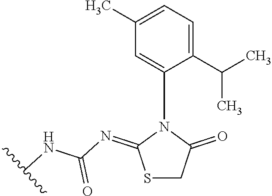 [SMILES: *NC(=O)/N=C1\SCC(=O)N1c1cc(C)ccc1C(C)C] | 625.4 (Method 2) | 1.34 |
| C-49 | 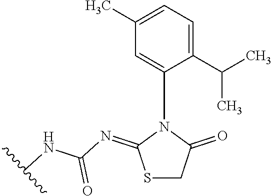 [SMILES: *NC(=O)/N=C1\SCC(=O)N1c1cc(C)ccc1C(C)C] | 581.4 (Method 2) | 1.289 |
| C-50 | 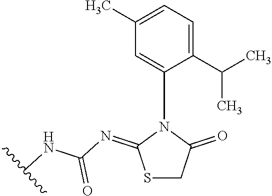 [SMILES: *NC(=O)/N=C1\SCC(=O)N1c1cc(C)ccc1C(C)C] | 646.2 (Method 2) | 1.146 |
| C-51 | 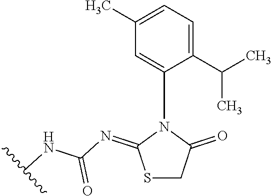 [SMILES: *NC(=O)/N=C1\SCC(=O)N1c1cc(C)ccc1C(C)C] | 561.3 (Method 2) | 0.987 |
| C-52 | 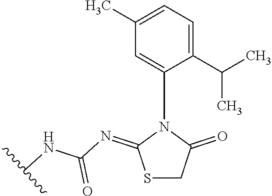 [SMILES: *NC(=O)/N=C1\SCC(=O)N1c1cc(C)ccc1C(C)C] | 623.4 (Method 2) | 1.208 |
| C-53 | 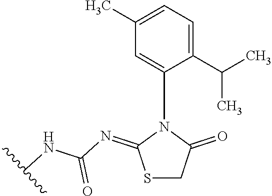 [SMILES: *NC(=O)/N=C1\SCC(=O)N1c1cc(C)ccc1C(C)C] | 645.3 (Method 2) | 1.167 |
| C-54 | 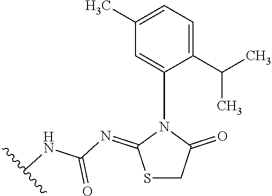 [SMILES: *NC(=O)/N=C1\SCC(=O)N1c1cc(C)ccc1C(C)C] | 612 (Method 2) | 1.268 |
| C-55 | 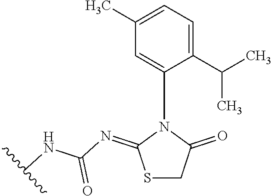 [SMILES: *NC(=O)/N=C1\SCC(=O)N1c1cc(C)ccc1C(C)C] | 658.3 (Method 2) | 1.108 |
| C-56 | 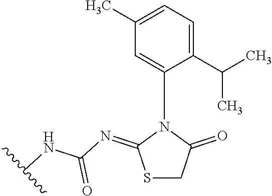 [SMILES: *NC(=O)/N=C1\SCC(=O)N1c1cc(C)ccc1C(C)C] | 634.4 (Method 2) | 1.285 |
| C-57 | 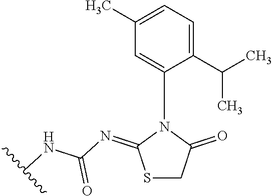 [SMILES: *NC(=O)/N=C1\SCC(=O)N1c1cc(C)ccc1C(C)C] | 663.3 (Method 2) | 1.195 |
| C-58 | 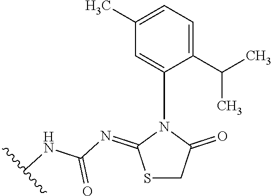 [SMILES: *NC(=O)/N=C1\SCC(=O)N1c1cc(C)ccc1C(C)C] | 621.4 (Method 2) | 1.42 |
| C-59 | 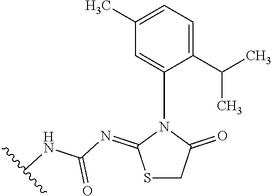 [SMILES: *NC(=O)/N=C1\SCC(=O)N1c1cc(C)ccc1C(C)C] | 568.3 (Method 2) | 1.02 |
| C-60 | 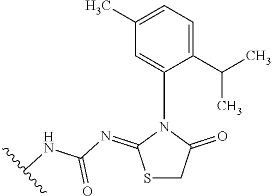 [SMILES: *NC(=O)/N=C1\SCC(=O)N1c1cc(C)ccc1C(C)C] | 625.4 (Method 2) | 1.128 |
| C-61 | 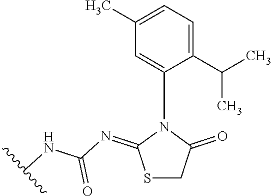 [SMILES: *NC(=O)/N=C1\SCC(=O)N1c1cc(C)ccc1C(C)C] | 639.4 (Method 2) | 1.311 |
| C-62 | 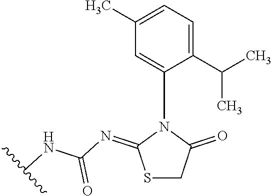 [SMILES: *NC(=O)/N=C1\SCC(=O)N1c1cc(C)ccc1C(C)C] | 607.4 (Method 2) | 1.348 |
| C-63 | 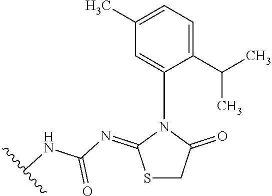 [SMILES: *NC(=O)/N=C1\SCC(=O)N1c1cc(C)ccc1C(C)C] | 660.3 (Method 2) | 1.141 |
| C-64 | 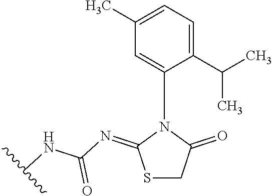 [SMILES: *NC(=O)/N=C1\SCC(=O)N1c1cc(C)ccc1C(C)C] | 624.4 (Method 2) | 1.11 |
| C-65 | 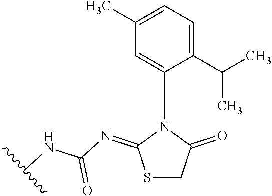 [SMILES: *NC(=O)/N=C1\SCC(=O)N1c1cc(C)ccc1C(C)C] | 643.3 (Method 2) | 1.303 |
| C-66 | 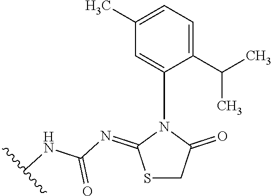 [SMILES: *NC(=O)/N=C1\SCC(=O)N1c1cc(C)ccc1C(C)C] | 602.3 (Method 2) | 1.24 |
| C-67 | 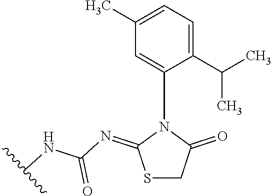 [SMILES: *NC(=O)/N=C1\SCC(=O)N1c1cc(C)ccc1C(C)C] | 621.3 (Method 2) | 1.254 |
| C-68 | 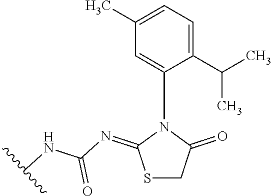 [SMILES: *NC(=O)/N=C1\SCC(=O)N1c1cc(C)ccc1C(C)C] | 569.4 (Method 2) | 1.093 |
| C-69 | 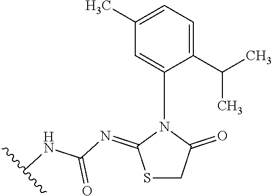 [SMILES: *NC(=O)/N=C1\SCC(=O)N1c1cc(C)ccc1C(C)C] | 609.3 (Method 2) | 1.214 |
| C-70 | 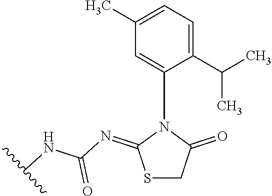 [SMILES: *NC(=O)/N=C1\SCC(=O)N1c1cc(C)ccc1C(C)C] | 609.4 (Method 2) | 1.375 |
| C-71 | 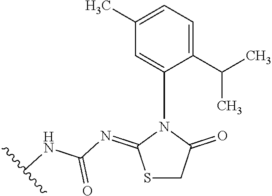 [SMILES: *NC(=O)/N=C1\SCC(=O)N1c1cc(C)ccc1C(C)C] | 693.2 (Method 2) | 1.351 |
| C-72 | 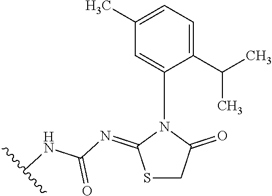 [SMILES: *NC(=O)/N=C1\SCC(=O)N1c1cc(C)ccc1C(C)C] | 631.4 (Method 2) | 1.379 |
| C-73 | 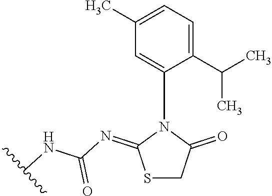 [SMILES: *NC(=O)/N=C1\SCC(=O)N1c1cc(C)ccc1C(C)C] | 623.4 (Method 2) | 1.417 |
| C-74 | 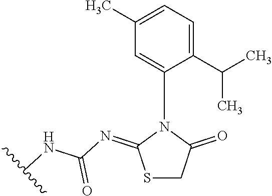 [SMILES: *NC(=O)/N=C1\SCC(=O)N1c1cc(C)ccc1C(C)C] | 587.3 (Method 2) | 1.192 |
| C-75 | 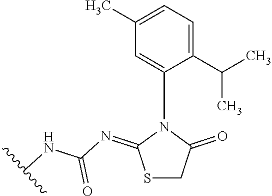 [SMILES: *NC(=O)/N=C1\SCC(=O)N1c1cc(C)ccc1C(C)C] | 610.4 (Method 2) | 1.224 |
| C-76 | 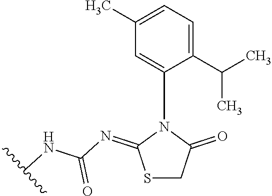 [SMILES: *NC(=O)/N=C1\SCC(=O)N1c1cc(C)ccc1C(C)C] | 622.3 (Method 2) | 1.192 |
| C-77 | 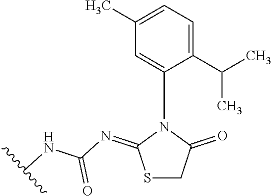 [SMILES: *NC(=O)/N=C1\SCC(=O)N1c1cc(C)ccc1C(C)C] | 641.3 (Method 2) | 1.23 |
| C-78 | 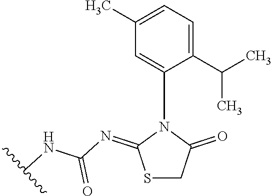 [SMILES: *NC(=O)/N=C1\SCC(=O)N1c1cc(C)ccc1C(C)C] | 606.2 (Method 2) | 1.16 |
| C-79 | 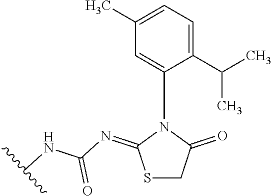 [SMILES: *NC(=O)/N=C1\SCC(=O)N1c1cc(C)ccc1C(C)C] | 699.2 (Method 2) | 1.332 |
| C-80 | 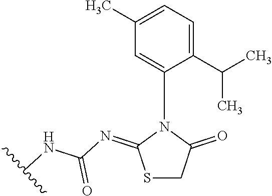 [SMILES: *NC(=O)/N=C1\SCC(=O)N1c1cc(C)ccc1C(C)C] | 595.4 (Method 2) | 1.321 |
| C-81 | 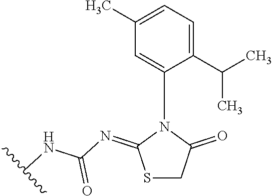 [SMILES: *NC(=O)/N=C1\SCC(=O)N1c1cc(C)ccc1C(C)C] | 569.3 (Method 2) | 1.188 |
| C-82 | 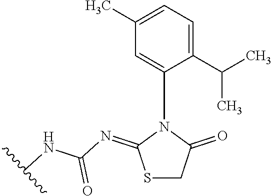 [SMILES: *NC(=O)/N=C1\SCC(=O)N1c1cc(C)ccc1C(C)C] | 617.3 (Method 2) | 1.288 |
| C-83 | 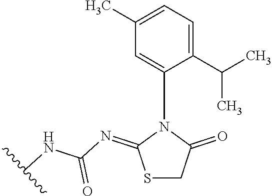 [SMILES: *NC(=O)/N=C1\SCC(=O)N1c1cc(C)ccc1C(C)C] | 613 (Method 2) | 1.262 |
| C-84 | 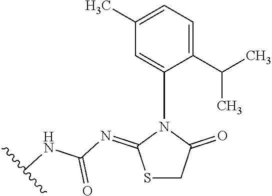 [SMILES: *NC(=O)/N=C1\SCC(=O)N1c1cc(C)ccc1C(C)C] | 594.3 (Method 2) | 1.233 |
| C-85 | 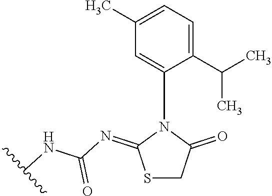 [SMILES: *NC(=O)/N=C1\SCC(=O)N1c1cc(C)ccc1C(C)C] | 611.3 (Method 2) | 1.287 |
| C-86 | 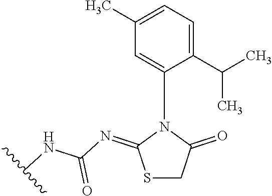 [SMILES: *NC(=O)/N=C1\SCC(=O)N1c1cc(C)ccc1C(C)C] | 632.3 (Method 2) | 1.268 |
| C-87 | 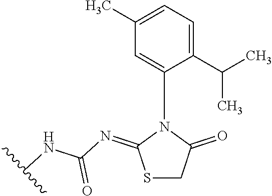 [SMILES: *NC(=O)/N=C1\SCC(=O)N1c1cc(C)ccc1C(C)C] | 593.3 (Method 2) | 1.302 |
| C-88 | 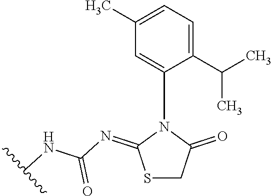 [SMILES: *NC(=O)/N=C1\SCC(=O)N1c1cc(C)ccc1C(C)C] | 625.3 (Method 2) | 1.323 |
| C-89 | 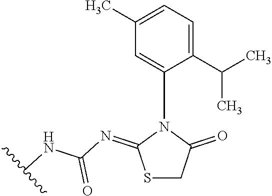 [SMILES: *NC(=O)/N=C1\SCC(=O)N1c1cc(C)ccc1C(C)C] | 675.3 (Method 2) | 1.372 |
| C-90 | 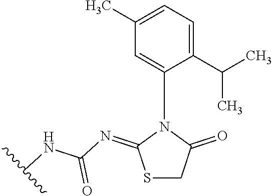 [SMILES: *NC(=O)/N=C1\SCC(=O)N1c1cc(C)ccc1C(C)C] | 593.1 (Method 2) | 1.191 |
| C-91 | 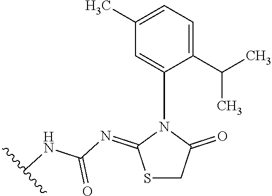 [SMILES: *NC(=O)/N=C1\SCC(=O)N1c1cc(C)ccc1C(C)C] | 663.3 (Method 2) | 1.188 |
| C-92 | 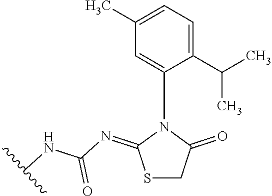 [SMILES: *NC(=O)/N=C1\SCC(=O)N1c1cc(C)ccc1C(C)C] | 637.4 (Method 2) | 1.226 |
| C-93 | 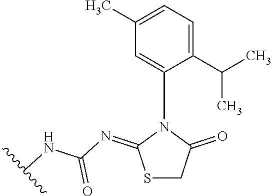 [SMILES: *NC(=O)/N=C1\SCC(=O)N1c1cc(C)ccc1C(C)C] | 587.3 (Method 2) | 1.281 |
| C-94 | 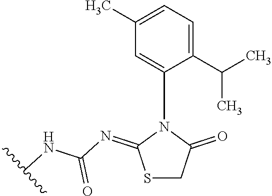 [SMILES: *NC(=O)/N=C1\SCC(=O)N1c1cc(C)ccc1C(C)C] | 587.3 (Method 2) | 1.275 |
| C-95 | 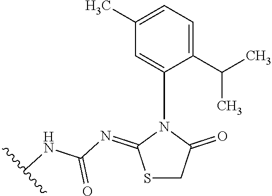 [SMILES: *NC(=O)/N=C1\SCC(=O)N1c1cc(C)ccc1C(C)C] | 573.3 (Method 2) | 1.232 |
| C-96 | 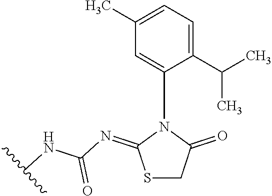 [SMILES: *NC(=O)/N=C1\SCC(=O)N1c1cc(C)ccc1C(C)C] | 705 (Method 2) | 1.374 |
| C-97 | 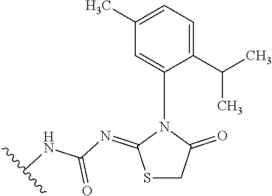 [SMILES: *NC(=O)/N=C1\SCC(=O)N1c1cc(C)ccc1C(C)C] | 728 (Method 1) | 2.34 |
| C-98 | 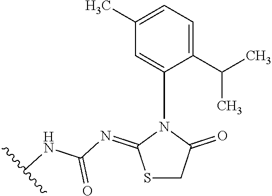 [SMILES: *NC(=O)/N=C1\SCC(=O)N1c1cc(C)ccc1C(C)C] | 711 (Method 1) | 2.2 |
| C-99 | 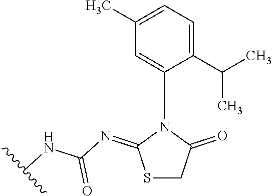 [SMILES: *NC(=O)/N=C1\SCC(=O)N1c1cc(C)ccc1C(C)C] | 713 (Method 1) | 2.22 |
| C-100 | 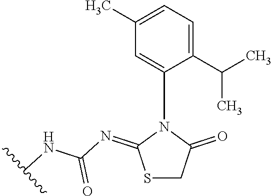 [SMILES: *NC(=O)/N=C1\SCC(=O)N1c1cc(C)ccc1C(C)C] | 752 (Method 2) | 1.23 |
| C-101 | 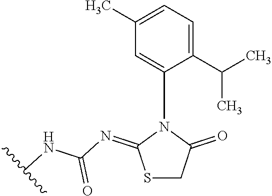 [SMILES: *NC(=O)/N=C1\SCC(=O)N1c1cc(C)ccc1C(C)C] | 666 (Method 2) | 1.14 |
| C-102 | 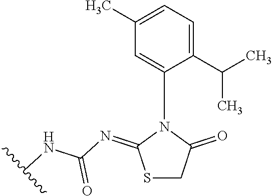 [SMILES: *NC(=O)/N=C1\SCC(=O)N1c1cc(C)ccc1C(C)C] | 685 (Method 1) | 2.3 |
| C-103 | 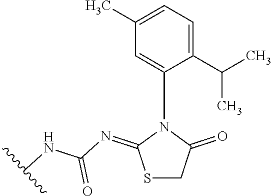 [SMILES: *NC(=O)/N=C1\SCC(=O)N1c1cc(C)ccc1C(C)C] | 713 (Method 1) | 2.5 |
| C-104 | 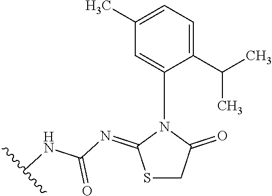 [SMILES: *NC(=O)/N=C1\SCC(=O)N1c1cc(C)ccc1C(C)C] | 717 (Method 1) | 2.3 |
| C-105 | 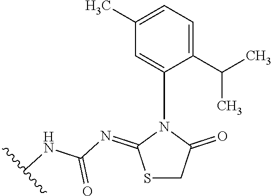 [SMILES: *NC(=O)/N=C1\SCC(=O)N1c1cc(C)ccc1C(C)C] | 713 (Method 1) | 2.2 |
| C-106 | 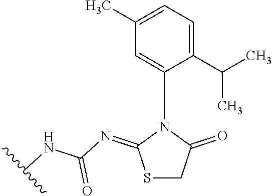 [SMILES: *NC(=O)/N=C1\SCC(=O)N1c1cc(C)ccc1C(C)C] | 667 (Method 1) | 2.2 |
| C-107 | 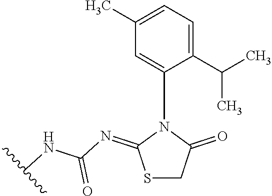 [SMILES: *NC(=O)/N=C1\SCC(=O)N1c1cc(C)ccc1C(C)C] | 624 (Method 2) | 1.2 |
| C-108 | 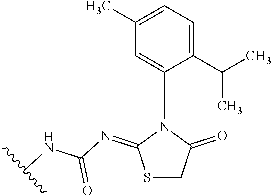 [SMILES: *NC(=O)/N=C1\SCC(=O)N1c1cc(C)ccc1C(C)C] | 624 (Method 2) | 1.2 |
| C-109 | 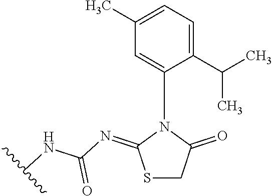 [SMILES: *NC(=O)/N=C1\SCC(=O)N1c1cc(C)ccc1C(C)C] | 633 (Method 2) | 1.25 |
| C-110 | 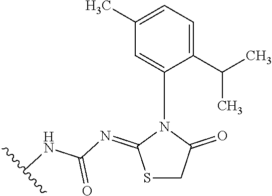 [SMILES: *NC(=O)/N=C1\SCC(=O)N1c1cc(C)ccc1C(C)C] | 633 (Method 2) | 1.27 |
| C-111 | 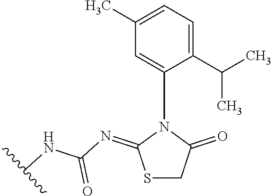 [SMILES: *NC(=O)/N=C1\SCC(=O)N1c1cc(C)ccc1C(C)C] | 615 (Method 2) | 1.25 |
| C-112 | 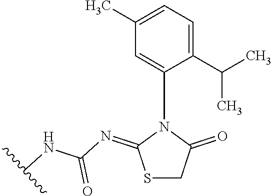 [SMILES: *NC(=O)/N=C1\SCC(=O)N1c1cc(C)ccc1C(C)C] | 599 (Method 2) | 1.20 |
| C-113 | 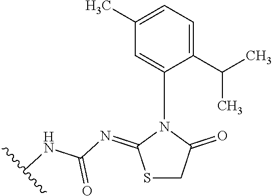 [SMILES: *NC(=O)/N=C1\SCC(=O)N1c1cc(C)ccc1C(C)C] | 633 (Method 2) | 1.27 |
| C-114 | 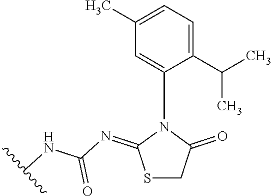 [SMILES: *NC(=O)/N=C1\SCC(=O)N1c1cc(C)ccc1C(C)C] | 617 (Method 2) | 1.23 |
| C-115 | 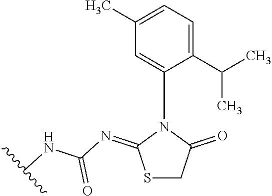 [SMILES: *NC(=O)/N=C1\SCC(=O)N1c1cc(C)ccc1C(C)C] | 649 (Method 2) | 1.32 |
| C-116 | 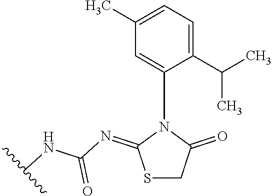 [SMILES: *NC(=O)/N=C1\SCC(=O)N1c1cc(C)ccc1C(C)C] | 661 (Method 2) | 1.26 |
| C-117 | 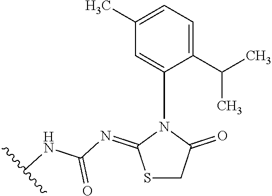 [SMILES: *NC(=O)/N=C1\SCC(=O)N1c1cc(C)ccc1C(C)C] | 635 (Method 2) | 1.27 |
| C-118 | 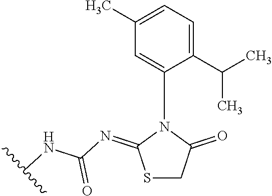 [SMILES: *NC(=O)/N=C1\SCC(=O)N1c1cc(C)ccc1C(C)C] | 640 (Method 2) | 1.23 |
| C-119 | 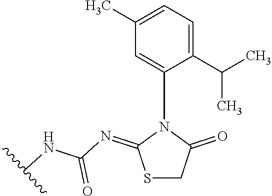 [SMILES: *NC(=O)/N=C1\SCC(=O)N1c1cc(C)ccc1C(C)C] | 690 (Method 2) | 1.30 |
| C-120 | 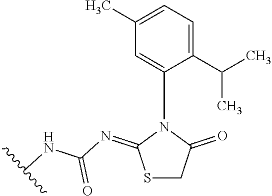 [SMILES: *NC(=O)/N=C1\SCC(=O)N1c1cc(C)ccc1C(C)C] | 635 (Method 2) | 1.25 |
| C-121 | 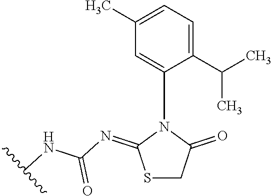 [SMILES: *NC(=O)/N=C1\SCC(=O)N1c1cc(C)ccc1C(C)C] | 686 (Method 2) | 1.24 |
| C-122 | 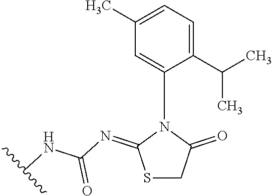 [SMILES: *NC(=O)/N=C1\SCC(=O)N1c1cc(C)ccc1C(C)C] | 663 (Method 2) | 1.27 |
| C-123 | 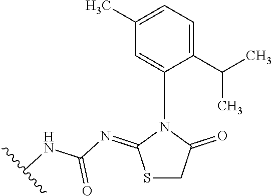 [SMILES: *NC(=O)/N=C1\SCC(=O)N1c1cc(C)ccc1C(C)C] | 733 (Method 1) | 2.27 |
| C-124 | 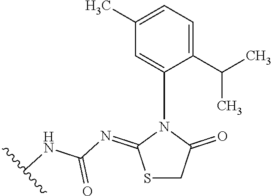 [SMILES: *NC(=O)/N=C1\SCC(=O)N1c1cc(C)ccc1C(C)C] | 633 (Method 1) | 2.14 |
| C-125 | 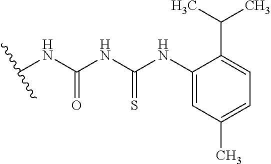 [SMILES: *NC(=O)NC(=S)Nc1cc(C)ccc1C(C)C] | — | — |
| C-126 | 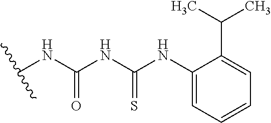 [SMILES: *NC(=O)NC(=S)Nc1ccccc1C(C)C] | 597 (Method 2) | 1.40 |
| C-127 | 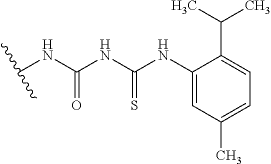 [SMILES: *NC(=O)NC(=S)Nc1cc(C)ccc1C(C)C] | 612 (Method 2) | 1.42 |
| C-128 | 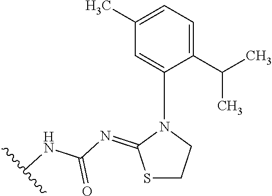 [SMILES: *NC(=O)/N=C1\SCCN1c1cc(C)ccc1C(C)C] | 684 (Method 2) | 1.40 |
| C-129 | 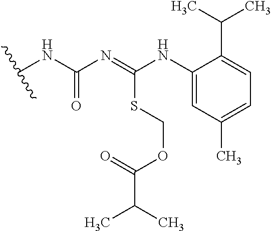 [SMILES: *NC(=O)/N=C(/Nc1cc(C)ccc1C(C)C)SCOC(=O)C(C)C] | 711 (Method 2) | 1.55 |
| C-130 | 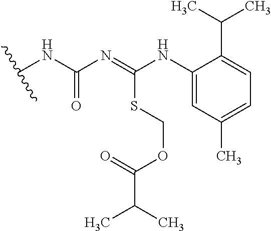 [SMILES: *NC(=O)/N=C(/Nc1cc(C)ccc1C(C)C)SCOC(=O)C(C)C] | 759 (Method 1) | 2.58 |
| C-131 | 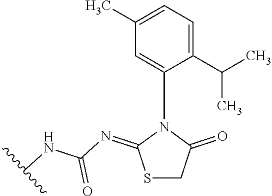 [SMILES: *NC(=O)/N=C1\SCC(=O)N1c1cc(C)ccc1C(C)C] | 685 (Method 1) | 2.33 |
| C-132 | 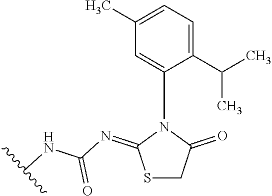 [SMILES: *NC(=O)/N=C1\SCC(=O)N1c1cc(C)ccc1C(C)C] | 671 (Method 1) | 2.34 |
| C-133 | 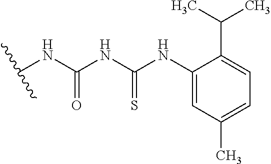 [SMILES: *NC(=O)NC(=S)Nc1cc(C)ccc1C(C)C] | 652 (Method 2) | 1.38 |
| C-134 | 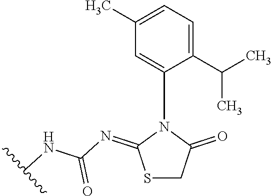 [SMILES: *NC(=O)/N=C1\SCC(=O)N1c1cc(C)ccc1C(C)C] | 649 (Method 2) | 1.32 |

### Biological Examples

### Example B1: Action on Yellow Fever Mosquito (Aedes aegypti)

For evaluating control of yellow fever mosquito (Aedes aegypti) the test unit consisted of 96-well-microtiter plates containing 200 μl of tap water per well and 5-15 freshly hatched A. aegypti larvae.

The active compounds or mixtures were formulated using a solution containing 75% (v/V) water and 25% (v/v) DMSO. Different concentrations of formulated compounds or mixtures were sprayed onto the insect diet at 2.5 μl, using a custom-built micro atomizer, at two replications. For experimental mixtures in these tests identical volumes of both mixing partners at the desired concentrations respectively, were mixed together.

After application, microtiter plates were incubated at 28±1 °C, 80±5% RH for 2 days. Larval mortality was then visually assessed.

In this test, compounds C-1, C-2, C-3, C-4, C-5, C-6, C-7, C-8, C-9, C-10, C-11, C-12, C-13, C-14, C-15, C-16, C-17, C-18, C-19, C-20, C-21, C-23, C-24, C-25, C-26, C-28, C-29, C-30, C-31, C-32, C-34, C-35, C-36, C-39, C-40, C-41, C-42, C-43, C-44, C-45, C-46, C-49, C-51, C-54, C-59, C-65, C-66, C-67, C-70, C-71, C-79, C-80, C-82, C-83, C-87, C-88, C-93, C-94, C-95, C-96, C-97, C-98, C-99, C-100, C-101, C-102, C-103, C-104, C-105, C-106, C-107, C-108, C-109, C-110, C-111, C-112, C-113, C-114, C-115, C-116, C-117, C-120, C-121, C-123, C-125, C-126, C-128, C-129 and C-130 at 800 ppm showed at least 50% mortality in comparison with untreated controls.

### Example B2: Action on Orchid Thrips (Dichromothrips corbetti)

Dichromothrips corbetti adults used for bioassay were obtained from a colony maintained continuously under laboratory conditions. For testing purposes, the test compound is diluted in a 1:1 mixture of acetone: water (vol: vol), plus Kinetic® HV at a rate of 0.01% v/v.

Thrips potency of each compound was evaluated by using a floral-immersion technique. All petals of individual, intact orchid flowers were dipped into treatment solution and allowed to dryin Petri dishes. Treated petals were placed into individual re-sealable plastic along with about 20 adult thrips. All test arenas were held under continuous light and a temperature of about 28 °C for duration of the assay. After 3 days, the numbers of live thrips were counted on each petal. The percent mortality was recorded 72 hours after treatment.

In this test, compounds C-1, C-2, C-3, C-4, C-6, C-7, C-9, C-10, C-11, C-17, C-18, C-19, C-20, C-22, C-23, C-25, C-44, C-46, C-97, C-104, C-123, C-125 and C-130 at 300 ppm showed at least 75% mortality in comparison with untreated controls.

### Example B3: Action on Boll Weevil (Anthonomus grandis)

For evaluating control of boll weevil (Anthonomus grandis) the test unit consisted of 96-well-microtiter plates containing an insect diet and 5-10 A. grandis eggs.

The compounds were formulated using a solution containing 75% (v/v) water and 25% (v/v) DMSO. Different concentrations of formulated compounds were sprayed onto the insect diet at 5 μl, using a custom-built micro atomizer, at two replications.

After application, microtiter plates were incubated at about 25±1 °C and about 75±5% relative humidity for 5 days. Egg and larval mortality were then visually assessed.

In this test, compounds C-1, C-2, C-3, C-4, C-5, C-6, C-7, C-8, C-9, C-10, C-11, C-12, C-13, C-14, C-15, C-16, C-17, C-18, C-19, C-20, C-21, C-23, C-24, C-25, C-26, C-27, C-28, C-29, C-30, C-31, C-32, C-33, C-34, C-35, C-36, C-38, C-39, C-40, C-41, C-42, C-43, C-44, C-45, C-46, C-49, C-51, C-52, C-54, C-55, C-58, C-59, C-65, C-66, C-68, C-71, C-75, C-76, C-77, C-78, C-79, C-80, C-82, C-83, C-87, C-88, C-90, C-91, C-92, C-93, C-94, C-95, C-96, C-97, C-98, C-99, C-100, C-101, C-102, C-103, C-104, C-105, C-106, C-107, C-108, C-109, C-110, C-111, C-112, C-113, C-114, C-115, C-116, C-117, C-118, C-119, C-120, C-122, C-121, C-123, C-125, C-126, C-127, C-128, C-129 and C-130 at 800 ppm showed at least 75% mortality in comparison with untreated controls.

### Example B4: Action on Silverleaf Whitefly (Bemisia argentifolii) (Adults)

The active compounds were formulated by a Tecan liquid handler in 100% cyclohexanone as a 10,000 ppm solution supplied in tubes. The 10,000 ppm solution was serially diluted in 100% cyclohexanone to make interim solutions. These served as stock solutions for which final dilutions were made by the Tecan in 50% acetone: 50% water (v/v) into 5 or 10 ml glass vials. A nonionic surfactant (Kinetic®) was included in the solution at a volume of 0.01% (v/v). The vials were then inserted into an automated electrostatic sprayer equipped with an atomizing nozzle for application to plants/insects.

Cotton plants at the cotyledon stage (one plant per pot) were sprayed by an automated electrostatic plant sprayer equipped with an atomizing spray nozzle. The plants were dried in the sprayer fume hood and then removed from the sprayer. Each pot was placed into a plastic cup and about 10 to 12 whitefly adults (approximately 3-5 days old) were introduced. The insects were collected using an aspirator and a nontoxic Tygon® tubing connected to a barrier pipette tip. The tip, containing the collected insects, was then gently inserted into the soil containing the treated plant, allowing insects to crawl out of the tip to reach the foliage for feeding. Cups were covered with a reusable screened lid. Test plants were maintained in a growth room at about 25 °C and about 20-40% relative humidity for 3 days, avoiding direct exposure to fluorescent light (24 hour photoperiod) to prevent trapping of heat inside the cup. Mortality was assessed 3 days after treatment, compared to untreated control plants.

In this test, compound C-6 at 300 ppm showed at least 75% mortality in comparison with untreated controls.

### Example B5: Action on Tobacco Budworm (Heliothis virescens)

For evaluating control of tobacco budworm (Heliothis virescens) the test unit consisted of 96-well-microtiter plates containing an insect diet and 15-25 H. virescens eggs.

The compounds were formulated using a solution containing 75% v/v water and 25% v/v DMSO. Different concentrations of formulated compounds were sprayed onto the insect diet at 10 μl, using a custom-built micro atomizer, at two replications.

After application, microtiter plates were incubated at about 28±1 °C and about 80±5% relative humidity for 5 days. Egg and larval mortality were then visually assessed.

In this test, compounds C-1, C-2, C-3, C-4, C-5, C-6, C-7, C-8, C-9, C-10, C-11, C-12, C-13, C-14, C-15, C-16, C-17, C-18, C-19, C-20, C-21, C-23, C-24, C-25, C-26, C-27, C-28, C-29, C-30, C-31, C-32, C-33, C-34, C-35, C-36, C-37, C-38, C-39, C-40, C-41, C-42, C-43, C-44, C-45, C-46, C-48, C-49, C-51, C-52, C-54, C-56, C-57, C-58, C-59, C-61, C-65, C-66, C-67, C-68, C-70, C-71, C-72, C-75, C-76, C-77, C-78, C-79, C-80, C-82, C-83, C-85, C-86, C-87, C-88, C-89, C-91, C-92, C-93, C-94, C-95, C-96, C-97, C-98, C-99, C-100, C-101, C-102, C-103, C-104, C-105, C-106, C-107, C-108, C-109, C-110, C-111, C-112, C-113, C-114, C-115, C-116, C-117, C-119, C-120, C-123, C-125, C-126, C-127, C-128, C-129 and C-130 at 800 ppm showed at least 75% mortality in comparison with untreated controls.

### Example B6: Action on Diamond Back Moth (Plutella xylostella)

The active compound is dissolved at the desired concentration in a mixture of 1:1 (v/v) distilled water:acetone. Surfactant (Kinetic® HV) is added at a rate of 0.01% (v/v). The test solution is prepared at the day of use.

Leaves of cabbage were dipped in test solution and air-dried. Treated leaves were placed in petri dishes lined with moist filter paper and inoculated with ten 3rd instar larvae. Mortality was recorded 72 hours after treatment. Feeding damages were also recorded using a scale of 0-100%.

In this test, compounds C-1, C-2, C-3, C-4, C-5, C-6, C-7, C-8, C-9, C-10, C-11, C-12, C-13, C-14, C-15, C-16, C-17, C-18, C-19, C-20, C-21, C-22, C-23, C-24, C-25, C-26, C-28, C-30, C-31, C-32, C-33, C-34, C-35, C-36, C-39, C-40, C-41, C-42, C-43, C-44, C-49, C-51, C-59, C-61, C-65, C-67, C-70, C-71, C-75, C-76, C-77, C-78, C-79, C-80, C-82, C-83, C-84, C-85, C-86, C-87, C-88, C-89, C-90, C-91, C-92, C-93, C-94, C-95, C-96, C-97, C-118 and C-129 at 300 ppm showed at least 75% mortality in comparison with untreated controls.

### Example B7: Action on Southern Armyworm (Spodoptera eridania), 2nd Instar Larvae

The active compounds were formulated by a Tecan liquid handler in 100% cyclohexanone as a 10,000 ppm solution supplied in tubes. The 10,000 ppm solution was serially diluted in 100% cyclohexanone to make interim solutions. These served as stock solutions for which final dilutions were made by the Tecan in 50% acetone: 50% water (v/v) into 10 or 20 ml glass vials. A nonionic surfactant (Kinetic®) was included in the solution at a volume of 0.01% (v/v). The vials were then inserted into an automated electrostatic sprayer equipped with an atomizing nozzle for application to plants/insects.

Lima bean plants (variety Sieva) were grown 2 plants to a pot and selected for treatment at the 1st true leaf stage. Test solutions were sprayed onto the foliage by an automated electrostatic plant sprayer equipped with an atomizing spray nozzle. The plants were dried in the sprayer fume hood and then removed from the sprayer. Each pot was placed into perforated plastic bags with a zip closure. About 10 to 11 armyworm larvae were placed into the bag and the bags zipped closed. Test plants were maintained in a growth room at about 25 °C and about 20-40% relative humidity for 4 days, avoiding direct exposure to fluorescent light (24 hour photoperiod) to prevent trapping of heat inside the bags. Mortality and reduced feeding were assessed 4 days after treatment, compared to untreated control plants.

In this test, compounds C-1, C-2, C-3, C-4, C-5, C-6, C-7, C-8, C-9, C-10, C-11, C-12, C-13, C-14, C-15, C-16, C-17, C-18, C-19, C-20, C-21, C-22, C-23, C-24, C-25, C-26, C-27, C-28, C-29, C-30, C-31, C-32, C-33, C-34, C-35, C-36, C-37, C-38, C-39, C-40, C-41, C-42, C-43, C-45, C-46, C-48, C-49, C-51, C-54, C-56, C-59, C-65, C-67, C-68, C-69, C-71, C-75, C-76, C-78, C-79, C-82, C-85, C-86, C-88, C-89, C-91, C-93, C-94, C-95, C-96, C-97, C-98, C-99, C-100, C-101, C-102, C-103, C-104, C-105, C-106, C-107, C-108, C-109, C-110, C-111, C-112, C-114, C-115, C-116, C-117, C-120, C-121, C-123, C-124, C-125, C-126, C-127, C-128, C-131 and C-132 at 300 ppm showed at least 75% mortality in comparison with untreated controls.

### Example B8: Action on Diamond Back Moth (Plutella xylostella)

For evaluating control of diamond back moth (Plutella xylostella) the test unit consisted of 96-well-microtiter plates containing an insect diet and 15-25 P. xylostella eggs. The compounds or mixtures were formulated using a solution containing 75% water and 25% DMSO. Different concentrations of formulated compounds or mixtures were sprayed onto the insect diet at 5 μl, using a custom-built micro atomizer, at two replications. For experimental mixtures in these tests identical volumes of both mixing partners at the desired concentrations respectively, were mixed together. After application, microtiter plates were incubated at 28±1 °C, 80±5% RH for 5 days. Egg and larval mortality was then visually assessed.

In this test, compounds C-27, C-29, C-37, C-38, C-45, C-46, C-48, C-52, C-53, C-54, C-55, C-56, C-57, C-58, C-60, C-66, C-68, C-73, C-98, C-99, C-100, C-101, C-102, C-103, C-104, C-105, C-106, C-107, C-108, C-109, C-110, C-111, C-112, C-113, C-114, C-115, C-116, C-117, C-118, C-119, C-120, C-121, C-122, C-123, C-125, C-126, C-127, C-128 and C-130 at 800 ppm showed at least 75% mortality in comparison with untreated controls.

### Comparative Data

Compounds disclosed in the document WO2021013561 were prepared according to procedures described therein and their pesticidal activities were determined by procedure analogues to the procedures as described above in examples B6 and B7. Results of comparison are as provided in Table R-1.

| Document | Compound | Structure |
|---|---|---|
| Compound of the present invention | C-2 | 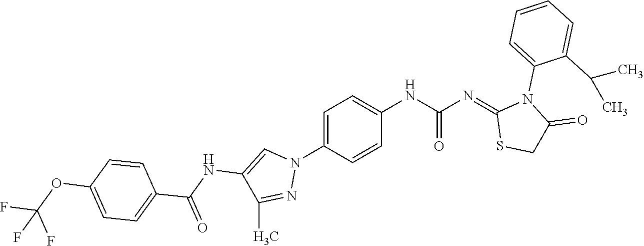 [SMILES: Cc1nn(-c2ccc(NC(=O)/N=C3\SCC(=O)N3c3ccccc3C(C)C)cc2)cc1NC(=O)c1ccc(OC(F)(F)F)cc1] |
| Compounds disclosed in WO2021013561 | C-24 | 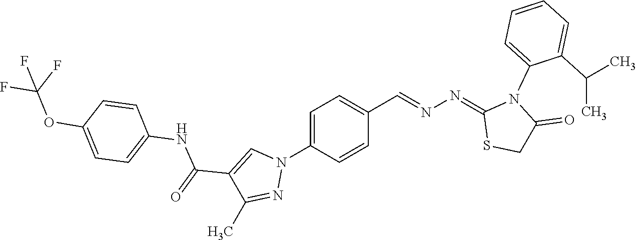 [SMILES: Cc1nn(-c2ccc(/C=N/N=C3\SCC(=O)N3c3ccccc3C(C)C)cc2)cc1C(=O)Nc1ccc(OC(F)(F)F)cc1] |
| | C-37 | 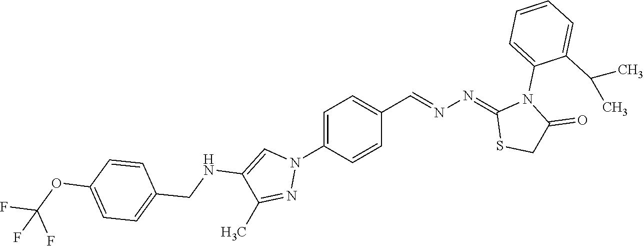 [SMILES: Cc1nn(-c2ccc(/C=N/N=C3\SCC(=O)N3c3ccccc3C(C)C)cc2)cc1NCc1ccc(OC(F)(F)F)cc1] |
| | C-41 | 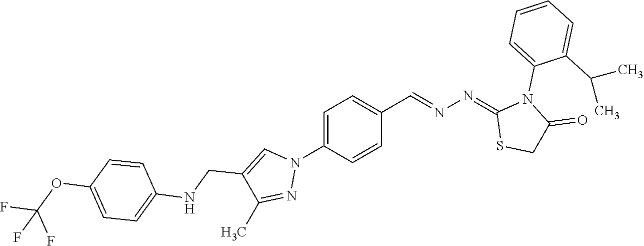 [SMILES: Cc1nn(-c2ccc(/C=N/N=C3\SCC(=O)N3c3ccccc3C(C)C)cc2)cc1CNc1ccc(OC(F)(F)F)cc1] |

TABLE R-1
pesticidal activities

| | | Dose | Test | |
|---|---|---|---|---|
| Document | Compound | (ppm) | species | Mortality % |
| Compound of the | C-2 | 10 | PLUTMA | 100 |
| present invention | | 10 | PRODER | 82.5 |
| Compounds | C-24 | 10 | PLUTMA | 0 |
| disclosed in | | 10 | PRODER | 0 |
| WO2021013561 | C-37 | 10 | PLUTMA | 50 |
| | | 10 | PRODER | 0 |
| | C-41 | 10 | PLUTMA | 25 |
| | | 10 | PRODER | 0 |

1. Compounds A compound of the formula I 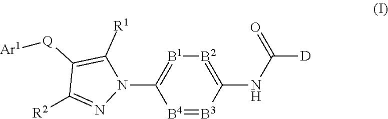
wherein
Q is —C(═O)—N(R⁵)—, or —N(R⁵)—C(═O)—;
R⁵ is H, C₁-C₆-alkyl, C₃-C₆-cycloalkyl, C₁-C₆-alkyl-C₁-C₆-alkoxy or C₁-C₆-alkyl-C₃-C₆-cycloalkyl, phenyl, 5- or 6-membered heteroaryl, —CH₂-phenyl, —CH₂-5- or 6-membered heteroaryl, 1,3-dioxolan-2-ylmethyl, or halogen, wherein the alkyl, cycloalkyl, phenyl, and heteroaryl moieties are unsubstituted or substituted with halogen or CN;
R¹ is H, C₁-C₆-alkyl, C₃-C₆-cycloalkyl, halogen, or NR⁶R⁷, wherein the alkyl and cycloalkyl moieties are unsubstituted or substituted with halogen or CN;
R² is H, C₁-C₆-alkyl, C₃-C₆-cycloalkyl, or halogen, wherein the alkyl and cycloalkyl moieties are unsubstituted or substituted with halogen or CN;
B¹ is N or CR^{B1};
B² is N or CR^{B2};
B³ is N or CR^{B3};
B⁴ is CR^{B4};
R^{B1}, R^{B2}, R^{B3}, and R^{B4} independently of each other are H, halogen, CN, C₁-C₆-alkyl, C₃-C₆-cycloalkyl, or C₁-C₆-alkoxy, wherein the alkyl, alkoxy, and cycloalkyl moieties are unsubstituted or substituted with halogen;
D is the moiety DA or DB, 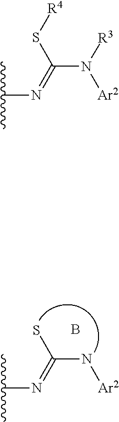
R³ is H, C₁-C₆-alkyl or C₃-C₆-cycloalkyl, wherein the alkyl and cycloalkyl moieties are unsubstituted or substituted with halogen or CN;
R⁴ is H, C₁-C₆-alkyl, or C₃-C₆-cycloalkyl, wherein the alkyl and cycloalkyl moieties are unsubstituted or substituted with halogen, —O—(C═O)—C₁-C₆-alkyl, —O—(CO)—C₁-C₆-alkoxy, or CN;
B is a 5- or 6-membered carbocyclic group, wherein 1 or 2 CH₂ moieties of the carbocyclic group may be optionally are replaced by a carbonyl group, O, or S, wherein the carbocyclic group is unsubstituted or substituted with R^{h};
Ar¹ is phenyl or 5- or 6-membered heteroaryl, which are unsubstituted or substituted with R^{Ar1}, wherein
R^{Ar1} is halogen, SF₅, NO₂, OH, CN, C₁-C₆-alkyl, C₁-C₆-alkoxy, C₃-C₆-cycloalkyl, C₃-C₆-heterocyclyl, C₃-C₆-cycloalkoxy, C₂-C₆-alkenyl, C₂-C₆-alkynyl wherein the alkyl, alkoxy, alkenyl, alkynyl, cycloalkyl, C₃-C₆-heterocyclyl, and cycloalkoxy moieties are unsubstituted or substituted with R^{f}; C(═O)—OR^{a}, NR^{b}R^{c}, C₁-C₆-alkylene-CN, C(═O)—NR^{b}R^{c}, C(═O)R^{d}, NHS(═O)ₘR^{e}, S(═O)ₘR^{e}, —N═S(═O)—(C₁-C₆-alkyl)₂, SO₂NR^{b}R^{c}, or S(═O)ₘR^{e};
R⁶ and R⁷ are, identical or different, H, C₁-C₆-alkyl, C₃-C₆-cycloalkyl, phenyl, —CH₂-phenyl, 5- or 6-membered heteroaryl, —CH₂-5- or 6-membered heteroaryl, 1,3-dioxolan-2-ylmethyl, or 2-(methylamino)-2-oxo-ethyl, wherein the alkyl, cycloalkyl, phenyl, and heteroaryl moieties are unsubstituted or substituted with halogen, CN, C₁-C₆-alkyl or C₁-C₆-alkoxy;
Ar² is phenyl or 5- or 6-membered heteroaryl, which are unsubstituted or substituted with R^{Ar2}, wherein
R^{Ar2} is halogen, CN, —SCN, —SF₅, C₁-C₆-alkyl, C₁-C₆-alkoxy, C₂-C₆-alkenyl, C₂-C₆-alkynyl, C₁-C₆-alkoxy-C₁-C₄-alkyl, C₁-C₆-alkoxy-C₁-C₄-alkoxy, C₃-C₆-cycloalkyl, C₃-C₆-cycloalkoxy, C₃-C₆-cycloalkyl-C₁-C₄-alkyl, C₃-C₆-cycloalkoxy-C₁-C₄-alkyl, wherein the alkyl, alkoxy, alkenyl, alkynyl, cycloalkyl, and cycloalkoxy moieties are unsubstituted or substituted with halogen, C(═O)—OR^{a}, NR^{b}R^{c}, C₁-C₆-alkylene-CN, C(═O)NR^{b}R^{c};
R^{a}, R^{b}, and R^{c} are, identical or different, H, C₁-C₆-alkyl, C₂-C₆-alkenyl, C₃-C₆-cycloalkyl, C₃-C₆-cycloalkyl-C₁-C₄-alkyl, —C(═O)—C₁-C₆-alkyl wherein the alkyl, alkenyl, and cycloalkyl moieties are unsubstituted or substituted with halogen,
R^{d} is H, C₁-C₆-alkyl;
R^{e} is C₁-C₆-alkyl, C₃-C₆-cycloalkyl, C₃-C₆-cycloalkyl-C₁-C₄-alkyl, wherein the alkyl and cycloalkyl moieties are unsubstituted or substituted with halogen or CN;
R^{f} is halogen, OH, CN, SCN, —SF₅, C₁-C₆-alkyl, C₁-C₆-alkoxy, C₂-C₆-alkenyl, C₂-C₆-alkynyl, C₁-C₆-alkoxy-C₁-C₄-alkyl, C₁-C₆-alkoxy-C₁-C₄-alkoxy, C₃-C₆-cycloalkyl, C₃-C₆-cycloalkoxy, C₃-C₆-cycloalkyl-C₁-C₄-alkyl, C₃-C₆-cycloalkoxy-C₁-C₄-alkyl, wherein the alkyl, alkoxy, alkenyl, alkynyl, cycloalkyl, and cycloalkoxy moieties are unsubstituted or substituted with halogen;
R^{h} is halogen, C₁-C₆-alkyl, or C₁-C₆-alkoxy;
m is 0, 1, or 2:
and N-oxides, stereoisomers, tautomers, and agriculturally or veterinarily acceptable salts thereof.

2. The compound of formula I according to claim 1, wherein D is DB.

3. The compound of formula I according to claim 1, wherein D is a group selected from the group consisting of D5 to D7 moeities, which are unsubstituted or substituted with 1 or 2 substituents R^{h}, wherein R^{h} is as defined in claim 1

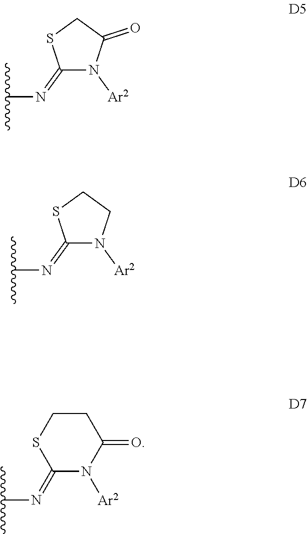

4. The compound of formula I according to claim 1 is selected from the compounds of formula I.1 to 1.4.

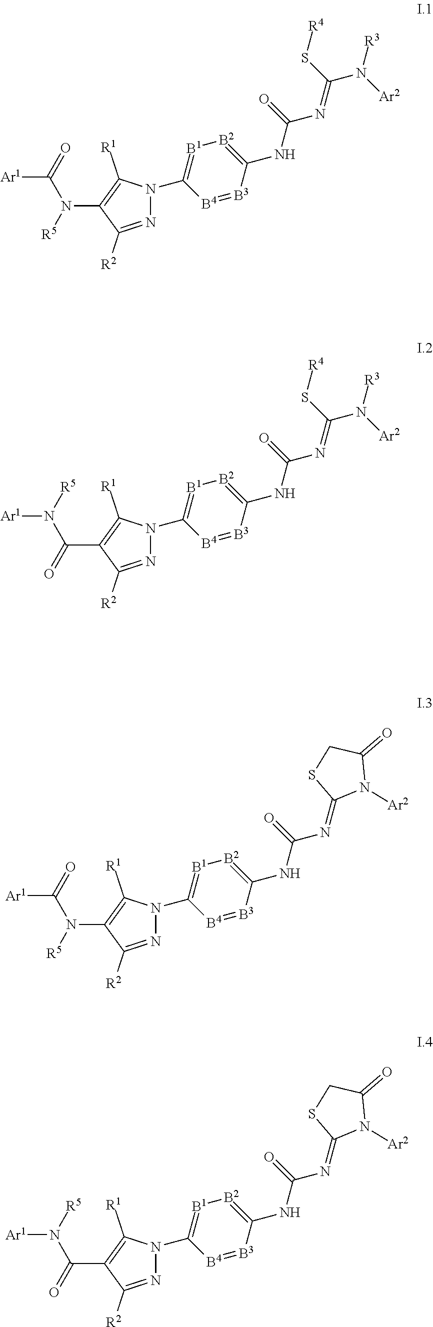

5. The compound of formula I according to claim 1, wherein
R⁵ is H, C₁-C₆-alkyl, or C₁-C₆-alkyl-C₃-C₆-cycloalkyl;
R¹ is H, halogen, C₁-C₆-alkyl, or C₃-C₆-cycloalkyl;
R² is H or C₁-C₆-alkyl.

6. The compound of formula I according to claim 1, wherein B¹ is CR^{B1}, B² is CR^{B2}, and B³ is CR^{B3}.

7. The compound of formula I according to claim 1, wherein B¹ is N, B² is CR^{B2}, and B³ is CR^{B3}.

8. The compound of formula I according to claim 1, wherein R^{B1}, R^{B2}, R^{B3}, and R^{B4} independently of each other are H, halogen, CN, C₁-C₆-alkyl, C₃-C₆-cycloalkyl, or C₁-C₆-alkoxy, wherein the alkyl, alkoxy, and cycloalkyl moieties are unsubstituted or substituted with halogen.

9. The compound of formula I according to claim 1, wherein
Ar¹ is phenyl, pyrimidinyl, pyridazinyl, thiophenyl, thiazolyl, or pyridyl, which are unsubstituted or substituted with R^{Ar1};
R^{Ar1} is halogen, SF₅, NO₂, OH, CN, C₁-C₆-alkyl, C₁-C₆-alkoxy, C₃-C₆-cycloalkyl, C₃-C₆-heterocyclyl, C₃-C₆-cycloalkoxy, C₂-C₆-alkenyl, or C₂-C₆-alkynyl wherein the alkyl, alkoxy, alkenyl, alkynyl, cycloalkyl, C₃-C₆-heterocyclyl, and cycloalkoxy moieties are unsubstituted or substituted with R^{f}; C(═O)—OR^{a}, NR^{b}R^{c}, C₁-C₆-alkylene-CN, C(═O)NR^{b}R^{c}, C(═O)—R^{d}, NHS(═O)ₘR^{e}, —N═S(═O)—(C₁-C₆-alkyl)₂, SO₂NR^{b}R^{c}, or S(═O)ₘR^{e};
R^{a}, R^{b} and R^{c} identical or different, are H or C₁-C₆-alkyl, which are unsubstituted or substituted with halogen;
R^{d} is H or C₁-C₆-alkyl;
R^{e} is C₁-C₆-alkyl or C₁-C₆-haloalkyl;
R^{f} is halogen, OH, CN, C₁-C₆-alkyl, C₁-C₆-alkoxy, C₂-C₆-alkenyl, C₂-C₆-alkynyl, C₃-C₆-cycloalkyl, or C₃-C₆-cycloalkoxy, which are unsubstituted or substituted with halogen;
m is 0, 1, or 2.

10. A composition, comprising at least one compound of formula I according to claim 1, an N-oxide or an agriculturally acceptable salt thereof, and a further active substance.

11. A method for combating or controlling an invertebrate pest comprising contacting said pest or its food supply, habitat, or breeding grounds with a pesticidally effective amount of at least one compound claim 1.

12. A method for protecting growing plants from attack or infestation by an invertebrate pest comprising contacting a plant, or soil or water wherein the plant is growing, with a pesticidally effective amount of at least one compound according to claim 1.

13. Seed A seed comprising a compound according to claim 1, or enantiomers, diastereomers, or salts thereof, in an amount of from 0.1 g to 10 kg per 100 kg of seed.

14. (canceled)

15. A method for treating or protecting an animal from infestation or infection by an invertebrate pest comprising bringing the animal in contact with a pesticidally effective amount of at least one compound of formula I according to claim 1, a stereoisomer thereof and/or at least one veterinarily acceptable salt thereof.
